(19) United States
(12) Patent Application Publication
(10) Pub. No.: US 2023/0192657 A1
(43) Pub. Date: Jun. 22, 2023

(54) 1,2,4-TRIAZOLINONE CB1 INHIBITORS

(71) Applicant: Goldfinch Bio, Inc., Cambridge, MA (US)

(72) Inventors: Maolin Yu, West Roxbury, MA (US); Mark W. Ledeboer, Acton, MA (US); Jean-Christophe P. Harmange, Andover, MA (US)

(21) Appl. No.: 17/908,768

(22) PCT Filed: Mar. 1, 2021
(86) PCT No.: PCT/US2021/020199

§ 371 Date: Sep. 1, 2022

Related U.S. Application Data
(60) Provisional application No. 62/983,922, filed on Mar. 2, 2020.

(51) Int. Cl. C07D 403/04; C07D 401/14; C07D 405/14

(52) U.S. Cl. CPC C07D 403/04; C07D 401/14; C07D 405/14

(57) ABSTRACT

Disclosed are compounds according to Formula (I), and related pharmaceutical compositions. Also disclosed are therapeutic methods, e.g., of treating diseases such as diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease, using the compounds of Formula (I).

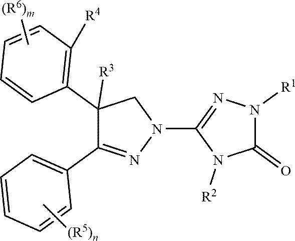

### CROSS-REFERENCE TO RELATED APPLICATIONS

This application claims the benefit of priority to U.S. Provisional Pat. Application No. 62/983,922, filed Mar. 2, 2020, which is incorporated by reference in its entirety.

### BACKGROUND

The CB1 cannabinoid receptor is one of the most abundant G-protein coupled receptors in the brain; it is highly expressed in the basal ganglia nuclei, hippocampus, cortex, and cerebellum. The distribution of this receptor within the central nervous system (CNS) correlated with its role in the control of motor function, cognition and memory, and analgesia. CB1 receptors are also expressed throughout the periphery, albeit at much lower levels than in the CNS. The receptor has also been detected in a variety of circulating immune cells and numerous peripheral tissues, including the adrenal gland, heart, lung, prostate, liver, bone marrow, and thymus. Endogenous ligands for the CB1 receptor include the arachidonic acid metabolites N-arachidonylethanolamide (anandamide) and 2-arachidonylglycerol (2-AG), and exogenous ligands include phytocannabinoids such as those found in cannabis.

Experimental studies have suggested that stimulation of the CB1 receptor using pharmacologic agents or its natural ligands could have deleterious effects on several different organs. CB1 receptor expression is altered in diabetic kidney disease, and preclinical studies have confirmed that the CB1 receptor is implicated in the pathogenesis of diabetic kidney disease. Several reports have also described the development of acute kidney injury in otherwise healthy patients exposed to synthetic cannabinoids. In the liver, the CB1 and CB2 receptors are faintly expressed under physiological conditions, but induction of these receptors and/or increased levels of cannabinoids are common features of liver injuries such as alcoholic liver disease and non-alcoholic fatty liver disease; the latter of these is characterized by upregulation of adipose tissue and hepatocyte CB1 receptors and increased liver synthesis of anandamide. The CB1 receptor is also implicated in diabetic nephropathy, obesity-related kidney disease, kidney fibrosis, Prader Willi syndrome, focal segmental glomerular sclerosis, IgA nephropathy, nephrotic syndrome, metabolic syndrome, non-alcoholic liver disease, and various gastrointestinal diseases.

As enhanced CB1 expression is associated with pathogenesis of numerous diseases, inhibition of CB1 is a promising therapeutic strategy. Thousands of orthosteric inhibitors of CB1, belonging to many different structural classes, have been synthesized and evaluated. However, this strategy has had only limited success in bringing such leads to the clinic, largely owing to unwanted side effects. Allosteric inhibition strategies have also been of limited value, as promising in vitro activity does not always translate into in vivo potency.

Hence, there is a need for new modulators of CB1 receptor activity.

### SUMMARY

This invention is based, at least in part, on the discovery that inhibition of the CB1 receptor by certain compounds may be useful to treat a disease or condition characterized by aberrant CB1 activity.

One aspect of the invention is compounds that are inhibitors of the CB1 receptor. In some embodiments, the compound of the invention is a compound of structural formula I:

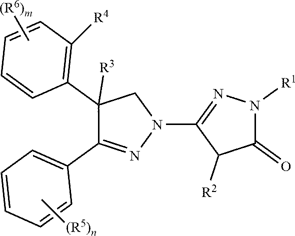

, or a pharmaceutically acceptable salt thereof, wherein:
R¹ is hydrogen, -C₁-C₄ alkyl, -(C₀-C₂ alkylene)-aryl, -(C₀-C₂ alkylene)-heteroaryl, -(C₀-C₂ alkylene)-heterocyclyl and -(C₀-C₂ alkylene)-carbocyclyl, wherein any alkyl, alkylene, aryl, heteroaryl, heterocyclyl, or carbocyclyl portion of R¹ is optionally substituted with 1 to 3 independently selected substituents;
R² is benzyl, -C₁-C₄ alkyl, -(C₁-C₄ alkylene)—C(O)—NH₂, -(C₁-C₄ alkylene)—C(O)—NH—(C₁-C₄ alkyl), -(C₁-C₄ alkylene)—S(O)₂—NH₂, -(C₁-C₄ alkylene)—S(O)₂—NH—(C₁-C₄ alkyl), -(C₁-C₄ alkylene)-O-(C₁-C₄ alkyl), or -(C₁-C₄ alkylene)—C(O)—O—(C₁-C₄ alkyl), wherein any alkyl portion of R² is optionally substituted with halo, —CN, —OH, or NO₂.
R³ is hydrogen; and
R⁴ is hydrogen, halo, or —CN; or
R³ and R⁴ are taken together with the carbon atoms to which they are bound and intervening atoms to form a 3-7 membered cycloalkyl moiety that is fused to the phenyl moiety bearing R⁴ and spirofused to the 4,5-dihydropyrazol-1,3,4-triyl moiety bearing R³;
each R⁵, if present, is independently halo, —CN, —OH, —NH₂, -NH(C₁-C₃ unsubstituted alkyl), -N(C₁-C₃ unsubstituted alkyl)₂, —NO₂, C₁-C₄ alkyl, C₂-C₄ alkenyl, C₂-C₄ alkynyl, or —O—C₁-C₄ alkyl, wherein the alkyl, alkenyl or alkynyl of R⁵ is optionally substituted with one or more substituents independently selected from halo, —CN, —OH, —O—(C₁-C₃ unsubstituted alkyl), —NH₂, -NH(C₁-C₃ unsubstituted alkyl), -N(C₁-C₃ unsubstituted alkyl)₂, and —NO₂;
each R⁶, if present is independently halo, —CN, —OH, —NH₂, -NH(C₁-C₃ unsubstituted alkyl), -N(C₁-C₃ unsubstituted alkyl)₂, —NO₂, C₁-C₄ alkyl, C₂-C₄ alkenyl, C₂-C₄ alkynyl, or —O—C₁-C₄ alkyl, wherein the alkyl, alkenyl or alkynyl of R⁶ is optionally substituted with one or more substituents independently selected from halo, —CN, —OH, —O—(C₁-C₃ unsubstituted alkyl), —NH₂, -NH(C₁-C₃ unsubstituted alkyl), -N(C₁-C₃ unsubstituted alkyl)₂, and —NO₂;
n is 0, 1, 2, 3, or 4;
m is 0, 1, 2, 3, or 4; and
m+n is greater than 0.

In one aspect, the invention features a pharmaceutical composition comprising a compound of the invention and a pharmaceutically acceptable carrier.

In one aspect, the invention relates to methods of treating a disease or condition characterized by aberrant CB1 activity comprising the step of administering to a subject in need thereof a compound or composition of the invention.

In some embodiments, the disease or condition is diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

In some embodiments, the disease or condition is diabetic nephropathy. In some embodiments, the disease or condition is focal segmental glomerular sclerosis. In some embodiments, the disease or condition is nonalcoholic steatohepatitis.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses.

The invention provides several advantages. The prophylactic and therapeutic methods described herein are effective in treating a disease or condition characterized by aberrant CB1 activity. Further, methods described herein are effective to identify compounds that treat or reduce risk of developing a disease or condition characterized by aberrant CB1 activity.

Other features, objects, and advantages of the invention will be apparent from the detailed description, and from the claims.

### DETAILED DESCRIPTION

### Definitions

Unless otherwise defined, all technical and scientific terms used herein have the same meaning as commonly understood by one of ordinary skill in the art to which this invention belongs. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000). Although methods and materials similar or equivalent to those described herein can be used in the practice or testing of the present invention, suitable methods and materials are described below.

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents, published patent applications, or other references referred to in this application are specifically incorporated by reference herein. In case of conflict, the present specification, including its specific definitions, will control. In addition, the materials, methods, and examples are illustrative only and not intended to be limiting.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit AR or promote AR degradation may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject’s size, health and age, and the nature and extent of the condition being treated. The skilled worker can readily determine the effective amount for a given situation by routine experimentation.

The term “acyl” is art-recognized and refers to a group represented by the general formula hydrocarbylC(O)-, preferably alkylC(O)-.

The term “acylamino” is art-recognized and refers to an amino group substituted with an acyl group and may be represented, for example, by the formula hydrocarbylC(O)NH-.

The term “acyloxy” is art-recognized and refers to a group represented by the general formula hydrocarbylC(O)O-, preferably alkylC(O)O-.

The term “alkoxy” refers to an alkyl group, preferably a lower alkyl group, having an oxygen attached thereto. Representative alkoxy groups include methoxy, trifluoromethoxy, ethoxy, propoxy, tert-butoxy and the like.

The term “alkoxyalkyl” refers to an alkyl group substituted with an alkoxy group and may be represented by the general formula alkyl-O-alkyl.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

An “alkyl” group or “alkane” is a straight chained or branched non-aromatic hydrocarbon which is completely saturated. Typically, a straight chained or branched alkyl group has from 1 to about 20 carbon atoms, preferably from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen (e.g., fluoro), a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

Unless otherwise specified, “alkylene” by itself or as part of another substituent refers to a saturated straight-chain or branched divalent group having the stated number of carbon atoms and derived from the removal of two hydrogen atoms from the corresponding alkane. Examples of straight chained and branched alkylene groups include —CH₂—(methylene), —CH₂—CH₂— (ethylene), —CH₂—CH₂—CH₂— (propylene), —CH(CH₃)—, —C(CH₃)₂—, —CH₂—CH(CH₃)—, —CH₂—CH₂—CH₂—CH₂—, —CH₂—CH₂—CH₂—CH₂—CH₂— (pentylene), —CH₂—CH(CH₃)—CH₂—, and —CH₂—C(CH₃)₂—CH₂—.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain, including haloalkyl groups. Preferred haloalkyl groups include trifluoromethyl, difluoromethyl, 2,2,2-trifluoroethyl, and pentafluoroethyl. C₀ alkyl indicates a hydrogen where the group is in a terminal position, a bond if internal. The terms “C_{2-y} alkenyl” and “C_{2-y} alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS-.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “amide”, as used herein, refers to a group

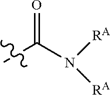

wherein each R^{A} independently represent a hydrogen or hydrocarbyl group, or two R^{A} are taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

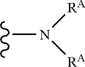

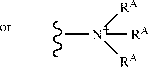

wherein each R^{A} independently represents a hydrogen or a hydrocarbyl group, or two R^{A} are taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 6- or 10-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, and/or aryls. Aryl groups include benzene, naphthalene, phenanthrene, phenol, aniline, and the like.

The term “carbamate” is art-recognized and refers to a group

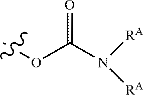

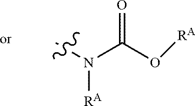

wherein each R^{A} independently represent hydrogen or a hydrocarbyl group, such as an alkyl group, or both R^{A} taken together with the intervening atom(s) complete a heterocycle having from 4 to 8 atoms in the ring structure.

The terms “carbocycle”, and “carbocyclic”, as used herein, refers to a saturated or non-aromatic unsaturated ring in which each atom of the ring is carbon. Carbocycles include both cycloalkane rings, in which all carbon atoms are saturated, and cycloalkene rings, which contain at least one double bond and have no aromatic character. “Carbocycle” includes 5-7 membered monocyclic and 8-12 membered bicyclic rings. Each ring of a bicyclic carbocycle may be selected from saturated and unsaturated non-aromatic rings in which each atom of each ring is carbon. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated and unsaturated non-aromatic rings. Any combination of saturated and unsaturated non-aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, and adamantane. Exemplary fused carbocycles include decalin, 1,2,3,4-tetrahydronaphthalene, bicyclo[4.2.0]octane, 4,5,6,7-tetrahydro-1H-indene and bicyclo[4.1.0]hept-3-ene. “Carbocycles” may be susbstituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl may be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “carbocyclylalkyl”, as used herein, refers to an alkyl group substituted with a carbocycle group.

The term “carbonate” is art-recognized and refers to a group —OCO₂—R^{A}, wherein R^{A} represents a hydrocarbyl group.

The term “carboxy”, as used herein, refers to a group represented by the formula —CO₂H.

The term “ester”, as used herein, refers to a group —C(O)OR^{A} wherein R^{A} represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O-. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, and the other cyclic rings can be cycloalkyls, cycloalkenyls, aryls, and/or heteroaryls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, and the other cyclic rings can be cycloalkyls, cycloalkenyls, aryls, heteroaryls, and/or heterocyclyls. Heterocyclyl groups include, for example, piperidine, piperazine, pyrrolidine, tetrahydropyran, tetrahydrofuran, morpholine, lactones, lactams, and the like.

The term “heterocyclylalkyl” or “heterocycloalkyl”, as used herein, refers to an alkyl group substituted with a heterocycle group.

The term “hydrocarbyl”, as used herein, refers to a group that is bonded through a carbon atom that does not have a =O or =S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a =O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “silyl” refers to a silicon moiety with three hydrocarbyl moieties attached thereto.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. In preferred embodiments, the substituents on substituted alkyls are selected from C₁₋₆ alkyl, C₃₋₆ cycloalkyl, halogen, carbonyl, cyano, or hydroxyl. In more preferred embodiments, the substituents on substituted alkyls are selected from fluoro, carbonyl, cyano, or hydroxyl. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

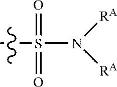

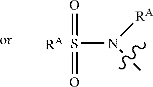

wherein each R^{A} independently represents hydrogen or hydrocarbyl, such as alkyl, or both R^{A} taken together with the intervening atom(s) complete a heterocycle having from 4 to 8 atoms in the ring structure.

The term “sulfoxide” is art-recognized and refers to the group —S(O)—R^{A}, wherein R^{A} represents a hydrocarbyl.

The term “sulfonate” is art-recognized and refers to the group SO₃H, or a pharmaceutically acceptable salt thereof.

The term “sulfone” is art-recognized and refers to the group —S(O)₂—R^{A}, wherein R^{A} represents a hydrocarbyl.

The term “thioalkyl”, as used herein, refers to an alkyl group substituted with a thiol group.

The term “thioester”, as used herein, refers to a group -C(O)SR^{A} or -SC(O)R^{A} wherein R^{A} represents a hydrocarbyl.

The term “thioether”, as used herein, is equivalent to an ether, wherein the oxygen is replaced with a sulfur.

The term “urea” is art-recognized and may be represented by the general formula

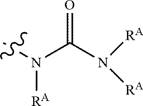

wherein each R^{A} independently represents hydrogen or a hydrocarbyl, such as alkyl, or any occurrence of R^{A} taken together with another and the intervening atom(s) complete a heterocycle having from 4 to 8 atoms in the ring structure.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

As used herein, a therapeutic that “prevents” or “reduces the risk of developing” a disease, disorder, or condition refers to a compound that, in a statistical sample, reduces the occurrence of the disease, disorder, or condition in the treated sample relative to an untreated control sample, or delays the onset or reduces the severity of one or more symptoms of the disorder or condition relative to the untreated control sample.

The term “treating” includes therapeutic treatments. A treatment is therapeutic, if it is intended to diminish, ameliorate, or stabilize the existing unwanted condition or side effects thereof.

In certain embodiments, compounds of the invention may be used alone or conjointly administered with another therapeutic agent. The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

In certain embodiments, conjoint administration of compounds of the invention with one or more additional therapeutic agent(s) provides improved efficacy relative to each individual administration of the compound of the invention or the one or more additional therapeutic agent(s). In certain such embodiments, the conjoint administration provides an additive effect, wherein an additive effect refers to the sum of each of the effects of individual administration of the compound of the invention and the one or more additional therapeutic agent(s).

The term “prodrug” is intended to encompass compounds which, under physiologic conditions, are converted into the therapeutically active agents of the present invention. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds of the invention in a formulation represented above can be replaced with the corresponding suitable prodrug, e.g., wherein a hydroxyl in the parent compound is presented as an ester or a carbonate or carboxylic acid present in the parent compound is presented as an ester.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

### Compounds of the Invention

One aspect of the invention provides small molecules that inhibit the CB1 receptor.

In some embodiments, the compound of the invention is a compound of structural formula I:

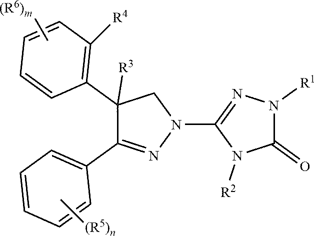

, or a pharmaceutically acceptable salt thereof, wherein:
R¹ is hydrogen, -C₁-C₄ alkyl, -(C₀-C₂ alkylene)-aryl, -(C₀-C₂ alkylene)-heteroaryl, -(C₀-C₂ alkylene)-heterocyclyl and -(C₀-C₂ alkylene)-carbocyclyl, wherein any alkyl, alkylene, aryl, heteroaryl, heterocyclyl, or carbocyclyl portion of R¹ is optionally substituted with 1 to 3 independently selected substituents;
R² is benzyl, -C₁-C₄ alkyl, -(C₁-C₄ alkylene)—C(O)—NH₂, -(C₁-C₄ alkylene)-C(O)- NH-(C₁-C₄ alkyl), -(C₁-C₄ alkylene)—S(O)₂—NH₂, -(C₁-C₄ alkylene)-S(O)₂-NH-(C₁-C₄ alkyl), -(C₁-C₄ alkylene)-O-(C₁-C₄ alkyl), or -(C₁-C₄ alkylene)-C(O)-O-(C₁-C₄ alkyl), wherein any alkyl portion of R² is optionally substituted with halo, —CN, —OH, or NO₂.
R³ is hydrogen; and
R⁴ is hydrogen, halo, or —CN; or
R³ and R⁴ are taken together with the carbon atoms to which they are bound and intervening atoms to form a 3-7 membered cycloalkyl moiety that is fused to the phenyl moiety bearing R⁴ and spirofused to the 4,5-dihydropyrazol-1,3,4-triyl moiety bearing R³;
each R⁵, if present, is independently halo, —CN, —OH, —NH₂, -NH(C₁-C₃ unsubstituted alkyl), -N(C₁-C₃ unsubstituted alkyl)₂, —NO₂, C₁-C₄ alkyl, C₂-C₄ alkenyl, C₂-C₄ alkynyl, or -O-C₁-C₄ alkyl, wherein the alkyl, alkenyl or alkynyl of R⁵ is optionally substituted with one or more substituents independently selected from halo, —CN, —OH, —O—(C₁-C₃ unsubstituted alkyl), —NH₂, -NH(C₁-C₃ unsubstituted alkyl), -N(C₁-C₃ unsubstituted alkyl)₂, and —NO₂;
each R⁶, if present is independently halo, —CN, —OH, —NH₂, -NH(C₁-C₃ unsubstituted alkyl), -N(C₁-C₃ unsubstituted alkyl)₂, —NO₂, C₁-C₄ alkyl, C₂-C₄ alkenyl, C₂-C₄ alkynyl, or —O—C₁-C₄ alkyl, wherein the alkyl, alkenyl or alkynyl of R⁶ is optionally substituted with one or more substituents independently selected from halo, —CN, —OH, —O—(C₁-C₃ unsubstituted alkyl), —NH₂, -NH(C₁-C₃ unsubstituted alkyl), -N(C₁-C₃ unsubstituted alkyl)₂, and —NO₂;
n is 0, 1, 2, 3, or 4;
m is 0, 1, 2, 3, or 4; and
m+n is greater than 0.

In some aspects, R¹ is hydrogen, -CH₃, phenyl, -CH₂-phenyl, -(CH₂)₂-phenyl, -CH(CH₃)-phenyl, -CH₂-pyridinyl, -CH₂-pyrimidinyl, or -CH₂-cyclohexyl; and wherein the phenyl, pyridinyl, pyrimidinyl, or cyclohexyl portion of R¹ is optionally substituted with up to 3 substituents independently selected from halo, -CN, C₁-C₄ alkyl, -O-(C₁-C₄ alkyl), -C(O)OH, -C(O)O-(C₁-C₄ alkyl), -C(O)NH₂, -C(O)NH-(C₁-C₄ alkyl) -(C₀-C₁ alkylene)-heterocyclyl, and -(C₀-C₁ alkylene)-O-heterocyclyl, wherein any alkyl, alkylene, or heterocyclyl portion of the R¹ substituent is further substituted with up to 3 substituents independently selected from halo, -OH, C₁-C₄ alkyl, C₁-C₄ hydroxyalkyl, C₁-C₄ haloalkyl, and -S(C₁-C₄ alkyl).

In some aspects, R¹ is hydrogen, -CH₃, benzyl, 2-(4-(2-hydroxyethan-1-ylcarbamoyl)phenyl)ethan-1-yl, 2-(4-carbamoylphenyl)ethan-1-yl, 2-(4-carboxyphenyl)ethan-1-yl, 2-(4-chlorophenyl)ethan-1-yl, 2-(4-chlorophenyl)ethan-2-yl, 2-chlorobenzyl, 2-methylpyrimidin-5-ylmethyl, 3,4-dimethoxybenzyl, 3-carbamoylbenzyl, 3-carboxybenzyl, 3-cyanobenzyl, 3-fluoro-4-methylbenzyl, 3-methoxycarbonylbenzyl, 3-methyl-4-chlorobenzyl, 4-(2-hydroxyethancarbamoyl)benzyl, 4-(4-methylpiperazin-1-ylmethyl)benzyl, 4-(morpholin-4-ylmethyl)benzyl, 4-(morpholin-4-ylmethyl)phenyl, 4-carbamoylbenzyl, 4-chlorobenzyl, 4-chlorophenyl, 4-cyanobenzyl, 4-methoxybenzyl, 4-methoxycarbonylbenzyl, 4-methylbenzyl, 5-chloropyridin-2-ylmethyl, 5-methylpyridin-2-yl, 6-methylpyridin-3-ylmethyl, cyclohexylmethyl, or tetrahydropyran-4-ylmethyl.

In some aspects, R² is hydrogen, C₁-C₄ alkyl, -(C₁-C₄ alkylene)-C(O)-NH₂, -(C₁-C₄ alkylene)-C(O)-NH-(C₁-C₄ alkyl), -(C₁-C₄ alkylene)-C(O)-OH, -(C₁-C₄ alkylene)-C(O)-O-(C₁-C₄ alkyl), -(C₁-C₄ alkylene)-C(O)-(C₁-C₄ alkyl), or (C₀-C₁ alkylene)-aryl, wherein any alkylene, alkyl, or aryl portion of R² is optionally substituted with up to 3 substituents independently selected from halo, —OH, or —CN.

In some aspects, R² is hydrogen, —CH₃, —CH(CH₃)—C(O)—NH₂, —CH(CH₃)—C(O)—NH—(CH₂)₂—OH, —(CH₂)₂—C(O)OH, —(CH₂)₂C(O)—NH₂, —CH₂—C(O)—NH₂, —(CH₂)₂—O—CH₃, — (CH₂)₂—OH, —CH₂—C(O)—O—CH₂CH₃, or benzyl.

In some aspects, R³ and R⁴ are hydrogen.

In some aspects, R³ and R⁴ are taken together with the carbon atoms to which they are bound and intervening atoms to form a cyclopentyl moiety that is fused to the phenyl moiety bearing R⁴ and spirofused to the 4,5-dihydropyrazol-1,3,4-triyl moiety bearing R³.

In some aspects, one R⁵ or one R⁶ is chloro.

In some aspects, m is 0, n is 1, and R⁵ is chloro.

In some aspects, the chloro is in the para position.

In some aspects, the compound is any one of the compounds set forth in the following table:

| # | Structure |
|---|---|
| 100 | 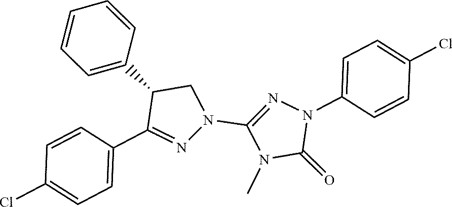 [SMILES: Cn1c(N2C[C@@H](c3ccccc3)C(c3ccc(Cl)cc3)=N2)nn(-c2ccc(Cl)cc2)c1=O] |
| 101 | 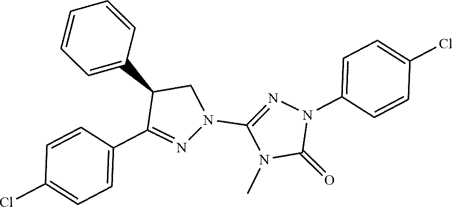 [SMILES: Cn1c(N2C[C@H](c3ccccc3)C(c3ccc(Cl)cc3)=N2)nn(-c2ccc(Cl)cc2)c1=O] |
| 102 | 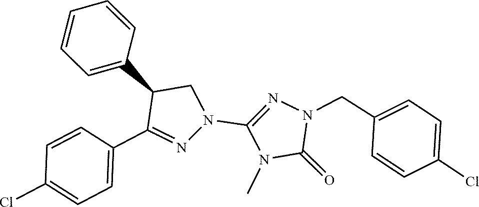 [SMILES: Cn1c(N2C[C@H](c3ccccc3)C(c3ccc(Cl)cc3)=N2)nn(Cc2ccc(Cl)cc2)c1=O] |
| 103 | 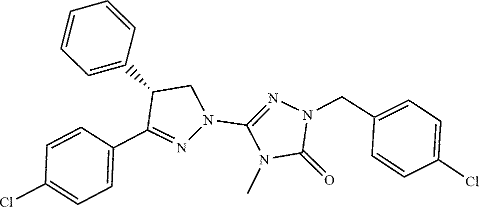 [SMILES: Cn1c(N2C[C@@H](c3ccccc3)C(c3ccc(Cl)cc3)=N2)nn(Cc2ccc(Cl)cc2)c1=O] |
| 104 | 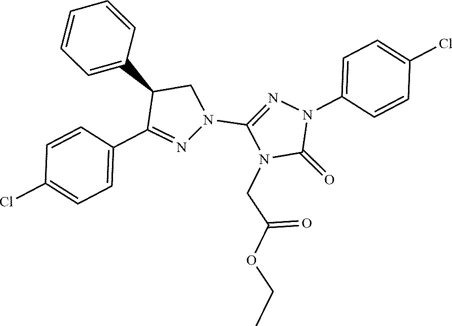 [SMILES: CCOC(=O)Cn1c(N2C[C@H](c3ccccc3)C(c3ccc(Cl)cc3)=N2)nn(-c2ccc(Cl)cc2)c1=O] |
| 105 | 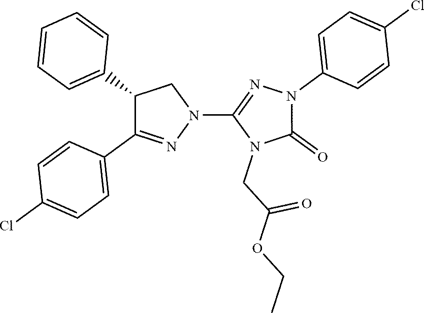 [SMILES: CCOC(=O)Cn1c(N2C[C@@H](c3ccccc3)C(c3ccc(Cl)cc3)=N2)nn(-c2ccc(Cl)cc2)c1=O] |
| 106 | 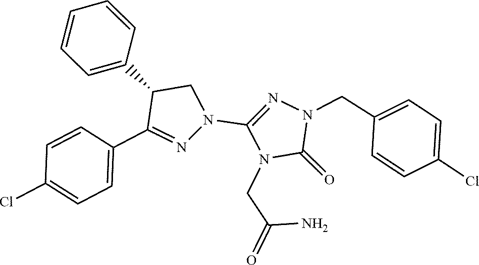 [SMILES: NC(=O)Cn1c(N2C[C@@H](c3ccccc3)C(c3ccc(Cl)cc3)=N2)nn(Cc2ccc(Cl)cc2)c1=O] |
| 107 | 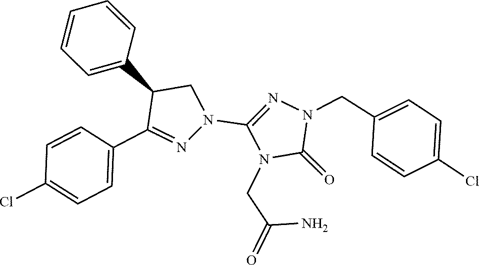 [SMILES: NC(=O)Cn1c(N2C[C@H](c3ccccc3)C(c3ccc(Cl)cc3)=N2)nn(Cc2ccc(Cl)cc2)c1=O] |
| 108 | 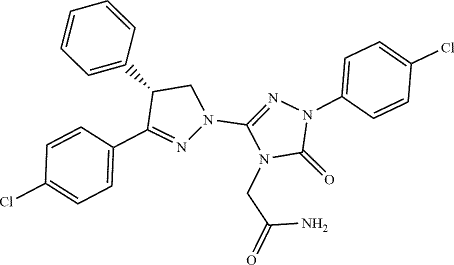 [SMILES: NC(=O)Cn1c(N2C[C@@H](c3ccccc3)C(c3ccc(Cl)cc3)=N2)nn(-c2ccc(Cl)cc2)c1=O] |
| 109 | 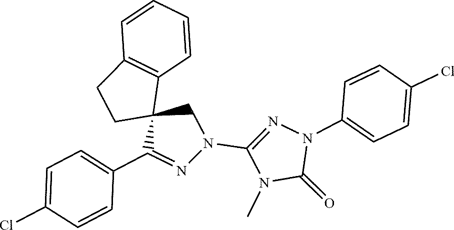 [SMILES: Cn1c(N2C[C@]3(CCc4ccccc43)C(c3ccc(Cl)cc3)=N2)nn(-c2ccc(Cl)cc2)c1=O] |
| 110 | 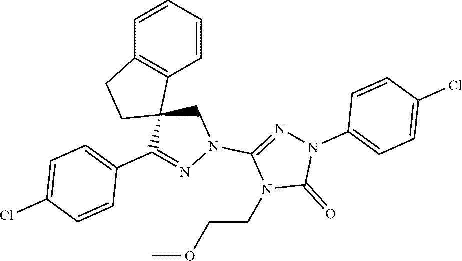 [SMILES: COCCn1c(N2C[C@]3(CCc4ccccc43)C(c3ccc(Cl)cc3)=N2)nn(-c2ccc(Cl)cc2)c1=O] |
| 111 | 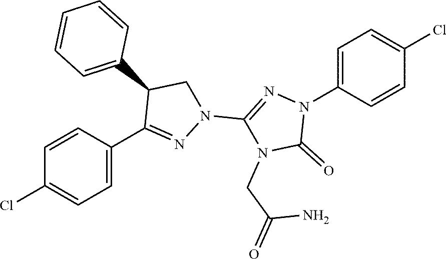 [SMILES: NC(=O)Cn1c(N2C[C@H](c3ccccc3)C(c3ccc(Cl)cc3)=N2)nn(-c2ccc(Cl)cc2)c1=O] |
| 112 | 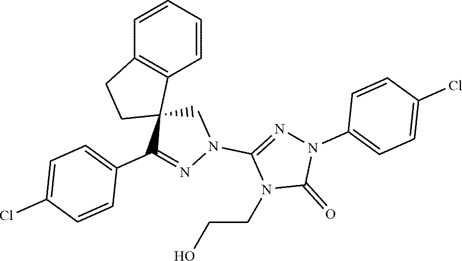 [SMILES: O=c1n(-c2ccc(Cl)cc2)nc(N2C[C@@]3(CCc4ccccc43)C(c3ccc(Cl)cc3)=N2)n1CCO] |
| 113 | 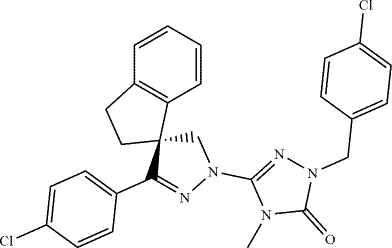 [SMILES: Cn1c(N2C[C@@]3(CCc4ccccc43)C(c3ccc(Cl)cc3)=N2)nn(Cc2ccc(Cl)cc2)c1=O] |
| 114 | 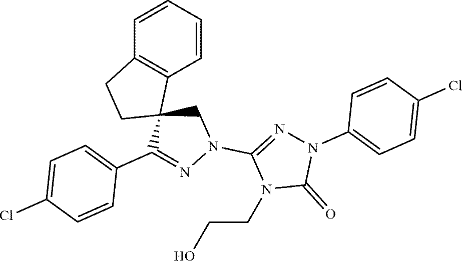 [SMILES: O=c1n(-c2ccc(Cl)cc2)nc(N2C[C@]3(CCc4ccccc43)C(c3ccc(Cl)cc3)=N2)n1CCO] |
| 115 | 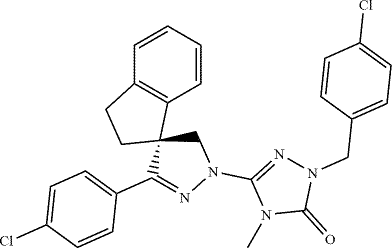 [SMILES: Cn1c(N2C[C@]3(CCc4ccccc43)C(c3ccc(Cl)cc3)=N2)nn(Cc2ccc(Cl)cc2)c1=O] |
| 116 | 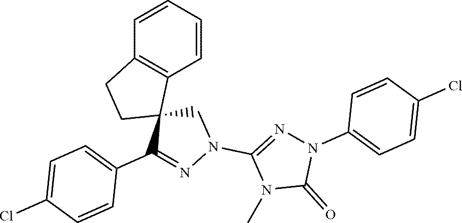 [SMILES: Cn1c(N2C[C@@]3(CCc4ccccc43)C(c3ccc(Cl)cc3)=N2)nn(-c2ccc(Cl)cc2)c1=O] |
| 117 | 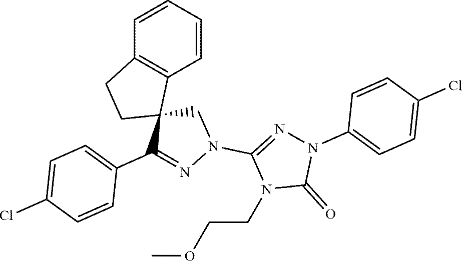 [SMILES: COCCn1c(N2C[C@@]3(CCc4ccccc43)C(c3ccc(Cl)cc3)=N2)nn(-c2ccc(Cl)cc2)c1=O] |
| 118 | 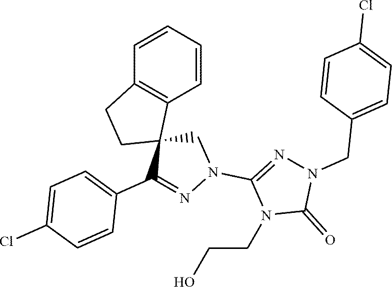 [SMILES: O=c1n(Cc2ccc(Cl)cc2)nc(N2C[C@@]3(CCc4ccccc43)C(c3ccc(Cl)cc3)=N2)n1CCO] |
| 119 | 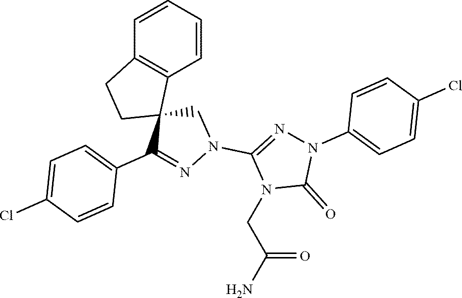 [SMILES: NC(=O)Cn1c(N2C[C@@]3(CCc4ccccc43)C(c3ccc(Cl)cc3)=N2)nn(-c2ccc(Cl)cc2)c1=O] |
| 120 | 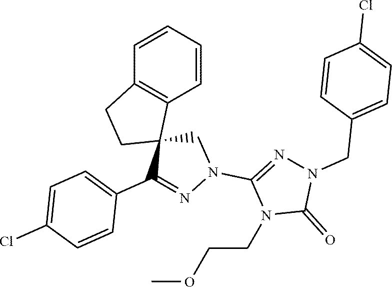 [SMILES: COCCn1c(N2C[C@@]3(CCc4ccccc43)C(c3ccc(Cl)cc3)=N2)nn(Cc2ccc(Cl)cc2)c1=O] |
| 121 | 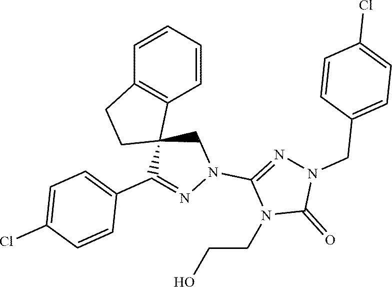 [SMILES: O=c1n(Cc2ccc(Cl)cc2)nc(N2C[C@]3(CCc4ccccc43)C(c3ccc(Cl)cc3)=N2)n1CCO] |
| 122 | 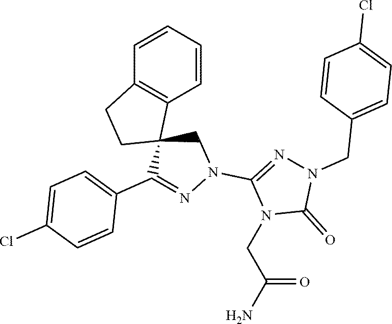 [SMILES: NC(=O)Cn1c(N2C[C@]3(CCc4ccccc43)C(c3ccc(Cl)cc3)=N2)nn(Cc2ccc(Cl)cc2)c1=O] |
| 123 | 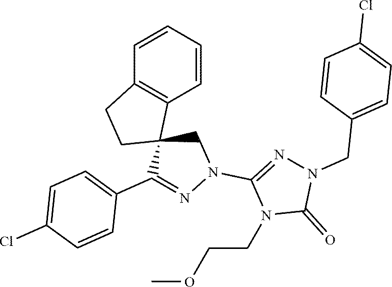 [SMILES: COCCn1c(N2C[C@]3(CCc4ccccc43)C(c3ccc(Cl)cc3)=N2)nn(Cc2ccc(Cl)cc2)c1=O] |
| 124 | 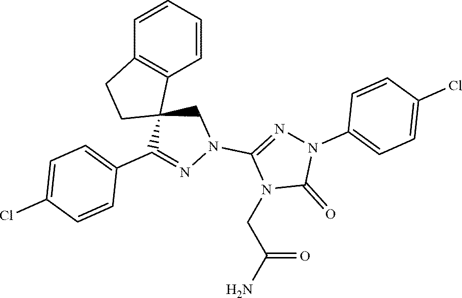 [SMILES: NC(=O)Cn1c(N2C[C@]3(CCc4ccccc43)C(c3ccc(Cl)cc3)=N2)nn(-c2ccc(Cl)cc2)c1=O] |
| 125 | 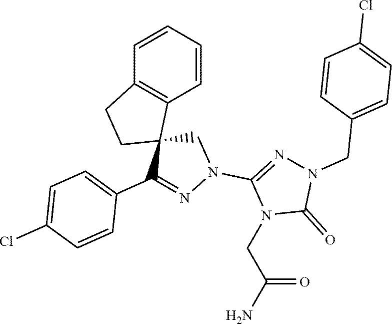 [SMILES: NC(=O)Cn1c(N2C[C@@]3(CCc4ccccc43)C(c3ccc(Cl)cc3)=N2)nn(Cc2ccc(Cl)cc2)c1=O] |
| 126 | 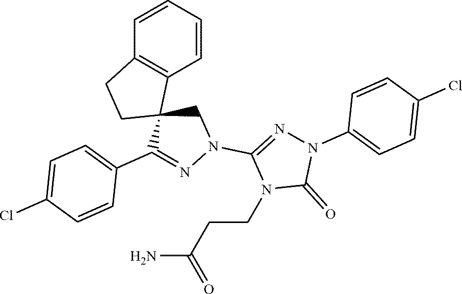 [SMILES: NC(=O)CCn1c(N2C[C@]3(CCc4ccccc43)C(c3ccc(Cl)cc3)=N2)nn(-c2ccc(Cl)cc2)c1=O] |
| 127 | 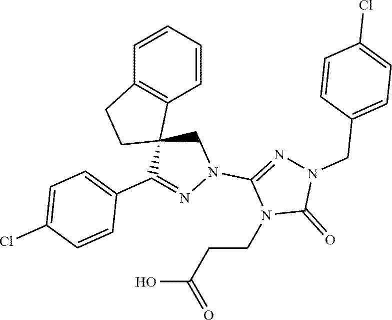 [SMILES: O=C(O)CCn1c(N2C[C@]3(CCc4ccccc43)C(c3ccc(Cl)cc3)=N2)nn(Cc2ccc(Cl)cc2)c1=O] |
| 128 | 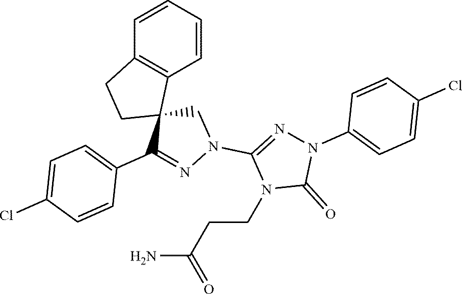 [SMILES: NC(=O)CCn1c(N2C[C@@]3(CCc4ccccc43)C(c3ccc(Cl)cc3)=N2)nn(-c2ccc(Cl)cc2)c1=O] |
| 129 | 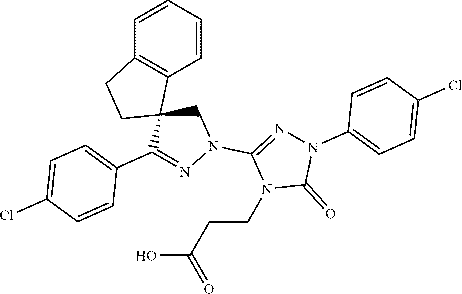 [SMILES: O=C(O)CCn1c(N2C[C@]3(CCc4ccccc43)C(c3ccc(Cl)cc3)=N2)nn(-c2ccc(Cl)cc2)c1=O] |
| 130 | 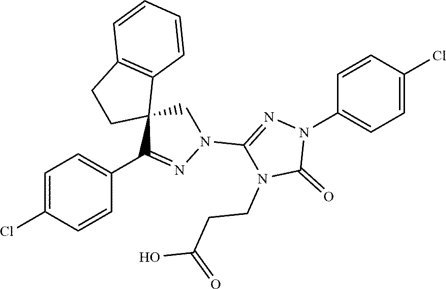 [SMILES: O=C(O)CCn1c(N2C[C@@]3(CCc4ccccc43)C(c3ccc(Cl)cc3)=N2)nn(-c2ccc(Cl)cc2)c1=O] |
| 131 | 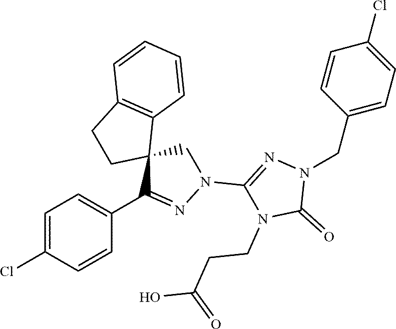 [SMILES: O=C(O)CCn1c(N2C[C@@]3(CCc4ccccc43)C(c3ccc(Cl)cc3)=N2)nn(Cc2ccc(Cl)cc2)c1=O] |
| 132 | 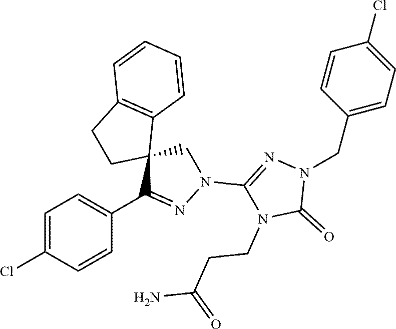 [SMILES: NC(=O)CCn1c(N2C[C@@]3(CCc4ccccc43)C(c3ccc(Cl)cc3)=N2)nn(Cc2ccc(Cl)cc2)c1=O] |
| 133 | 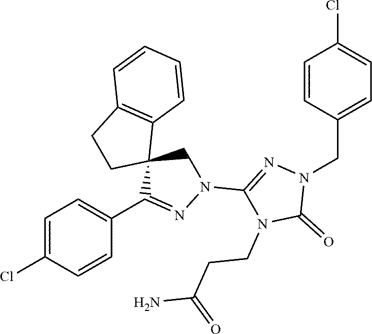 [SMILES: NC(=O)CCn1c(N2C[C@]3(CCc4ccccc43)C(c3ccc(Cl)cc3)=N2)nn(Cc2ccc(Cl)cc2)c1=O] |
| 134 | 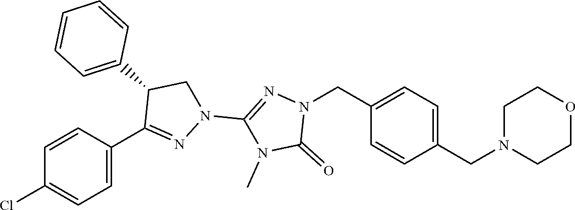 [SMILES: Cn1c(N2C[C@@H](c3ccccc3)C(c3ccc(Cl)cc3)=N2)nn(Cc2ccc(CN3CCOCC3)cc2)c1=O] |
| 135 | 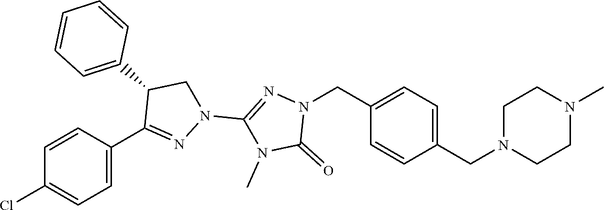 [SMILES: CN1CCN(Cc2ccc(Cn3nc(N4C[C@@H](c5ccccc5)C(c5ccc(Cl)cc5)=N4)n(C)c3=O)cc2)CC1] |
| 136 | 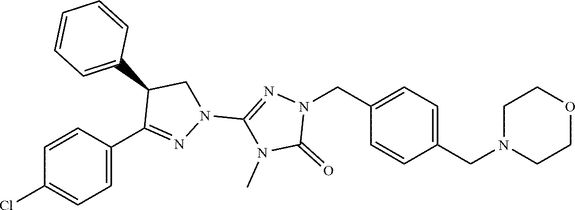 [SMILES: Cn1c(N2C[C@H](c3ccccc3)C(c3ccc(Cl)cc3)=N2)nn(Cc2ccc(CN3CCOCC3)cc2)c1=O] |
| 137 | 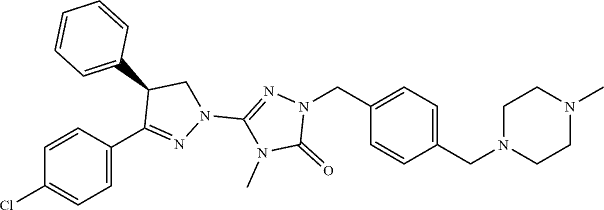 [SMILES: CN1CCN(Cc2ccc(Cn3nc(N4C[C@H](c5ccccc5)C(c5ccc(Cl)cc5)=N4)n(C)c3=O)cc2)CC1] |
| 138 | 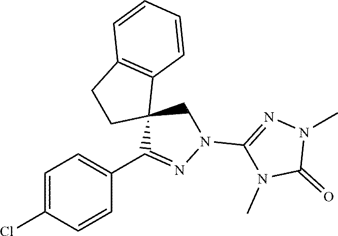 [SMILES: Cn1nc(N2C[C@]3(CCc4ccccc43)C(c3ccc(Cl)cc3)=N2)n(C)c1=O] |
| 139 | 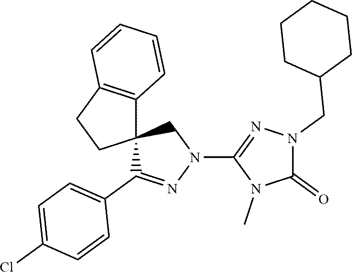 [SMILES: Cn1c(N2C[C@]3(CCc4ccccc43)C(c3ccc(Cl)cc3)=N2)nn(CC2CCCCC2)c1=O] |
| 140 | 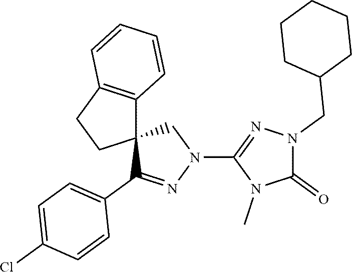 [SMILES: Cn1c(N2C[C@@]3(CCc4ccccc43)C(c3ccc(Cl)cc3)=N2)nn(CC2CCCCC2)c1=O] |
| 141 | 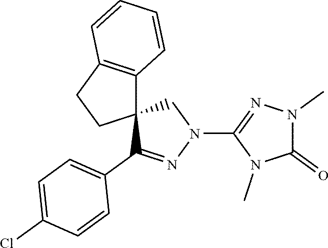 [SMILES: Cn1nc(N2C[C@@]3(CCc4ccccc43)C(c3ccc(Cl)cc3)=N2)n(C)c1=O] |
| 142 | 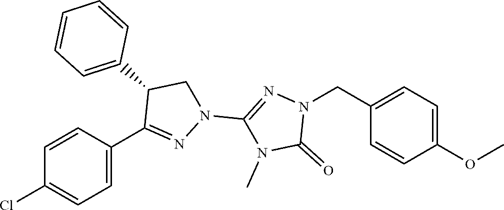 [SMILES: COc1ccc(Cn2nc(N3C[C@@H](c4ccccc4)C(c4ccc(Cl)cc4)=N3)n(C)c2=O)cc1] |
| 143 | 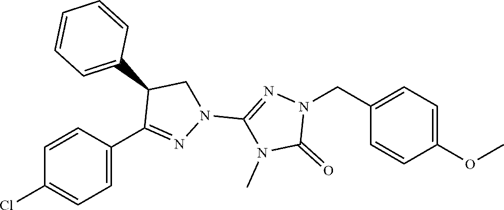 [SMILES: COc1ccc(Cn2nc(N3C[C@H](c4ccccc4)C(c4ccc(Cl)cc4)=N3)n(C)c2=O)cc1] |
| 144 | 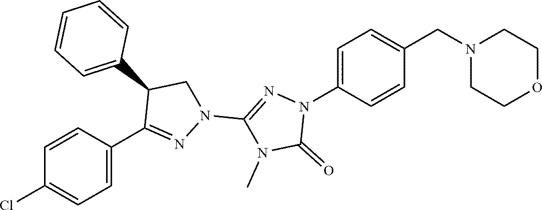 [SMILES: Cn1c(N2C[C@H](c3ccccc3)C(c3ccc(Cl)cc3)=N2)nn(-c2ccc(CN3CCOCC3)cc2)c1=O] |
| 145 | 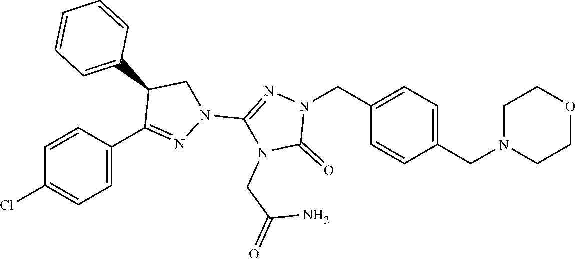 [SMILES: NC(=O)Cn1c(N2C[C@H](c3ccccc3)C(c3ccc(Cl)cc3)=N2)nn(Cc2ccc(CN3CCOCC3)cc2)c1=O] |
| 146 | 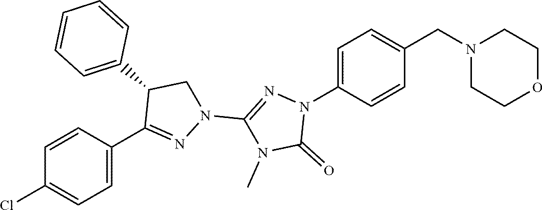 [SMILES: Cn1c(N2C[C@@H](c3ccccc3)C(c3ccc(Cl)cc3)=N2)nn(-c2ccc(CN3CCOCC3)cc2)c1=O] |
| 147 | 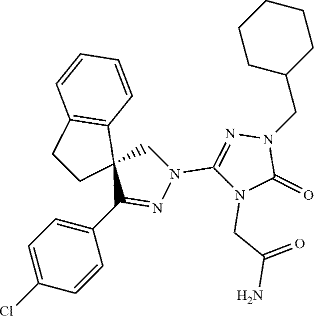 [SMILES: NC(=O)Cn1c(N2C[C@@]3(CCc4ccccc43)C(c3ccc(Cl)cc3)=N2)nn(CC2CCCCC2)c1=O] |
| 149 | 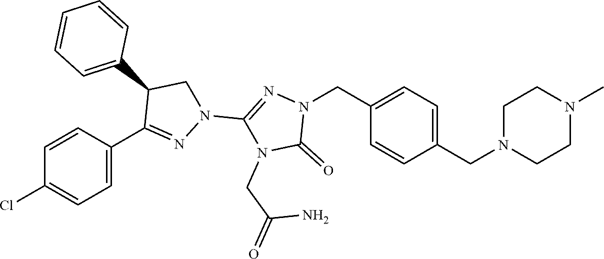 [SMILES: CN1CCN(Cc2ccc(Cn3nc(N4C[C@H](c5ccccc5)C(c5ccc(Cl)cc5)=N4)n(CC(N)=O)c3=O)cc2)CC1] |
| 150 | 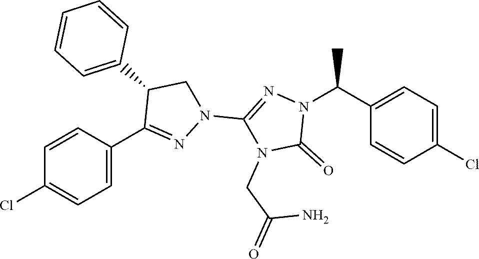 [SMILES: C[C@@H](c1ccc(Cl)cc1)n1nc(N2C[C@@H](c3ccccc3)C(c3ccc(Cl)cc3)=N2)n(CC(N)=O)c1=O] |
| 152 | 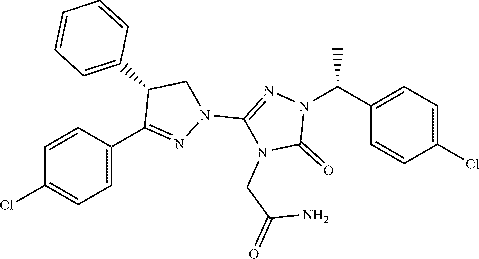 [SMILES: C[C@H](c1ccc(Cl)cc1)n1nc(N2C[C@@H](c3ccccc3)C(c3ccc(Cl)cc3)=N2)n(CC(N)=O)c1=O] |
| 153 | 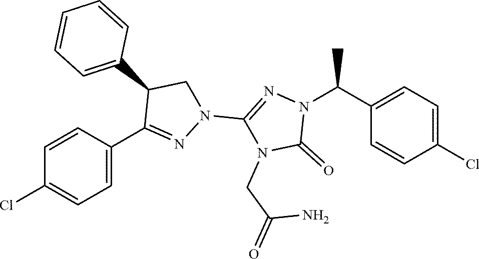 [SMILES: C[C@@H](c1ccc(Cl)cc1)n1nc(N2C[C@H](c3ccccc3)C(c3ccc(Cl)cc3)=N2)n(CC(N)=O)c1=O] |
| 154 | 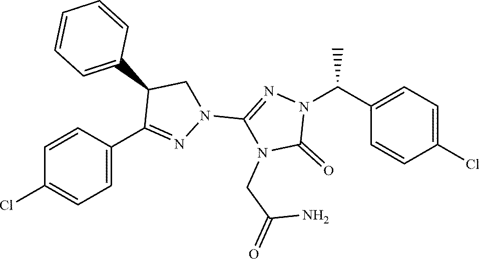 [SMILES: C[C@H](c1ccc(Cl)cc1)n1nc(N2C[C@H](c3ccccc3)C(c3ccc(Cl)cc3)=N2)n(CC(N)=O)c1=O] |
| 158 | 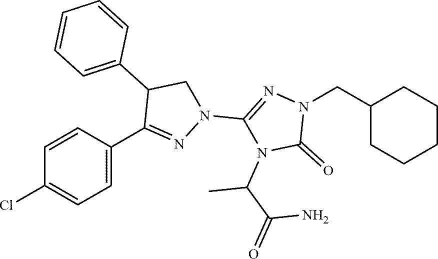 [SMILES: CC(C(N)=O)n1c(N2CC(c3ccccc3)C(c3ccc(Cl)cc3)=N2)nn(CC2CCCCC2)c1=O] |
| 159 | 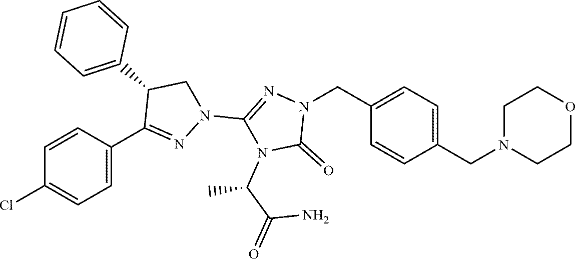 [SMILES: C[C@@H](C(N)=O)n1c(N2C[C@@H](c3ccccc3)C(c3ccc(Cl)cc3)=N2)nn(Cc2ccc(CN3CCOCC3)cc2)c1=O] |
| 160 | 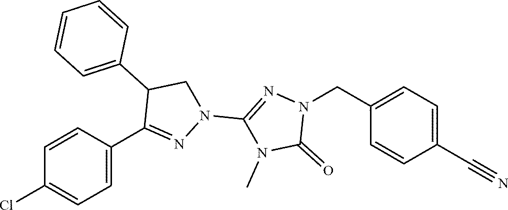 [SMILES: Cn1c(N2CC(c3ccccc3)C(c3ccc(Cl)cc3)=N2)nn(Cc2ccc(C#N)cc2)c1=O] |
| 161 | 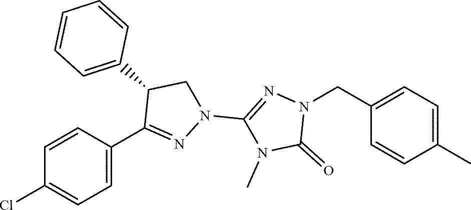 [SMILES: Cc1ccc(Cn2nc(N3C[C@@H](c4ccccc4)C(c4ccc(Cl)cc4)=N3)n(C)c2=O)cc1] |
| 163 | 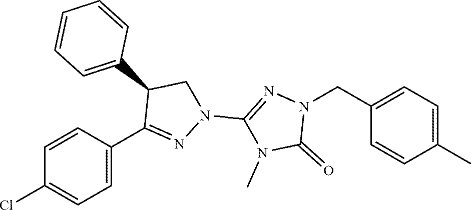 [SMILES: Cc1ccc(Cn2nc(N3C[C@H](c4ccccc4)C(c4ccc(Cl)cc4)=N3)n(C)c2=O)cc1] |
| 164 | 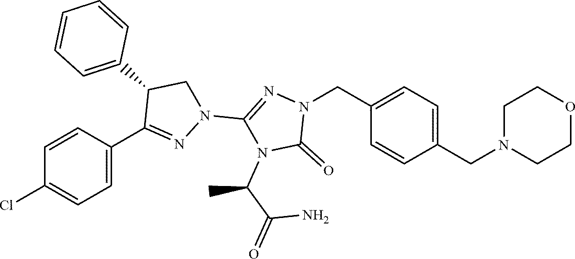 [SMILES: C[C@H](C(N)=O)n1c(N2C[C@@H](c3ccccc3)C(c3ccc(Cl)cc3)=N2)nn(Cc2ccc(CN3CCOCC3)cc2)c1=O] |
| 166 | 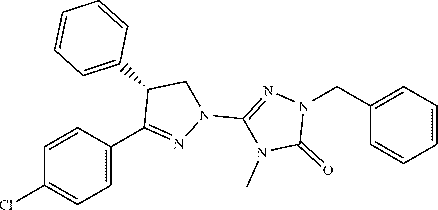 [SMILES: Cn1c(N2C[C@@H](c3ccccc3)C(c3ccc(Cl)cc3)=N2)nn(Cc2ccccc2)c1=O] |
| 167 | 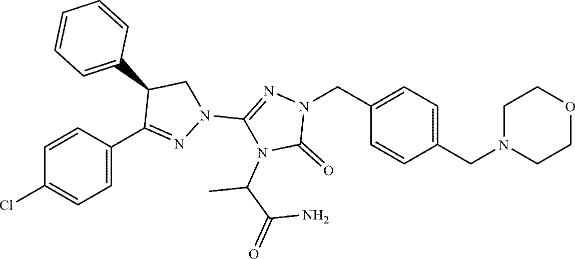 [SMILES: CC(C(N)=O)n1c(N2C[C@H](c3ccccc3)C(c3ccc(Cl)cc3)=N2)nn(Cc2ccc(CN3CCOCC3)cc2)c1=O] |
| 168 | 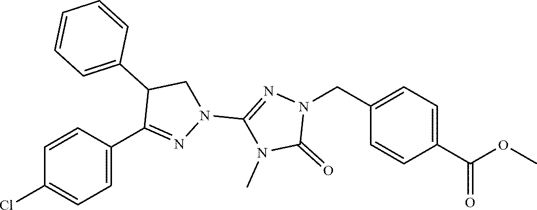 [SMILES: COC(=O)c1ccc(Cn2nc(N3CC(c4ccccc4)C(c4ccc(Cl)cc4)=N3)n(C)c2=O)cc1] |
| 169 | 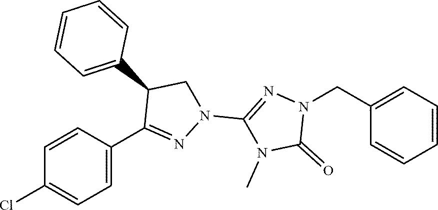 [SMILES: Cn1c(N2C[C@H](c3ccccc3)C(c3ccc(Cl)cc3)=N2)nn(Cc2ccccc2)c1=O] |
| 170 | 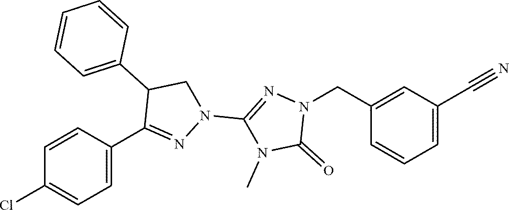 [SMILES: Cn1c(N2CC(c3ccccc3)C(c3ccc(Cl)cc3)=N2)nn(Cc2cccc(C#N)c2)c1=O] |
| 171 | 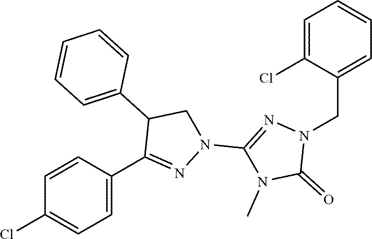 [SMILES: Cn1c(N2CC(c3ccccc3)C(c3ccc(Cl)cc3)=N2)nn(Cc2ccccc2Cl)c1=O] |
| 172 | 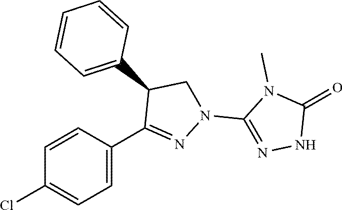 [SMILES: Cn1c(N2C[C@H](c3ccccc3)C(c3ccc(Cl)cc3)=N2)n[nH]c1=O] |
| 173 | 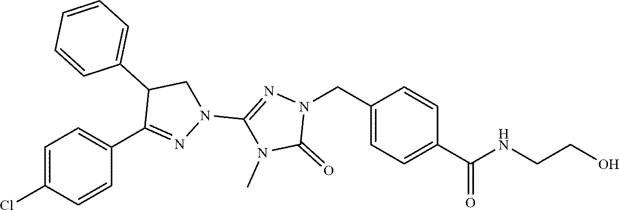 [SMILES: Cn1c(N2CC(c3ccccc3)C(c3ccc(Cl)cc3)=N2)nn(Cc2ccc(C(=O)NCCO)cc2)c1=O] |
| 174 | 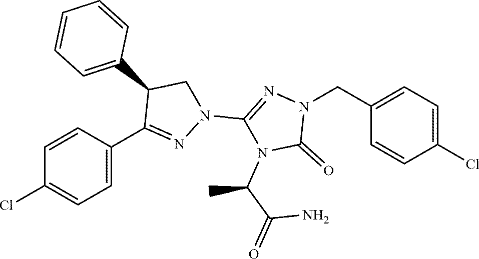 [SMILES: C[C@H](C(N)=O)n1c(N2C[C@H](c3ccccc3)C(c3ccc(Cl)cc3)=N2)nn(Cc2ccc(Cl)cc2)c1=O] |
| 175 | 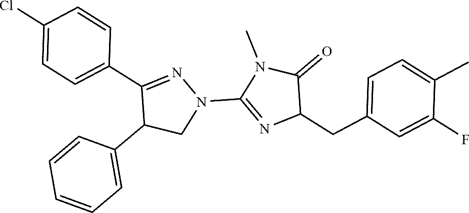 [SMILES: Cc1ccc(CC2N=C(N3CC(c4ccccc4)C(c4ccc(Cl)cc4)=N3)N(C)C2=O)cc1F] |
| 176 | 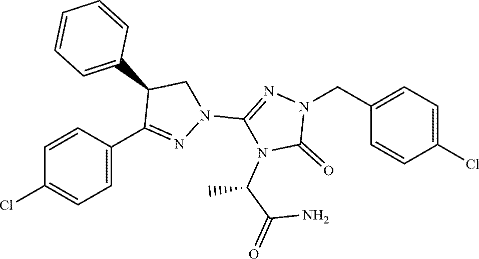 [SMILES: C[C@@H](C(N)=O)n1c(N2C[C@H](c3ccccc3)C(c3ccc(Cl)cc3)=N2)nn(Cc2ccc(Cl)cc2)c1=O] |
| 177 | 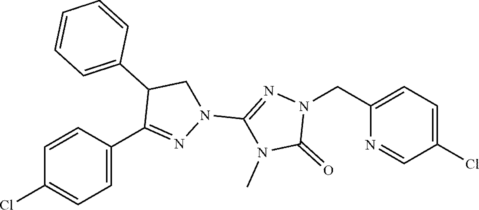 [SMILES: Cn1c(N2CC(c3ccccc3)C(c3ccc(Cl)cc3)=N2)nn(Cc2ccc(Cl)cn2)c1=O] |
| 178 | 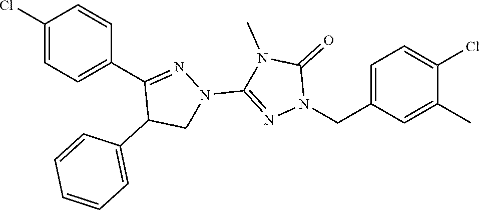 [SMILES: Cc1cc(Cn2nc(N3CC(c4ccccc4)C(c4ccc(Cl)cc4)=N3)n(C)c2=O)ccc1Cl] |
| 179 | 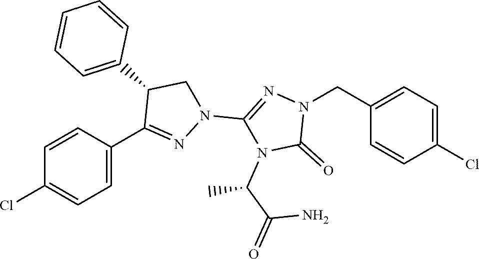 [SMILES: C[C@@H](C(N)=O)n1c(N2C[C@@H](c3ccccc3)C(c3ccc(Cl)cc3)=N2)nn(Cc2ccc(Cl)cc2)c1=O] |
| 180 | 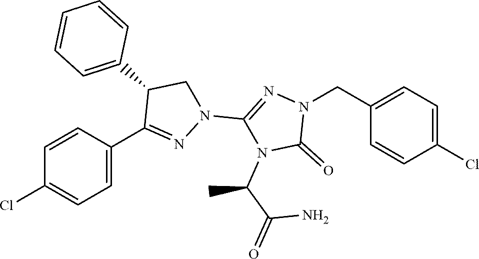 [SMILES: C[C@H](C(N)=O)n1c(N2C[C@@H](c3ccccc3)C(c3ccc(Cl)cc3)=N2)nn(Cc2ccc(Cl)cc2)c1=O] |
| 181 | 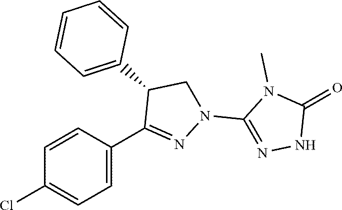 [SMILES: Cn1c(N2C[C@@H](c3ccccc3)C(c3ccc(Cl)cc3)=N2)n[nH]c1=O] |
| 182 | 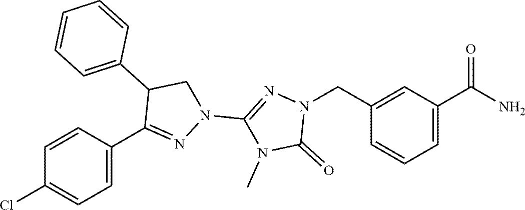 [SMILES: Cn1c(N2CC(c3ccccc3)C(c3ccc(Cl)cc3)=N2)nn(Cc2cccc(C(N)=O)c2)c1=O] |
| 183 | 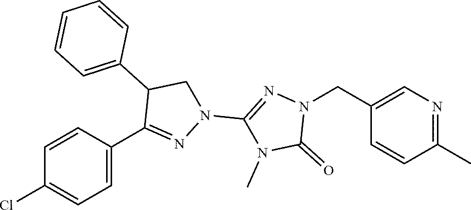 [SMILES: Cc1ccc(Cn2nc(N3CC(c4ccccc4)C(c4ccc(Cl)cc4)=N3)n(C)c2=O)cn1] |
| 184 | 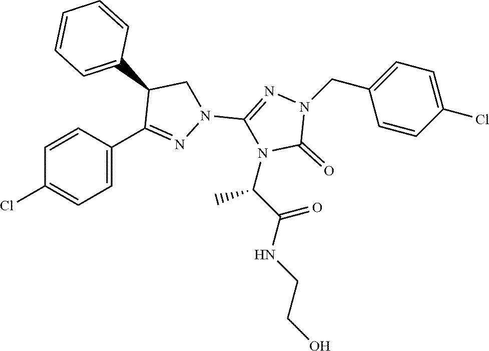 |
| 186 | 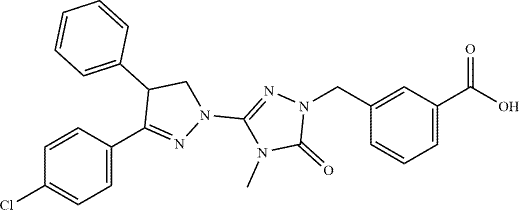 [SMILES: Cn1c(N2CC(c3ccccc3)C(c3ccc(Cl)cc3)=N2)nn(Cc2cccc(C(=O)O)c2)c1=O] |
| 187 | 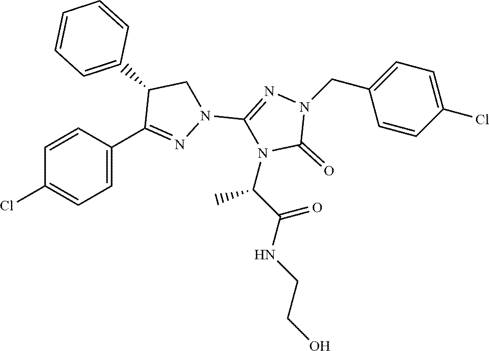 |
| 189 | 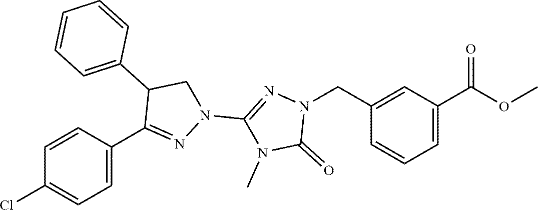 [SMILES: COC(=O)c1cccc(Cn2nc(N3CC(c4ccccc4)C(c4ccc(Cl)cc4)=N3)n(C)c2=O)c1] |
| 190 | 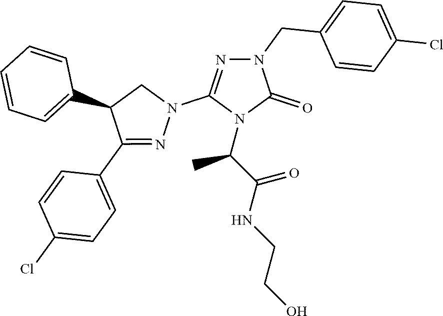 [SMILES: C[C@H](C(=O)NCCO)n1c(N2C[C@H](c3ccccc3)C(c3ccc(Cl)cc3)=N2)nn(Cc2ccc(Cl)cc2)c1=O] |
| 191 | 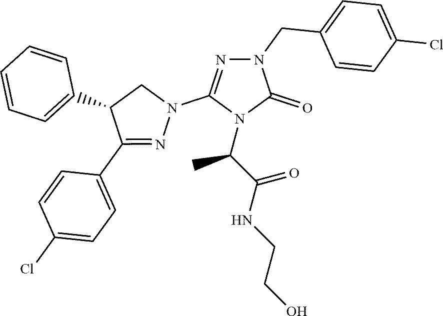 [SMILES: C[C@H](C(=O)NCCO)n1c(N2C[C@@H](c3ccccc3)C(c3ccc(Cl)cc3)=N2)nn(Cc2ccc(Cl)cc2)c1=O] |
| 192 | 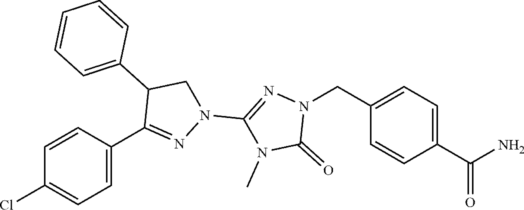 [SMILES: Cn1c(N2CC(c3ccccc3)C(c3ccc(Cl)cc3)=N2)nn(Cc2ccc(C(N)=O)cc2)c1=O] |
| 193 | 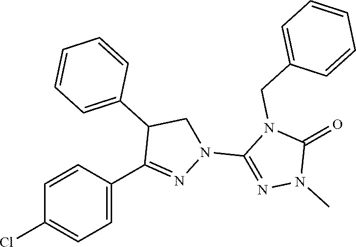 [SMILES: Cn1nc(N2CC(c3ccccc3)C(c3ccc(Cl)cc3)=N2)n(Cc2ccccc2)c1=O] |
| 194 | 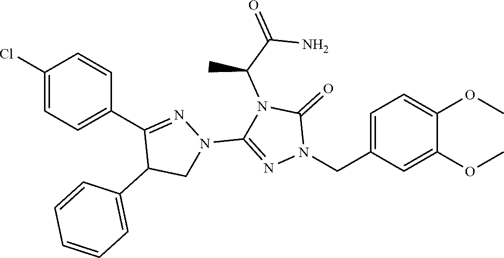 [SMILES: COc1ccc(Cn2nc(N3CC(c4ccccc4)C(c4ccc(Cl)cc4)=N3)n([C@@H](C)C(N)=O)c2=O)cc1OC] |
| 195 | 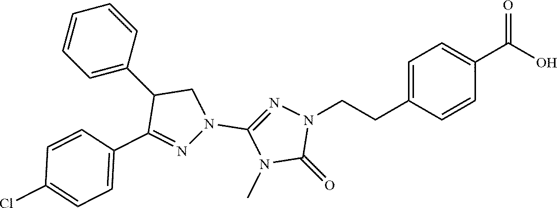 [SMILES: Cn1c(N2CC(c3ccccc3)C(c3ccc(Cl)cc3)=N2)nn(CCc2ccc(C(=O)O)cc2)c1=O] |
| 196 | 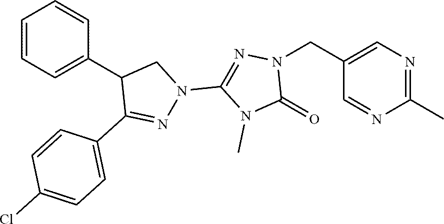 [SMILES: Cc1ncc(Cn2nc(N3CC(c4ccccc4)C(c4ccc(Cl)cc4)=N3)n(C)c2=O)cn1] |
| 197 | 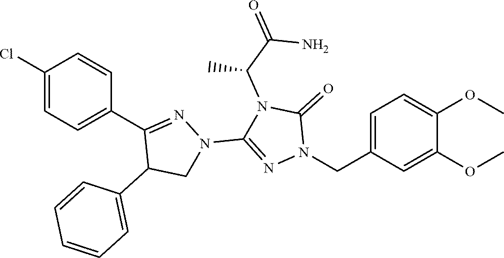 [SMILES: COc1ccc(Cn2nc(N3CC(c4ccccc4)C(c4ccc(Cl)cc4)=N3)n([C@H](C)C(N)=O)c2=O)cc1OC] |
| 199 | 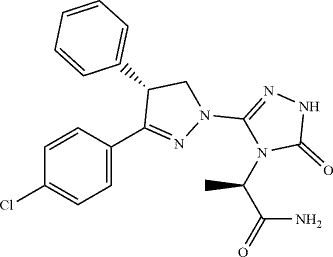 [SMILES: C[C@H](C(N)=O)n1c(N2C[C@@H](c3ccccc3)C(c3ccc(Cl)cc3)=N2)n[nH]c1=O] |
| 200 | 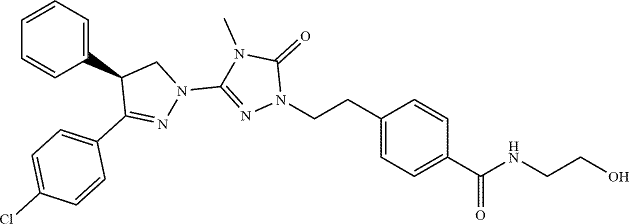 [SMILES: Cn1c(N2C[C@H](c3ccccc3)C(c3ccc(Cl)cc3)=N2)nn(CCc2ccc(C(=O)NCCO)cc2)c1=O] |
| 201 | 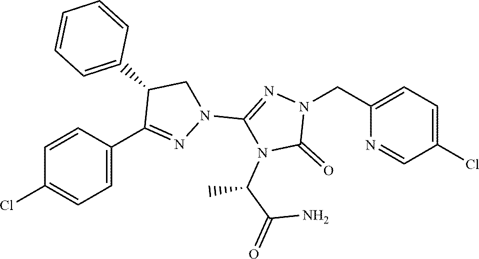 [SMILES: C[C@@H](C(N)=O)n1c(N2C[C@@H](c3ccccc3)C(c3ccc(Cl)cc3)=N2)nn(Cc2ccc(Cl)cn2)c1=O] |
| 202 | 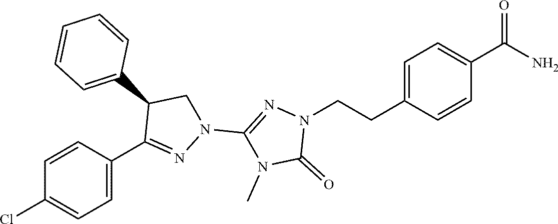 [SMILES: Cn1c(N2C[C@H](c3ccccc3)C(c3ccc(Cl)cc3)=N2)nn(CCc2ccc(C(N)=O)cc2)c1=O] |
| 203 | 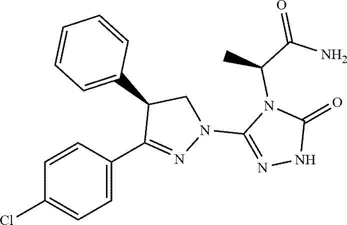 [SMILES: C[C@@H](C(N)=O)n1c(N2C[C@H](c3ccccc3)C(c3ccc(Cl)cc3)=N2)n[nH]c1=O] |
| 205 | 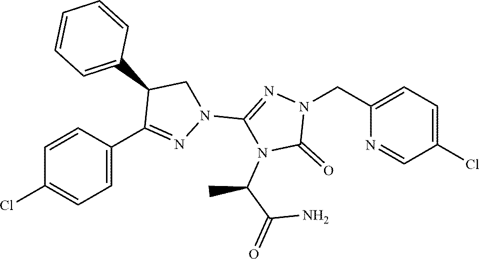 [SMILES: C[C@H](C(N)=O)n1c(N2C[C@H](c3ccccc3)C(c3ccc(Cl)cc3)=N2)nn(Cc2ccc(Cl)cn2)c1=O] |
| 206 | 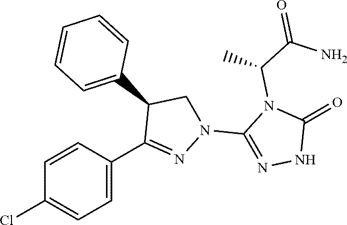 [SMILES: C[C@H](C(N)=O)n1c(N2C[C@H](c3ccccc3)C(c3ccc(Cl)cc3)=N2)n[nH]c1=O] |
| 207 | 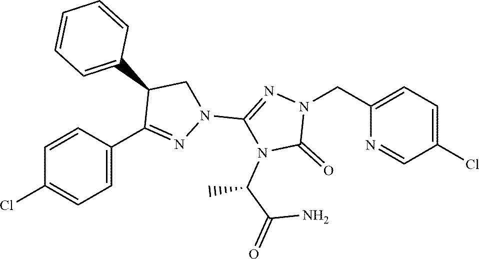 [SMILES: C[C@@H](C(N)=O)n1c(N2C[C@H](c3ccccc3)C(c3ccc(Cl)cc3)=N2)nn(Cc2ccc(Cl)cn2)c1=O] |
| 208 | 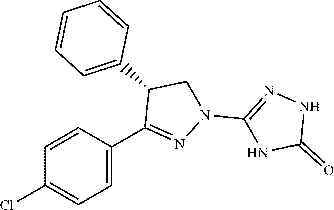 [SMILES: O=c1[nH]nc(N2C[C@@H](c3ccccc3)C(c3ccc(Cl)cc3)=N2)[nH]1] |
| 209 | 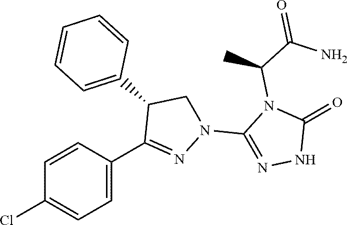 [SMILES: C[C@@H](C(N)=O)n1c(N2C[C@@H](c3ccccc3)C(c3ccc(Cl)cc3)=N2)n[nH]c1=O] |
| 210 | 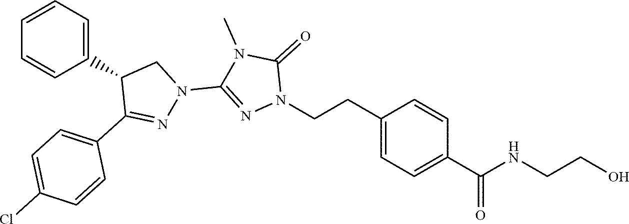 [SMILES: Cn1c(N2C[C@@H](c3ccccc3)C(c3ccc(Cl)cc3)=N2)nn(CCc2ccc(C(=O)NCCO)cc2)c1=O] |
| 211 | 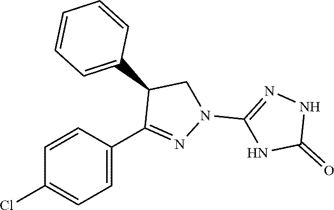 [SMILES: O=c1[nH]nc(N2C[C@H](c3ccccc3)C(c3ccc(Cl)cc3)=N2)[nH]1] |
| 212 | 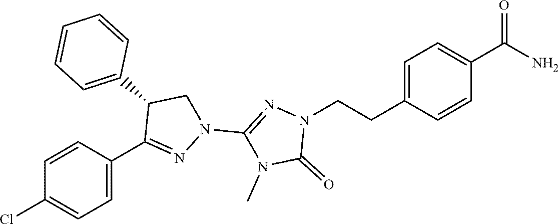 [SMILES: Cn1c(N2C[C@@H](c3ccccc3)C(c3ccc(Cl)cc3)=N2)nn(CCc2ccc(C(N)=O)cc2)c1=O] |
| 213 | 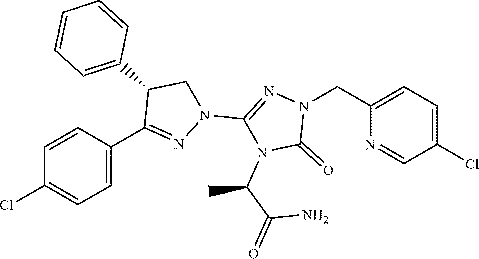 [SMILES: C[C@H](C(N)=O)n1c(N2C[C@@H](c3ccccc3)C(c3ccc(Cl)cc3)=N2)nn(Cc2ccc(Cl)cn2)c1=O] |
| 214 | 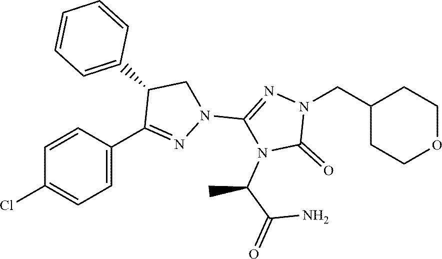 [SMILES: C[C@H](C(N)=O)n1c(N2C[C@@H](c3ccccc3)C(c3ccc(Cl)cc3)=N2)nn(CC2CCOCC2)c1=O] |
| 215 | 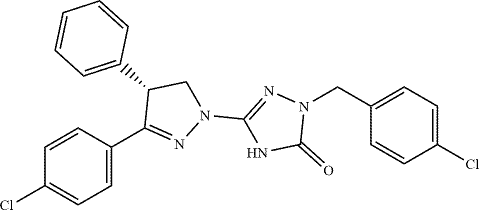 [SMILES: O=c1[nH]c(N2C[C@@H](c3ccccc3)C(c3ccc(Cl)cc3)=N2)nn1Cc1ccc(Cl)cc1] |
| 216 | 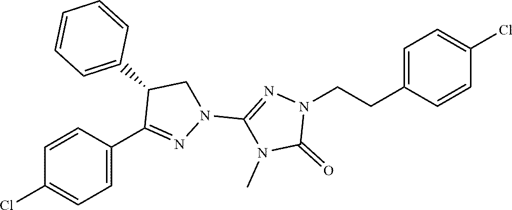 [SMILES: Cn1c(N2C[C@@H](c3ccccc3)C(c3ccc(Cl)cc3)=N2)nn(CCc2ccc(Cl)cc2)c1=O] |
| 217 | 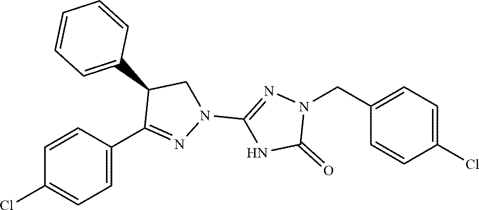 [SMILES: O=c1[nH]c(N2C[C@H](c3ccccc3)C(c3ccc(Cl)cc3)=N2)nn1Cc1ccc(Cl)cc1] |
| 218 | 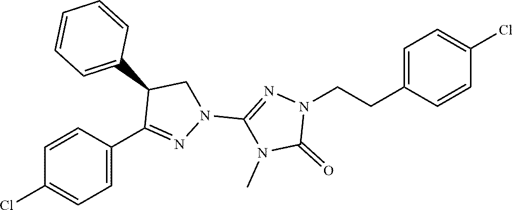 [SMILES: Cn1c(N2C[C@H](c3ccccc3)C(c3ccc(Cl)cc3)=N2)nn(CCc2ccc(Cl)cc2)c1=O] |
| 219 | 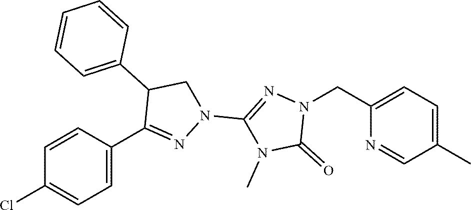 [SMILES: Cc1ccc(Cn2nc(N3CC(c4ccccc4)C(c4ccc(Cl)cc4)=N3)n(C)c2=O)nc1] |
| 220 | 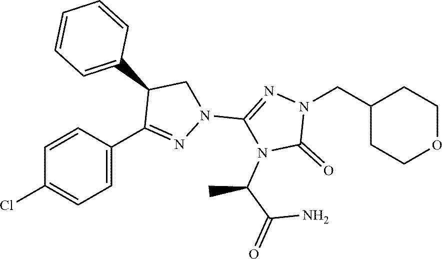 [SMILES: C[C@H](C(N)=O)n1c(N2C[C@H](c3ccccc3)C(c3ccc(Cl)cc3)=N2)nn(CC2CCOCC2)c1=O] |
| 221 | 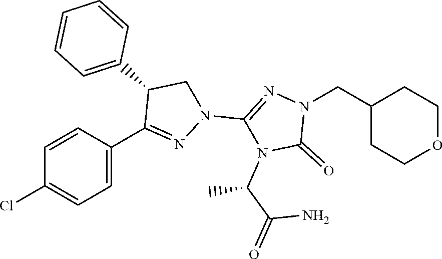 [SMILES: C[C@@H](C(N)=O)n1c(N2C[C@@H](c3ccccc3)C(c3ccc(Cl)cc3)=N2)nn(CC2CCOCC2)c1=O] |
| 222 | 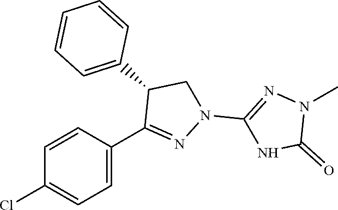 [SMILES: Cn1nc(N2C[C@@H](c3ccccc3)C(c3ccc(Cl)cc3)=N2)[nH]c1=O] |
| 223 | 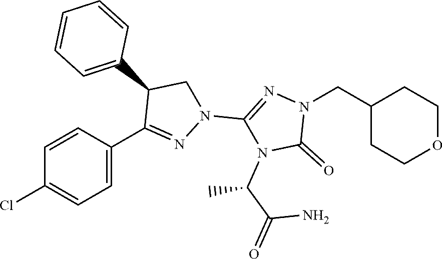 [SMILES: C[C@@H](C(N)=O)n1c(N2C[C@H](c3ccccc3)C(c3ccc(Cl)cc3)=N2)nn(CC2CCOCC2)c1=O] |
| 224 | 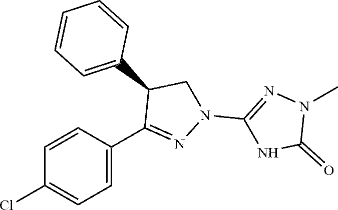 [SMILES: Cn1nc(N2C[C@H](c3ccccc3)C(c3ccc(Cl)cc3)=N2)[nH]c1=O] |

In certain embodiments, the compounds of the invention may be racemic. In certain embodiments, the compounds of the invention may be enriched in one enantiomer. For example, a compound of the invention may have greater than 30% ee, 40% ee, 50% ee, 60% ee, 70% ee, 80% ee, 90% ee, or even 95% or greater ee.

The compounds of the invention have more than one stereocenter. Accordingly, the compounds of the invention may be enriched in one or more diastereomers. For example, a compound of the invention may have greater than 30% de, 40% de, 50% de, 60% de, 70% de, 80% de, 90% de, or even 95% or greater de. In certain embodiments, the compounds of the invention have substantially one isomeric configuration at one or more stereogenic centers, and have multiple isomeric configurations at the remaining stereogenic centers.

In certain embodiments, the enantiomeric excess of the stereocenter is at least 40% ee, 50% ee, 60% ee, 70% ee, 80% ee, 90% ee, 92% ee, 94% ee, 95% ee, 96% ee, 98% ee or greater ee.

As used herein, single bonds drawn without stereochemistry do not indicate the stereochemistry of the compound.

As used herein, hashed or bolded non-wedge bonds indicate relative, but not absolute, stereochemical configuration (e.g., do not distinguish between enantiomers of a given diastereomer).

As used herein, hashed or bolded wedge bonds indicate absolute stereochemical configuration.

In some embodiments, the invention relates to pharmaceutical composition comprising a compound of the invention and a pharmaceutically acceptable carrier. In certain embodiments, a therapeutic preparation or pharmaceutical composition of the compound of the invention may be enriched to provide predominantly one enantiomer of a compound. An enantiomerically enriched mixture may comprise, for example, at least 60 mol percent of one enantiomer, or more preferably at least 75, 90, 95, or even 99 mol percent. In certain embodiments, the compound enriched in one enantiomer is substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the composition or compound mixture. For example, if a composition or compound mixture contains 98 grams of a first enantiomer and 2 grams of a second enantiomer, it would be said to contain 98 mol percent of the first enantiomer and only 2% of the second enantiomer.

In certain embodiments, a therapeutic preparation or pharmaceutical composition may be enriched to provide predominantly one diastereomer of the compound of the invention. A diastereomerically enriched mixture may comprise, for example, at least 60 mol percent of one diastereomer, or more preferably at least 75, 90, 95, or even 99 mol percent.

The compounds of this invention may be used in treating the conditions described herein, in the form of the free base, salts (preferably pharmaceutically acceptable salts), solvates, hydrates, prodrugs, isomers, or mixtures thereof. All forms are within the scope of the disclosure. Acid addition salts may be formed and provide a more convenient form for use; in practice, use of the salt form inherently amounts to use of the base form. The acids which can be used to prepare the acid addition salts include preferably those which produce, when combined with the free base, pharmaceutically acceptable salts, that is, salts whose anions are non-toxic to the subject organism in pharmaceutical doses of the salts, so that the beneficial properties inherent in the free base are not vitiated by side effects ascribable to the anions. Although pharmaceutically acceptable salts of the basic compounds are preferred, all acid addition salts are useful as sources of the free base form even if the particular salt per se is desired only as an intermediate product as, for example, when the salt is formed only for the purposes of purification and identification, or when it is used as an intermediate in preparing a pharmaceutically acceptable salt by ion exchange procedures.

Pharmaceutically acceptable salts within the scope of the disclosure include those derived from the following acids; mineral acids such as hydrochloric acid, sulfuric acid, phosphoric acid and sulfamic acid; and organic acids such as acetic acid, citric acid, lactic acid, tartaric acid, malonic acid, methanesulfonic acid, ethanesulfonic acid, benzenesulfonic acid, p-toluenesulfonic acid, cyclohexylsulfamic acid, quinic acid, and the like.

The compounds of the present invention can be formulated as pharmaceutical compositions and administered to a subject in need of treatment, for example a mammal, such as a human patient, in a variety of forms adapted to the chosen route of administration, for example, orally, nasally, intraperitoneally, or parenterally (e.g., by intravenous, intraperitoneal, subcutaneous, intramuscular, transepithelial, nasal, intrapulmonary, intrathecal, rectal or topical routes). Parenteral administration may be by continuous infusion over a selected period of time.

In accordance with the methods of the disclosure, the described compounds may be administered to a patient in a variety of forms depending on the selected route of administration, as will be understood by those skilled in the art. The compositions containing the compounds of the disclosure can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington’s Pharmaceutical Sciences (Remington’s Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

A composition comprising a compound of the present disclosure may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

A person skilled in the art would know how to prepare suitable formulations. Conventional procedures and ingredients for the selection and preparation of suitable formulations are described, for example, in Remington’s Pharmaceutical Sciences (1990 -18th edition) and in The United States Pharmacopeia: The National Formulary (USP 24 NF19) published in 1999.

Thus, compounds of the invention may be systemically administered, e.g., orally, in combination with a pharmaceutically acceptable vehicle such as an inert diluent or an assimilable edible carrier; or by inhalation or insufflation. They may be enclosed in hard or soft shell gelatin capsules, may be compressed into tablets, or may be incorporated directly with the food of the patient’s diet. For oral therapeutic administration, the compounds may be combined with one or more excipients and used in the form of ingestible tablets, buccal tablets, troches, capsules, elixirs, suspensions, syrups, wafers, and the like. The compounds may be combined with a fine inert powdered carrier and inhaled by the subject or insufflated. Such compositions and preparations should contain at least 0.1% of compounds of formula I. The percentage of the compositions and preparations may, of course, be varied and may conveniently be between about 2% to about 60% of the weight of a given unit dosage form. The amount of the compounds in such therapeutically useful compositions is such that an effective dosage level will be obtained.

In certain embodiments of the disclosure, compositions comprising a compound of the present disclosure for oral administration include capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and the like, each containing a predetermined amount of the compound of the present disclosure as an active ingredient.

In solid dosage forms for oral administration (capsules, tablets, troches, pills, dragees, powders, granules, and the like), one or more compositions comprising the compound of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, gum tragacanth, corn starch, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the compounds may be incorporated into sustained-release preparations and devices. For example, the compounds may be incorporated into time release capsules, time release tablets, and time release pills.

Liquid dosage forms for oral administration include pharmaceutically acceptable emulsions, microemulsions, solutions, suspensions, syrups, and elixirs. In addition to the compound of the present disclosure, the liquid dosage forms may contain inert diluents commonly used in the art, such as water or other solvents, solubilizing agents and emulsifiers, such as ethyl alcohol (ethanol), isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, salts and/or prodrugs thereof, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In certain embodiments, pharmaceutical compositions suitable for parenteral administration may comprise the compound of the present disclosure in combination with one or more pharmaceutically acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The compounds may be administered intravenously or intraperitoneally by infusion or injection. Solutions of the compounds or their salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the compounds which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the compounds in the required amount in the appropriate solvent with various of the other ingredients enumerated above, as required, followed by filter sterilization. In the case of sterile powders for the preparation of sterile injectable solutions, the preferred methods of preparation are vacuum drying and freeze drying techniques, which yield a powder of the active ingredient plus any additional desired ingredient present in the previously sterile-filtered solutions.

For topical administration, the compounds may be applied in pure form. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Other solid carriers include nontoxic polymeric nanoparticles or microparticles. Useful liquid carriers include water, alcohols or glycols or water/alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508), all of which are hereby incorporated by reference.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949, which is hereby incorporated by reference.

For example, the concentration of the compounds in a liquid composition, such as a lotion, can be from about 0.1-25% by weight, or from about 0.5-10% by weight. The concentration in a semi-solid or solid composition such as a gel or a powder can be about 0.1-5% by weight, or about 0.5-2.5% by weight.

The amount of the compounds required for use in treatment will vary not only with the particular salt selected but also with the route of administration, the nature of the condition being treated and the age and condition of the patient and will be ultimately at the discretion of the attendant physician or clinician.

Effective dosages and routes of administration of agents of the invention are conventional. The exact amount (effective dose) of the agent will vary from subject to subject, depending on, for example, the species, age, weight and general or clinical condition of the subject, the severity or mechanism of any disorder being treated, the particular agent or vehicle used, the method and scheduling of administration, and the like. A therapeutically effective dose can be determined empirically, by conventional procedures known to those of skill in the art. See, e.g., The Pharmacological Basis of Therapeutics, Goodman and Gilman, eds., Macmillan Publishing Co., New York. For example, an effective dose can be estimated initially either in cell culture assays or in suitable animal models. The animal model may also be used to determine the appropriate concentration ranges and routes of administration. Such information can then be used to determine useful doses and routes for administration in humans. A therapeutic dose can also be selected by analogy to dosages for comparable therapeutic agents.

The particular mode of administration and the dosage regimen will be selected by the attending clinician, taking into account the particulars of the case (e.g., the subject, the disease, the disease state involved, and whether the treatment is prophylactic). Treatment may involve daily or multi-daily doses of compound(s) over a period of a few days to months, or even years.

The compounds may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator.

The dosage of the compounds and/or compositions of the disclosure can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. The compounds of the disclosure may be administered initially in a suitable dosage that may be adjusted as required, depending on the clinical response. To calculate the human equivalent dose (HED) from a dosage used in the treatment of age-dependent cognitive impairment in rats, the formula HED (mg/kg) = rat dose (mg/kg) × 0.16 may be employed (see Estimating the Safe Starting Dose in Clinical Trials for Therapeutics in Adult Healthy Volunteers, December 2002, Center for Biologics Evaluation and Research). For example, using that formula, a dosage of 10 mg/kg in rats is equivalent to 1.6 mg/kg in humans. This conversion is based on a more general formula HED = animal dose in mg/kg × (animal weight in kg/human weight in kg) 0.33. Similarly, to calculate the HED from a dosage used in the treatment in mouse, the formula HED (mg/kg) = mouse dose (mg/kg) × 0.08 may be employed (see Estimating the Safe Starting Dose in Clinical Trials for Therapeutics in Adult Healthy Volunteers, December 2002, Center for Biologics Evaluation and Research).

### Methods of Treatment

Aberrant activity of the CB1 reeptor has been implicated in numerous diseases and conditions, including diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

Accordingly, in certain embodiments, the invention provides methods for treating a disease or condition characterized by aberrant CB1 activity, or the reducing risk of developing, a disease or condition characterized by aberrant CB1 activity. In certain embodiments, the disease or condition is diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

In some embodiments, the disease or condition is diabetic nephropathy.

In some embodiments, the disease or condition is focal segmental glomerular sclerosis.

In some embodiments, the disease or condition is nonalcoholic steatohepatitis.

### Subjects to Be Treated

In one aspect of the invention, a subject is selected on the basis that they have, or are at risk of developing, a disease or condition characterized by aberrant CB1 activity.

The methods are effective for a variety of subjects including mammals, e.g., humans and other animals, such as laboratory animals, e.g., mice, rats, rabbits, or monkeys, or domesticated and farm animals, e.g., cats, dogs, goats, sheep, pigs, cows, or horses. In some embodiments, the subject is a mammal. In some embodiments, the subject is a human.

### EXAMPLES

The invention is further described in the following examples, which do not limit the scope of the invention described in the claims.

### Example 1. Preparation of Intermediates Used in Syntheses

### A. 3-Chlorophenyl)-4-Phenyl-4,5-Dihydro-1H-Pyrazole

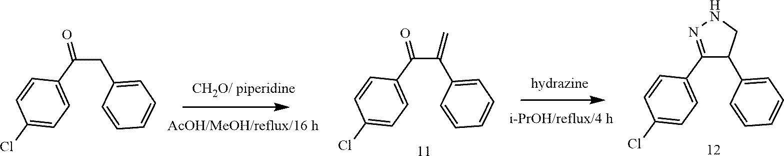

### 1-Chlorophenyl)-2-Phenylprop-2-En-1-One

A mixture of 1-(4-chlorophenyl)-2-phenylethan-1-one (380 mg, 1.647 mmol, 1 equiv.), HCHO (247.30 mg, 8.236 mmol, 5 equiv.), AcOH (21.76 mg, 0.362 mmol, 0.22 equiv.) and piperidine (18.23 mg, 0.214 mmol, 0.13 equiv.) in MeOH (20 mL) were heated for 4 h at 80° C. under N₂ atmosphere. The mixture was allowed to cool down to room temperature. Desired product could be detected by LCMS. The resulting mixture was concentrated under reduced pressure. The residue was purified by silica gel column chromatography, eluted with PE/EA (10 / 1) to afford 1-(4-chlorophenyl)-2-phenylprop-2-en-1-one (335 mg, 83.80%) as a brown oil.

### 3-Chlorophenyl)-4-Phenyl-4,5-Dihydro-1H-Pyrazole

To a stirred solution of 1-(4-chlorophenyl)-2-phenylprop-2-en-1-one (0.5 g, 2.060 mmol, 1 equiv.) in EtOH (20 mL) was added NH₂NH₂·H₂O (1.03 g, 16.460 mmol, 7.99 equiv, 80%) dropwise at room temperature under N₂ atmosphere. The mixture was stirred for 4 h at 80° C. The mixture was allowed to cool down to room temperature. Desired product could be detected by LCMS. The mixture was concentrated under reduced pressure, then the white precipitations formed. The precipitated solids were collected by filtration and washed with EtOH (1 × 5 mL). The crude product (3-(4-chlorophenyl)-4-phenyl-4,5-dihydro-1H-pyrazole (0.3 g, 56.72%)) was used in the next step directly without further purification.

### B. 3-chlorophenyl)-1,2,3,5-tetrahydrospiro[indene-1,4-pyrazole]

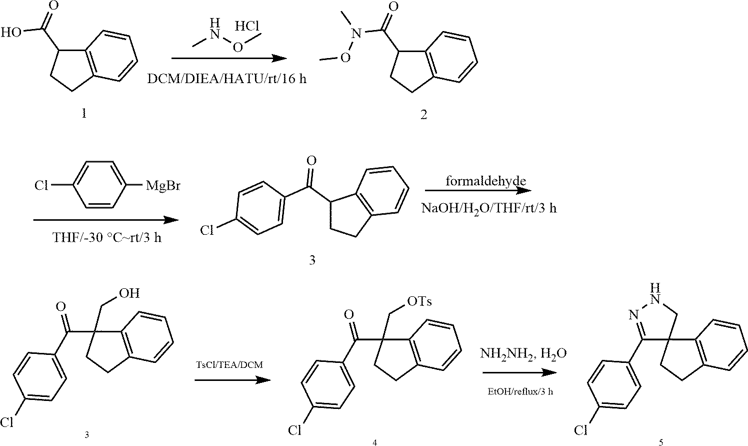

### N-Methoxy-N-Methyl-2,3-Dihydro-1H-Indene-1-Carboxamide

Into a 500 mL round-bottom flask were added 2,3-dihydro-1H-indene-1-carboxylic acid (10.0 g, 61.657 mmol, 1 equiv.) and HATU (35.17 g, 92.485 mmol, 1.5 equiv.) and 300 mL DCM at room temperature. To the above mixture was added DIEA (23.91 g, 184.971 mmol, 3.0 equiv.) dropwise over 5 min at room temperature. The resulting mixture was stirred for additional 10 min at room temperature. To the above mixture was added methoxy(methyl)amine hydrochloride (7216.81 mg, 73.988 mmol, 1.20 equiv.) over 5 min at room temperature. The resulting mixture was stirred for 16 hours at room temperature. The mixture was neutralized to pH 7 with saturated NaHCO₃ (aq.). The resulting mixture was extracted with CH₂Cl₂ (2 × 200 mL). The combined organic layers were washed with brine (2×50 mL), dried over anhydrous Na₂SO₄. After filtration, the filtrate was concentrated under reduced pressure to give residue. The residue was purified by silica gel column chromatography, eluted with PE/ EtOAc (5:1) to afford N-methoxy-N-methyl-2,3-dihydro-1H-indene-1-carboxamide (10.0 g, 79.02%) as a light brown oil.

### Chlorophenyl-2,3-Dihydro-1H-Inden-1-yl)Methanone

To a stirred solution of N-methoxy-N-methyl-2,3-dihydro-1H-indene-1-carboxamide (7.7 g, 38 mmol, 1 equiv.) in THF was added bromo(4-chlorophenyl)magnesium (57 mL, 57 mmol, 1.5 equiv.) dropwise at -30° C. under nitrogen atmosphere. The resulting mixture was stirred for 30 min at -30° C. under nitrogen atmosphere and then was allowed to warm to room temperature. After 2 hours the reaction was quenched with Water at room temperature. The resulting mixture was extracted with EtOAc (2 × 100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/ EtOAc (20:1) to afford (4-chlorophenyl-2,3-dihydro-1H-inden-1-yl)methanone (7.1 g) as a light yellow solid.

### (4-Chlorobenzoyl)-2,3-Dihydro-1H-Inden-1-yl] Methanol

A solution of (4-chlorophenyl)(₂,3-dihydro-1H-inden-1-yl)methanone (7.1 g, 27.7 mmol, 1 equiv.) in 150 mL THF was treated with NaOH (54 mL, 27.7 mmol, 1 equiv, 0.5 mol/L in water) for 5 min at room temperature under air atmosphere followed by the addition of formaldehyde (5.6 mL, 69.3 mmol, 2.5 equiv, 37 wt. % in water) dropwise at room temperature. The resulting mixture was stirred for 3 h at room temperature under air atmosphere. The reaction was monitored by TLC. The mixture was acidified to pH 5 with HCl (aq.). The resulting mixture was extracted with EtOAc (2 × 100 mL). The combined organic layers were washed with brine (2 × 50 mL), dried over anhydrous Na₂SO₄. After filtration, the filtrate was concentrated under reduced pressure. The residue was purified by silica gel column chromatography, eluted with PE/ EtOAc (5:1) to afford [1-(4-chlorobenzoyl)-2,3-dihydro-1H-inden-1-yl]methanol (6.57 g) as a light yellow oil.

### (4-Chlorobenzoyl)-2,3-Dihydro-1H-Inden-1-yl]Methyl 4-Methylbenzene-1-Sulfonate

To a stirred solution of [1-(4-chlorobenzoyl)-2,3-dihydro-1H-inden-1-yl]methanol (858 mg, 3 mmol, 1.00 equiv.) and DMAP (36.55 mg, 0.299 mmol, 0.1 equiv.) and triethylamine (1816.65 mg, 17.952 mmol, 6.00 equiv.) in 20 mL DCM was added 4-methylbenzene-1-sulfonyl chloride (855.61 mg, 4.488 mmol, 1.50 equiv.) at 0° C. under air atmosphere. The resulting mixture was stirred for 16 h at room temperature under air atmosphere. The reaction was monitored by TLC. The resulting mixture was concentrated under reduced pressure and was diluted with ethyl acetate (50 mL). The resulting mixture was extracted with EtOAc (2 ×50 mL). The combined organic layers were washed with brine (2×10 mL), dried over anhydrous Na₂SO₄. After filtration, the filtrate was concentrated under reduced pressure. The residue was purified by silica gel column chromatography, eluted with PE/ EtOAc (12:1) to afford [1-(4-chlorobenzoyl)-2,3-dihydro-1H-inden-1-yl]methyl 4-methylbenzene-1-sulfonate (1.26 g) as a brown oil.

### 3-Chlorophenyl)-1,2,3,5-Tetrahydrospiro[Indene-1,4-Pyrazole

To a stirred solution of [1-(4-chlorobenzoyl)-2,3-dihydro-1H-inden-1-yl]methyl 4-methylbenzene-1-sulfonate (6.5 g, 14.741 mmol, 1 equiv.) in EtOH (100 mL) were added hydrazine hydrate (80%) (7.38 g, 147.422 mmol, 10.00 equiv.) in portions at room temperature. The resulting mixture was stirred for 3 h at 80° C. under nitrogen atmosphere. The resulting mixture was concentrated under reduced pressure and extracted with CH₂Cl₂ (2 × 300 mL). The combined organic layers were washed with brine (2× 50 mL), dried over anhydrous Na₂SO₄. After filtration, the filtrate was concentrated under reduced pressure. This resulted in 3-(4-chlorophenyl)-1,2,3,5-tetrahydrospiro[indene-1,4-pyrazole] (3.9 g, 93.56%) as a light brown oil. The crude product was used in the next step directly without further purification.

### C. 3-Bromo-4-Methyl-4,5-Dihydro-1H-1,2,4-Triazol-5-One

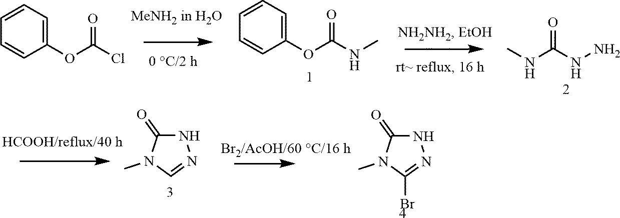

### Phenyl N-Methylcarbamate

To a stirred solution of methylamine (10 g) was added phenyl carbonochloridate (10 g, 63.869 mmol, 1 equiv.) dropwise at 0° C. The above mixture was stirred for 2 hours at 0° C. Desired product could be detected by LCMS. The precipitated solids were collected by filtration and washed with water (2 × 1 mL). The resulting solid was dried under nitrogen atmosphere to afford phenyl N-methylcarbamate (5.6 g, 58 %) as a white solid. The crude product was used in the next step directly without further purification.

### 1-AminoMethylurea

A solution of phenyl N-methylcarbamate (5.6 g, 37.046 mmol, 1 equiv.) and NH₂NH₂•H₂O (4.64 g, 74.150 mmol, 2.00 equiv, 80%) in EtOH (40 mL) was stirred for 3 h at 80° C. Desired product could be detected by LCMS. The resulting mixture was concentrated under reduced pressure to afford 1-amino-3-methylurea (2.5 g, 75.74%) as a brown oil. The crude product was used in the next step directly without further purification.

### 4-Methyl-4,5-Dihydro-1H-1,2,4-Triazol-5-One

A solution of 1-amino-3-methylurea (2.5 g, 28.059 mmol, 1 equiv.) in HCOOH (20 mL) was stirred for 36 h at 105° C. Desired product could be detected by LCMS. The resulting solution was concentrated under reduced pressure. The residue was purified by reverse phase flash chromatography with the following conditions: Column: Spherical C₁₈, 20-35 um, 330; Mobile Phase A: water (plus 1.7 mM FA); Mobile Phase B: ACN; Flow rate: 80 mL/min; Gradient: 0%-0% B, 10 min, 0% B-10% B gradient in 10 min; 10% B-30% B gradient in 10 min; 30% B-98% B gradient in 0 min; 98% B-98% B gradient in 5 min; Detector: 220 nm. The fractions containing the desired product were collected at 0% B and concentrated under reduced pressure to afford 4-methyl-4,5-dihydro-1H-1,2,4-triazol-5-one (660 mg, 23.74%) as a white solid.

### 3-BromoMethyl-4,5-Dihydro-1H-1,2,4-Triazol-5-One

To a stirred solution of 4-methyl-4,5-dihydro-1H-1,2,4-triazol-5-one (2.26 g, 22.807 mmol, 1 equiv.) in AcOH (20 mL) was added Br₂ (10.93 g, 68.421 mmol, 3 equiv.) dropwise at room temperature under N₂ atmosphere. The mixture was stirred for 4 h at 60° C. The mixture was allowed to cool down to room temperature. The mixture was basified to pH = 9 with Na₂CO₃ aqueous solution. Desired product could be detected by LCMS. The resulting mixture was extracted with EA (3 × 200 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 330 g; Mobile Phase A: Water (plus 5 mM FA); Mobile Phase B: ACN; Flow rate: 45 mL/min; Gradient: 0% - 5% B, 20 min, 5% B - 5% B gradient in 5 min;5% B - 95% B gradient in 5 min; 95% B - 5% B gradient in 5 min; Detector: 220 nm. The fractions containing the desired product were collected at 5% B) to afford 3-bromo-4-methyl-4,5-dihydro-1H-1,2,4-triazol-5-one (2 g, 49.27%) as an off-white solid.

### D. Ethyl (S)-2-(3-Bromo-1-(1-(4-Chlorophenyl)Ethyl)-5-Oxo-1,5-Dihydro-4h-1,2,4-Triazol-4-Yl)Acetate And Ethyl (R)-2-(3-Bromo-1-(1-(4-Chlorophenyl)Ethyl)-5-Oxo-1,5-Dihydro-4h-1,2,4-Triazol-4-Yl)Acetate

### 1-Bromoethyl)-4-chlorobenzene

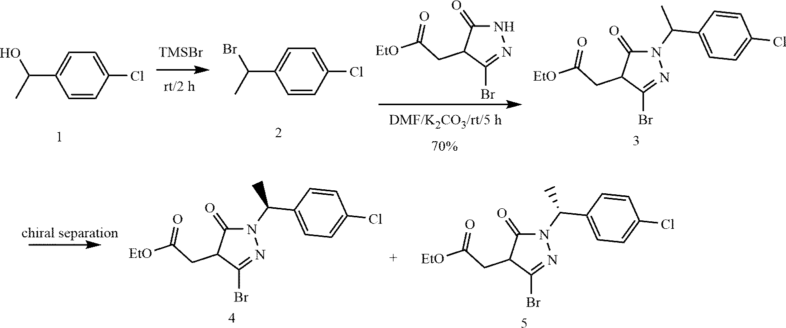

A mixture of 1-(4-chlorophenyl)ethan-1-ol (100.00 mg, 0.639 mmol, 1.00 equiv.) and bromotrimethylsilane (1 mL) was stirred for 3 h at room temperature. Desired product could be detected by LCMS. The reaction was quenched with water (20 mL) at room temperature. The resulting mixture was extracted with EA (3 × 100 mL). The combined organic layers were washed with water (1 × 50 mL), dried over anhydrous Na₂SO₄. After filtration, the filtrate was concentrated under reduced pressure. The residue was purified by Prep-TLC (PE/EA = 2/1) to afford 1-(1-bromoethyl)-4-chlorobenzene (120 mg, 85.61%) as a colorless oil.

### Ethyl (s)-2-(3-bromo-1-(1-(4-Chlorophenyl)Ethyl)-5-Oxo-1,5-Dihydro-4h-1,2,4-Triazol-4-yl)Acetate and Ethyl (r)-2-(3-Bromo-1-(1-(4-chlorophenyl)ethyl)-5-Oxo-1,5-Dihydro-4h-1,2,4-Triazol-4-yl)Acetate

A mixture of ethyl 2-(3-bromo-5-oxo-4,5-dihydro-1H-1,2,4-triazol-4-yl)acetate (700.00 mg, 2.799 mmol, 1.00 equiv.),1-(1-bromoethyl)-4-chlorobenzene (737.40 mg, 3.359 mmol, 1.20 equiv.) and Cs₂CO₃ (1.09 g, 3.359 mmol, 1.20 equiv.) in DMF (20.00 mL) was stirred at room temperature under N₂ atmosphere for 16 hours. The reaction was quenched by the addition of HOAc (2 mL) at room temperature and water (100 mL) was added. The resulting mixture was extracted with EtOAc (200 mL). The combined organic layers were washed with brine (3×50 mL), the filtrate was concentrated under reduced pressure. The residue was purified by reverse flash chromatography with the following conditions: column, C₁₈ silica gel; mobile phase, HCOOH in water, 45% to 55% gradient in 20 min; detector, UV 254 nm, which was delivered for chiral separation with the following condition (Column: CHIRALPAK IG, 20 \*250mm, 5 um; Mobile Phase A:undefined, Mobile Phase B: undefined; Flow rate: 20 mL/min; Gradient: 10 B to 10 B in 18 min; 220/254 nm; RT1:11.087; RT2:13.216) to afford ethyl 2-[3-bromo-1-[1-(4-chlorophenyl)ethyl]-5-oxo-4,5-dihydro-1H-1,2,4-triazol-4-yl]acetate (422.7 mg) as an white oil and ethyl 2-[3-bromo-1-[1-(4-chlorophenyl)ethyl]-5-oxo-4,5-dihydro-1H-1,2,4-triazol-4-yl]acetate (421.5 mg) as an white oil.

### E. Ethyl (2r)-2-[3-Bromo-1-(Cyclohexylmethyl)-5-Oxo-4,5-Dihydro-1h-1,2,4-Triazol-4-yl]Propanoate and Ethyl (2s)-2-[3-Bromo-1-(Cyclohexylmethyl)-5-Oxo-4,5-Dihydro-1h-1,2,4-Triazol-4-yl]Propanoate

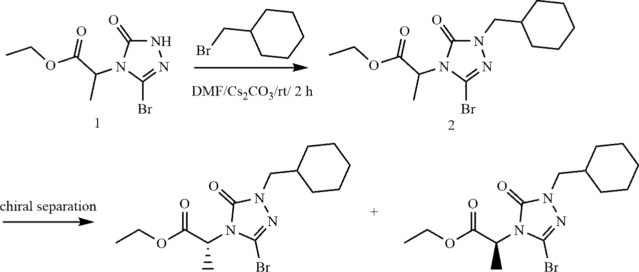

To a stirred solution of ethyl 2-(3-bromo-5-oxo-4,5-dihydro-1H-1,2,4-triazol-4-yl)propanoate (500.00 mg, 1.893 mmol, 1.00 equiv.) and (bromomethyl)cyclohexane (402.35 mg, 2.272 mmol, 1.20 equiv.) in DMF (10.00 mL) was added Cs₂CO₃ (740.28 mg, 2.272 mmol, 1.20 equiv.) at room temperature. The solution was stirred at 60° C. for 4 h. The residue was purified by reverse phase flash chromatography with the following conditions: Column: Spherical C₁₈, 20 - 40 um, 330 g; Mobile Phase A: Water (plus 10 mM NH₄HCO₃); Mobile Phase B: ACN; Flow rate: 80 mL/min; Gradient: 5% - 5% B, 10 min, 70% B - 95% B gradient in 20 min; Detector: 254 nm. The fractions containing the desired product were collected at 82% B and concentrated under reduced pressure to afford ethyl 2-[3-bromo-1-(cyclohexylmethyl)-5-oxo-4,5-dihydro-1H-1,2,4-triazol-4-yl]propanoate (550 mg, 80.63%) as yellow oil. The mixture product (550 mg) was purified by PREP CHIRAL HPLC with the following conditions (Column: CHIRALPAK IE, 2 ^{∗}25cm, 5 umy; Mobile Phase A:Hex(0.2%IPA), Mobile Phase B: EtOH; Flow rate: 20 mL/min; Gradient: 30 B to 30 B in 13.5 min; 220/254 nm; RT1:5.151; RT2:9.185) to afford ethyl (2R)-2-[3-bromo-1-(cyclohexylmethyl)-5-oxo-4,5-dihydro-1H-1,2,4-triazol-4-yl]propanoate (250 mg) as a yellow oil and afford ethyl (2S)-2-[3-bromo-1-(cyclohexylmethyl)-5-oxo-4,5-dihydro-1H-1,2,4-triazol-4-yl]propanoate (260 mg) as a yellow oil.

### F. Ethyl (2R)-2-(3-Bromo-1-[[4-(Morpholin-4-Ylmethyl)Phenyl]Methyl]-5-Oxo-1,2,4-Triazol-4-yl)Propanoate and Ethyl (2S)-2-(3-Bromo-1-[[4-(Morpholin-4-Ylmethyl)Phenyl]Methyl]-5-Oxo-1,2,4-Triazol-4-yl)Propanoate

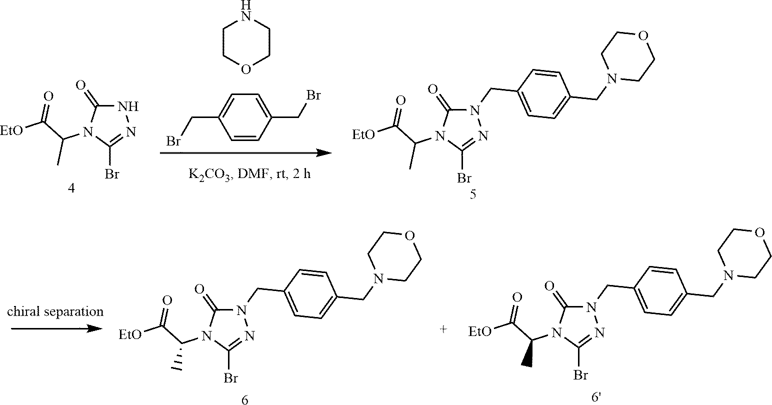

To a stirred mixture of ethyl 2-(3-bromo-5-oxo-1H-1,2,4-triazol-4-yl)propanoate (500.00 mg, 1.893 mmol, 1.00 equiv.) and Cs₂CO₃ (925.35 mg, 2.840 mmol, 1.50 equiv.) in DMF (15.00 mL) was added benzene, 1,4-bis(bromomethyl)- (749.66 mg, 2.840 mmol, 1.50 equiv.) in portions at room temperature under N₂ atmosphere. The mixture was stirred for 2 h at room temperature, and then added morpholine (329.91 mg, 3.787 mmol, 2.00 equiv.) at room temperature. The mixture was stirred for 16 h at room temperature. Desired product could be detected by LCMS. The reaction was quenched with water (100 mL) at room temperature. The resulting mixture was extracted with EA (4 × 150 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 45 mL/min; Gradient: 0% - 5% B, 8 min, 5% B - 20% B gradient in 0 min; 20% B - 80% B gradient in 35 min; 80% B - 95% B gradient in 0 min; 95% B- 95% B gradient in 5 min; Detector: 220 nm. The fractions containing the desired product were collected at 50% B) to afford ethyl-2-(3-bromo-1-[[4-(morpholin-4-ylmethyl)phenyl]methyl]-5-oxo-1,2,4-triazol-4-yl)propanoate (420 mg) as a white solid. The solid was separated by CHIRAL-HPLC with the following conditions (Column: CHIRALPAK IE, 2 ^{∗}25cm, 5 um; Mobile Phase A: HEX:DCM=3:1(0.2%IPA), Mobile Phase B: EtOH; Flow rate: 20 mL/min; Gradient:30% B to 30% B in 9 min; Detector: 220/254 nm; RT1: 5.528 min; RT2: 6.672 min; Injection Volumn:0.3 mL; Number Of Runs:13;) to afford ethyl (2R)-2-(3-bromo-1-[[4-(morpholin-4-ylmethyl)phenyl]methyl]-5-oxo-1,2,4-triazol-4-yl)propanoate (200 mg, 23.30%) as a white solid and ethyl (2S)-2-(3-bromo-1-[[4-(morpholin-4-ylmethyl)phenyl]methyl]-5-oxo-1,2,4-triazol-4-yl)propanoate (198 mg, 23.07%) as a white solid.

### G. (2R)[3-Bromo-]-[(3,4-Dimethoxyphenyl)Methyl]-5-Oxo-1,2,4-Triazol-4-yl]Propanamide and (2S)[3-Bromo-1-[(3,4-Dimethoxyphenyl)Methyl]-5-Oxo-1,2,4-Triazol-4-yl]Propanamide-4,5-Dihydro-1H-1,2,4-Triazol-5-One

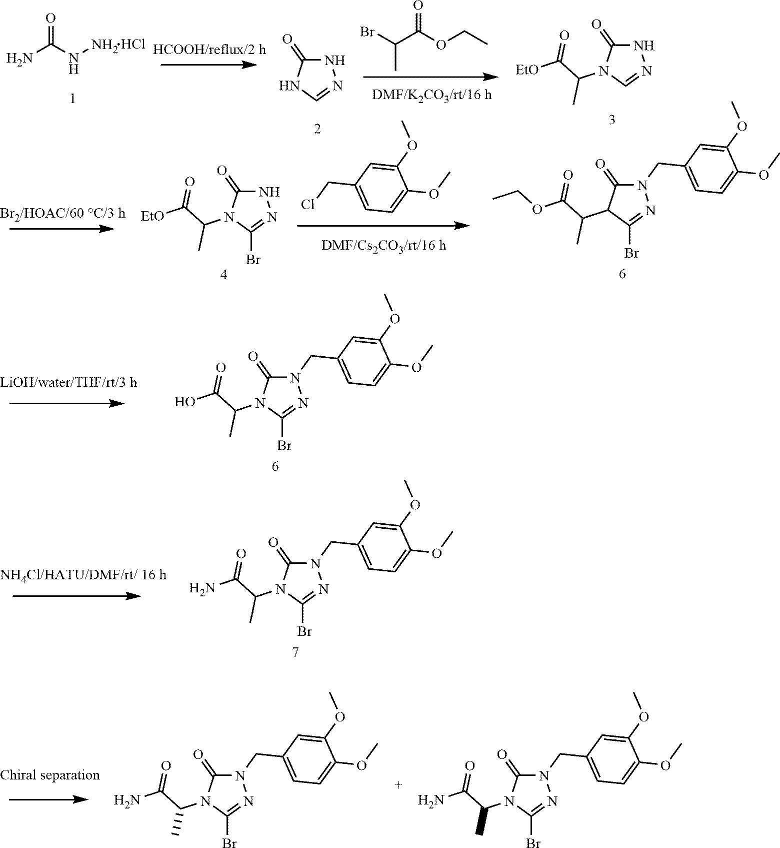

### 4,5-Dihydro-1H-1,2,4-Triazol-5-One

A solution of aminourea hydrochloride (5 g, 44.831 mmol, 1 equiv.) in HCOOH (10 mL) was stirred for 2 h at 110° C. under N₂ atmosphere. The mixture was allowed to cool down to room temperature. The resulting mixture was filtered, the filter cake was washed with EtOH (2 × 5 mL). The filtrate cake was concentrated under reduced pressure to afford 4,5-dihydro-1H-1,2,4-triazol-5-one (3.6 g, 94.40%) as a white solid. The crude product was used in the next step directly without further purification.

### Ethyl 2-(5-Oxo-4,5-Dihydro-1H-1,2,4-Triazol-4-yl)Propanoate

To a stirred mixture of 4,5-dihydro-1H-1,2,4-triazol-5-one (1.88 g, 22.096 mmol, 2.00 equiv.) and K₂CO₃ (3.05 g, 21.875 mmol, 1.98 equiv.) in DMF (8.00 mL) were added the mixture of ethyl 2-bromopropanoate (2.00 g, 11.048 mmol, 1.00 equiv.) and DMF (8.00 mL) dropwise at room temperature under nitrogen atmosphere. The mixture was stirred for 4 h at room temperature. Desired product could be detected by LCMS. The resulting mixture was diluted with 300 mL of water, then adjusted to PH 6-8 with AcOH and extracted with EtOAc (4 × 400 mL). The combined organic layers were washed with water (1×300 mL), dried over anhydrous Na₂SO₄. After filtration, the filtrate was concentrated under reduced pressure. The residue was purified by reverse phase flash chromatography with the following conditions: Column: Spherical C₁₈, 20-35 um, 330; Mobile Phase A: water (plus 1.7 mM FA); Mobile Phase B: ACN; Flow rate: 80 mL/min; Gradient: 0%-0% B, 10 min, 0% B-20% B gradient in 20 min; 98% B-98% B gradient in 8 min; Detector: 220 nm. The fractions containing the desired product were collected at 4% B and concentrated under reduced pressure to afford ethyl 2-(5-oxo-4,5-dihydro-1H-1,2,4-triazol-4-yl)propanoate (1.25 g, 61.10%) as a white solid.

### Ethyl 2-(3-Bromo-5-Oxo-4,5-Dihydro-1H-1,2,4-Triazol-4-yl)Propanoate

To a stirred solution of ethyl 2-(5-oxo-4,5-dihydro-1H-1,2,4-triazol-4-yl)propanoate (2.50 g, 13.500 mmol, 1.00 equiv.) in acetic acid (40 mL) was added Br₂ (1.4 mL) dropwise at 60° C. The solution was stirred for 4 h at 60° C. Desired product could be detected by LCMS. The resulting solution was diluted with 100 mL of EA, then adjusted to PH 7 with saturated Na₂CO₃ (aq.). Then the resulting solution was extracted with EtOAc (4 × 400 mL). The combined organic layers were washed with water (1×400 mL), dried over anhydrous Na₂SO₄. After filtration, the filtrate was concentrated under reduced pressure. The residue was purified by reverse phase flash chromatography with the following conditions: Column: Spherical C₁₈, 20-35 um, 330; Mobile Phase A: water (plus 1.7 mM FA); Mobile Phase B: ACN; Flow rate: 80 mL/min; Gradient: 0%-0% B, 8 min, 20% B-45% B gradient in 20 min; 98% B-98% B gradient in 8 min; Detector: 220 nm. The fractions containing the desired product were collected at 34% B and concentrated under reduced pressure to afford ethyl 2-(3-bromo-5-oxo-4,5-dihydro-1H-1,2,4-triazol-4-yl)propanoate (2.38 g, 66.76%) as a white solid.

### Ethyl[3-Bromo-1-[(4-Chlorophenyl)Methyl]-5-Oxo-4,5-Dihydro-1H-1,2,4-Triazol-4-yl]Propanoate

To a stirred mixture of ethyl 2-(3-bromo-5-oxo-4,5-dihydro-1H-1,2,4-triazol-4-yl)propanoate (800.00 mg, 3.029 mmol, 1.00 equiv.) and Cs₂CO₃ (1.48 g, 4.542 mmol, 1.50 equiv.) in DMF (7 mL) was added the mixture of 1-(bromomethyl)-4-chlorobenzene (747.00 mg, 3.635 mmol, 1.20 equiv.) and DMF (7 mL) dropwise at room temperature. The mixture was stirred for 4 h at room temperature. Desired product could be detected by LCMS. The resulting mixture was diluted with 100 mL of water, then adjusted to pH 7 with AcOH and extracted with EtOAc (4 × 250 mL). The combined organic layers were washed with water (1× 200 mL), dried over anhydrous Na₂SO₄. After filtration, the filtrate was concentrated under reduced pressure. The residue was purified by reverse phase flash chromatography with the following conditions: Column: Spherical C₁₈, 20-35 um, 330; Mobile Phase A: water (plus 1.7 mM FA); Mobile Phase B: ACN; Flow rate: 100 mL/min; Gradient: 0%-0% B, 8 min, 40%-70% B gradient in 25 min; 98%-98% B, 8 min, Detector: 220 nm. The fractions containing the desired product were collected at 57% B and concentrated under reduced pressure to afford ethyl-2-[3-bromo-1-[(4-chlorophenyl)methyl]-5-oxo-4,5-dihydro-1H-1,2,4-triazol-4-yl]propanoate (1.1 g).

### 2-Bromo-1-[(3,4-Dimethoxyphenyl)Methyl]-5-Oxo-1,2,4-Triazol-4-yl]Propanoic Acid

To a stirred mixture of ethyl 2-[3-bromo-1-[(3,4-dimethoxyphenyl)methyl]-5-oxo-1,2,4-triazol-4-yl]propanoate (200.00 mg, 0.483 mmol, 1.00 equiv.) in H₂O (5 mL) and THF (5 mL) was added LiOH (115.62 mg, 4.828 mmol, 10.00 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 200 mL). The combined organic layers were washed with water (1 × 100 mL), dried over anhydrous Na₂SO₄. The organic layers were concentrated under reduced pressure to afford 2-[3-bromo-1-[(3,4-dimethoxyphenyl)methyl]-5-oxo-1,2,4-triazol-4-yl]propanoic acid (160 mg, 85.81%). The crude product was used in the next step directly without further purification.

### (2R)[3-Bromo-1-[(3,4-Dimethoxyphenyl)Methyl]-5-Oxo-1,2,4-Triazol-4-yl]Propanamide and (2S)[3-Bromo-1-[(3,4-Dimethoxyphenyl)Methyl]-5-Oxo-1,2,4-Triazol-4-yl]Propanamide

To a stirred solution of 2-[3-bromo-1-[(3,4-dimethoxyphenyl)methyl]-5-oxo-1,2,4-triazol-4-yl]propanoic acid (910.00 mg, 2.356 mmol, 1.00 equiv.) in DMA (15.00 mL) was added HATU (1343.89 mg, 3.534 mmol, 1.50 equiv.) at room temperature under N₂ atmosphere. The mixture was stirred for 20 min at room temperature. And then the mixture was added NH₄Cl (630.20 mg, 11.781 mmol, 5.00 equiv.) and TEA (715.30 mg, 7.069 mmol, 3.00 equiv.). The mixture was stirred for 8 h at room temperature. Desired product could be detected by LCMS. The mixture was purified by reverse phase flash with the following conditions (Column: Spherical C₁₈, 20 - 40 um, 120 g; Mobile Phase A: Water (plus 5 mM NH₄HCO₃); Mobile Phase B: ACN; Flow rate: 70 mL/min; Gradient: 0% - 5% B, 8 min, 5% B - 10% B gradient in 0 min; 10% B - 80% B gradient in 40 min; 80% B - 95% B gradient in 0 min; 95% - 95% B gradient in 5 min; Detector: 220 nm. The fractions containing the desired product were collected at 35% B) to afford 2-[3-bromo-1-[(3,4-dimethoxyphenyl)methyl]-5-oxo-1,2,4-triazol-4-yl]propanamide (770 mg) as a white solid. The solid was separated by CHIRAL-HPLC with the following conditions (Column: CHIRALPAK IC, 2 ^{∗}25cm, 5 um; Mobile Phase A: Hex, Mobile Phase B: EtOH; Flow rate: 20 mL/min; Gradient:50% B to 50% B in 24 min; Detector: 220/254 nm; RT¹:11.949 min; RT²:19.022 min; Injection Volumn:1.45 mL; Number Of Runs:4;) to afford (2R)-2-[3-bromo-1-[(3,4-dimethoxyphenyl)methyl]-5-oxo-1,2,4-triazol-4-yl]propanamide (318 mg, 35.03%) as a white solid and (2S)-2-[3-bromo-1-[(3,4-dimethoxyphenyl)methyl]-5-oxo-1,2,4-triazol-4-yl]propanamide (350 mg, 38.56%) as a white solid.

### H. Bromo-1-Methyl-5-Oxo-1,2,4-Triazol-4-yl)Methyl 2,2-Dimethylpropanoate

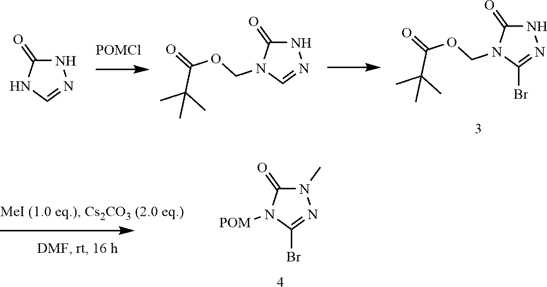

### Oxo-2H-1,2,4-Triazol-4-yl)Methyl 2,2-Dimethylpropanoate

To a stirred mixture of 2,4-dihydro-1,2,4-triazol-3-one (50.00 g, 587.779 mmol, 1.00 equiv.) and DIEA (379.83 g, 2938.878 mmol, 5.00 equiv.) in DMF (500.00 mL) was added POM (42.36 g, 470.223 mmol, 0.8 equiv.) at room temperature. The resulting mixture was stirred for 16 h at 50° C. The reaction was monitored by LCMS. The mixture was allowed to cool down to room temperature. The resulting mixture was extracted with EtOAc (3 × 200 mL). The combined organic layers were washed with brine (3 × 100 mL), dried over anhydrous Na₂SO₄. After filtration, the filtrate was concentrated under reduced pressure. The residue was purified by silica gel column chromatography, eluted with PE/EtOAc (10:1 to 1:1) to afford (3-oxo-2H-1,2,4-triazol-4-yl)methyl 2,2-dimethylpropanoate (20 g, 17.08%) as a white solid.

### Bromo-5-Oxo-1H-1,2,4-Triazol-4-yl)Methyl 2,2-Dimethylpropanoate

To a stirred solution of (3-oxo-2H-1,2,4-triazol-4-yl)methyl 2,2-dimethylpropanoate (1.00 g, 5.020 mmol, 1.00 equiv.) in HFIP (15.00 mL) was added NBS (2.68 g, 15.059 mmol, 3.00 equiv.) at room temperature. The resulting mixture was stirred for 16 h at 60° C. The reaction was monitored by LCMS. The mixture was allowed to cool down to room temperature. The resulting mixture was concentrated under reduced pressure. The residue was purified by silica gel column chromatography, eluted with PE/EtOAc (10:1 to 1:1) to afford (3-bromo-5-oxo-1H-,2,4-triazol-4-yl)methyl 2,2-dimethylpropanoate (130 mg, 9.31%) as a brown solid.

### Bromo-1-Methyl-5-Oxo-1,2,4-Triazol-4-yl)Methyl 2,2-Dimethylpropanoate

To a stirred solution of (3-bromo-5-oxo-1H-1,2,4-triazol-4-yl)methyl 2,2-dimethylpropanoate (130.00 mg, 0.467 mmol, 1.00 equiv.) and K₂CO₃ (129.21 mg, 0.935 mmol, 2.0 equiv.) in DMF (2.00 mL) was added K₂CO₃ (129.21 mg, 0.935 mmol, 2.0 equiv.) at room temperature. The resulting mixture was stirred for 2 h at room temperature. The reaction was monitored by LCMS. The resulting mixture was extracted with CH₂Cl₂ (3 × 20 mL). The combined organic layers were washed with brine (3 × 30 mL), dried over anhydrous Na₂SO₄. After filtration, the filtrate was concentrated under reduced pressure. The residue was purified by silica gel column chromatography, eluted with PE/EtOAc (10:1 to 1:1) to afford (3-bromo-1-methyl-5-oxo-1,2,4-triazol-4-yl)methyl 2,2-dimethylpropanoate (150 mg, 109.84%) as a brown solid.

### Example 2. Preparation of Compound 101

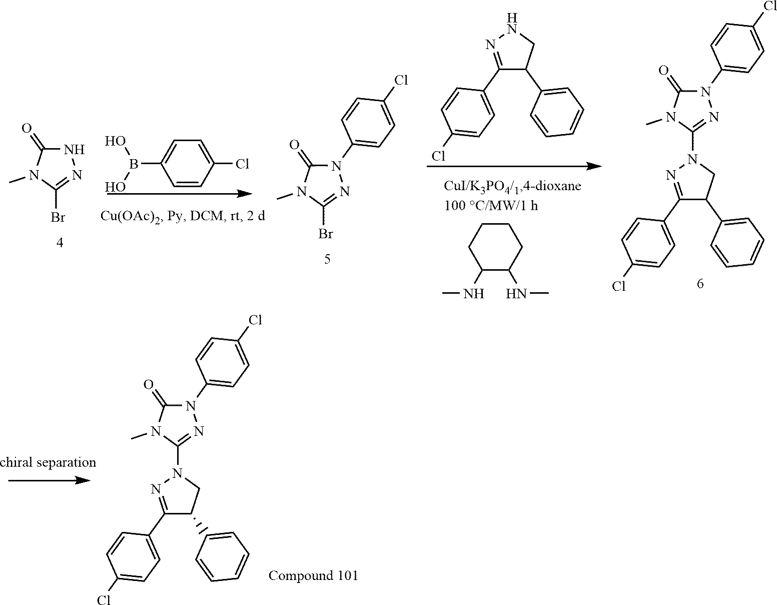

### 3-Bromo(4-Chlorophenyl)-4-Methyl-4,5-Dihydro-1H-1,2,4-Triazol-5-One

A mixture of 3-bromo-4-methyl-4,5-dihydro-1H-1,2,4-triazol-5-one (250 mg, 1.405 mmol, 1 equiv.), (4-chlorophenyl)boronic acid (439.27 mg, 2.809 mmol, 2.00 equiv.), Cu(AcO)₂ (102.05 mg, 0.562 mmol, 0.4 equiv.), and pyridine (333.31 mg, 4.214 mmol, 3 equiv.) in DCE (15 mL) was stirred for 16 h at room temperature in air. Desired product could be detected by LCMS. The reaction was quenched with water at room temperature. The resulting mixture was extracted with DCM (4 × 100 mL). The combined organic layers were washed with water (1 × 100 mL), dried over anhydrous Na₂SO₄. After filtration, the filtrate was concentrated under reduced pressure. The residue was purified by silica gel column chromatography, eluted with PE/EA (3/1) to afford 3-bromo-1-(4-chlorophenyl)-4-methyl-4,5-dihydro-1H-1,2,4-triazol-5-one (190 mg, 46.88%) as an off-white solid.

### 1-Chlorophenyl)-3-[(4S)-3-Chlorophenyl)-4-Phenyl-4,5-Dihydro-1H-Pyrazol-1-yl]-4-Methyl-4,5-Dihydro-1H-1,2,4-Triazol-5-One

A mixture of 3-bromo-1-(4-chlorophenyl)-4-methyl-4,5-dihydro-1H-1,2,4-triazol-5-one (206 mg, 0.714 mmol, 1 equiv.), 3-(4-chlorophenyl)-4-phenyl-4,5-dihydro-1H-pyrazole (183.30 mg, 0.714 mmol, 1.00 equiv.), N₁,N₂-dimethylcyclohexane-1,2-diamine (40.62 mg, 0.286 mmol, 0.40 equiv.), K₃PO₄ (454.65 mg, 2.142 mmol, 3 equiv.), and CuI (27.19 mg, 0.143 mmol, 0.2 equiv.) in dioxane (5 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product (130 mg) was purified by Prep-HPLC with the following conditions (Column: XBridge Shield RP₁₈ OBD Column 30 \*150mm,5 um ;Mobile Phase A:Water(0.1%FA), Mobile Phase B: ACN; Flow rate: 60 mL/min; Gradient: 60% B to 95% B in 7 min; 254/220 nm; Rt: 6.78 min) to afford 1-(4-chlorophenyl)-3-[3-(4-chlorophenyl)-4-phenyl-4,5-dihydro-1H-pyrazol-1-yl]-4-methyl-4,5-dihydro-1H-1,2,4-triazol-5-one (60 mg) as a white solid. The solid was separated by CHIRAL-HPLC with the following conditions (Column: CHIRALPAK IG, 20*250mm, 5 um; Mobile Phase A: Hex., Mobile Phase B: EtOH; Flow rate: 18 mL/min; Gradient: 50% B to 50% B in 25 min; 220/254 nm; RT1:18.801 min; RT2:22.558 min) to afford 1-(4-chlorophenyl)-3-[(4S)-3-(4-chlorophenyl)-4-phenyl-4, 5-dihydro-1H-pyrazol-1-yl]-4-methyl-4, 5-dihydro-1H-1,2,4-triazol-5-one (33 mg, 9.95%) as an off-white solid.

Compound 100 was prepared in a similar manner using the appropriate reagents.

### Example 3. Preparation of Compounds 102 and 103

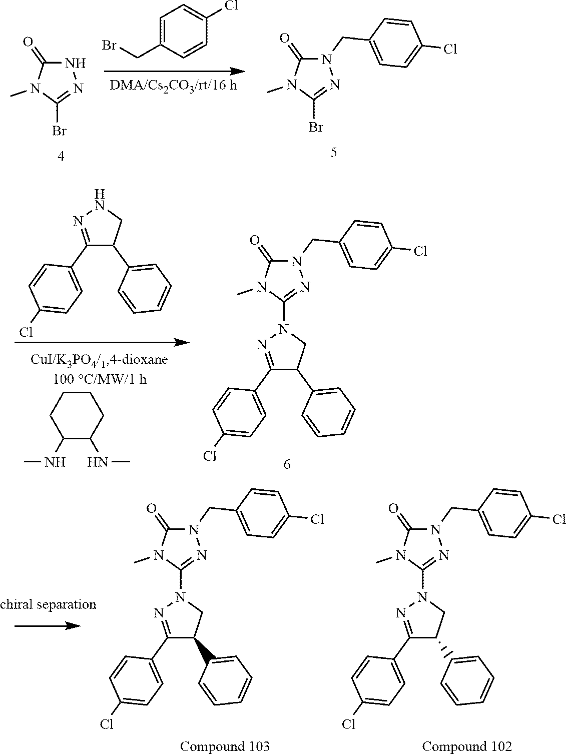

### 3-Bromo[(4-Chlorophenyl)Methyl]-4-Methyl-4,5-Dihydro-1H-1,2,4-Triazol-5-One

To a stirred mixture of 3-bromo-4-methyl-4,5-dihydro-1H-1,2,4-triazol-5-one (120 mg, 0.674 mmol, 1 equiv.) and Cs₂CO₃ (329.50 mg, 1.011 mmol, 1.5 equiv.) in DMF (5 mL) was added 1-(bromomethyl)-4-chlorobenzene (166.24 mg, 0.809 mmol, 1.20 equiv.) in portions at room temperature under N₂ atmosphere. The mixture was stirred for 16 h at room temperature. The reaction was quenched with water at room temperature. The mixture was purified by reverse phase flash with the following conditions (Column: Spherical C₁₈, 20 - 40 um,120 g; Mobile Phase A: Water (plus 5 mM FA); Mobile Phase B: ACN; Flow rate: 65 mL/min; Gradient: 0% - 5% B, 6 min, 5% B - 70% B gradient in 30 min; 70% B -95%B gradient in 0 min; 95% B - 95% B gradient in 5 min; 95% B - 5% B gradient in 5 min; Detector: 220 nm. The fractions containing the desired product were collected at 40% B) to afford 3-bromo-1-[(4-chlorophenyl)methyl]-4-methyl-4,5-dihydro-1H-1,2,4-triazol-5-one (140 mg, 68.63%) as an off-white solid.

### 3-[(4R)(4-Chlorophenyl)-4-Phenyl-4,5-Dihydro-1H-Pyrazol-1-yl]-1-[(4-Chlorophenyl)Methyl]-4-Methyl-4,5-Dihydro-1H-1,2,4-Triazol-5-One and 3-[(4S)(4-Chlorophenyl)-4-Phenyl-4,5-Dihydro-1H-Pyrazol-1-yl]-1-[(4-Chlorophenyl)Methyl]-4-Methyl-4,5-Dihydro-1H-1,2,4-Triazol-5-One

A mixture of 3-bromo-1-[(4-chlorophenyl)methyl]-4-methyl-4,5-dihydro-1H-1,2,4-triazol-5-one (140 mg, 0.463 mmol, 1 equiv.), 3-(4-chlorophenyl)-4-phenyl-4,5-dihydro-1H-pyrazole (142 mg, 0.553 mmol, 1.20 equiv.), N¹,N²-dimethylcyclohexane-1,2-diamine (26.33 mg, 0.185 mmol, 0.4 equiv.), K₃PO₄ (294.66 mg, 1.388 mmol, 3 equiv.), and CuI (17.62 mg, 0.093 mmol, 0.2 equiv.) in dioxane (5 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product (130 mg) was purified by Prep-HPLC with the following conditions (Column: XBridge Shield RP₁₈ OBD Column 30 \*150mm,5 um ;Mobile Phase A:Water(0.1%FA), Mobile Phase B: ACN; Flow rate: 60 mL/min; Gradient: 60% B to 95% B in 7 min; 254/220 nm; Rt: 6.78 min) to afford 3-[3-(4-chlorophenyl)-4-phenyl-4,5-dihydro-1H-pyrazol-1-yl]-1-[(4-chlorophenyl)methyl]-4-methyl-4,5-dihydro-1H-1,2,4-triazol-5-one (75 mg) as a white solid. The solid was separated by CHIRAL-HPLC with the following conditions (Column: CHIRALPAK IC, 2*25cm, 5um; Mobile Phase A: Hex., Mobile Phase B: EtOH; Flow rate: 20 mL/min; Gradient: 30% B to 30% B in 10.5 min; 254/220 nm; RT1:7.558 min ; RT2:8.749 min) to afford 3-[(4R)-3-(4-chlorophenyl)-4-phenyl-4,5-dihydro-1H-pyrazol-1-yl]-1-[(4-chlorophenyl)methyl]-4-methyl-4,5-dihydro-1H-1,2,4-triazol-5-one (15 mg, 6.78%) as a white solid and 3-[(4S)-3-(4-chlorophenyl)-4-phenyl-4,5-dihydro-1H-pyrazol-1-yl]-1-[(4-chlorophenyl)methyl]-4-methyl-4,5-dihydro-1H-1,2,4-triazol-5-one (15 mg, 6.78%) as a white solid.

### Example 4. Preparation of Compounds 104 and 105

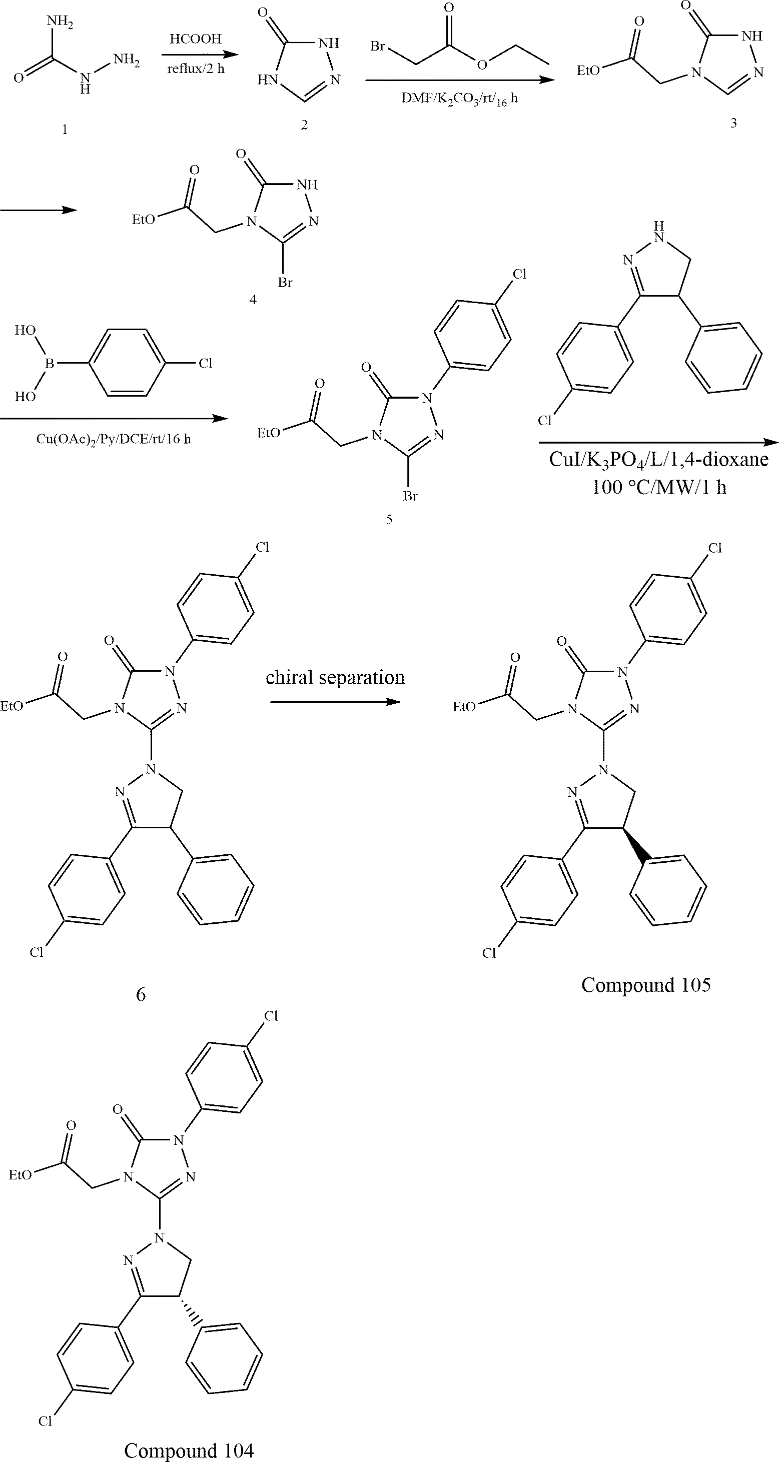

### 4,5-Dihydro-1H-1,2,4-Triazol-5-One

A solution of aminourea hydrochloride (1 g, 8.966 mmol, 1 equiv.) in HCOOH (20 mL) was stirred for 2 h at 110° C. under nitrogen atmosphere. The mixture was allowed to cool down to room temperature. The resulting mixture was concentrated under vacuum. The precipitated solids were collected by filtration and washed with EtOH (3x3 mL). The resulting solid was dried under vacuum. H-NMR analysis indicated the resulting solid was the desired product 4,5-dihydro-1H-1,2,4-triazol-5-one (400 mg, 52.44%)).

### Ethyl 2-(5-Oxo-4,5-Dihydro-1H-1,2,4-Triazol-4-yl)Acetate

To a stirred mixture of 4,5-dihydro-1H-1,2,4-triazol-5-one (3.33 g, 39.146 mmol, 1 equiv.) and K₂CO₃ (8.12 g, 58.719 mmol, 1.5 equiv.) in DMF (40 mL) was added ethyl 2-bromoacetate (7.84 g, 46.975 mmol, 1.20 equiv.) dropwise at room temperature under N₂ atmosphere. The mixture was stirred for 16 h at room temperature. Desired product could be detected by LCMS. The mixture was acidified to pH = 6 with AcOH solution. The resulting mixture was diluted with water (200 mL). The resulting mixture was extracted with EA (3 × 500 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 330 g; Mobile Phase A: Water (plus 5 mM FA); Mobile Phase B: ACN; Flow rate: 80 mL/min; Gradient: 0% - 5% B, 10 min, 5% B - 40% B gradient in 20 min; 40% B - 95% B gradient in 0 min; 95% B - 95% B gradient in 5 min; 95% B - 5% B gradient in 5 min; Detector: 220 nm) to afford ethyl 2-(5-oxo-4,5-dihydro-1H-1,2,4-triazol-4-yl)acetate (2.5 g, 37.31%) as an off-white solid.

### Ethyl 2-(3-Bromo-5-Oxo-4,5-Dihydro-1H-1,2,4-Triazol-4-yl)Acetate

To a stirred solution of ethyl 2-(5-oxo-4,5-dihydro-1H-1,2,4-triazol-4-yl)acetate (226 mg, 1.320 mmol, 1 equiv.) in AcOH (20 mL) was added Br₂ (633.05 mg, 3.961 mmol, 3 equiv.) dropwise at room temperature. The mixture was stirred for 3 h at 60° C. Desired product could be detected by LCMS. The mixture was allowed to cool down to room temperature. The reaction was quenched with water (200 mL) at 0° C. The mixture was basified to pH = 8 with Na₂CO₃ aqueous solution. The resulting mixture was extracted with EA (4 × 200 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 45 mL/min; Gradient: 0% - 35% B, 30 min, 35% B - 95% B gradient in 0 min; 95% B - 95% B gradient in 5 min; 95% B - 5% B gradient in 0 min; 55% B - 5% B gradient in 5 min; Detector: 220 nm. The fractions containing the desired product were collected at 40% B) to afford ethyl 2-(3-bromo-5-oxo-4,5-dihydro-1H-1,2,4-triazol-4-yl)acetate (114 mg, 34.53%) as an off-white solid.

### Ethyl 2-[3-Bromo-1-(4-Chlorophenyl)-5-Oxo-4,5-Dihydro-1H-1,2,4-Triazol-4-yl] Acetate

A mixture of (4-chlorophenyl)boronic acid (500 mg, 3.198 mmol, 2.00 equiv.), ethyl 2-(3-bromo-5-oxo-4,5-dihydro-1H-1,2,4-triazol-4-yl)acetate (400 mg, 1.600 mmol, 1 equiv.), Cu(AcO)₂ (58 mg, 0.319 mmol, 0.20 equiv.), and Pyridine (380 mg, 4.804 mmol, 3.00 equiv.) in DCE (20 mL) was stirred for 4 h under air atmosphere. Desired product could be detected by LCMS. The reaction was quenched with water (100 mL) at room temperature. The resulting mixture was extracted with DCM (3 × 300 mL). The combined organic layers were washed with water (1 × 100 mL), dried over anhydrous Na₂SO₄. After filtration, the filtrate was concentrated under reduced pressure. The residue was purified by silica gel column chromatography, eluted with PE/EA (3/1) to afford ethyl 2-[3-bromo-1-(4-chlorophenyl)-5-oxo-4,5-dihydro-1H-1,2,4-triazol-4-yl]acetate (620 mg, 107.49%) as a white solid.

### Ethyl 2-[1-(4-Chlorophenyl)-3-[(4S)-3-(4-Chlorophenyl)-4-Phenyl-4,5-Dihydro-1H-Pyrazol-1-yl]-5-Oxo-4,5-Dihydro-1H-1,2,4-Triazol-4-yl]Acetate and Ethyl 2-[1-(4-Chlorophenyl)-3-[(4R)-3-(4-Chlorophenyl)-4-Phenyl-4,5-dihydro-1H-pyrazol-1-yl]-5-Oxo-4,5-Dihydro-1H-1,2,4-Triazol-4-yl]Acetate

A mixture of (4S)-3-(4-chlorophenyl)-4-phenyl-4,5-dihydro-1H-pyrazole (256 mg, 0.997 mmol, 1.20 equiv.), ethyl 2-[3-bromo-1-(4-chlorophenyl)-5-oxo-4,5-dihydro-1H-1,2,4-triazol-4-yl]acetate (300 mg, 0.832 mmol, 1 equiv.), N¹,N²-dimethylcyclohexane-1,2-diamine (47.34 mg, 0.333 mmol, 0.4 equiv.), K₃PO₄ (529.80 mg, 2.496 mmol, 3 equiv.), and CuI (21.13 mg, 0.111 mmol, 0.20 equiv.) in dioxane (6 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC with the following conditions (Column: XBridge Shield RP₁₈ OBD Column 30 \*150mm,5 um ;Mobile Phase A:Water(0.1%FA), Mobile Phase B: ACN; Flow rate: 60 mL/min; Gradient: 60% B to 95% B in 20 min; 254/220 nm; Rt: 6.78 min) to afford ethyl 2-(1-(4-chlorophenyl)-3-(3-(4-chlorophenyl)-4-phenyl-4,5-dihydro-1H-pyrazol-1-yl)-5-oxo-1,5-dihydro-4H-1,2,4-triazol-4-yl)acetate (190 mg) as a yellow solid.

The solid was separated by CHIRAL-HPLC with the following conditions (Column: CHIRAL ART Cellulose-SB S-5um, 2 ^{∗}25cm, 5 um; Mobile Phase A: Hex., Mobile Phase B: EtOH; Flow rate: 20 mL/min; Gradient: 50% B to 50% B in 14 min; 220/254 nm; RT1:7.439 min; RT2:12.325 min) to afford ethyl 2-[1-(4-chlorophenyl)-3-[(4S)-3-(4-chlorophenyl)-4-phenyl-4, 5-dihydro-1H-pyrazol-1-yl]-5-oxo-4,5-dihydro-1H-1 ,2,4-triazol-4-yl]acetate (77 mg, 17.25%) as an off-white solid and ethyl 2-[1-(4-chlorophenyl)-3-[(4R)-3-(4-chlorophenyl)-4-phenyl-4,5-dihydro-1H-pyrazol-1-yl]-5-oxo-4,5-dihydro-1H-1,2,4-triazol-4-yl]acetate (97 mg, 21.74%) as a light yellow solid.

### Example 5. Preparation of Compounds 106 and 107

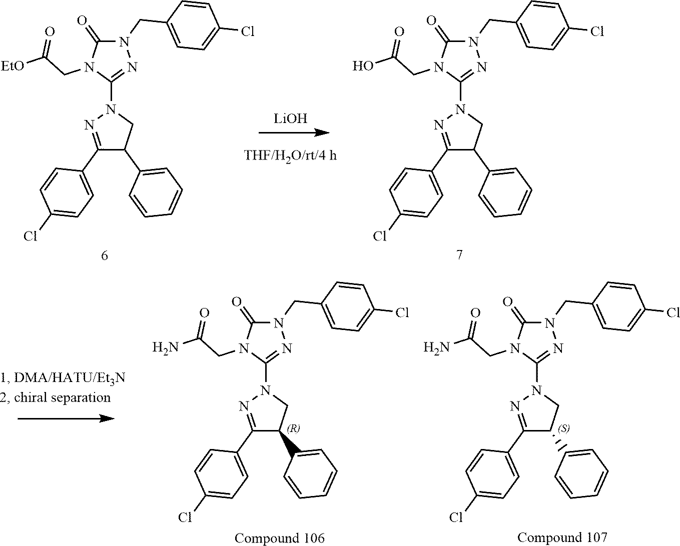

### 2-(4-Chlorophenyl)-4-Phenyl-4,5-Dihydro-1H-Pyrazol-1-yl]-1-[(4-Chlorophenyl)Methyl]-5-Oxo-4,5-Dihydro-1H-1,2,4-Triazol-4-yl]Acetic Acid

To a stirred mixture of ethyl 2-[3-[3-(4-chlorophenyl)-4-phenyl-4,5-dihydro-1H-pyrazol-1-yl]-1-[(4-chlorophenyl)methyl]-5-oxo-4,5-dihydro-1H-1,2,4-triazol-4-yl]acetate (460 mg, 0.836 mmol, 1 equiv.) in H₂O (2 mL) and THF (3 mL) was added LiOH (200.13 mg, 8.357 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 300 mL). The combined organic layers were washed with water (1 × 100 mL), dried over anhydrous Na₂SO₄. After filtration, the filtrate was concentrated under reduced pressure to afford 2-[3-[3-(4-chlorophenyl)-4-phenyl-4,5-dihydro-1H-pyrazol-1-yl]-1-[(4-chlorophenyl)methyl]-5-oxo-4,5-dihydro-1H-,2,4-triazol-4-yl]acetic acid (420 mg, 96.21%) as a yellow solid. The crude product was used in the next step directly without further purification. H-NMR analysis indicated it was the desired product.

### 2-[(4R)-3-(4-Chlorophenyl)-4-Phenyl-4,5-Dihydro-1H-Pyrazol-1-yl]-1-[(4-Chlorophenyl)Methyl]-5-Oxo-4,5-Dihydro-1H-1,2,4-Triazol-4-yl] and 2-[(4S)-3-(4-Chlorophenyl)-4-Phenyl-4,5-Dihydro-1H-Pyrazol-1-yl]-1-[(4-Chlorophenyl)Methyl]-5-Oxo-4,5-Dihydro-1H-1,2,4-Triazol-4-yl]Acetamide

To a stirred solution of 2-[3-[3-(4-chlorophenyl)-4-phenyl-4,5-dihydro-1H-pyrazol-1-yl]-1-[(4-chlorophenyl)methyl]-5-oxo-4,5-dihydro-1H-1,2,4-triazol-4-yl]acetic acid (450 mg, 0.861 mmol, 1 equiv.) in DMA (15 mL) was added HATU (426 mg, 1.120 mmol, 1.30 equiv.) at room temperature. The mixture was stirred for 20 min at room temperature, and added NH₄Cl (138 mg, 2.580 mmol, 2.99 equiv.) and TEA (266 mg, 2.629 mmol, 3.05 equiv.). The mixture was stirred for 4 h at room temperature. Desired product could be detected by LCMS. The reaction was quenched with water (200 mL) at room temperature. The resulting mixture was extracted with EA (3 × 200 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 5 min, 5% B -20% B gradient in 0 min; 20% B - 70% B gradient in 30 min; 70% B - 95% B gradient in 0 min; 95% B - 5% B gradient in 8 min; Detector: 220 nm. The fractions containing the desired product were collected at 50% B) to afford 2-[3-(4-chlorophenyl)-4-phenyl-4, 5-dihydro-1H-pyrazol-1-yl]-1-[(4-chlorophenyl)methyl]-5-oxo-4,5-dihydro-1H-1,2,4-triazol-4-yl]acetamide (380 mg) as a white solid. The solid was separated by CHIRAL-HPLC with the following conditions (Column: CHIRALPAK IC, 2 \*25cm, 5 um;Mobile Phase A: Hex. : DCM = 3/1, Mobile Phase B: EtOH; Flow rate: 20 mL/min; Gradient: 30% B to 30% B in 30 min; 220/254 nm; RT1:19.5 min; RT2:25 min) to afford 2-[3-[(4R)-3-(4-chlorophenyl)-4-phenyl-4,5-dihydro-1H-pyrazol-1-yl]-1-[(4-chlorophenyl)methyl]-5-oxo-4,5-dihydro-1H-1,2,4-triazol-4-yl]acetamide (140.5 mg, 31.28%) as a white solid and 2-[3-[(4S)-3-(4-chlorophenyl)-4-phenyl-4,5-dihydro-1H-pyrazol-1-yl]-1-[(4-chlorophenyl)methyl]-5-oxo-4,5-dihydro-1H-1,2,4-triazol-4-yl]acetamide (140.4 mg, 31.26%) as a white solid.

### Example 6. Preparation of Compound 108

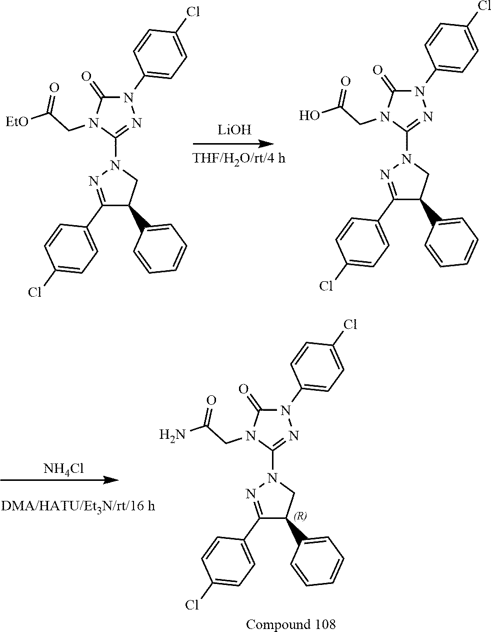

### 2-(4-Chlorophenyl)-3-[(4R)-3-(4-Chlorophenyl)-4-Phenyl-4,5-Dihydro-1H-Pyrazol-1-yl]-5-Oxo-4,5-Dihydro-1H-1,2,4-Triazol-4-yl]Acetic Acid

To a stirred mixture of ethyl 2-[1-(4-chlorophenyl)-3-[(4R)-3-(4-chlorophenyl)-4-phenyl-4,5-dihydro-1H-pyrazol-1-yl]-5-oxo-4,5-dihydro-1H-1,2,4-triazol-4-yl]acetate (90 mg, 0.168 mmol, 1 equiv.) in H₂O (5 mL) and THF (5 mL) was added LiOH (40.18 mg, 1.678 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 300 mL). The combined organic layers were washed with water (1 × 100 mL), dried over anhydrous Na₂SO₄. After filtration, the filtrate was concentrated under reduced pressure to afford 2-[1-(4-chlorophenyl)-3-[(4R)-3-(4-chlorophenyl)-4-phenyl-4,5-dihydro-1H-pyrazol-1-yl]-5-oxo-4,5-dihydro-1H-1,2,4-triazol-4-yl]acetic acid (80 mg, 66.59%) as an off-white solid. The crude product was used in the next step directly without further purification.

### 2-(4-Chlorophenyl)-3-[(4R)-3-(4-Chlorophenyl)-4-Phenyl-4,5-Dihydro-1H-Pyrazol-1-yl]-5-Oxo-4,5-Dihydro-1H-1,2,4-Triazol-4-yl] Acetamide

To a stirred solution of 2-[1-(4-chlorophenyl)-3-[(4R)-3-(4-chlorophenyl)-4-phenyl-4,5-dihydro-1H-pyrazol-1-yl]-5-oxo-4,5-dihydro-1H-1,2,4-triazol-4-yl]acetic acid (60 mg, 0.118 mmol, 1 equiv.) in DMA (8 mL) was added HATU (53.85 mg, 0.142 mmol, 1.2 equiv.) at room temperature. The mixture was stirred for 20 min at room temperature, and added NH₄Cl (18.94 mg, 0.354 mmol, 3 equiv.) and TEA (35.83 mg, 0.354 mmol, 3 equiv.). The mixture was stirred for 4 h at room temperature. Desired product could be detected by LCMS. The reaction was quenched with water (200 mL) at room temperature. The resulting mixture was extracted with EA (3 × 200 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B - 85% B gradient in 40 min; 85% B - 95% B gradient in 5 min; Detector: 220 nm. The fractions containing the desired product were collected at 75% B) to afford 2-[1-(4-chlorophenyl)-3-[(4R)-3-(4-chlorophenyl)-4-phenyl-4, 5-dihydro-1H-pyrazol-1-yl]-5-oxo-4,5-dihydro-1H-1 ,2,4-triazol-4-yl]acetamide (40.2 mg, 67.13%) as an off-white solid.

### Example 7. Preparation of Compound 111

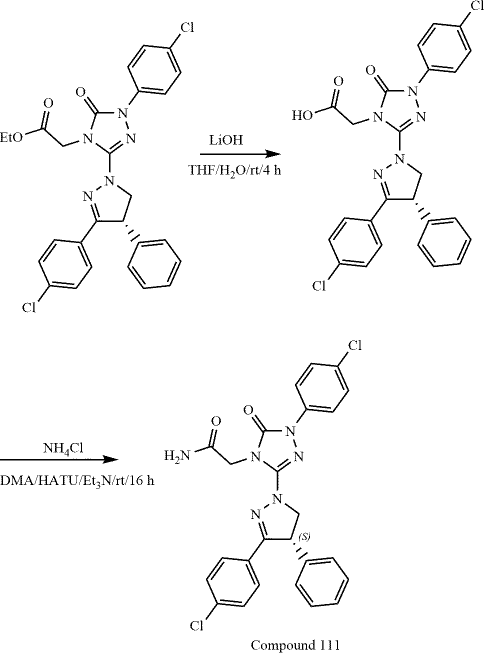

### 2-(4-Chlorophenyl)-3-[(4S)-3-(4-chlorophenyl)-4-Phenyl-4,5-Dihydro-1H-Pyrazol-1-yl]-5-Oxo-4,5-Dihydro-1H-1,2,4-Triazol-4-yl]Acetic Acid

To a stirred mixture of ethyl 2-[1-(4-chlorophenyl)-3-[(4S)-3-(4-chlorophenyl)-4-phenyl-4,5-dihydro-1H-pyrazol-1-yl]-5-oxo-4,5-dihydro-1H-1,2,4-triazol-4-yl]acetate (80 mg, 0.149 mmol, 1 equiv.) in H₂O (5 mL) and THF (5 mL) was added LiOH (35.72 mg, 1.491 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 300 mL). The combined organic layers were washed with water (1 × 100 mL), dried over anhydrous Na₂SO₄. After filtration, the filtrate was concentrated under reduced pressure to afford 2-[1-(4-chlorophenyl)-3-[(4S)-3-(4-chlorophenyl)-4-phenyl-4,5-dihydro-1H-pyrazol-1-yl]-5-oxo-4,5-dihydro-1H-1,2,4-triazol-4-yl]acetic acid (70 mg, 82.17%) as an off-white solid. The crude product was used in the next step directly without further purification.

### (S)(1-(4-Chlorophenyl)-3-(3-(4-Chlorophenyl)-4-Phenyl-4,5-dihydro-1H-Pyrazol-1-yl)-5-Oxo-1,5-Dihydro-4H-1,2,4-Triazol-4-yl)Acetamide

To a stirred solution of 2-[1-(4-chlorophenyl)-3-[(4S)-3-(4-chlorophenyl)-4-phenyl-4,5-dihydro-1H-pyrazol-1-yl]-5-oxo-4,5-dihydro-1H-1,2,4-triazol-4-yl]acetic acid (70.00 mg, 0.138 mmol, 1.00 equiv.) in DMA (8 mL) was added HATU (53.85 mg, 0.142 mmol, 1.2 equiv.) at room temperature. The mixture was stirred for 20 min at room temperature, and added NH₄Cl (18.94 mg, 0.354 mmol, 3 equiv.) and TEA (35.83 mg, 0.354 mmol, 3 equiv.). The mixture was stirred for 4 h at room temperature. Desired product could be detected by LCMS. The reaction was quenched with water (200 mL) at room temperature. The resulting mixture was extracted with EA (3 × 200 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B - 85% B gradient in 40 min; 85% B - 95% B gradient in 5 min; Detector: 220 nm. The fractions containing the desired product were collected at 80% B) to afford (S)-2-(1-(4-chlorophenyl)-3-(3-(4-chlorophenyl)-4-phenyl-4,5-dihydro-1H-pyrazol-1-yl)-5-oxo-1,5-dihydro-4H-1,2,4-triazol-4-yl)acetamide (49 mg, 70.14%) as an off-white solid.

### Example 8. Preparation of Compounds 118 and 121

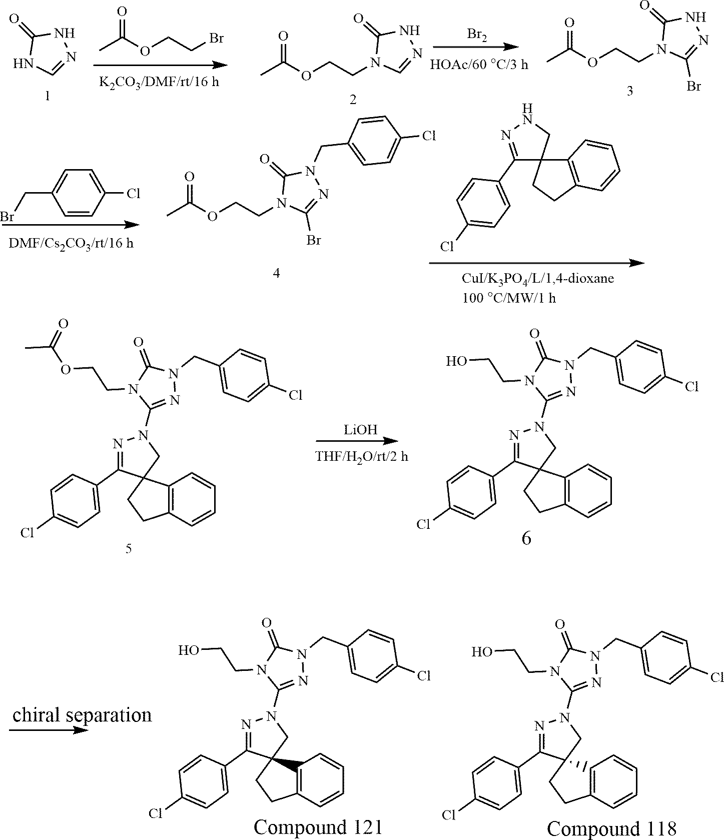

### 2-Oxo-4,5-Dihydro-1H-1,2,4-Triazol-4-yl)Ethyl Acetate

To a stirred mixture of 4,5-dihydro-1H-1,2,4-triazol-5-one (2.00 g, 23.473 mmol, 2 equiv.) and K₂CO₃ (2.43 g, 17.605 mmol, 1.5 equiv.) in DMF (20 mL) was added 2-bromoethyl acetate (1.96 g, 11.736 mmol, 1 equiv.) dropwise at room temperature under N₂ atmosphere. The mixture was stirred for 16 h at room temperature. Desired product could be detected by LCMS. The mixture was acidified to pH = 5 with AcOH solution. The resulting mixture was extracted with EA (4 × 300 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 45 mL/min; Gradient: 0% - 5% B, 20 min, 5% B - 5% B gradient in 8 min;5% B - 95% B gradient in 5 min; 95% B - 5% B gradient in 5 min; Detector: 220 nm. The fractions containing the desired product were collected at 5% B) to afford 2-(5-oxo-4,5-dihydro-1H-1,2,4-triazol-4-yl)ethyl acetate (0.8 g, 39.83%) as a white solid.

### 2-Bromo-5-Oxo-4,5-Dihydro-1H-1,2,4-Triazol-4-yl)Ethyl Acetate

To a stirred solution of 2-(5-oxo-4,5-dihydro-1H-1,2,4-triazol-4-yl)ethyl acetate (0.8 g, 4.674 mmol, 1 equiv.) in AcOH (20 mL) was added Br₂ (1.49 g, 9.324 mmol, 1.99 equiv.) dropwise at room temperature. The mixture was stirred for 3 h at 60° C. The mixture was allowed to cool down to room temperature. The mixture was neutralized to pH =7 with Na₂CO₃ aqueous solution. Desired product could be detected by LCMS. The resulting mixture was extracted with EA (4 × 400 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 20 min, 5% B - 5% B gradient in 8 min; 5% B - 95% B gradient in 5 min; 95% B - 5% B gradient in 5 min; Detector: 220 nm. The fractions containing the desired product were collected at 5% B) to afford 2-(3-bromo-5-oxo-4,5-dihydro-1H-1,2,4-triazol-4-yl)ethyl acetate (0.35 g, 29.95%) as an off-white solid.

### 2-Bromo-1-[(4-Chlorophenyl)Methyl]-5-Oxo-4,5-Dihydro-1H-1,2,4-Triazol-4-yl]Ethyl Acetate

To a stirred mixture of 2-(3-bromo-5-oxo-4,5-dihydro-1H-1,2,4-triazol-4-yl)ethyl acetate (310 mg, 1.240 mmol, 1 equiv.) and Cs₂CO₃ (707 mg, 2.170 mmol, 1.75 equiv.) in DMF (10 mL, 129.218 mmol, 104.23 equiv.) was added 1-(bromomethyl)-4-chlorobenzene (305 mg, 1.484 mmol, 1.20 equiv.) in portions at room temperature under N₂ atmosphere. The mixture was stirred for 16 h at room temperature. Desired product could be detected by LCMS. The reaction was quenched with water (100 mL) at room temperature. The mixture was acidified to pH = 6 with AcOH solution. The resulting mixture was extracted with EA (3 × 100 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 45 mL/min; Gradient: 0% - 5% B, 6 min, 5% B - 25% B gradient in 0 min; 25% B - 80% B gradient in 30 min; 80% B - 95% B gradient in 15 min; Detector: 220 nm. The fractions containing the desired product were collected at 55% B) to afford crude product 2-[3-bromo-1-[(4-chlorophenyl)methyl]-5-oxo-4,5-dihydro-1H-1,2,4-triazol-4-yl]ethyl acetate (430 mg, 77.77%) as a white solid. The crude product was used in the next step directly without further purification.

### 2-(4-Chlorobenzyl)-3-(3′-(4-Chlorophenyl)-2,3-Dihydrospiro[Indene-1,4′-Pyrazol]-1′(5′H)-yl)-5-Oxo-1,5-Dihydro-4H-1,2,4-Triazol-4-yl)Ethyl Acetate

A mixture of 2-[3-bromo-1-[(4-chlorophenyl)methyl]-5-oxo-4,5-dihydro-1H-1,2,4-triazol-4-yl]ethyl acetate (220 mg, 0.587 mmol, 1 equiv.), (1S)-3-(4-chlorophenyl)-1,2,3,5-tetrahydrospiro[indene-1,4-pyrazole] (220 mg, 0.778 mmol, 1.32 equiv.), N₁,N₂-dimethylcyclohexane-1,2-diamine (67 mg, 0.471 mmol, 0.80 equiv.),K₃PO₄ (125 mg, 0.589 mmol, 1.00 equiv.) and CuI (45 mg, 0.236 mmol, 0.40 equiv.) in dioxane (8 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B - 90% B gradient in 30 min; 90% B - 95% B gradient in 5 min; Detector: 220 nm. The fractions containing the desired product were collected at 78% B) to afford 2-(1-(4-chlorobenzyl)-3-(3′-(4-chlorophenyl)-2,3-dihydrospiro[indene-1,4′-pyrazol]-1′(5′H)-yl)-5-oxo-1,5-dihydro-4H-1,2,4-triazol-4-yl)ethyl acetate (88 mg, 25.99%) as a light yellow solid.

### 3-[(1R)(4-Chlorophenyl)-1,2,3,5-Tetrahydrospiro[Indene-1,4-Pyrazol]-1-yl]-1-[(4-Chlorophenyl)Methyl]-4-(2-Hydroxyethyl)-4,5-Dihydro-1H-1,2,4-Triazol-5-One and 3-[(1S)(4-Chlorophenyl)-1,2,3,5-Tetrahydrospiro [Indene-1,4-Pyrazol]-1-yl]-1-[(4-Chlorophenyl)Methyl]-4-(2-Hydroxyethyl)-4,5-Dihydro-1H-1,2,4-Triazol-5-One

A mixture of 2-(1-(4-chlorobenzyl)-3-(3′-(4-chlorophenyl)-2,3-dihydrospiro[indene-1,4′-pyrazol]-1′(5′H)-yl)-5-oxo-1,5-dihydro-4H-1,2,4-triazol-4-yl)ethyl acetate (88 mg, 0.153 mmol, 1 equiv.) and LiOH (36 mg, 1.503 mmol, 9.85 equiv.) in THF (5 mL) and H₂O (5 mL) was stirred for 2 h at room temperature under nitrogen atmosphere. Desired product could be detected by LCMS. The residue was purified by silica gel column chromatography, eluted with PE/EtOAc (1:1) to afford 3-[3-(4-chlorophenyl)-1,2,3,5-tetrahydrospiro[indene-1,4-pyrazol]-1-yl]-1-[(4-chlorophenyl)methyl]-4-(2-hydroxyethyl)-4,5-dihydro-1H-1,2,4-triazol-5-one as a white solid. The mixture was purified by reverse phase flash chromatography with the following conditions: Column: Spherical C₁₈, 20-40 um, 120; Mobile Phase A: water (plus 1.7 mM FA); Mobile Phase B: ACN; Flow rate: 60 mL/min; Gradient: 0%-0% B, 8 min, 0% B-30% B gradient in 0 min; 30% B-90% B gradient in 40 min; 90% B-98% B gradient in 0 min; 98% B-98% B gradient in 6 min; Detector: 220 nm. The fractions containing the desired product were collected at 72% B and concentrated under reduced pressure to afford 3-[3-(4-chlorophenyl)-1,2,3,5-tetrahydrospiro[indene-1,4-pyrazol]-1-yl]-1-[(4-chlorophenyl)methyl]-4-(2-hydroxyethyl)-4,5-dihydro-1H-1,2,4-triazol-5-one as a white solid (60 mg). The mixture (60 mg) was purified by Prep-Chiral-HPLC with the following conditions (Column: CHIRALPAK IG, 2 ^{∗}25cm,5 um;Mobile Phase A: Hexane, Mobile Phase B: EtOH; Flow rate: 20 mL/min; Gradient: 10 B to 10 B in 23 min; 254/220 nm ; RT1:16.961 ; RT2:18.874) to afford 3-[(1R)-3-(4-chlorophenyl)-1,2,3,5-tetrahydrospiro[indene-1,4-pyrazol]-1-yl]-1-[(4-chlorophenyl)methyl]-4-(2-hydroxyethyl)-4,5-dihydro-1H-1,2,4-triazol-5-one (14.6 mg, 17.90%) as an off-white solid and 3-[(1S)-3-(4-chlorophenyl)-1,2,3,5-tetrahydrospiro[indene-1,4-pyrazol]-1-yl]-1-[(4-chlorophenyl)methyl]-4-(2-hydroxyethyl)-4,5-dihydro-1H-1,2,4-triazol-5-one (17.6 mg, 21.57%) as a white solid.

### Example 9. Preparation of Compound 114

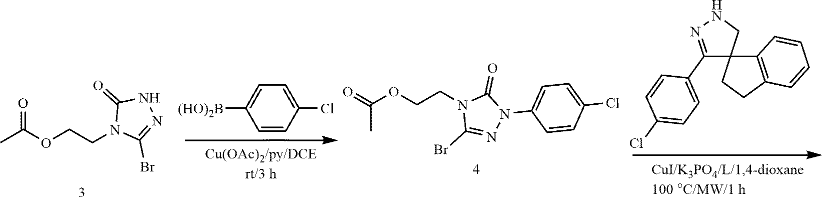

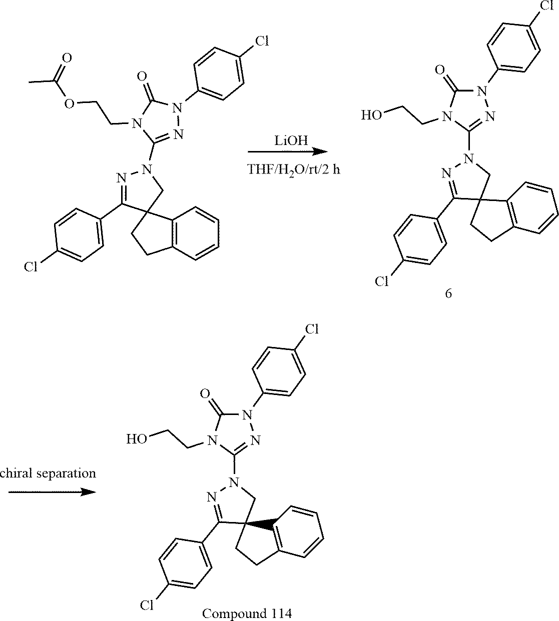

### 2-Bromo-1-(4-Chlorophenyl)-5-Oxo-4,5-Dihydro-1H-1,2,4-Triazol-4-yl]Ethyl Acetate

To a stirred mixture of 2-(3-bromo-5-oxo-4,5-dihydro-1H-1,2,4-triazol-4-yl)ethyl acetate (350 mg, 1.400 mmol, 1 equiv.), (4-chlorophenyl)boronic acid (440 mg, 2.814 mmol, 2.01 equiv.), and Pyridine (333 mg, 4.210 mmol, 3.01 equiv.) in DCE (15 mL) was added Cu(AcO)₂ (51 mg, 0.281 mmol, 0.20 equiv.) at room temperature under air atmosphere. The mixture was stirred for 3 h at room temperature. Desired product could be detected by LCMS. The mixture was purified by silica gel column chromatography, eluted with PE/EA (3/1) to afford 2-[3-bromo-1-(4-chlorophenyl)-5-oxo-4,5-dihydro-1H-1,2,4-triazol-4-yl]ethyl acetate (340 mg, 67.36%) as a white solid.

### 2-(4-Chlorophenyl)-3-[3-(4-Chlorophenyl)-1,2,3,5-Tetrahydrospiro[Indene-1,4-Pyrazol]-1-yl]-5-Oxo-4,5-Dihydro-1H-1,2,4-Triazol-4-yl]Ethyl Acetate

Into a 50 mL sealed tube were added CuI (71.83 mg, 0.377 mmol, 0.4 equiv.) , 2-[3-bromo-1-(4-chlorophenyl)-5-oxo-4,5-dihydro-1H-1,2,4-triazol-4-yl]ethyl acetate (340 mg, 0.943 mmol, 1 equiv.) , 3-(4-chlorophenyl)-1,2,3,5-tetrahydrospiro[indene-1,4-pyrazole] (533.25 mg, 1.886 mmol, 2.00 equiv.), N,N-dimethylcyclohexanamine (95.97 mg, 0.754 mmol, 0.8 equiv.), K₃PO₄ (600.44 mg, 2.829 mmol, 3 equiv.), 1,4-dioxane (10 mL), the tube was charged with N₂, and then the sealed mixture was stirred for 3 hours at 90° C. under nitrogen atmosphere. The resulting mixture was extracted with EtOAc (2 × 50 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (5:1) to afford 2-[1-(4-chlorophenyl)-3-[3-(4-chlorophenyl)-1,2,3,5-tetrahydrospiro[indene-1,4-pyrazol]-1-yl]-5-oxo-4,5-dihydro-1H-1,2,4-triazol-4-yl]ethyl acetate (210 mg, 39.60%) as a light yellow solid.

### 1-chlorophenyl)-3-[(1R)-3-Chlorophenyl)-1,2,3,5-Tetrahydrospiro[Indene-1,4-Pyrazol]-1-yl]-4-(2-Hydroxyethyl)-4,5-Dihydro-1H-1,2,4-Triazol-5-One

To a stirred solution of 2-[1-(4-chlorophenyl)-3-[3-(4-chlorophenyl)-1,2,3,5-tetrahydrospiro[indene-1,4-pyrazol]-1-yl]-5-oxo-4,5-dihydro-1H-1,2,4-triazol-4-yl]ethyl acetate (200 mg, 0.356 mmol, 1 equiv.) in 5 mL THF was added a solution of LiOH (42.58 mg, 1.778 mmol, 5 equiv.) in 5 mL H₂O in portions at room temperature under air atmosphere. The resulting mixture was stirred for 2 h at room temperature. The reaction was monitored by TLC. The resulting mixture was extracted with EtOAc (2 × 100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (5:1) to afford 1-(4-chlorophenyl)-3-[3-(4-chlorophenyl)-1,2,3,5-tetrahydrospiro[indene-1,4-pyrazol]-1-yl]-4-(2-hydroxyethyl)-4,5-dihydro-1H-1,2,4-triazol-5-one (180 mg, 97.27%) as a white solid. The racemic compound (100 mg) was delivered for prep-chiral separation to afford 1-(4-chlorophenyl)-3-[(1R)-3-(4-chlorophenyl)-1,2,3,5-tetrahydrospiro[indene-1,4-pyrazol]-1-yl]-4-(2-hydroxyethyl)-4,5-dihydro-1H-1,2,4-triazol-5-one (31.3 mg, 16.91%) as a light yellow solid.

Compounds 109, 110, 112, and 117 were prepared by similar methods using the appropriate reagents.

### Example 10. Preparation of Compounds 122 and 125

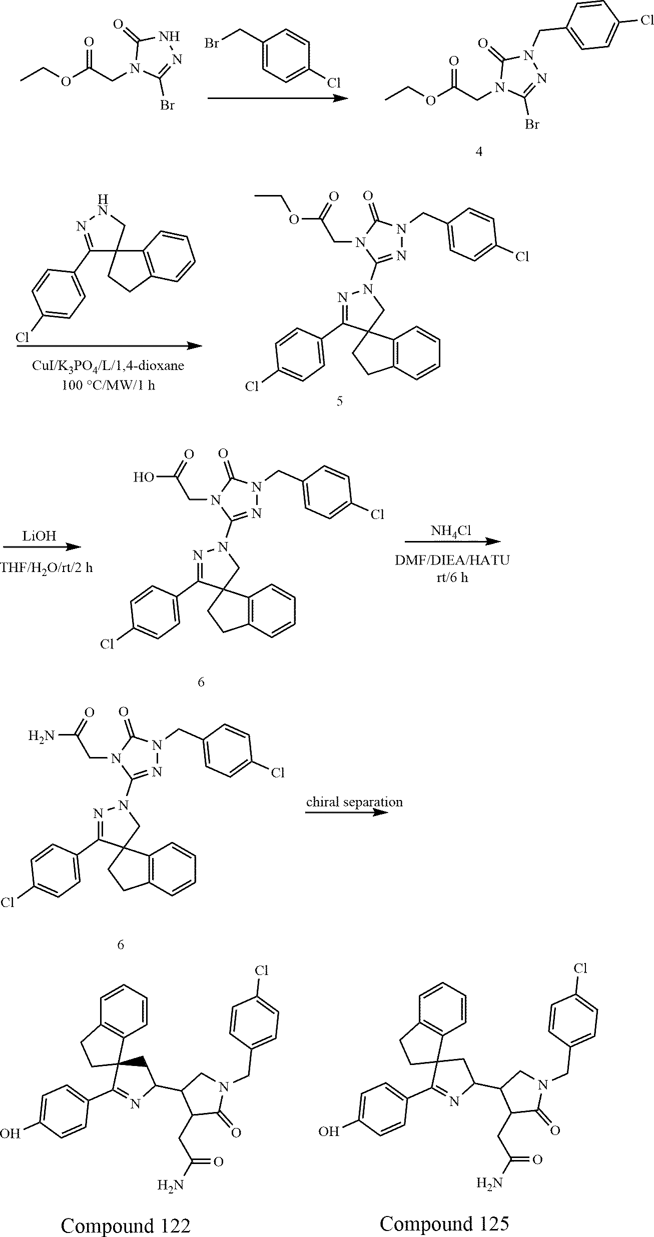

### Ethyl 2-[3-Bromo-1-[(4-Chlorophenyl)Methyl]-5-Oxo-4,5-Dihydro-1H-1,2,4-Triazol-4-yl]Acetate

To a stirred mixture of ethyl 2-(3-bromo-5-oxo-4,5-dihydro-1H-1,2,4-triazol-4-yl)acetate (1.6 g, 6.399 mmol, 1 equiv.) and Cs₂CO₃ (3.65 g, 11.203 mmol, 1.75 equiv.) in DMF (15 mL) was added 1-(bromomethyl)-4-chlorobenzene (1.59 g, 7.738 mmol, 1.21 equiv.) in portions at room temperature under N₂ atmosphere. The mixture was stirred for 16 h at room temperature. Desired product could be detected by LCMS. The mixture was acidified to pH = 5 with AcOH solution. The resulting mixture was diluted with water (100 mL). The resulting mixture was extracted with EA (3 × 400 mL). The combined organic layers were washed with water (2 × 100 mL), dried over anhydrous Na₂SO₄. After filtration, the filtrate was concentrated under reduced pressure. The residue/crude product was purified by reverse phase flash with the following conditions (Column: Spherical C₁₈, 20 - 40 um, 120 g; Mobile Phase A: Water (plus 5 mM FA); Mobile Phase B: ACN; Flow rate: 65 mL/min; Gradient: 0% - 5% B, 20 min, 5% B - 45% B gradient in 0 min; 45% B - 90% B gradient in 30 min; 90% B - 95% B gradient in 0 min; 95% B - 95% B gradient in 5 min; Detector: 220 nm. The fractions containing the desired product were collected at 40% B) to afford ethyl 2-[3-bromo-1-[(4-chlorophenyl)methyl]-5-oxo-4,5-dihydro-1H-1,2,4-triazol-4-yl]acetate (1.96 g, 81.77%) as a white solid.

### Ethyl 2-[3-[3-(4-Chlorophenyl)-1,2,3,5-Tetrahydrospiro[Indene-1,4-Pyrazol]-1-yl]-1-[(4-Chlorophenyl)Methyl]-5-Oxo-4,5-Dihydro-1H-1,2,4-Triazol-4-yl]Acetate

Into a 50 mL sealed tube were added CuI (34.57 mg, 0.182 mmol, 0.40 equiv.), ethyl 2-[3-bromo-1-[(4-chlorophenyl)methyl]-5-oxo-4,5-dihydro-1H-,2,4-triazol-4-yl]acetate170 mg, 0.454 mmol, 1 equiv.), 3-(4-chlorophenyl)-1,2,3,5-tetrahydrospiro[indene-1,4-pyrazole] (256.64 mg, 0.908 mmol, 2.00 equiv.), N,N-dimethylcyclohexanamine (46.19 mg, 0.363 mmol, 0.80 equiv.), K₃PO₄ (288.97 mg, 1.361 mmol, 3.00 equiv.), 1,4-dioxane(5 mL), the tube was charged with N₂, and then the sealed mixture was stirred for 3 hours at 90° C. under nitrogen atmosphere. The resulting mixture was extracted with EtOAc (2 × 50 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (5:1) to afford ethyl 2-[3-[3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]acetate (129 mg,49.31%) as a light brown oil.

### 2-(4-Chlorophenyl)-1,2,3,5-Tetrahydrospiro[Indene-1,4-Pyrazol]-1-yl]-1-[(4-Chlorophenyl)meThyl]-5-Oxo-4,5-Dihydro-1H-1,2,4-triazol-4-yl]Acetic Acid

To a stirred solution of ethyl 2-[3-[3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]acetate (120 mg, 0.208 mmol, 1 equiv.) in 3 mL THF was added a solution of LiOH (24.93 mg, 1.041 mmol, 5 equiv.) in 3 mL H₂O in portions at room temperature under air atmosphere. The resulting mixture was stirred for 2 h at room temperature. The reaction was monitored by LCMS. The mixture was acidified to pH 5 with acetic acid and extracted with EtOAc (2 × 100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (1:1) to afford 2-[3-[3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]acetic acid (110 mg, 96.36%) as a white solid.

### 2-[(1R)-3-(4-Chlorophenyl)-1,2,3,5-Tetrahydrospiro[Indene-1,4-Pyrazol]-1-yl]-1-[(4-Chlorophenyl)Methyl]-5-Oxo-4,5-Dihydro-1H-1,2,4-Triazol-4-yl]Acetamide and 2-[(1S)-3-(4-Chlorophenyl)-1,2,3,5-Tetrahydrospiro [Indene-1,4-Pyrazol]-1-yl]-1-[(4-Chlorophenyl)Methyl]-5-Oxo-4,5-Dihydro-1H-1,2,4-Triazol-4-yl]Acetamide

To a stirred mixture of 2-[3-[3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]acetic acid (110 mg, 0.201 mmol, 1 equiv.) and HATU (114.40 mg, 0.301 mmol, 1.5 equiv.) in DMF was added DIEA (77.77 mg, 0.602 mmol, 3 equiv.) dropwise at room temperature under air atmosphere. 10 minutes later, to the above mixture was added NH₄Cl (21.46 mg, 0.401 mmol, 2 equiv.) in portions. The resulting mixture was stirred for additional 3 h at room temperature. The reaction was monitored by LCMS. The reaction was quenched with saturated NaHCO₃ (aq.). The resulting mixture was extracted with EtOAc (2 × 50 mL). The combined organic layers were washed with water (2×20 mL) and brine (2×20 mL), dried over anhydrous Na₂SO₄. After filtration, the filtrate was concentrated under reduced pressure. The residue was purified by silica gel column chromatography, eluted with PE/EtOAc (5:1) to afford 2-[3-[3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]acetamide (100 mg, 91.07%) as a light yellow solid. The racemic compound was delivered for prep-chiral separation to afford 2-[3-[(1R)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]acetamide (38.4 mg, 34.97%) as a white solid and 2-[3-[(1S)-3-(4-chlorophenyl)-1,2,3, 5-tetrahydrospiro[indene-1,4-pyrazol]-1-yl]-1-[(4-chlorophenyl)methyl]-5-oxo-4,5-dihydro-1H-1,2,4-triazol-4-yl]acetamide (44.3 mg, 40.34%) as an off-white solid.

### Example 11. Preparation of Compounds 120 and 123

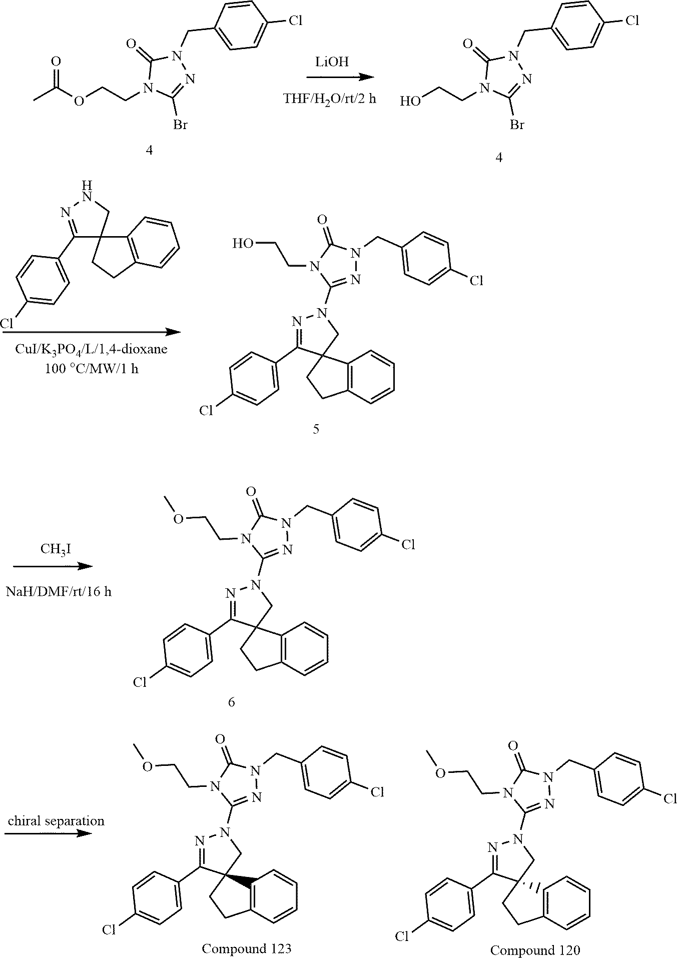

### 3-Bromo[(4-Chlorophenyl)Methyl]-4-(2-Hydroxyethyl)-4,5-Dihydro-1H-1,2,4-Triazol-5-One

To a stirred mixture of 2-[3-bromo-1-[(4-chlorophenyl)methyl]-5-oxo-4,5-dihydro-1H-1,2,4-triazol-4-yl]ethyl acetate (210 mg, 0.561 mmol, 1 equiv.) in H₂O (5 mL) and THF (5 mL) was added LiOH (134.24 mg, 5.606 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 300 mL). The combined organic layers were washed with water (1 × 100 mL), dried over anhydrous Na₂SO₄. After filtration, the filtrate was concentrated under reduced pressure to afford 3-bromo-1-[(4-chlorophenyl)methyl]-4-(2-hydroxyethyl)-4,5-dihydro-1H-1,2,4-triazol-5-one (170 mg, 47.42%) as a white solid. The crude product was used in the next step directly without further purification.

### 3-(4-Chlorophenyl)-1,2,3,5-Tetrahydrospiro[Indene-1,4-Pyrazol]-1-yl]-1-[(4-Chlorophenyl)Methyl]-4-(2-Hydroxyethyl)-4,5-Dihydro-1H-1,2,4-Triazol-5-One

A mixture of 3-bromo-1-[(4-bromophenyl)methyl]-4-(2-hydroxyethyl)-4,5-dihydro-1H-1,2,4-triazol-5-one (170 mg, 0.451 mmol, 1 equiv.), 3-(4-chlorophenyl)-1,2,3,5-tetrahydrospiro[indene-1,4-pyrazole] (191.25 mg, 0.676 mmol, 1.50 equiv.), N₁,N₂-dimethylcyclohexane-1,2-diamine (25.65 mg, 0.180 mmol, 0.40 equiv.), K₃PO₄ (287.12 mg, 1.353 mmol, 3 equiv.), and CuI (17.17 mg, 0.090 mmol, 0.2 equiv.) in dioxane (8 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by reverse phase flash with the following conditions (Column: Spherical C₁₈, 20 -40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B - 90% B gradient in 30 min; 90% B - 95% B gradient in 5 min; Detector: 220 nm. The fractions containing the desired product were collected at 76% B) to afford 3-[3-(4-chlorophenyl)-1,2,3,5-tetrahydrospiro[indene-1,4-pyrazol]-1-yl]-1-[(4-chlorophenyl)methyl]-4-(2-hydroxyethyl)-4,5-dihydro-1H-1,2,4-triazol-5-one (70 mg, 29.05%) as a white solid. 3-[(1R)-3-(4-chlorophenyl)-1,2,3,5-tetrahydrospiro[indene-1,4-pyrazol]-1-yl]-1-[(4-chlorophenyl)methyl]-4-(2-methoxyethyl)-4,5-dihydro-1H-1,2,4-triazol-5-one and 3-[(1S)-3-(4-chlorophenyl)-1,2,3,5-tetrahydrospiro [indene-1,4-pyrazol]-1-yl]-1-[(4-chlorophenyl)methyl]-4-(2-methoxyethyl)-4,5-dihydro-1H-1,2,4-triazol-5-one To a stirred solution of 3-[3-(4-chlorophenyl)-1,2,3,5-tetrahydrospiro[indene-1,4-pyrazol]-1-yl]-1-[(4-chlorophenyl)methyl]-4-(2-hydroxyethyl)-4,5-dihydro-1H-1,2,4-triazol-5-one (70 mg, 0.131 mmol, 1 equiv.) in DMF (10 mL) was added NaH (10.48 mg, 0.262 mmol, 2 equiv, 60%) at 0° C. The mixture was stirred for 30 min at 0° C. The mixture was added MeI (92.95 mg, 0.655 mmol, 5 equiv.). The mixture was stirred for 2 h at room temperature. Desired product could be detected by LCMS. The reaction was quenched with AcOH solution. The mixture was purified by reverse phase to afford 3-[3-(4-chlorophenyl)-1,2,3,5-tetrahydrospiro[indene-1,4-pyrazol]-1-yl]-1-[(4-chlorophenyl)methyl]-4-(2-methoxyethyl)-4,5-dihydro-1H-1,2,4-triazol-5-one (50 mg) as a white solid. The solid was separated by CHIRAL-HPLC with the following conditions (Column: Phenomenex Lux 5u Cellulose-4 AXIA Packed, 2.12 \*25cm, 5 um; Mobile Phase A: Hex., Mobile Phase B:EtOH; Flow rate: 20 mL/min; Gradient: 30% B to 30% B in 20 min; 220/254 nm; RT1:12.568 min; RT2:15.24 min) to afford 3-[(1R)-3-(4-chlorophenyl)-1,2,3,5-tetrahydrospiro[indene-1,4-pyrazol]-1-yl]-1-[(4-chlorophenyl)methyl]-4-(2-methoxyethyl)-4,5-dihydro-1H-1,2,4-triazol-5-one (18 mg, 25.06%) as a white solid and 3-[(1S)-3-(4-chlorophenyl)-1,2,3, 5-tetrahydrospiro[indene-1,4-pyrazol]-1-yl]-1-[(4-chlorophenyl)methyl]-4-(2-methoxyethyl)-4,5-dihydro-1H-1,2,4-triazol-5-one (10 mg, 13.92%).

Compound 113 was prepared in a similar manner using the appropriate reagents.

### Example 12. Preparation of Compound 124

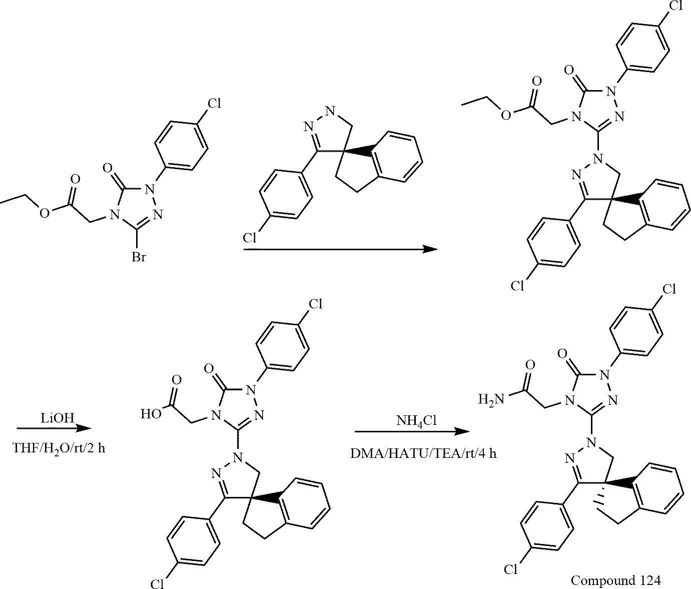

### Ethyl 2-[1-(4-Chlorophenyl)-3-[(1R)-3-(4-Chlorophenyl)-1,2,3,5-Tetrahydrospiro[Indene-1,4-Pyrazol]-1-yl]-5-Oxo-4,5-Dihydro-1H-1,2,4-Triazol-4-yl]Acetate

A mixture of ethyl 2-[3-bromo-1-(4-chlorophenyl)-5-oxo-4,5-dihydro-1H-1,2,4-triazol-4-yl]acetate (380 mg, 1.054 mmol, 1 equiv.), 3-(4-chlorophenyl)-1,2,3,5-tetrahydrospiro[indene-1,4-pyrazole] (446.99 mg, 1.581 mmol, 1.50 equiv.), N₁,N₂-dimethylcyclohexane-1,2-diamine (59.96 mg, 0.422 mmol, 0.40 equiv.), K₃PO₄ (671.08 mg, 3.161 mmol, 3 equiv.) and CuI (40.14 mg, 0.211 mmol, 0.2 equiv.) in dioxane (8 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by reverse phase flash with the following conditions (Column: Spherical C₁₈, 20 -40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B - 90% B gradient in 30 min; 90% B - 95% B gradient in 5 min; Detector: 220 nm. The fractions containing the desired product were collected at 82% B) to afford ethyl 2-[1-(4-chlorophenyl)-3-[3-(4-chlorophenyl)-1,2,3,5-tetrahydrospiro[indene-1,4-pyrazol]-1-yl]-5-oxo-4,5-dihydro-1H-1,2,4-triazol-4-yl]acetate (260 mg) as a white solid. The solid was separated by CHIRAL-HPLC with the following conditions (Column: CHIRAL ART Cellulose-SB, 2 \*25cm, 5 um; Mobile Phase A: Hex.; Mobile Phase B: EtOH.; Flow rate: 20 mL/min; Gradient: 30% B to 30 B% in 12 min; 254/220 nm; RT1:7.02 min; RT2:9.831 min) to afford ethyl 2-[1-(4-chlorophenyl)-3-[(1R)-3-(4-chlorophenyl)-1,2,3,5-tetrahydrospiro[indene-1,4-pyrazol]-1-yl]-5-oxo-4,5-dihydro-1H-1,2,4-triazol-4-yl]acetate (120 mg, 20.25%) as a white solid.

### 2-(4-Chlorophenyl)-3-[(1R)-3-(4-Chlorophenyl)-1,2,3,5-Tetrahydrospiro[Indene-1,4-Pyrazol]-1-yl]-5-Oxo-4,5-Dihydro-1H-1,2,4-Triazol-4-yl]acetic Acid

To a stirred mixture of ethyl 2-[1-(4-chlorophenyl)-3-[(1R)-3-(4-chlorophenyl)-1,2,3,5-tetrahydrospiro[indene-1,4-pyrazol]-1-yl]-5-oxo-4,5-dihydro-1H-1,2,4-triazol-4-yl]acetate (120 mg, 0.213 mmol, 1 equiv.) in H₂O (5 mL) and THF (5 mL) was added LiOH (51.09 mg, 2.134 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 300 mL). The combined organic layers were washed with water (1 × 100 mL), dried over anhydrous Na₂SO₄. After filtration, the filtrate was concentrated under reduced pressure to afford 2-[1-(4-chlorophenyl)-3-[(1R)-3-(4-chlorophenyl)-1,2,3,5-tetrahydrospiro[indene-1,4-pyrazol]-1-yl]-5-oxo-4,5-dihydro-1H-1,2,4-triazol-4-yl]acetic acid (85 mg, 74.55%) as a white solid. The crude product was used in the next step directly without further purification.

### 2-(4-Chlorophenyl)-3-[(1R)-3-(4-Chlorophenyl)-1,2,3,5-Tetrahydrospiro[Indene-1,4-Pyrazol]-1-yl]-5-Oxo-4,5-Dihydro-1H-1,2,4-Triazol-4-yl] Acetamide

To a stirred solution of 2-[1-(4-chlorophenyl)-3-[(1R)-3-(4-chlorophenyl)-1,2,3,5-tetrahydrospiro[indene-1,4-pyrazol]-1-yl]-5-oxo-4,5-dihydro-1H-1,2,4-triazol-4-yl]acetic acid (120 mg, 0.225 mmol, 1 equiv.) in DMA (10 mL) was added HATU (102.46 mg, 0.269 mmol, 1.2 equiv.) at room temperature. The mixture was stirred for 20 min at room temperature, and added NH₄Cl (36.03 mg, 0.674 mmol, 3 equiv.) and TEA (68.17 mg, 0.674 mmol, 3 equiv.). The mixture was stirred for 4 h at room temperature. Desired product could be detected by LCMS. The reaction was quenched with water (200 mL) at room temperature. The resulting mixture was extracted with EA (3 × 200 mL). The combined organic layers were washed with water (1 × 200 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B -85% B gradient in 40 min; 85% B - 95% B gradient in 5 min; Detector: 220 nm. The fractions containing the desired product were collected at 82% B) to afford 2-[1-(4-chlorophenyl)-3-[(1R)-3-(4-chlorophenyl)-1,2,3,5-tetrahydrospiro[indene-1,4-pyrazol]-1-yl]-5-oxo-4,5-dihydro-1H-1,2,4-triazol-4-yl]acetamide (60 mg, 50.09%) as a white solid. Compound 119 was prepared in a similar manner using the appropriate reagents.

### Example 13. Preparation of Compounds 132 and 133

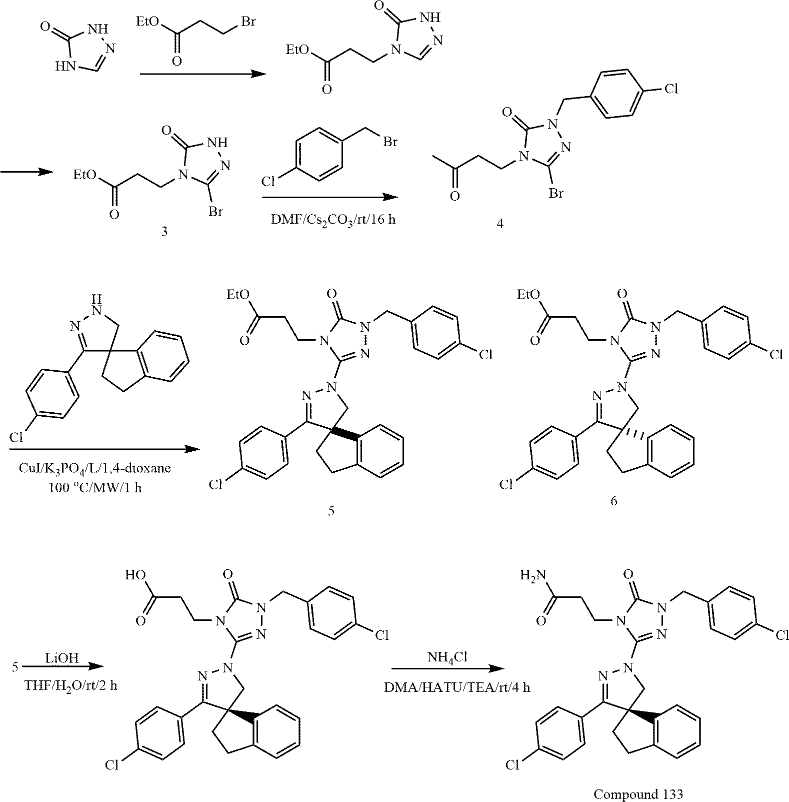

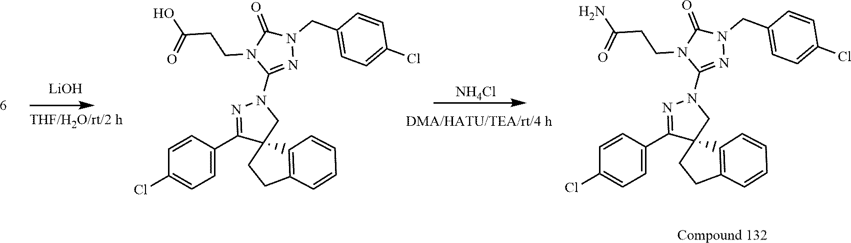

### Ethyl 3-(5-Oxo-4,5-Dihydro-1H-1,2,4-Triazol-4-yl)Propanoate

To a stirred mixture of 4,5-dihydro-1H-1,2,4-triazol-5-one (1.33 g, 2 equiv.) and K₂CO₃ (2.15 g, 2 equiv.) in DMF (13 mL) were added ethyl 3-bromopropanoate (1 mL, 1 equiv.) and DMF (9 mL) dropwise at room temperature under nitrogen atmosphere for 4 h. Desired product could be detected by LCMS. The resulting mixture was diluted with 200 mL of water, then adjusted to PH 6-7 with AcOH. The resulting mixture was extracted with EtOAc (4 × 300 mL). The combined organic layers were washed with Water (1 × 200 mL), dried over anhydrous Na₂SO₄. After filtration, the filtrate was concentrated under reduced pressure. The residue was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 15 min, 0% B-25% B gradient in 15 min, 25% B-25% B gradient in 3 min, 25% B-98% B gradient in 0 min, 98% B-98% B gradient in 6 min; Detector: 220 nm. The fractions containing the desired product were collected at 19% B and concentrated under reduced pressure to afford ethyl 3-(5-oxo-4,5-dihydro-1H-1,2,4-triazol-4-yl)propanoate (1.22 g, 84.72%) as a white solid.

### Ethyl 3-(3-Bromo-5-Oxo-4,5-Dihydro-1H-1,2,4-Triazol-4-yl)Propanoate

To a stirred mixture of ethyl 3-(5-oxo-4,5-dihydro-1H-1,2,4-triazol-4-yl)propanoate (300 mg, 1.620 mmol, 1 equiv.) and AcOH (20 mL) was added Br₂ (0.18 mL, 3.513 mmol, 2.17 equiv.) dropwise at 60° C. The mixture was stirred for 4 h. Desired product could be detected by LCMS. The mixture was allowed to cool down to room temperature. The resulting mixture was diluted with 50 mL of EtOAc, then adjusted to PH 7-8 with saturated Na₂CO₃ (aq.). The resulting mixture was extracted with EtOAc (3 × 200 mL). The combined organic layers were washed with water (1× 150 mL), dried over anhydrous Na₂SO₄. After filtration, the filtrate was concentrated under reduced pressure. The residue was purified by reverse phase flash chromatography with the following conditions: Column: Spherical C₁₈, 20-40 um, 120; Mobile Phase A: water (plus 1.7 mM FA); Mobile Phase B: ACN; Flow rate: 60 mL/min; Gradient: 0%-0% B, 8 min, 0% B-25% B gradient in 25 min; 25% B-98% B gradient in 0 min; 98% B-98% B gradient in 6 min; Detector: 220 nm. The fractions containing the desired product were collected at 20% B and concentrated under reduced pressure to afford ethyl 3-(3-bromo-5-oxo-4,5-dihydro-1H-1,2,4-triazol-4-yl)propanoate (350 mg, 81.81%) as a reddish brown solid. LCMS and H-NMR analysis indicated it was the desired product.

### Ethyl 3-[3-Bromo-1-[(4-Chlorophenyl)Methyl]-5-Oxo-4,5-Dihydro-1H-1,2,4-Triazol-4-yl]Propanoate

To a stirred mixture of ethyl 3-(3-bromo-5-oxo-4,5-dihydro-1H-1,2,4-triazol-4-yl)propanoate (1 g, 3.787 mmol, 1 equiv.) and Cs₂CO₃ (1.85 g, 5.680 mmol, 1.5 equiv.) in DMA (12 mL) was added 1-(bromomethyl)-4-chlorobenzene (0.93 g, 4.544 mmol, 1.20 equiv.) in portions at room temperature under N₂ atmosphere. The mixture was stirred for 12 h at room temperature. Desired product could be detected by LCMS. The reaction was quenched with AcOH solution (2 mL) at room temperature. The resulting mixture was diluted with water (100 mL). The resulting mixture was extracted with EA (4 × 400 mL). The combined organic layers were washed with water (1 × 200 mL), dried over anhydrous Na₂SO₄. After filtration, the filtrate was concentrated under reduced pressure. The crude product was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 30% B gradient in 0 min;30% B - 70% B gradient in 30 min; 70% B - 95% B gradient in 5 min; Detector: 220 nm. The fractions containing the desired product were collected at 55% B) to afford ethyl 3-[3-bromo-1-[(4-chlorophenyl)methyl]-5-oxo-4,5-dihydro-1H-1,2,4-triazol-4-yl]propanoate (1.07 g, 72.70%) as a white solid.

### Ethyl 3-[3-[(1R)-3-(4-Chlorophenyl)-1,2,3,5-Tetrahydrospiro[Indene-1,4-Pyrazol]-1-yl]-1-[(4-Chlorophenyl)Methyl]-5-Oxo-4,5-Dihydro-1H-1,2,4-Triazol-4-yl]Propanoate and Ethyl 3-[3-[(1S)-3-(4-Chlorophenyl)-,2,3,5-Tetrahydrospiro[Indene-1,4-Pyrazol]-1-yl]-1-[(4-Chlorophenyl)Methyl]-5-Oxo-4,5-Dihydro-1H-1,2,4-Triazol-4-yl] Propanoate

A mixture of N₁,N₂-dimethylcyclohexane-1,2-diamine (87.84 mg, 0.618 mmol, 0.4 equiv.), ethyl 3-[3-bromo-1-[(4-chlorophenyl)methyl]-5-oxo-4,5-dihydro-1H-1,2,4-triazol-4-yl]propanoate (600 mg, 1.544 mmol, 1 equiv.), 3-(4-chlorophenyl)-1,2,3,5-tetrahydrospiro[indene-1,4-pyrazole] (654.81 mg, 2.316 mmol, 1.5 equiv.), K₃PO₄ (983.09 mg, 4.631 mmol, 3 equiv.), and CuI (58.80 mg, 0.309 mmol, 0.2 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 400 mL). The combined organic layers were washed with water (1 × 100 mL), dried over anhydrous Na₂SO₄. After filtration, the filtrate was concentrated under reduced pressure. The crude product was purified by reverse phase flash with the following conditions (Column: Spherical C₁₈, 20 -40 um, 120 g; Mobile Phase A: Water (plus 5 mM FA); Mobile Phase B: ACN; Flow rate: 70 mL/min; Gradient: 0% - 5% B, 8 min, 5% B - 35% B gradient in 0 min; 35% B - 90% B gradient in 30 min; 90% B - 95% B gradient in 5 min; Detector: 220 nm. The fractions containing the desired product were collected at 82% B) to afford ethyl 3-[3-[3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoate (240 mg) as a brown solid. The solid was separated by CHIRAL-HPLC with the following conditions (Column: Phenomenex Lux 5u Cellulose-4 AXIA Packed, 2.12 \*25cm, 5 um; Mobile Phase A: Hex., Mobile Phase B: EtOH; Flow rate: 20 mL/min; Gradient: 40% B to 40% B in 16 min; 254/220 nm; RT1:8.734 min; RT2:12.492 min) to afford ethyl 3-[3-[(1R)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoate (110 mg, 12.07%) as a brown solid and ethyl 3-[3-[(1S)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoate (110 mg, 12.07%) as a brown solid.

### 3-[(1R)-3-(4-Chlorophenyl)-,2,3,5-Tetrahydrospiro[Indene-1,4-Pyrazol]-1-yl]-1-[(4-Chlorophenyl)Methyl]-5-Oxo-4,5-Dihydro-1H-1,2,4-Triazol-4-yl]Propanoic Acid

To a stirred mixture of ethyl 3-[3-[(1R)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoate (120 mg, 0.203 mmol, 1 equiv.) in H₂O (10 mL) and THF (10 mL) was added LiOH (48.67 mg, 2.032 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 200 mL). The combined organic layers were washed with water (1 x× 100 mL), dried over anhydrous Na₂SO₄. The organic layers were concentrated under reduced pressure. The residue was purified by reverse phase flash with the following conditions (Column: XBridge Shield RP18 OBD Column, 5 um,19 ^{∗}150mm; Mobile Phase A:undefined, Mobile Phase B: undefined; Flow rate: 25 mL/min; Gradient: 68% B to 72% B in 7 min; 220/254 nm; Rt: 6.5 min) to afford 3-[3-[(1R)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoic acid (80 mg, 69.99%) as a white solid.

### 3-[(1S)-3-(4-Chlorophenyl)-1,2,3,5-Tetrahydrospiro [Indene-1,4-Pyrazol]-1-yl]-1-[(4-Chlorophenyl)Methyl]-5-Oxo-4,5-Dihydro-1H-1,2,4-Triazol-4-yl]Propanoic Acid

To a stirred mixture of ethyl 3-[3-[(1S)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoate (120 mg, 0.203 mmol, 1 equiv.) in H₂O (10 mL) and THF (10 mL) was added LiOH (48.67 mg, 2.032 mmol, 10 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 200 mL). The combined organic layers were washed with water (1 × 100 mL), dried over anhydrous Na₂SO₄. The organic layers were concentrated under reduced pressure. The residue was purified by reverse phase flash with the following conditions (Column: XBridge Shield RP18 OBD Column, 5 um,19 \*150mm; Mobile Phase A: undefined, Mobile Phase B: undefined; Flow rate: 25 mL/min; Gradient: 74% B to 82% B in 7 min; 220/254 nm; Rt: 6.5 min) to afford 3-[3-[(1S)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoic acid (80 mg, 69.99%) as a white solid.

### 3-[(1R)-3-(4-Chlorophenyl)-1,2,3,5-Tetrahydrospiro [Indene-1,4-Pyrazol]-1-yl]-1-[(4-Chlorophenyl)Methyl]-5-Oxo-4,5-Dihydro-1H-1,2,4-Triazol-4-yl]Propanamide

A solution of 3-[3-[(1R)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoic acid (80 mg, 0.142 mmol, 1 equiv.) and HATU (81 mg, 0.213 mmol, 1.50 equiv.) in DMA (8 mL) was stirred for 20 min at room temperature. Then the solution was added NH₄Cl (23 mg, 0.430 mmol, 3.02 equiv.) and TEA (44 mg, 0.435 mmol, 3.06 equiv.) at room temperature. The mixture was stirred for 4 h at room temperature. Desired product could be detected by LCMS. The resulting mixture was diluted with 50 mL of water, then extracted with EtOAc (4 × 100 mL). The combined organic layers were washed with water (1× 50 mL), dried over anhydrous Na₂SO₄. After filtration, the filtrate was concentrated under reduced pressure. The crude product was purified by Prep-HPLC with the following conditions (Column: XBridge Shield RP18 OBD Column, 5 um, 19 ^{∗}150mm; Mobile Phase A:undefined, Mobile Phase B: undefined; Flow rate: 25 mL/min; Gradient: 60% B to 65% B in 7 min; 220 nm; Rt: 6.23 min) to afford 3-[3-[(1R)-3-(4-chlorophenyl)-1,2,3,5-tetrahydrospiro[indene-1,4-pyrazol]-1-yl]-1-[(4-chlorophenyl)methyl]-5-oxo-4, 5-dihydro-1H-1,2,4-triazol-4-yl]propanamide (41.2 mg, 51.59%) as a white solid.

### 3-[(1S)-3-(4-Chlorophenyl)-1,2,3,5-Tetrahydrospiro [Indene-1,4-Pyrazol]-1-yl]-1-[(4-Chlorophenyl)Methyl]-5-Oxo-4,5-Dihydro-1H-1,2,4-Triazol-4-yl]Propanamide

A solution of 3-[3-[(1S)-3-(4-chlorophenyl)-1,2,3,5-tetrahydrospiro[indene-1,4-pyrazol]-1-yl]-1-[(4-chlorophenyl)methyl]-5-oxo-4,5-dihydro-1H-1,2,4-triazol-4-yl]propanoic acid (80 mg, 0.142 mmol, 1 equiv.) and HATU (81 mg, 0.213 mmol, 1.50 equiv.) in DMA (8 mL) was stirred for 20 min at room temperature. Then the solution was added NH₄Cl (23 mg, 0.430 mmol, 3.02 equiv.) and TEA (44 mg, 0.435 mmol, 3.06 equiv.) at room temperature. The mixture was stirred for 4 h at room temperature. Desired product could be detected by LCMS. The resulting mixture was diluted with 50 mL of water, then extracted with EtOAc (4 × 100 mL). The combined organic layers were washed with water (1x 50 mL), dried over anhydrous Na₂SO₄. After filtration, the filtrate was concentrated under reduced pressure. The crude product was purified by Prep-HPLC with the following conditions (Column: XBridge Shield RP₁₈ OBD Column, 5 um, 19 ^{∗}150mm; Mobile Phase A:undefined, Mobile Phase B: undefined; Flow rate: 25 mL/min; Gradient: 60% B to 65% B in 7 min; 220 nm; Rt: 6.18 min) to afford 3-[3-[(1S)-3-(4-chlorophenyl)-1,2,3,5-tetrahydrospiro[indene-1,4-pyrazol]-1-yl]-1-[(4-chlorophenyl)methyl]-5-oxo-4, 5-dihydro-1H-1,2,4-triazol-4-yl]propanamide (39.7 mg, 49.71%) as a white solid.

Compounds 127 and 131 were prepared in a similar manner using the appropriate reagents.

### Example 14. Preparation of Compound 126

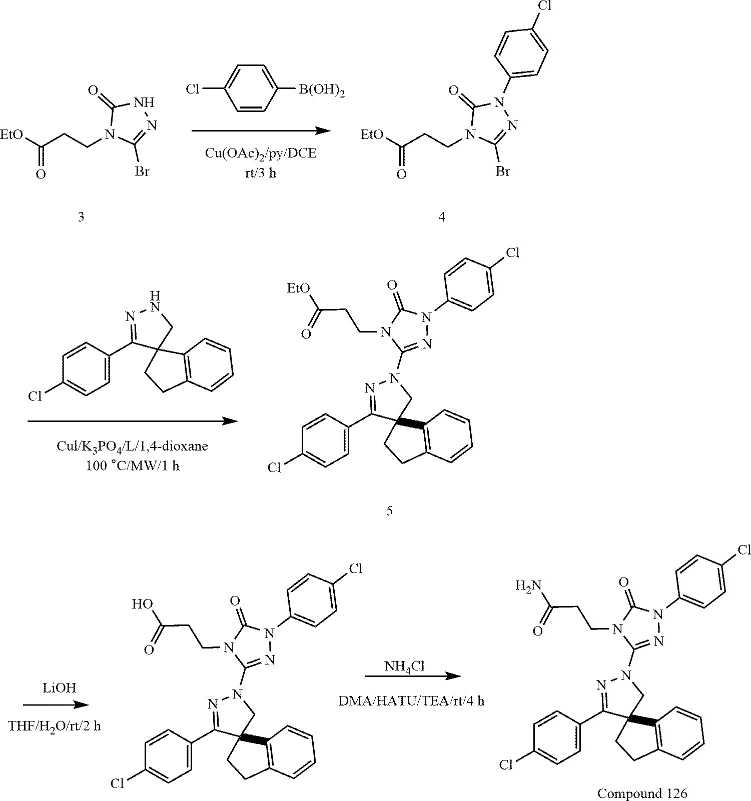

### Ethyl 3-[3-Bromo-1-(4-Chlorophenyl)-5-Oxo-4,5-Dihydro-1H-1,2,4-Triazol-4-yl]Propanoate

To a stirred mixture of ethyl 3-(3-bromo-5-oxo-4,5-dihydro-1H-1,2,4-triazol-4-yl)propanoate (1 g, 3.787 mmol, 1 equiv.), (4-chlorophenyl)boronic acid (1.18 g, 7.573 mmol, 2.00 equiv.), and Pyridine (0.90 g, 11.360 mmol, 3 equiv.) in DCE (20 mL) was added Cu(AcO)₂ (0.28 g, 1.515 mmol, 0.4 equiv.) at room temperature under air atmosphere. The mixture was stirred for 3 h at room temperature. Desired product could be detected by LCMS. The mixture was purified by silica gel column chromatography, eluted with PE/EA (4/1) to afford ethyl 3-[3-bromo-1-(4-chlorophenyl)-5-oxo-4,5-dihydro-1H-1,2,4-triazol-4-yl]propanoate (1.27 g, 89.53%) as a white solid.

### Ethyl 3-[1-(4-Chlorophenyl)-3-[(1R)-3-(4-Chlorophenyl)-1,2,3,5-Tetrahydrospiro[Indene-1,4-Pyrazol]-1-yl]-5-Oxo-4,5-Dihydro-1H-1,2,4-Triazol-4-yl]Propanoate

Into a 50 mL sealed tube were added ethyl 3-[3-bromo-1-(4-chlorophenyl)-5-oxo-4,5-dihydro-1H-1,2,4-triazol-4-yl]propanoate (100 mg, 0.267 mmol, 1 equiv.), 3-(4-chlorophenyl)-1,2,3,5-tetrahydrospiro[indene-1,4-pyrazole] (150.96 mg, 0.534 mmol, 2.00 equiv.), Pd₂(dba)₃ (24.44 mg, 0.027 mmol, 0.1 equiv.), XantPhos (30.89 mg, 0.053 mmol, 0.2 equiv.), Cs₂CO₃ (173.95 mg, 0.534 mmol, 2.00 equiv.) and dioxane (3 mL, 35.412 mmol, 132.66 equiv.) at room temperature. The final reaction mixture was irradiated with microwave radiation for 4h at 120° C. The resulting mixture was diluted with ethyl acetate (10 mL). The resulting mixture was extracted with EtOAc (2 × 20 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EtOAc (10:1) to afford ethyl 3-[1-(4-chlorophenyl)-3-[3-(4-chlorophenyl)-1,2,3,5-tetrahydrospiro[indene-1,4-pyrazol]-1-yl]-5-oxo-4,5-dihydro-1H-1,2,4-triazol-4-yl]propanoate (110 mg, 71.48%) as an off-white solid. The racemic compound was delivered for prep-chiral separation with the following condition (Column: CHIRALPAK IG, 20 \*250mm,5 um;Mobile Phase A:Hexane, Mobile Phase B: EtOH; Flow rate: 20 mL/min; Gradient: 30 B to 30 B in 20 min; 220/254 nm; RT1:12.373; RT2:15.194) to afford ethyl 3-[1-(4-chlorophenyl)-3-[(1R)-3-(4-chlorophenyl)-1,2,3,5-tetrahydrospiro[indene-1,4-pyrazol]-1-yl ]-5-oxo-4,5-dihydro-1H-1,2,4-triazol-4-yl]propanoate (44 mg, 28.59%) as an off-white solid

### 3-(4-Chlorophenyl)-3-[(1R)-3-(4-Chlorophenyl)-1,2,3,5-Tetrahydrospiro [Indene-1,4-Pyrazol]-1-yl]-5-Oxo-4,5-Dihydro-1H-1,2,4-Triazol-4-yl]Propanoic Acid

To a stirred solution of ethyl 3-[1-(4-chlorophenyl)-3-[(1R)-3-(4-chlorophenyl)-1,2,3,5-tetrahydrospiro[indene-1,4-pyrazol]-1-yl ]-5-oxo-4,5-dihydro-1H-1,2,4-triazol-4-yl]propanoate (44 mg, 0.076 mmol, 1 equiv.) in 3 mL THF and 3 mL H₂O was added LiOH (18.28 mg, 0.763 mmol, 10 equiv.) in portions at room temperature under air atmosphere. The resulting mixture was stirred for 3 h at room temperature under air atmosphere. The reaction was monitored by TLC. When the reaction is done, the mixture was acidified to pH 4 with acetic acid. The resulting mixture was extracted with EtOAc (2 × 50 mL). The combined organic layers were washed with brine (2×10 mL), dried over anhydrous Na₂SO₄. After filtration, the filtrate was concentrated under reduced pressure. This resulted in 3-[1-(4-chlorophenyl)-3-[(1R)-3-(4-chlorophenyl)-1,2,3, 5-tetrahydrospiro[indene-1,4-pyrazol]-1-yl]-5-oxo-4,5-dihydro-1H-1,2,4-triazol-4-yl]propanoic acid (37 mg, 88.39%) as a light yellow oil. The crude product was used in the next step directly without further purification.

### 3-(4-Chlorophenyl)-3-[(1R)-3-(4-Chlorophenyl)-1,2,3,5-Tetrahydrospiro [Indene-1,4-Pyrazol]-1-yl]-5-Oxo-4,5-Dihydro-1H-1,2,4-Triazol-4-yl] Propanamide

To a stirred mixture of 3-[1-(4-chlorophenyl)-3-[(1R)-3-(4-chlorophenyl)-1,2,3,5-tetrahydrospiro[indene-1 ,4-pyrazol]-1-yl ]-5-oxo-4,5-dihydro-1H-1,2,4-triazol-4-yl]propanoic acid (37 mg, 0.067 mmol, 1 equiv.) and HATU (38.48 mg, 0.101 mmol, 1.50 equiv.) in DMF were added DIEA (21.80 mg, 0.169 mmol, 2.50 equiv.) and NH₄Cl (7.22 mg, 0.135 mmol, 2.00 equiv.) in portions at room temperature under air atmosphere. The resulting mixture was stirred for 3 h at room temperature under air atmosphere. The reaction was monitored by LCMS. The residue was purified by reverse flash chromatography with the following conditions: column, C₁₈ spherical 120 g; mobile phase, acetonitrile in water, 80% to 90% gradient in 10 min; detector, UV 220/254 nm. This resulted in 3-[1-(4-chlorophenyl)-3-[(1R)-3-(4-chlorophenyl)-1,2,3,5-tetrahydrospiro[indene-1,4-pyrazol]-1-yl ]-5-oxo-4,5-dihydro-1H-1,2,4-triazol-4-yl]propanamide (27.8 mg, 75.27%) as a white solid.

Compounds 128, 129, and 130 were prepared in a similar manner using the appropriate reagents.

### Example 15. Preparation of Compound 136

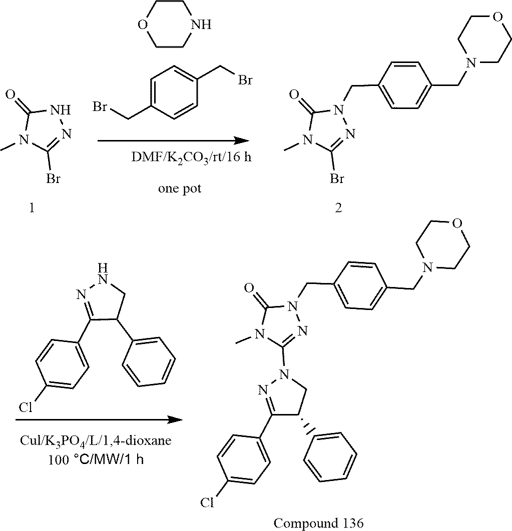

### 3-BromoMethyl-1-([4-[(Morpholinyl)Methyl]Phenyl]methyl)-4,5-dihydro-1H-1,2,4-Triazol-5-One

To a stirred mixture of 3-bromo-4-methyl-4,5-dihydro-1H-1,2,4-triazol-5-one (0.50 g, 2.809 mmol, 1.00 equiv.) and Cs₂CO₃ (1.37 g, 4.214 mmol, 1.50 equiv.) in DMA (10.00 mL) was added 1,4-bis(bromomethyl)benzene (1.12 g, 4.243 mmol, 1.51 equiv.) in portions at room temperature under N₂ atmosphere. The mixture was stirred for 2 h at room temperature, and then added morpholine (0.49 g, 5.618 mmol, 2.00 equiv.) at room temperature. The mixture was stirred for 16 h at room temperature. Desired product could be detected by LCMS. The reaction was quenched with water (100 mL) at room temperature. The resulting mixture was extracted with EA (4 × 150 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 45 mL/min; Gradient: 0% - 5% B, 8 min, 5% B - 20% B gradient in 0 min; 20% B - 80% B gradient in 35 min; 80% B - 95% B gradient in 0 min; 95% B- 95% B gradient in 5 min; Detector: 220 nm. The fractions containing the desired product were collected at 50% B) to afford 3-bromo-4-methyl-1-([4-[(morpholin-4-yl)methyl]phenyl]methyl)-4,5-dihydro-1H-1,2,4-triazol-5-one (0.53 g, 51.37%) as a colorless oil.

### 3-[(4S)(4-Chlorophenyl)-4-Phenyl-4,5-Dihydro-1H-Pyrazol-1-yl]-4-Methyl-1-([4-[(Morpholin-4-yl)Methyl]Phenyl]Methyl)-4,5-Dihydro-1H-1,2,4-Triazol-5-One

A mixture of 3-bromo-4-methyl-1-([4-[(morpholin-4-yl)methyl]phenyl]methyl)-4,5-dihydro-1H-1,2,4-triazol-5-one (290.00 mg, 0.790 mmol, 1.00 equiv.), 3-(4-chlorophenyl)-4-phenyl-4,5-dihydro-1H-pyrazole (405.46 mg, 1.579 mmol, 2.00 equiv.), N¹,N²-dimethylcyclohexane-1,2-diamine (44.93 mg, 0.316 mmol, 0.40 equiv.), K₃PO₄ (502.85 mg, 2.369 mmol, 3.00 equiv.), and CuI (30.08 mg, 0.158 mmol, 0.20 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 ×x 100 mL), dried over anhydrous Na₂SO₄. After filtration, the filtrate was concentrated under reduced pressure. The crude product was purified by Prep-HPLC with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 25% B gradient in 0 min; 25% B - 80% B gradient in 35 min; 80% B - 95% B gradient in 0 min; 95% B - 95% B gradient in 5 min; Detector: 220 nm. The fractions containing the desired product were collected at 50% B) to afford 3-[3-(4-chlorophenyl)-4-phenyl-4,5-dihydro-1H-pyrazol-1-yl]-4-methyl-1-([4-[(morpholin-4-yl)methyl]phenyl]methyl)-4,5-dihydro-1H-1,2,4-triazol-5-one (100 mg) as a white solid. The solid was separated by CHIRAL-HPLC with the following conditions (Column: CHIRALPAK IC, 2 ^{∗}25cm,5 um;Mobile Phase A: undefined, Mobile Phase B: undefined; Flow rate: 20 mL/min; Gradient: 30 B to 30 B in 29 min; 254/220 nm; RT1:22.358; RT2:24.978) to afford 3-[(4S)-3-(4-chlorophenyl)-4-phenyl-4,5-dihydro-1H-pyrazol-1-yl]-4-methyl-1-([4-[(morpholin-4-yl)methyl]phenyl]methyl)-4,5-dihydro-1H-1,2,4-triazol-5-one (30 mg, 7.00%) as a white solid.

Compounds 134, 135, 137, and 146 were prepared in a similar manner using the appropriate reagents.

### Example 16. Preparation of Compounds 139 and 140

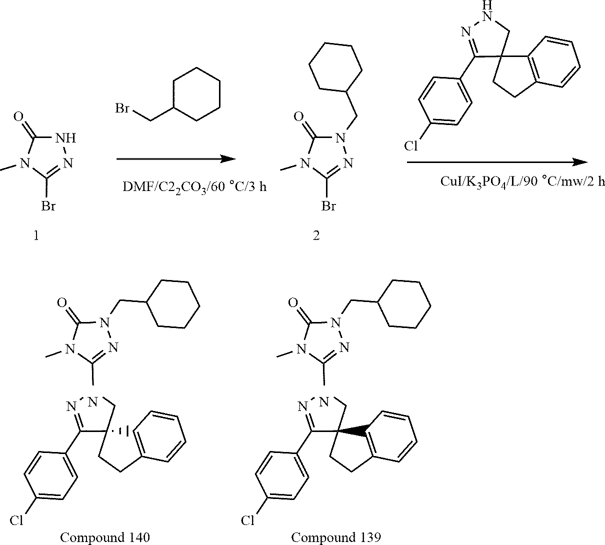

### 3-Bromo(Cyclohexylmethyl)-4-Methyl-4,5-Dihydro-1H-1,2,4-Triazol-5-One

To a stirred mixture of 3-bromo-4-methyl-4,5-dihydro-1H-1,2,4-triazol-5-one (530.00 mg, 2.978 mmol, 1.00 equiv.) and Cs₂CO₃ (1455.30 mg, 4.467 mmol, 1.50 equiv.) in DMA (10.00 mL) was added (bromomethyl)cyclohexane (790.96 mg, 4.467 mmol, 1.50 equiv.) at room temperature under N₂ atmosphere. The mixture was stirred for 5 h at 50° C. The mixture was allowed to cool down to room temperature. Desired product could be detected by LCMS. The reaction was quenched with water (100 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The residue/crude product was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 20% B gradient in 0 min; 20% B - 80% B gradient in 35 min; 80% B - 95% B gradient in 10 min; Detector: 220 nm. The fractions containing the desired product were collected at 55% B) to afford 3-bromo-1-(cyclohexylmethyl)-4-methyl-4,5-dihydro-1H-1,2,4-triazol-5-one (380 mg, 46.55%) as a white solid.

### 3-[(1S)(4-Chlorophenyl)-1,2,3,5-Tetrahydrospiro[Indene-1,4-Pyrazol]-1-yl]-1-(Cyclohexylmethyl)-4-Methyl-4,5-Dihydro-1H-1,2,4-Triazol-5-One and 3-[(1R)(4-Chlorophenyl)-1,2,3,5-Tetrahydrospiro[Indene-1,4-Pyrazol]-1-yl]-1-(Cyclohexylmethyl)-4-Methyl-4,5-Dihydro-1H-1,2,4-Triazol-5-One

A mixture of 3-bromo-1-(cyclohexylmethyl)-4-methyl-4,5-dihydro-1H-1,2,4-triazol-5-one (388.00 mg, 1.415 mmol, 1.00 equiv.), 3-(4-chlorophenyl)-1,2,3,5-tetrahydrospiro[indene-1,4-pyrazole] (800.36 mg, 2.830 mmol, 2.00 equiv.), N¹,N²-dimethylcyclohexane-1,2-diamine (80.52 mg, 0.566 mmol, 0.40 equiv.), K₃PO₄ (901.21 mg, 4.246 mmol, 3.00 equiv.), and CuI (53.91 mg, 0.283 mmol, 0.20 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 25% B gradient in 0 min; 25% B - 80% B gradient in 35 min; 80% B -95% B gradient in 0 min; 95% B - 95% B gradient in 5 min; Detector: 220 nm. The fractions containing the desired product were collected at 60% B) to afford 3-[3-(4-chlorophenyl)-1,2,3,5-tetrahydrospiro[indene-1,4-pyrazol]-1-y1]-1-(cyclohexylmethyl)-4-methyl-4,5-dihydro-1H-1,2,4-triazol-5-one (380 mg) as a yellow solid. The solid was separated by CHIRAL-HPLC to afford 3-[(1S)-3-(4-chlorophenyl)-1,2,3,5-tetrahydrospiro[indene-1,4-pyrazol]-1-yl]-1-(cyclohexylmethyl)-4-methyl-4, 5-dihydro-1H-1,2,4-triazol-5-one (90.4 mg, 13.42%) as a light yellow solid and 3-[(1R)-3-(4-chlorophenyl)-1,2,3,5-tetrahydrospiro[indene-1,4-pyrazol]-1-yl]-1-(cyclohexylmethyl)-4-methyl-4,5-dihydro-1H-1,2,4-triazol-5-one (130.7 mg, 19.40%) as an off-white solid.

### Example 17. Preparation of Compounds 142 and 143

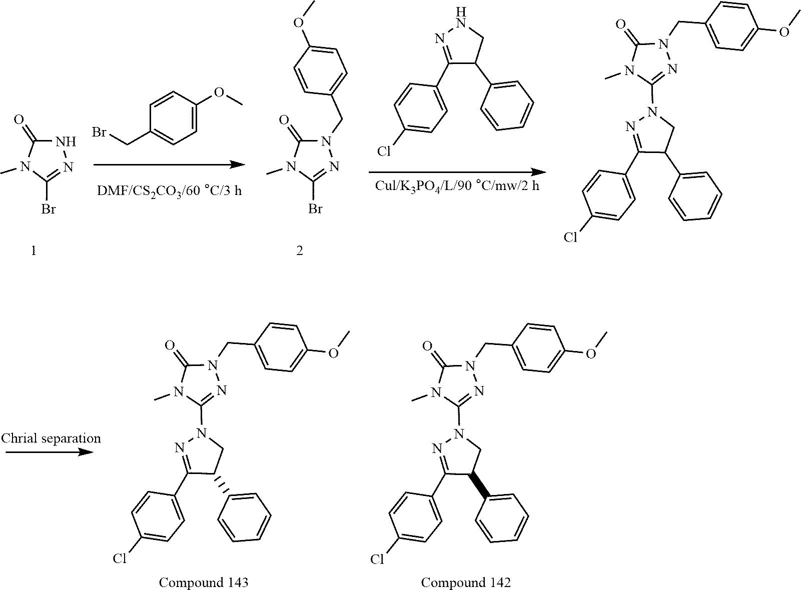

### 3-Bromo[(4-methoxyphenyl)methyl]-4-methyl-4,5-dihydro-1H-1,2,4-triazol-5-one

A mixture of 3-bromo-4-methyl-4,5-dihydro-1H-1,2,4-triazol-5-one (600.00 mg, 3.371 mmol, 1.00 equiv.), 1-(chloromethyl)-4-methoxybenzene (550.00 mg, 3.512 mmol, 1.04 equiv.) and Cs₂CO₃ (1.60 g, 4.911 mmol, 1.46 equiv.) in DMF (10.00 mL) was stirred for 4 h at room temperature under nitrogen atmosphere. Desired product could be detected by LCMS. The resulting mixture was diluted with 100 mL of water, then adjusted to pH 7 with AcOH and extracted with EtOAc (4 × 200 mL). The combined organic layers were washed with water (1×200 mL), dried over anhydrous Na₂SO₄. After filtration, the filtrate was concentrated under reduced pressure. The residue was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 6 min, 20% B-45% B gradient in 25 min; 98% B-98% B gradient in 8 min; Detector: 220 nm. The fractions containing the desired product were collected at 29% B and concentrated under reduced pressure to afford 3-bromo-1-[(4-methoxyphenyl)methyl]-4-methyl-4,5-dihydro-1H-1,2,4-triazol-5-one (840.6 mg, 83.64%) as a white solid. H-NMR analysis indicated the white solid was the desired product.

### 3-[(4S)(4-Chlorophenyl)-4-Phenyl-4,5-Dihydro-1H-Pyrazol-1-yl]-1-[(4-Methoxyphenyl)Methyl]-4-Methyl-4,5-Dihydro-1H-1,2,4-Triazol-5-One and 3-[(4R)(4-Chlorophenyl)-4-Phenyl-4,5-Dihydro-1H-Pyrazol-1-yl]-1-[(4-Methoxyphenyl)Methyl]-4-Methyl-4,5-Dihydro-1H-1,2,4-Triazol-5-One

Into a 20 mL sealed tube were added 3-bromo-1-[(4-methoxyphenyl)methyl]-4-methyl-4,5-dihydro-1H-1,2,4-triazol-5-one (400.00 mg, 1.342 mmol, 1.00 equiv.), 3-(4-chlorophenyl)-4-phenyl-4,5-dihydro-1H-pyrazole (760.00 mg, 2.960 mmol, 2.21 equiv.), N₁,N₂-dimethylcyclohexane-1,2-diamine (85.00 mg, 0.598 mmol, 0.45 equiv.), CuI (57.00 mg, 0.299 mmol, 0.22 equiv.), K₃PO₄ (950.00 mg, 4.476 mmol, 3.34 equiv.) and dioxane (10.00 mL) at room temperature. The mixture was irradiated with microwave radiation for 2 h at 90° C. Desired product could be detected by LCMS. The resulting mixture was diluted with 150 mL of water, then extracted with EtOAc (4 × 300 mL). The combined organic layers were washed with water (1× 300 mL), dried over anhydrous Na₂SO₄. After filtration, the filtrate was concentrated under reduced pressure. The residue was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 40% B-80% B gradient in 35 min; 98% B-98% B gradient in 6 min; Detector: 220 nm. The fractions containing the desired product were collected at 62% B and concentrated under reduced pressure. The residue was purified by silica gel column chromatography, eluted with PE/EtOAc (7:3) to afford 3-[3-(4-chlorophenyl)-4-phenyl-4,5-dihydro-1H-pyrazol-1-yl]-1-[(4-methoxyphenyl)methyl]-4-methyl-4,5-dihydro-1H-1,2,4-triazol-5-one (200 mg). The crude product (mg) was purified by Prep-Chiral-HPLC with the following conditions (Column: Chiralpak IC, 2 \*25cm, 5 um; Mobile Phase A: Hexane, Mobile Phase B: EtOH; Flow rate: 16 mL/min; Gradient: 50 B to 50 B in 15 min; 220/254 nm; RT1:10.55; RT2:11.865) to afford 3-[(4S)-3-(4-chlorophenyl)-4-phenyl-4,5-dihydro-1H-pyrazol-1-yl]-1-[(4-methoxyphenyl)methyl]-4-methyl-4,5-dihydro-1H-1,2,4-triazol-5-one (52.8 mg, 8.30%) as a yellow green solid and 3-[(4R)-3-(4-chlorophenyl)-4-phenyl-4,5-dihydro-1H-pyrazol-1-yl]-1-[(4-methoxyphenyl)methyl]-4-methyl-4,5-dihydro-1H-1,2,4-triazol-5-one (77.5 mg, 12.19%) as a yellow green solid.

Compound 160 was prepared by similar methods as described for Compounds 142 and 143 using the appropriate reagents without chiral separation.

Compounds 161 and 163 were prepared by similar methods as described for Compounds 142 and 143 using the appropriate reagents and the following chiral separation conditions:
Prep-HPLC conditions: Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 0 min, 5% B - 15% B gradient in 0 min; 15% B - 85% B gradient in 35 min; 75% B - 95% B gradient in 10 min; Detector: 220 nm)
Compounds 166 and 169 were prepared by the similar methods as described for Compounds 142 and 143 using the appropriate reagents and the following chiral separation conditions: Prep-HPLC conditions: Column: CHIRALPAK IC, 2 ^{∗}25 cm, 5 um; Mobile Phase A: Hex (0.2% IPA), Mobile Phase B: EtOH; Flow rate: 20 mL/min; Gradient: 25% B to 25% B in 15 min; 220/254 nm; RT1: 10.402; RT2: 11.782
Compounds 168, 170, 171, 175, 177, 178, 183, 189, 196, and 219 were prepared by similar methods as described for Compounds 142 and 143 using the appropriate reagents and without chiral separation.

### Example 18. Preparation of Compound 144

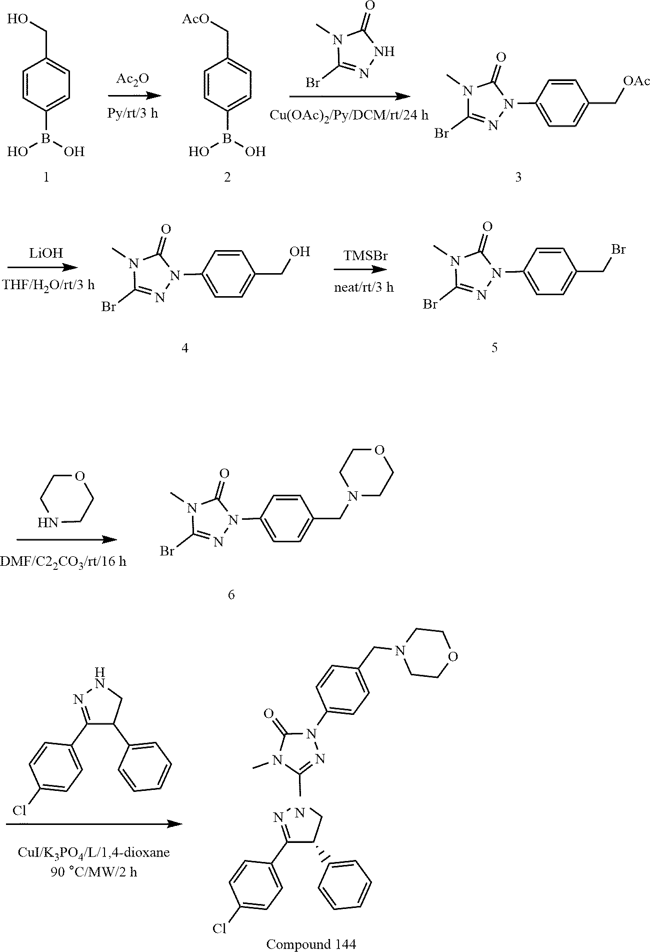

### (3-Bromo-4-Methyl-5-Oxo-4,5-Dihydro-1H-1,2,4-Triazol-1-yl)Phenyl]Methyl Acetate

To a stirred solution of [4-(hydroxymethyl)phenyl]boronic acid (500.00 mg, 3.290 mmol, 1.01 equiv.) in Pyridine (11 mL) was added AcOAc (1680.00 mg, 16.456 mmol, 5.05 equiv.) dropwise at room temperature under N₂ atmosphere. The mixture was stirred for 4 h at room temperature, and concentrated under vacuum. The residue was diluted with EA (200 mL) and washed with water (1 × 100 mL). The organic layer was concentrated under reduced pressure. The residue was dissolved in DCE (20.00 mL) and added 3-bromo-4-methyl-4,5-dihydro-1H-1,2,4-triazol-5-one (580.00 mg, 3.259 mmol, 1.00 equiv.), Pyridine (1 mL), Cu(AcO)₂ (240.00 mg, 1.321 mmol, 0.41 equiv.). The mixture was stirred for 16 h in the air. Desired product could be detected by LCMS. The resulting mixture was concentrated under vacuum. The residue was purified by silica gel column chromatography, eluted with PE/EA (3/1) to afford [4-(3-bromo-4-methyl-5-oxo-4,5-dihydro-1H-1,2,4-triazol-1-yl)phenyl]methyl acetate (85 mg, 8.00%) as a white solid.

### 3-Bromo[4-(Hydroxymethyl)phenyl]-4-Methyl-4,5-Dihydro-1H-1,2,4-Triazol-5-One

To a stirred mixture of [4-(3-bromo-4-methyl-5-oxo-4,5-dihydro-1H-1,2,4-triazol-1-yl)phenyl]methyl acetate (85.00 mg, 0.261 mmol, 1.00 equiv.) in H₂O (5 mL) and THF (2 mL) was added LiOH (31.21 mg, 1.303 mmol, 5.00 equiv.) at room temperature under N₂ atmosphere. The mixture was stirred for 4 h at room temperature. The mixture was acidified to pH = 6 with AcOH solution. Desired product could be detected by LCMS. The resulting mixture was extracted with EA (3 × 200 mL). The combined organic layers were washed with water (1 × 100 mL), dried over anhydrous Na₂SO₄. The organic layers were concentrated under reduced pressure to afford 3-bromo-1-[4-(hydroxymethyl)phenyl]-4-methyl-4,5-dihydro-1H-1,2,4-triazol-5-one (60 mg, 81.03%). The crude product was used in the next step directly without further purification.

### 3-Bromo[4-(Bromomethyl)Phenyl]-4-Methyl-4,5-Dihydro-1H-1,2,4-Triazol-5-One

A mixture of 3-bromo-1-[4-(hydroxymethyl)phenyl]-4-methyl-4,5-dihydro-1H-1,2,4-triazol-5-one (230.00 mg, 0.810 mmol, 1.00 equiv.) and bromotrimethylsilane (2 mL) was stirred for 3 h at room temperature. Desired product could be detected by LCMS. The reaction was quenched with water (20 mL) at room temperature. The resulting mixture was extracted with EA (3 × 100 mL). The combined organic layers were washed with water (1 × 50 mL), dried over anhydrous Na₂SO₄. After filtration, the filtrate was concentrated under reduced pressure. The residue was purified by Prep-TLC (PE/EA = 2/1) to afford 3-bromo-1-[4-(bromomethyl)phenyl]-4-methyl-4,5-dihydro-1H-1,2,4-triazol-5-one (280 mg, 99.67%) as an off-white solid.

### 3-BromoMethyl-1-[4-[(Morpholinyl)Methyl] Phenyl]-4,5-Dihydro-1H-1,2,4-Triazol-5-One

To a stirred mixture of 3-bromo-1-[4-(bromomethyl)phenyl]-4-methyl-4,5-dihydro-1H-1,2,4-triazol-5-one (280.00 mg, 0.807 mmol, 1.00 equiv.) and Cs₂CO₃ (460.00 mg, 1.412 mmol, 1.75 equiv.) in DMF (8.00 mL) was added morpholine (140.00 mg, 1.607 mmol, 1.99 equiv.) in portions at room temperature under N₂ atmosphere. The mixture was stirred for 4 h at room temperature. The reaction was quenched with water (100 mL) at room temperature. The resulting mixture was extracted with EA (3 × 100 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 20 min, 5% B - 5% B gradient in 8 min; 5% B - 95% B gradient in 5 min; 95% B - 5% B gradient in 5 min; Detector: 220 nm. The fractions containing the desired product were collected at 5% B) to afford 3-bromo-4-methyl-1-[4-[(morpholin-4-yl)methyl]phenyl]-4,5-dihydro-1H-1,2,4-triazol-5-one (260 mg, 91.22%) as a light yellow solid.

### 3-[(4S)(4-Chlorophenyl)-4-Phenyl-4,5-Dihydro-1H-Pyrazol-1-yl]-4-Methyl-1-[4-[(Morpholin-4-yl)Methyl] Phenyl]-4,5-Dihydro-1H-1,2,4-Triazol-5-One

A mixture of 3-bromo-4-methyl-1-[4-[(morpholin-4-yl)methyl]phenyl]-4,5-dihydro-1H-1,2,4-triazol-5-one (260.00 mg, 0.736 mmol, 1.00 equiv.), 3-(4-chlorophenyl)-4-phenyl-4,5-dihydro-1H-pyrazole (377.95 mg, 1.472 mmol, 2.00 equiv.), N1,N2-dimethylcyclohexane-1,2-diamine (41.88 mg, 0.294 mmol, 0.40 equiv.), K₃PO₄ (468.74 mg, 2.208 mmol, 3.00 equiv.), and CuI (28.04 mg, 0.147 mmol, 0.20 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 400 mL). The combined organic layers were washed with water (1 × 100 mL), dried over anhydrous Na₂SO₄. After filtration, the filtrate was concentrated under reduced pressure. The crude product was purified by reverse phase flash with the following conditions (Column: Spherical C18, 20 -40 um, 120 g; Mobile Phase A: Water (plus 5 mM FA); Mobile Phase B: ACN; Flow rate: 70 mL/min; Gradient: 0% - 5% B, 8 min, 5% B - 20% B gradient in 0 min; 20% B - 80% B gradient in 35 min; 80% B - 95% B gradient in 0 min; 95% B - 95% B gradient in 5 min; Detector: 220 nm. The fractions containing the desired product were collected at 50% B) to afford 3-[(4-chlorophenyl)-4-phenyl-4,5-dihydro-1H-pyrazol-1-yl]-4-methyl-1-[4-[(morpholin-4-yl)methyl]phenyl]-4,5-dihydro-1H-1,2,4-triazol-5-one (106 mg) as a light green solid. The solid was separated by CHIRAL-HPLC to afford 3-[(4S)-3-(4-chlorophenyl)-4-phenyl-4, 5-dihydro-1H-pyrazol-1-yl]-4-methyl-1-[4-[(morpholin-4-yl)methyl]phenyl]-4,5-dihydro-1H-1,2,4-triazol-5-one (37.4 mg, 9.60%) as a light green solid.

### Example 19. Preparation of Compound 145

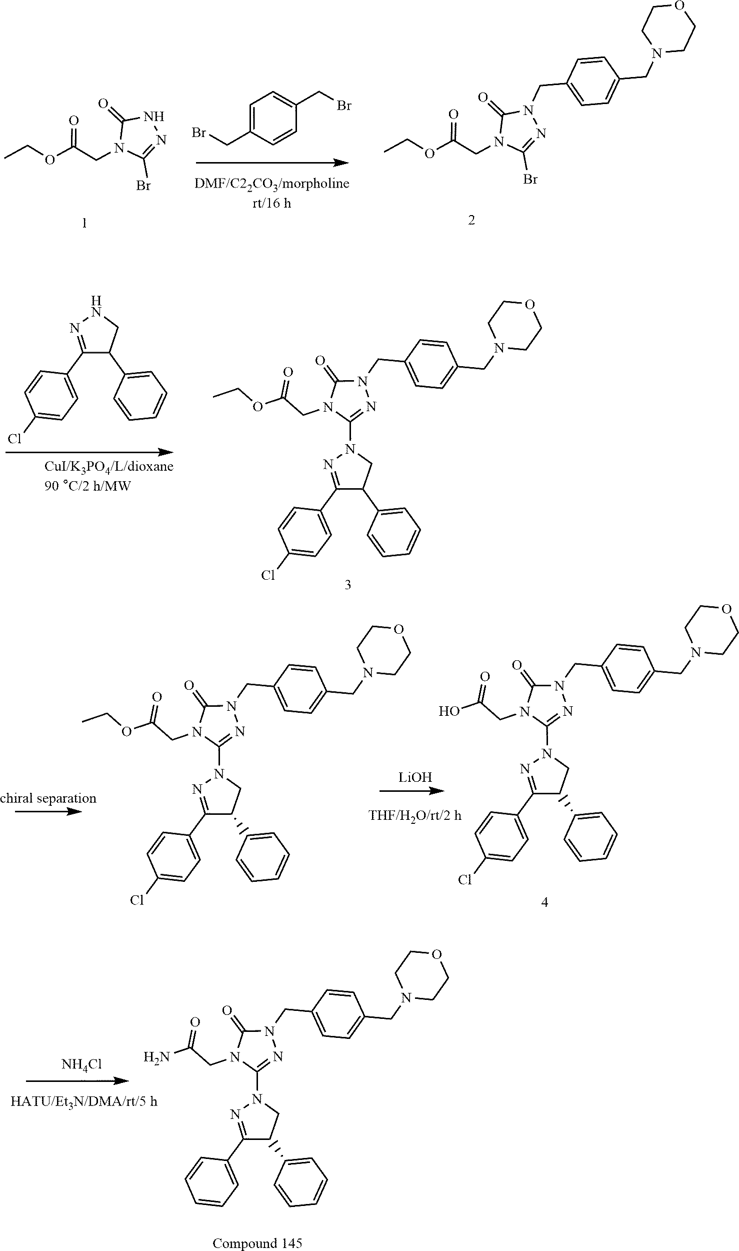

### Ethyl 2-[3-Bromo-1-([4-[(Morpholin-4-yl)Methyl] PHENYL] Methyl)-5-Oxo-4,5-Dihydro-1H-1,2,4-Triazol-4-yl] Acetate

To a stirred mixture of ethyl 2-(3-bromo-5-oxo-4,5-dihydro-1H-1,2,4-triazol-4-yl)acetate (500.00 mg, 2.000 mmol, 1.00 equiv.) and 1,4-bis(bromomethyl)benzene (791.72 mg, 2.999 mmol, 1.50 equiv.) in DMA (10.00 mL) was added Cs₂CO₃ (977.26 mg, 2.999 mmol, 1.50 equiv.) in portions at room temperature under N₂ atmosphere. The mixture was stirred for 2 h at room temperature, and then added morpholine (348.42 mg, 3.999 mmol, 2.00 equiv.) at room temperature. The mixture was stirred for 16 h at room temperature. Desired product could be detected by LCMS. The reaction was quenched with water (100 mL) at room temperature. The resulting mixture was extracted with EA (4 × 150 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 45 mL/min; Gradient: 0% - 5% B, 8 min, 5% B - 20% B gradient in 0 min; 20% B - 80% B gradient in 35 min; 80% B - 95% B gradient in 0 min; 95% B- 95% B gradient in 5 min; Detector: 220 nm. The fractions containing the desired product were collected at 50% B) to afford ethyl 2-[3-bromo-1-([4-[(morpholin-4-yl)methyl]phenyl]methyl)-5-oxo-4, 5-dihydro-1H-1,2,4-triazol-4-yl]acetate (430 mg, 48.95%) as a colorless oil.

### Ethyl 2-[3-[(4S)-3-(4-Chlorophenyl)-4-Phenyl-4,5-Dihydro-1H-Pyrazol-1-yl]-1-([4-[(Morpholin-4-yl)Methyl] Phenyl] Methyl)-5-Oxo-4,5-Dihydro-1H-1,2,4-Triazol-4-yl] Acetate

A mixture of ethyl 2-[3-bromo-1-([4-[(4-methylpiperazin-1-yl)methyl]phenyl]methyl)-5-oxo-4,5-dihydro-1H-1,2,4-triazol-4-yl]acetate (430.00 mg, 0.951 mmol, 1.00 equiv.), 3-(4-chlorophenyl)-4-phenyl-4,5-dihydro-1H-pyrazole (380.00 mg, 1.480 mmol, 1.56 equiv.), N₁,N₂-dimethylcyclohexane-1,2-diamine (56.00 mg, 0.394 mmol, 0.41 equiv.), K₃PO₄ (630.00 mg, 2.968 mmol, 3.12 equiv.), and CuI (38.00 mg, 0.200 mmol, 0.21 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 0 min, 5% B - 15% B gradient in 0 min; 15% B -75% B gradient in 35 min; 75% B - 95% B gradient in 10 min; Detector: 220 nm. The fractions containing the desired product were collected at 55% B) to afford ethyl 2-[3-(4-chlorophenyl)-4-phenyl-4, 5-dihydro-1H-pyrazol-1-yl]-1-([4-[(morpholin-4-yl)methyl]phenyl]methyl)-5-oxo-4,5-dihydro-1H-1,2,4-triazol-4-yl]acetate (260 mg) as a yellow solid. The solid was separated by CHIRAL-HPLC with the following conditions (Column: CHIRALPAK IG, 2*25cm,5um;Mobile Phase A: Hex (0.1%DEA):(EtOH:MeOH=1:1), Mobile Phase B: (EtOH:MeOH=1:1); Flow rate: 20 mL/min; Gradient: 60% B to 60% B in 20 min; 254/220 nm; RT1:12.164 min; RT2:15.51 min) to afford ethyl 2-[3-[(4S)-3-(4-chlorophenyl)-4-phenyl-4,5-dihydro-1H-pyrazol-1-yl]-1-([4-[(morpholin-4-yl)methyl]phenyl]methyl)-5-oxo-4, 5-dihydro-1H-1,2,4-triazol-4-yl]acetate (93 mg, 15.90%) as a light yellow solid.

### 2-[(4S)-3-(4-Chlorophenyl)-4-Phenyl-4,5-Dihydro-1H-Pyrazol-1-yl]-1-([4-[(Morpholin-4-yl)Methyl] Phenyl] Methyl)-5-Oxo-4,5-Dihydro-1H-1,2,4-Triazol-4-yl] Acetic Acid

A solution of ethyl 2-[3-[(4S)-3-(4-chlorophenyl)-4-phenyl-4,5-dihydro-1H-pyrazol-1-yl]-1-([4-[(morpholin-4-yl)methyl]phenyl]methyl)-5-oxo-4, 5-dihydro-1H-1,2,4-triazol-4-yl]acetate (100.00 mg, 0.163 mmol, 1.00 equiv.) and LiOH (20.00 mg, 0.835 mmol, 5.14 equiv.) in THF (8.00 mL) and H₂O (4.00 mL) was stirred for 2 h at room temperature. Desired product could be detected by LCMS. The resulting solution was added with 2 mL of AcOH. The resulting solution was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 10 min, 15% B-45% B gradient in 25 min; 98% B-98% B gradient in 8 min; Detector: 220 nm. The fractions containing the desired product were collected at 32% B and concentrated under reduced pressure to afford 2-[3-[(4S)-3-(4-chlorophenyl)-4-phenyl-4,5-dihydro-1H-pyrazol-1-yl]-1-([4-[(morpholin-4-yl)methyl]phenyl]methyl)-5-oxo-4,5-dihydro-1H-1,2,4-triazol-4-yl]acetic acid (86.1 mg, 90.21%) as a brown oil. H-NMR analysis indicated the brown oil was the desired product.

### 2-[(4S)-3-(4-Chlorophenyl)-4-Phenyl-4,5-Dihydro-1H-Pyrazol-1-yl]-1-([4-[(Morpholin-4-yl)Methyl] Phenyl] Methyl)-5-Oxo-4,5-Dihydro-1H-1,2,4-Triazol-4-yl]Acetamide

A solution of 2-[3-[(4S)-3-(4-chlorophenyl)-4-phenyl-4,5-dihydro-1H-pyrazol-1-yl]-1-([4-[(morpholin-4-yl)methyl]phenyl]methyl)-5-oxo-4,5-dihydro-1H-1,2,4-triazol-4-yl]acetic acid (86.00 mg, 0.146 mmol, 1.00 equiv.) and HATU (84.00 mg, 0.221 mmol, 1.51 equiv.) in DMA (8.00 mL) was stirred for 20 min at room temperature. Then the solution was added with NH₄Cl (39.00 mg, 0.729 mmol, 4.98 equiv.) and TEA (29.60 mg, 0.293 mmol, 2.00 equiv.) at room temperature. The solution was stirred for 4 h at room temperature. Desired product could be detected by LCMS. The resulting solution was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 10 min, 15% B-45% B gradient in 25 min; 98% B-98% B gradient in 8 min; Detector: 220 nm. The fractions containing the desired product were collected at 28% B and concentrated under reduced pressure to afford 2-[3-[(4S)-3-(4-chlorophenyl)-4-phenyl-4, 5-dihydro-1H-pyrazol-1-yl]-1-([4-[(morpholin-4-yl)methyl]phenyl]methyl)-5-oxo-4,5-dihydro-1H-1,2,4-triazol-4-yl]acetamide (42.2 mg, 49.15%) as a white solid. H-NMR analysis indicated the white solid was the desired product.

Compounds 149, 159, 164, and 167 was prepared by methods as described for Compound 145 using the appropriate reagents.

### Example 20. Preparation of Compound 147

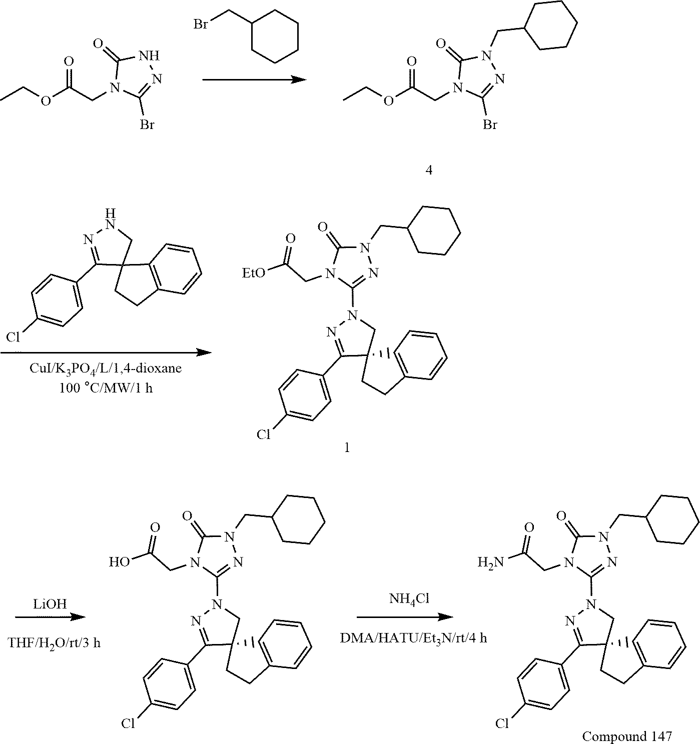

### Ethyl 2-[3-Bromo-1-(Cyclohexylmethyl)-5-Oxo-4,5-Dihydro-1H-1,2,4-Triazol-4-yl]Acetate

To a stirred mixture of ethyl 2-(3-bromo-5-oxo-4,5-dihydro-1H-1,2,4-triazol-4-yl)acetate (500.00 mg, 2.000 mmol, 1.00 equiv.) and Cs₂CO₃ (977.26 mg, 2.999 mmol, 1.50 equiv.) in DMF (10.00 mL) was added (bromomethyl)cyclohexane (425.00 mg, 2.400 mmol, 1.20 equiv.) at room temperature under N₂ atmosphere. The mixture was stirred for 6 h at 60° C. The mixture was allowed to cool down to room temperature. Desired product could be detected by LCMS. The reaction was quenched with water (100 mL) at room temperature. The resulting mixture was extracted with EA (3 × 300 mL). The combined organic layers were washed with water (1 × 200 mL), dried over anhydrous Na₂SO₄. After filtration, the filtrate was concentrated under reduced pressure. The residue/crude product was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 25% B gradient in 0 min; 25% B - 85% B gradient in 35 min; 85% B - 95% B gradient in 5 min; Detector: 220 nm. The fractions containing the desired product were collected at 65% B) to afford ethyl 2-[3-bromo-1-(cyclohexylmethyl)-5-oxo-4,5-dihydro-1H-1,2,4-triazol-4-yl]acetate (474 mg, 68.47%) as a light yellow solid.

### Ethyl 2- [3- [(1S)-3-(4-Chlorophenyl)-1,2,3,5-Tetrahydrospiro [Indene-1,4-Pyrazol]-1-yl]-1-(Cyclohexylmethyl)-5-Oxo-4,5-Dihydro-1H-1,2,4-Triazol-4-yl] Acetate

A mixture of ethyl 2-[3-bromo-1-(cyclohexylmethyl)-5-oxo-4,5-dihydro-1H-1,2,4-triazol-4-yl]acetate (470.00 mg, 1.357 mmol, 1.00 equiv.), 3-(4-chlorophenyl)-1,2,3,5-tetrahydrospiro[indene-1,4-pyrazole] (575.79 mg, 2.036 mmol, 1.50 equiv.), N¹,N²-dimethylcyclohexane-1,2-diamine (77.24 mg, 0.543 mmol, 0.40 equiv.), K₃PO₄ (864.45 mg, 4.072 mmol, 3.00 equiv.), and CuI (51.71 mg, 0.271 mmol, 0.20 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC to afford ethyl 2-[3-(4-chlorophenyl)-1,2,3,5-tetrahydrospiro[indene-1,4-pyrazol]-1-yl]-1-(cyclohexylmethyl)-5-oxo-4,5-dihydro-1H-1,2,4-triazol-4-yl]acetate (480 mg) as a white solid. The solid was separated by CHIRAL-HPLC with the following conditions (Column: Lux 5u Cellulose-4, 2.12 ^{∗}25cm,5 um; Mobile Phase A: Hex., Mobile Phase B: EtOH; Flow rate: 20 mL/min; Gradient: 50% B to 50% B in 18 min; Detector: 220/254 nm; RT1:7.941 min; RT2:12.985 min) to afford ethyl 2-[3-[(1S)-3-(4-chlorophenyl)-1,2,3,5-tetrahydrospiro[indene-1,4-pyrazol]-1-yl]-1-(cyclohexylmethyl)-5-oxo-4,5-dihydro-1H-1,2,4-triazol-4-yl]acetate (200 mg, 26.88%) as a white solid.

### 2-[(1S)-3-(4-Chlorophenyl)-1,2,3,5-Tetrahydrospiro[Indene-1,4-Pyrazol]-1-yl]-1-(Cyclohexylmethyl)-5-Oxo-4,5-Dihydro-1H-1,2,4-Triazol-4-yl]Acetic Acid

A solution of ethyl 2-[3-[(1S)-3-(4-chlorophenyl)-1,2,3,5-tetrahydrospiro[indene-1,4-pyrazol]-1-yl]-1-(cyclohexylmethyl)-5-oxo-4,5-dihydro-1H-1,2,4-triazol-4-yl]acetate (200.00 mg, 0.365 mmol, 1.00 equiv.) and LiOH (43.70 mg, 1.825 mmol, 5.00 equiv.) in THF (8.00 mL) and H₂O (4.00 mL) was stirred for 2 h at room temperature. Desired product could be detected by LCMS. The resulting solution was added with 2 mL of AcOH. The solution was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 40% B-85% B gradient in 25 min; 98% B-98% B gradient in 8 min; Detector: 220 nm. The fractions containing the desired product were collected at 82% B and concentrated under reduced pressure to afford 2-[3-[(1S)-3-(4-chlorophenyl)-1,2,3,5-tetrahydrospiro[indene-1,4-pyrazol]-1-yl]-1-(cyclohexylmethyl)-5-oxo-4, 5-dihydro-1H-1,2,4-triazol-4-yl] acetic acid (170 mg, 89.58%) as a white solid. The product was used in the next step directly.

### 2-[(1S)-3-(4-Chlorophenyl)-1,2,3,5-Tetrahydrospiro[Indene-1,4-Pyrazol]-1-yl]-1-(Cyclohexylmethyl)-5-Oxo-4,5-Dihydro-1H-1,2,4-Triazol-4-yl]Acetamide

A solution of 2-[3-[(1S)-3-(4-chlorophenyl)-1,2,3,5-tetrahydrospiro[indene-1,4-pyrazol]-1-yl]-1-(cyclohexylmethyl)-5-oxo-4,5-dihydro-1H-1,2,4-triazol-4-yl]acetic acid (180.00 mg, 0.346 mmol, 1.00 equiv.) and HATU (198.00 mg, 0.521 mmol, 1.50 equiv.) in DMA (8.00 mL) was stirred for 20 min at room temperature. Then the solution was added with NH₄C1 (93.00 mg, 1.739 mmol, 5.02 equiv.) and TEA (70.00 mg, 0.692 mmol, 2.00 equiv.) at room temperature. The mixture was stirred for 4 h at room temperature. Desired product could be detected by LCMS. The mixture was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 40% B-85% B gradient in 30 min; 98% B-98% B gradient in 8 min; Detector: 220 nm. The fractions containing the desired product were collected at 79% B and concentrated under reduced pressure. Then the residue was purified by silica gel column chromatography, eluted with CH₂Cl₂/ MeOH (40:1) to afford 2-[3-[(1S)-3-(4-chlorophenyl)-1,2,3,5-tetrahydrospiro[indene-1,4-pyrazol]-1-y1]-1-(cyclohexylmethyl)-5-oxo-4,5-dihydro-1H-1,2,4-triazol-4-yl]acetamide (59 mg, 32.84%) as a white solid. H-NMR analysis indicated the white solid was the desired product.

### Example 21. Preparation of Compounds 150 and 153

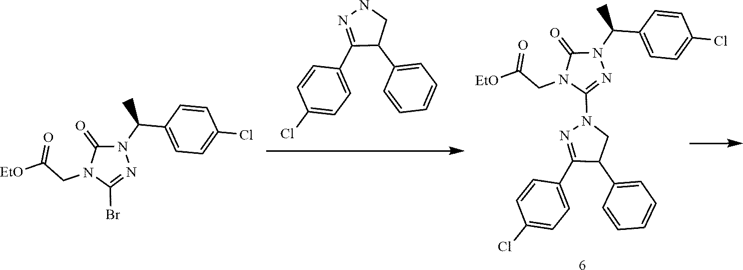

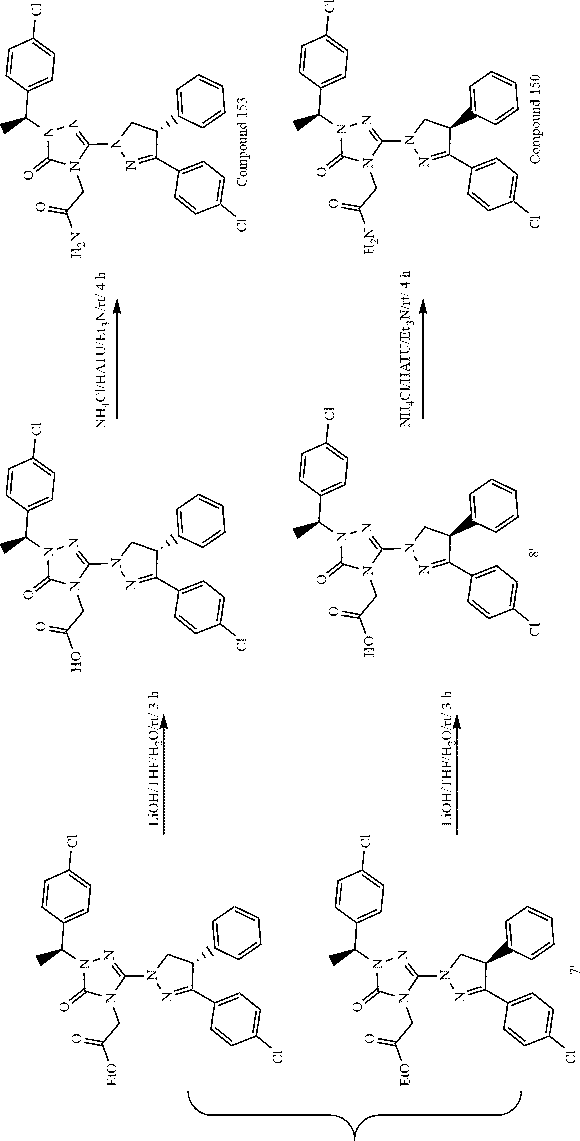

### Ethyl 2-[3-[(4S)-3-(4-Chlorophenyl)-4-Phenyl-4,5-Dihydropyrazol-1-yl]-1-[(1S)-1-(4-Chlorophenyl)Ethyl]-5-Oxo-1,2,4-Triazol-4-yl] Acetate and Ethyl 2-[3-[(4R)-3-(4-Chlorophenyl)-4-Phenyl-4,5-Dihydropyrazol-1-yl]-1-[(1S)-1-(4-Chlorophenyl)Ethyl]-5-Oxo-1,2,4-Triazol-4-yl] Acetate

Into a 25 mL sealed tube were added ethyl 2-[3-bromo-1-[(1S)-1-(4-chlorophenyl)ethyl]-5-oxo-1,2,4-triazol-4-yl]acetate (422.00 mg, 1.086 mmol, 1.00 equiv.), dioxane (10 mL), CuI (41.40 mg, 0.217 mmol, 0.20 equiv.), K₃PO₄ (691.50 mg, 3.258 mmol, 3.00 equiv.), 3-(4-chlorophenyl)-4-phenyl-4,5-dihydro-1H-pyrazole (557.00 mg, 2.170 mmol, 2.00 equiv.) and N1,N2-dimethylcyclohexane-1,2-diamine (61.80 mg, 0.434 mmol, 0.40 equiv.) at room temperature. The final mixture was irradiated with microwave radiation for 2 h at 90° C. Desired product could be detected by LCMS. The resulting mixture was added with 3 g silica gel and was concentrated under reduced pressure. The residue was purified by silica gel column chromatography, eluted with PE/EtOAc (4:1) to afford ethyl 2-[3-[3-(4-chlorophenyl)-4-phenyl-4, 5-dihydropyrazol-1-yl]-1-[1-(4-chlorophenyl)ethyl]-5-oxo-1,2,4-triazol-4-yl]acetate (274 mg). The crude product was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 50% B-90% B gradient in 40 min; Detector: 220 nm. The fractions containing the desired product were collected at 85% B and concentrated under reduced pressure to afford ethyl 2-[3-[3-(4-chlorophenyl)-4-phenyl-4,5-dihydropyrazol-1-yl]-1-[1-(4-chlorophenyl)ethyl]-5-oxo-1,2,4-triazol-4-yl]acetate (222 mg). The product (222 mg) was purified by Prep-Chiral-HPLC with the following conditions (Column: CHIRALPAK IG, 20^{∗}250 mm, 5 um; Mobile Phase A: Hex (0.1%DEA), Mobile Phase B: IPA; Flow rate: 20 mL/min; Gradient: 30 B to 30 B in 20 min; 220/254 nm; RT1:12.71; RT2:16.743) to afford ethyl 2-[3-[(4S)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetate (90 mg, 14.68%) as a white solid and ethyl 2-[3-[(4R)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetate (90 mg, 14.68%) as a white solid. H-NMR analysis indicated they were the desired product.

### [(4S)-3-(4-Chlorophenyl)-4-Phenyl-4,5-Dihydropyrazol-1-yl]-1-[(1S)-1-(4-Chlorophenyl)Ethyl]-5-Oxo-1,2,4-Triazol-4-yl]Acetic Acid

A mixture of ethyl 2-[3-[(4S)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetate (90.00 mg, 0.159 mmol, 1.00 equiv.) and LiOH (20.00 mg, 0.835 mmol, 5.24 equiv.) in THF (8 mL) and H₂O (4 mL) was stirred for 2 h at room temperature. Desired product could be detected by LCMS. The mixture was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 60%-80% B gradient in 20 min; 98%-98% B, 8 min, Detector: 220 nm. The fractions containing the desired product were collected at 72% B and concentrated under reduced pressure to afford [3-[(4S)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetic acid (80 mg, 93.54%) as a white solid.

### [(4R)-3-(4-Chlorophenyl)-4-Phenyl-4,5-Dihydropyrazol-1-yl]-1-[(1S)-1-(4-Chlorophenyl)Ethyl]-5-Oxo-1,2,4-Triazol-4-yl]Acetic Acid

A mixture of ethyl 2-[3-[(4R)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetate (90.00 mg, 0.159 mmol, 1.00 equiv.) and LiOH (20.00 mg, 0.835 mmol, 5.24 equiv.) in THF (8 mL) and H₂O (4 mL) was stirred for 2 h at room temperature. Desired product could be detected by LCMS. The mixture was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 60%-80% B gradient in 20 min; 98%-98% B, 8 min, Detector: 220 nm. The fractions containing the desired product were collected at 72% B and concentrated under reduced pressure to afford [3-[(4R)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetic acid (80 mg, 93.54%) as a white solid.

### 2-[(4S)-3-(4-Chlorophenyl)-4-Phenyl-4,5-Dihydropyrazol-1-yl]-1-[(1S)-1-(4-Chlorophenyl)Ethyl]-5-Oxo-1,2,4-Triazol-4-yl]Acetamide

A solution of [3-[(4S)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetic acid (80.00 mg, 0.149 mmol, 1.00 equiv.) and HATU (85.00 mg, 0.224 mmol, 1.50 equiv.) in DMA (8.00 mL) was stirred for 20 min at room temperature. Then the solution was added with NH₄Cl (40.00 mg, 0.748 mmol, 5.01 equiv.) and TEA (30.00 mg, 0.296 mmol, 1.99 equiv.) and stirred for 4 h at room temperature. Desired product could be detected by LCMS. The mixture was purified by reverse phase flash chromatography with the following conditions: Column: Spherical Cis, 20-40 um, 120; Mobile Phase A: water (plus 1.7 mM FA); Mobile Phase B: ACN; Flow rate: 70 mL/min; Gradient: 0%-0% B, 8 min, 60%-80% B gradient in 20 min; 98%-98% B, 8 min, Detector: 220 nm. The fractions containing the desired product were collected at 68% B and concentrated under reduced pressure to afford 2-[3-[(4S)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetamide (42.9 mg, 53.72%) as an off-white solid.

### 2-[(4R)-3-(4-Chlorophenyl)-4-Phenyl-4,5-Dihydropyrazol-1-yl]-1-[(1S)-1-(4-Chlorophenyl)Ethyl]-5-Oxo-1,2,4-Triazol-4-yl]Acetamide

A solution of [3-[(4R)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetic acid (80.00 mg, 0.149 mmol, 1.00 equiv.) and HATU (85.00 mg, 0.224 mmol, 1.50 equiv.) in DMA (8 mL) was stirred for 20 min at room temperature. Then the solution was added with NH₄Cl (40.00 mg, 0.748 mmol, 5.01 equiv.) and TEA (30.00 mg, 0.296 mmol, 1.99 equiv.) and stirred for 4 h at room temperature. Desired product could be detected by LCMS. The mixture was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 60%-80% B gradient in 20 min; 98%-98% B, 8 min, Detector: 220 nm. The fractions containing the desired product were collected at 68% B and concentrated under reduced pressure to afford 2-[3-[(4R)-3-(4-chlorophenyl)-4-phenyl-4,5-dihydropyrazol-1-yl]-1-[(1S)-1-(4-chlorophenyl)ethyl]-5-oxo-1,2,4-triazol-4-yl]acetamide (70 mg, 87.66%) as a white solid.

Compounds 152 and 154 were prepared by the same methods described for Compounds 150 and 153.

### Example 22. Preparation of Compound 158

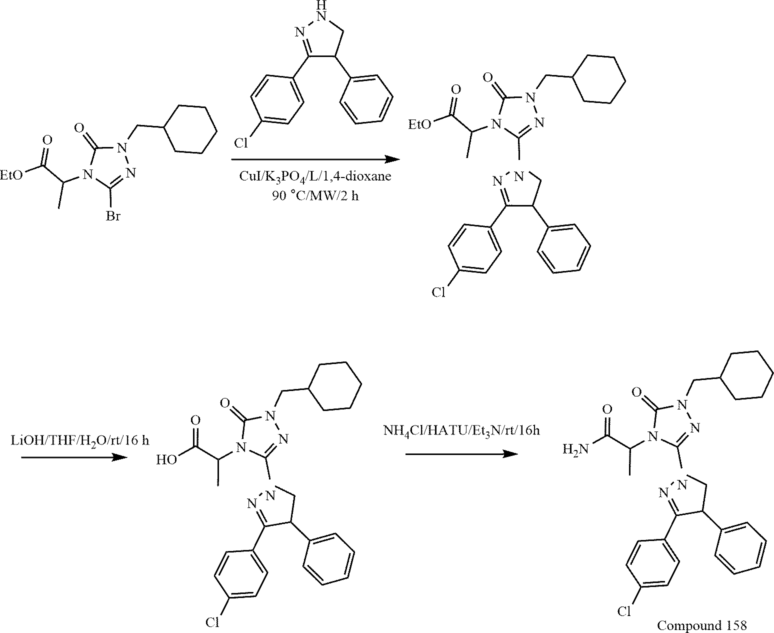

### Ethyl 2-[3-[3-(4-Chlorophenyl)-4-Phenyl-4,5-Dihydropyrazol-l-yl]-1-(Cyclohexylmethyl)-5-Oxo-1,2,4-Triazol-4-yl]Propanoate

To a stirred solution of 3-(4-chlorophenyl)-4-phenyl-4,5-dihydro-1H-pyrazole (356.32 mg, 1.388 mmol, 2.00 equiv.) and N¹,N²-dimethylcyclohexane-1,2-diamine (39.49 mg, 0.278 mmol, 0.40 equiv.) in dioxane (5.00 mL, 59.020 mmol, 85.05 equiv.) were added CuI (26.43 mg, 0.139 mmol, 0.20 equiv.) and K₃PO₄ (441.91 mg, 2.082 mmol, 3.00 equiv.) at room temperature under nitrogen atmosphere. To the mixture was added ethyl (2R)-2-[3-bromo-1-(cyclohexylmethyl)-5-oxo-1,2,4-triazol-4-yl]propanoate (250.00 mg, 0.694 mmol, 1.00 equiv.) at rt. The mixture was stirred at 90° C. for 2 h. The residue was purified by reverse phase flash chromatography with the following conditions: Column: Spherical C₁₈, 20 - 40 um, 330 g; Mobile Phase A: Water (plus 10 mM NH4HCO3); Mobile Phase B: ACN; Flow rate: 80 mL/min; Gradient: 5% - 5% B, 10 min, 75% B - 95% B gradient in 20 min; Detector: 254 nm. The fractions containing the desired product were collected at 90% B and concentrated under reduced pressure to afford ethyl 2-[3-[3-(4-chlorophenyl)-4-phenyl-4,5-dihydropyrazol-1-yl]-1-(cyclohexylmethyl)-5-oxo-1,2,4-triazol-4-yl]propanoate (120 mg, 32.26%) as yellow oil.

### 2-(4-Chlorophenyl)-4-Phenyl-4,5-Dihydropyrazol-1-yl]-1-(Cyclohexylmethyl)-5-Oxo-1,2,4-Triazol-4-yl]Propanoic Acid

To a stirred solution of ethyl 2-[3-[3-(4-chlorophenyl)-4-phenyl-4,5-dihydropyrazol-1-yl]-1-(cyclohexylmethyl)-5-oxo-1,2,4-triazol-4-yl]propanoate (48.00 mg, 0.090 mmol, 1.00 equiv) in THF (3.13 mL, 43.414 mmol, 431.52 equiv) and H2O (3.13 mL, 173.765 mmol, 1940.63 equiv) was added LiOH (21.44 mg, 0.895 mmol, 10.00 equiv) at room temperature. The solution was stirred at rt for 16 h. To the mixture was added glacial acetic acid (2 mL). The residue was purified by reverse phase flash chromatography with the following conditions: Column: Spherical C18, 20 - 40 um, 120 g; Mobile Phase A: Water (plus 10 mM FA); Mobile Phase B: ACN; Flow rate: 60 mL/min; Gradient: 5% - 5% B, 10 min, 75% B - 95% B gradient in 20 min; Detector: 254 nm. The fractions containing the desired product were collected at 85% B and concentrated under reduced pressure to afford 2-[3-[3-(4-chlorophenyl)-4-phenyl-4,5-dihydropyrazol-1-yl]-1-(cyclohexylmethyl)-5-oxo-1,2,4-triazol-4-yl]propanoic acid (45 mg, 98.93%) as yellow oil.

### 2-(4-Chlorophenyl)-4-Phenyl-4,5-Dihydropyrazol-1-yl]-1-(Cyclohexylmethyl)-5-Oxo-1,2,4-Triazol-4-yl]Propanamide

To a stirred solution of 2-[3-[3-(4-chlorophenyl)-4-phenyl-4,5-dihydropyrazol-1-yl]-1-(cyclohexylmethyl)-5-oxo-1,2,4-triazol-4-yl]propanoic acid (100.00 mg, 0.197 mmol, 1.00 equiv) in DMA (4.00 mL, 43.021 mmol, 218.55 equiv) was added HATU (112.27 mg, 0.295 mmol, 1.50 equiv) at rt. The solution was stirred at rt for 0.5 h. To the mixture was added NH4Cl (52.65 mg, 0.984 mmol, 5.00 equiv) and TEA (59.76 mg, 0.591 mmol, 3.00 equiv). The mixture was stirred at 2 h. The resulting mixture was concentrated under reduced pressure. The crude product (100 mg) was purified by Prep-HPLC with the following conditions (Column: XBridge Prep OBD C18 Column 30 × 150 mm 5um;Mobile Phase A:undefined, Mobile Phase B: undefined; Flow rate: 60 mL/min; Gradient: 45% B to 70% B in 8 min; 220 nm; Rt: 7.03 min) to afford 2-[3-[3-(4-chlorophenyl)-4-phenyl-4,5-dihydropyrazol-1-yl]-1-(cyclohexylmethyl)-5-oxo-1,2,4-triazol-4-yl]propanamide (88.5 mg) as a white solid.

### Example 23. Preparation of Compound 172

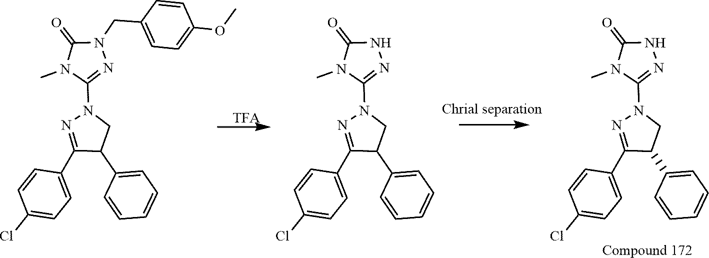

A solution of 5-[3-(4-chlorophenyl)-4-phenyl-4,5-dihydropyrazol-1-yl]-2-[(4-methoxyphenyl)methyl]-4-methyl-1,2,4-triazol-3-one (400.00 mg, 0.844 mmol, 1.00 equiv.) in TFA (20.00 mL) was stirred for 16 h at 85° C. under N₂ atmosphere. The mixture was allowed to cool down to room temperature. Desired product could be detected by LCMS. The resulting mixture was concentrated under reduced pressure. The residue was purified by reverse phase flash with the following conditions (Column: Spherical C₁₈, 20 -40 um, 120 g; Mobile Phase A: Water (plus 5 mM FA); Mobile Phase B: ACN; Flow rate: 45 mL/min; Gradient: 0% - 5% B, 8 min, 5% B - 25% B gradient in 0 min; 25% B - 85% B gradient in 35 min; 85% B - 95% B gradient in 5 min; Detector: 220 nm. The fractions containing the desired product were collected at 65% B) to afford 5-[3-(4-chlorophenyl)-4-phenyl-4,5-dihydropyrazol-1-yl]-4-methyl-2H-1,2,4-triazol-3-one (140 mg) as a white solid. The solid (40 mg) was separated by CHIRAL-HPLC with the following conditions (Column: CHIRALPAK IG, 2^{∗}25cm, 5um; Mobile Phase A:Hex, Mobile Phase B: IPA; Flow rate: 20 mL/min; Gradient: 50% B to 50% B in 13 min; Detector: 220/254 nm; RT1:8.197 min; RT2:11.195 min) to afford 5-[(4S)-3-(4-chlorophenyl)-4-phenyl-4,5-dihydropyrazol-1-yl]-4-methyl-2H-1,2,4-triazol-3-one (7.4 mg, 2.48%) as a white solid. Compound 181 was prepared in a similar manner using the appropriate reagents.

### Example 24. Preparation of Compound 194

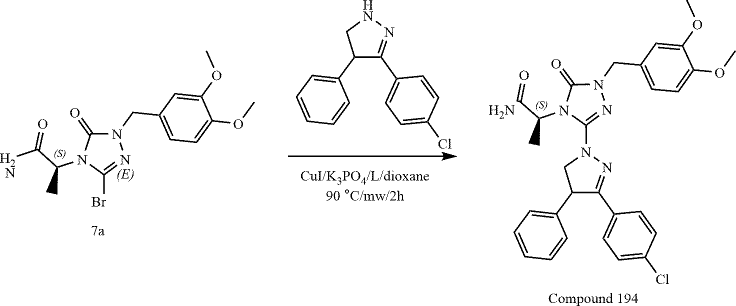

A mixture of (2S)-2-[3-bromo-1-[(3,4-dimethoxyphenyl)methyl]-5-oxo-1,2,4-triazol-4-yl]propanamide (320.00 mg, 0.831 mmol, 1.00 equiv.), 3-(4-chlorophenyl)-4-phenyl-4,5-dihydro-1H-pyrazole (319.90 mg, 1.246 mmol, 1.50 equiv.), N₁,N₂-dimethylcyclohexane-1,2-diamine (47.27 mg, 0.332 mmol, 0.40 equiv.), K₃PO₄ (528.99 mg, 2.492 mmol, 3.00 equiv.) and CuI (31.64 mg, 0.166 mmol, 0.20 equiv.) in dioxane (10 mL) was irradiated with microwave radiation for 2 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC with the following conditions (Column: XBridge Shield RP₁₈ OBD Column 30^{∗} 150 mm, 5 um; Mobile Phase A:Water (10 mMOL/L NH₄HCO₃), Mobile Phase B: ACN; Flow rate: 60 mL/min; Gradient: 40% B to 55% B in 7 min; 254 nm; Rt: 6.5 min) to afford (2S)-2-[3-[3-(4-chlorophenyl)-4-phenyl-4,5-dihydropyrazol-1-yl]-1-[(3,4-dimethoxyphenyl)methyl]-5-oxo-1,2,4-triazol-4-yl]propanamide (110 mg, 23.60%) as a white solid.

### Example 25. Preparation of Compound 197

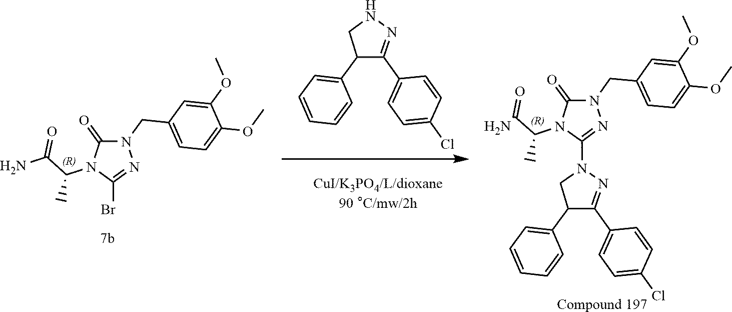

A mixture of (2R)-2-[3-bromo-1-[(3,4-dimethoxyphenyl)methyl]-5-oxo-1,2,4-triazol-4-yl]propanamide (318.00 mg, 0.826 mmol, 1.00 equiv.), 3-(4-chlorophenyl)-4-phenyl-4,5-dihydro-1H-pyrazole (317.90 mg, 1.238 mmol, 1.50 equiv.), N¹,N²-dimethylcyclohexane-1,2-diamine (46.97 mg, 0.330 mmol, 0.40 equiv.), K₃PO₄ (525.68 mg, 2.477 mmol, 3.00 equiv.) and CuI (31.44 mg, 0.165 mmol, 0.20 equiv.) in dioxane (10 mL) was irradiated with microwave radiation for 2 h at 100° C. under N2 atmosphere. The mixture was allowed to cool down to room temperature. Desired product could be detected by LCMS. The reaction was quenched by the addition of water (50 mL) at room temperature. The resulting mixture was extracted with EA (3 x 200 mL). The combined organic layers were washed with water (1 x 100 mL), dried over anhydrous Na₂SO₄. After filtration, the filtrate was concentrated under reduced pressure. The crude product was purified by Prep-HPLC with the following conditions (Column: Sunfire Prep C₁₈ OBD Column, 10 um, 19 ^{∗}250mm; Mobile Phase A:Water (0.05%TFA), Mobile Phase B: ACN; Flow rate: 25 mL/min; Gradient: 45% B to 55% B in 7 min; 254 nm; Rt: 6.5 min) to afford (2R)-2-[3-[3-(4-chlorophenyl)-4-phenyl-4,5-dihydropyrazol-1-yl]-1-[(3,4-dimethoxyphenyl)methyl]-5-oxo-1,2,4-triazol-4-yl]propanamide (70 mg, 15.11%) as a white solid.

### Example 26. Preparation of Compounds 184, 187, 190, and 191

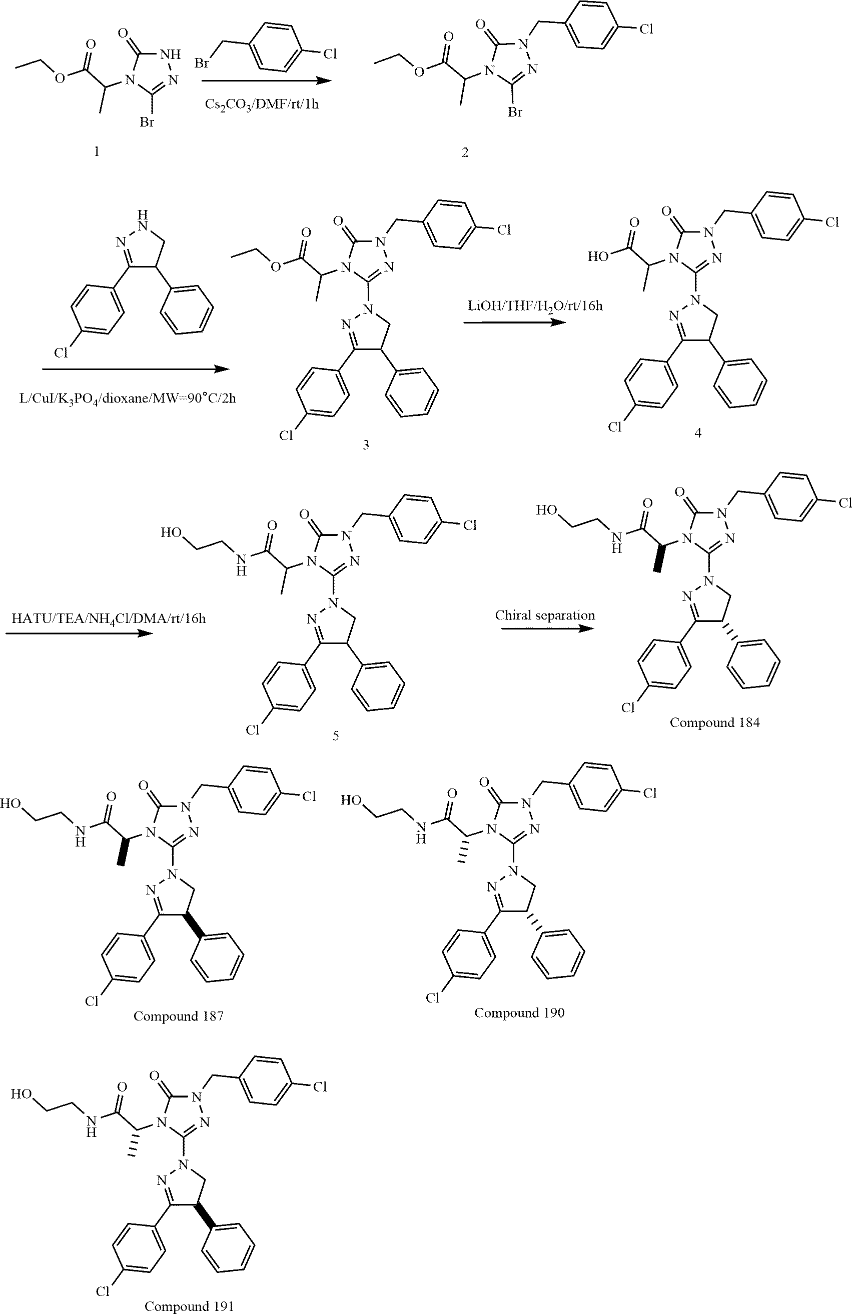

### Ethyl 2-[3-Bromo-1-[(4-Chlorophenyl)Methyl]-5-Oxo-1,2,4-Triazol-4-yl] Propanoate

A mixture of ethyl 2-(3-bromo-5-oxo-1H-1,2,4-triazol-4-yl)propanoate (500.00 mg, 1.893 mmol, 1.00 equiv.), C_{S2}CO₃ (925.30 mg, 2.840 mmol, 1.50 equiv.) and 1-(bromomethyl)-4-chlorobenzene (466.90 mg, 2.272 mmol, 1.20 equiv.) in DMF (5 mL) was stirred for 4 h at room temperature under nitrogen atmosphere. Desired product could be detected by LCMS. The mixture was neutralized to pH 7 with AcOH. The mixture was purified by reverse phase flash chromatography with the following conditions: Column: Spherical C18, 20-40 um, 120; Mobile Phase A: water (plus 1.7 mM FA); Mobile Phase B: ACN; Flow rate: 70 mL/min; Gradient: 0%-0% B, 8 min, 40%-60% B gradient in 15 min; 98%-98% B, 8 min, Detector: 220 nm. The fractions containing the desired product were collected at 55% B and concentrated under reduced pressure to afford ethyl 2-[3-bromo-1-[(4-chlorophenyl)methyl]-5-oxo-1,2,4-triazol-4-yl]propanoate (725 mg, 98.52%) as a white solid. H-NMR analysis indicated the white solid was the desired product.

### Ethyl 2-[3-[3-(4-Chlorophenyl)-4-Phenyl-4,5-Dihydropyrazol-1-yl]-1-[(4-Chlorophenyl)Methyl]-5-Oxo-1,2,4-Triazol-4-yl]Propanoate

Into a 25 mL sealed tube were added ethyl 2-[3-bromo-1-[(4-chlorophenyl)methyl]-5-oxo-1,2,4-triazol-4-yl]propanoate (720.00 mg, 1.853 mmol, 1.00 equiv.), dioxane (10 mL), CuI (70.00 mg, 0.368 mmol, 0.20 equiv.), K₃PO₄ (1.18 g, 5.559 mmol, 3.00 equiv.), 3-(4-chlorophenyl)-4-phenyl-4,5-dihydro-1H-pyrazole (720.00 mg, 2.805 mmol, 1.51 equiv.) and N¹,N²-dimethylcyclohexane-1,2-diamine (105.00 mg, 0.738 mmol, 0.40 equiv.) at room temperature. The final reaction mixture was irradiated with microwave radiation for 2 h at 100° C. Desired product could be detected by LCMS. The mixture was allowed to cool down to room temperature. The mixture was added with EA and 4 g silica gel. The resulting mixture was concentrated under reduced pressure. The residue was purified by silica gel column chromatography, eluted with PE/EtOAc (7:3) to afford ethyl 2-[3-[3-(4-chlorophenyl)-4-phenyl-4,5-dihydropyrazol-1-yl]-1-[(4-chlorophenyl)methyl]-5-oxo-1,2,4-triazol-4-yl]propanoate (337.2 mg, 32.25%) as a white solid. The crude product was used in the next step directly without further purification.

### 2-(4-Chlorophenyl)-4-Phenyl-4,5-Dihydropyrazol-1-yl]-1-[(4-Chlorophenyl)Methyl]-5-Oxo-1,2,4-Triazol-4-yl]Propanoic Acid

A mixture of ethyl 2-[3-[3-(4-chlorophenyl)-4-phenyl-4,5-dihydropyrazol-1-yl]-1-[(4-chlorophenyl)methyl]-5-oxo-1,2,4-triazol-4-yl]propanoate (337.00 mg, 0.597 mmol, 1.00 equiv.) and LiOH (142.00 mg, 5.930 mmol, 9.93 equiv.) in THF (8.00 mL) and H₂O (6.00 mL) was stirred for 7 h at 50° C. Desired product could be detected by LCMS. The solution was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 57%-77% B gradient in 15 min; 98%-98% B, 8 min, Detector: 220 nm. The fractions containing the desired product were collected at 65% B and concentrated under reduced pressure to afford 2-[3-[3-(4-chlorophenyl)-4-phenyl-4,5-dihydropyrazol-1-yl]-1-[(4-chlorophenyl)methyl]-5-oxo-1,2,4-triazol-4-yl]propanoic acid (230 mg, 71.82%) as a white solid. The product was used in the next step directly.

### (2S)[3-[(4S)-3-(4-Chlorophenyl)-4-Phenyl-4,5-Dihydropyrazol-1-yl]-1-[(4-Chlorophenyl)Methyl]-5-Oxo-1,2,4-Triazol-4-yl]-N-(2-Hydroxyethyl)Propanamide, (2S)[3-[(4R)-3-(4-Chlorophenyl)-4-Phenyl-4,5-Dihydropyrazol-1-yl]-1-[(4-Chlorophenyl)Methyl]-5-Oxo-1,2,4-Triazol-4-yl]-N-(2-Hydroxyethyl)Propanamide, (2R)[3-[(4S)-3-(4-Chlorophenyl)-4-Phenyl-4,5-Dihydropyrazol-1-yl]-1-[(4-Chlorophenyl)Methyl]-5-Oxo-1,2,4-Triazol-4-yl]-N-(2-Hydroxyethyl)Propanamide and (2R)[3-[(4R)-3-(4-Chlorophenyl)-4-Phenyl-4,5-Dihydropyrazol-1-yl]-1-[(4-Chlorophenyl)Methyl]-5-Oxo-1,2,4-Triazol-4-yl]-N-(2-Hydroxyethyl)Propanamide

A mixture of 2-[3-[3-(4-chlorophenyl)-4-phenyl-4,5-dihydropyrazol-1-yl]-1-[(4-chlorophenyl)methyl]-5-oxo-1,2,4-triazol-4-yl]propanoic acid (230.00 mg, 0.429 mmol, 1.00 equiv.) and HATU (244.60 mg, 0.643 mmol, 1.50 equiv.) in DMA (8.00 mL) was stirred for 25 min at room temperature under nitrogen atmosphere. Then the mixture was added with ethanolamine (78.60 mg, 1.287 mmol, 3.00 equiv.) and TEA (130.20 mg, 1.287 mmol, 3.00 equiv.), and was stirred for 2 h. Desired product could be detected by LCMS. The mixture was purified by reverse phase flash chromatography with the following conditions: Column: Spherical C18, 20-40 um, 120; Mobile Phase A: water (plus 1.7 mM FA); Mobile Phase B: ACN; Flow rate: 70 mL/min; Gradient: 0%-0% B, 8 min, 48%-68% B gradient in 15 min; 98%-98% B, 8 min, Detector: 220 nm. The fractions containing the desired product were collected at 60% B and concentrated under reduced pressure to afford 2-[3-[3-(4-chlorophenyl)-4-phenyl-4,5-dihydropyrazol-1-yl]-1-[(4-chlorophenyl)methyl]-5-oxo-1.2,4-triazol-4-yl]-N-(2-hydroxyethyl)propanamide (236.2 mg). The crude product (236.2 mg) was purified by Prep-Chiral-HPLC with the following conditions (Column: CHIRALPAK IG, 2 ^{∗}25 cm,5 um; Mobile Phase A: Hex(0.2%DEA), Mobile Phase B: IPA; Flow rate: 20 mL/min; Gradient: 30% B to 30% B in 16 min; Detector: 220/254 nm; RT1: 7.529 min; RT2: 8.923 min; RT3: 13.674 min) to afford (2S)-2-[3-[(4S)-3-(4-chlorophenyl)-4-phenyl-4,5-dihydropyrazol-1-yl]-1-[(4-chlorophenyl)methyl]-5-oxo-1,2,4-triazol-4-yl]-N-(2-hydroxyethyl)propanamide (34 mg, 13.68%) as a white solid and (2S)-2-[3-[(4R)-3-(4-chlorophenyl)-4-phenyl-4,5-dihydropyrazol-1-yl]-1-[(4-chlorophenyl)methyl]-5-oxo-1,2,4-triazol-4-yl]-N-(2-hydroxyethyl)propanamide (38.7 mg, 15.58%) as a white solid and the mixture of (2R)-2-[3-[(4S)-3-(4-chlorophenyl)-4-phenyl-4,5-dihydropyrazol-1-yl]-1-[(4-chlorophenyl)methyl]-5-oxo-1,2,4-triazol-4-yl]-N-(2-hydroxyethyl)propanamide and (2R)-2-[3-[(4R)-3-(4-chlorophenyl)-4-phenyl-4,5-dihydropyrazol-1-yl]-1-[(4-chlorophenyl)methyl]-5-oxo-1,2,4-triazol-4-yl]-N-(2-hydroxyethyl)propanamide. That mixture (116.2 mg) was purified by Prep-Chiral-HPLC with the following conditions (Column: CHIRAL ART Cellulose-SB, 2^{∗}25 cm, 5 um; Mobile Phase A: Hex(0.2%DEA), Mobile Phase B: EtOH; Flow rate: 20 mL/min; Gradient: 15% B to 15% B in 21 min; 220/254 nm; RT1: 15.576 min; RT2: 18.131 min) to afford (2R)-2-[3-[(4S)-3-(4-chlorophenyl)-4-phenyl-4,5-dihydropyrazol-1-yl]-1-[(4-chlorophenyl)methyl]-5-oxo-1,2,4-triazol-4-yl]-N-(2-hydroxyethyl)propanamide (38.6 mg, 15.54%) as an off-white solid and (2R)-2-[3-[(4R)-3-(4-chlorophenyl)-4-phenyl-4,5-dihydropyrazol-1-yl]-1-[(4-chlorophenyl)methyl]-5-oxo-1,2,4-triazol-4-yl]-N-(2-hydroxyethyl)propanamide (40.9 mg, 16.46%) as an off-white solid. H-NMR and SFC analysis indicated they were the desired product.

Compounds 174, 176, 179, 180, 201, 205, 207, and 213 were prepared by methods as described in this example using the appropriate reagents.

### Example 27. Preparation of Compound 192

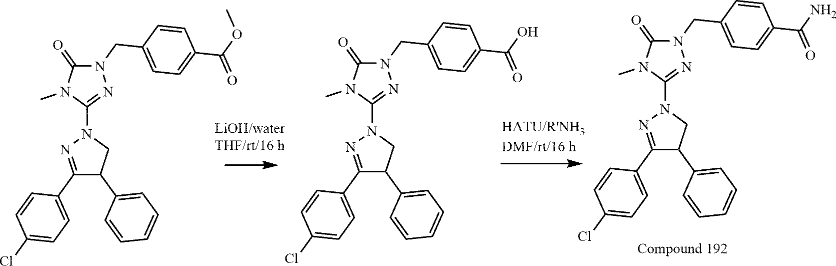

### 4-((4-Chlorophenyl)-4-Phenyl-4,5-Dihydropyrazol-1-yl]-4-Methyl-5-Oxo-1,2,4-Triazol-1-yl]Methyl)Benzoic Acid

To a stirred solution of methyl 4-([3-[3-(4-chlorophenyl)-4-phenyl-4,5-dihydropyrazol-1-yl]-4-methyl-5-oxo-1,2,4-triazol-1-yl]methyl)benzoate (440.00 mg, 0.877 mmol, 1.00 equiv.) in THF (4.00 mL, 49.372 mmol, 56.33 equiv.) and H₂O (4.00 mL, 222.033 mmol, 253.30 equiv.) was added LiOH (209.92 mg, 8.765 mmol, 10.00 equiv.) at room temperature. The solution was stirred at rt for 16 h. The resulting mixture was concentrated under reduced pressure. The residue was purified by reverse phase flash chromatography with the following conditions: Column: Spherical C18, 20 - 40 um, 330 g; Mobile Phase A: Water (plus 10 mM NH₄HCO₃); Mobile Phase B: ACN; Flow rate: 80 mL/min; Gradient: 5% - 5% B, 10 min, 33% B - 45% B gradient in 20 min; Detector: 254 nm. The fractions containing the desired product were collected at 40% B and concentrated under reduced pressure to afford 4-([3-[3-(4-chlorophenyl)-4-phenyl-4,5-dihydropyrazol-1-yl]-4-methyl-5-oxo-1,2,4-triazol-1-yl]methyl)benzoic acid (350 mg, 81.83%) as yellow oil.

### 4-((4-Chlorophenyl)-4-Phenyl-4,5-Dihydropyrazol-1-yl]-4-Methyl-5-Oxo-1,2,4-Triazol-1-yl] Methyl)Benzamide

To a stirred solution of 4-([3-[3-(4-chlorophenyl)-4-phenyl-4,5-dihydropyrazol-1-yl]-4-methyl-5-oxo-1,2,4-triazol-1-yl]methyl)benzoic acid (130.00 mg, 0.266 mmol, 1.00 equiv.) in DMA (5.00 mL, 53.776 mmol, 201.84 equiv.) was added HATU (151.95 mg, 0.400 mmol, 1.50 equiv.) at room temperature. The solution was stirred at rt for 0.5 h. To the mixture were added NH₄C1 (71.26 mg, 1.332 mmol, 5.00 equiv.) and TEA (80.88 mg, 0.799 mmol, 3.00 equiv.) at rt. The mixture was stirred at rt for 2 h. The crude product (150 mg) was purified by Prep-HPLC with the following conditions (Column: Sunfire Prep C₁₈ OBD Column, 10 um,19 \*250mm;Mobile Phase A:Water(0.1%FA), Mobile Phase B: ACN; Flow rate: 25 mL/min; Gradient: 45% B to 95% B in 7 min; 254 nm; Rt: 6.5 min) to afford 4-([3-[3-(4-chlorophenyl)-4-phenyl-4,5-dihydropyrazol-1-yl]-4-methyl-5-oxo-1,2,4-triazol-1-yl]methyl)benzamide (33.1 mg, 25.51%) as a white solid.

Compounds and 173, 182, and 186 were prepared by methods as described for Compound 192 using the appropriate reagents.

### Example 28. Preparation of Compound 193

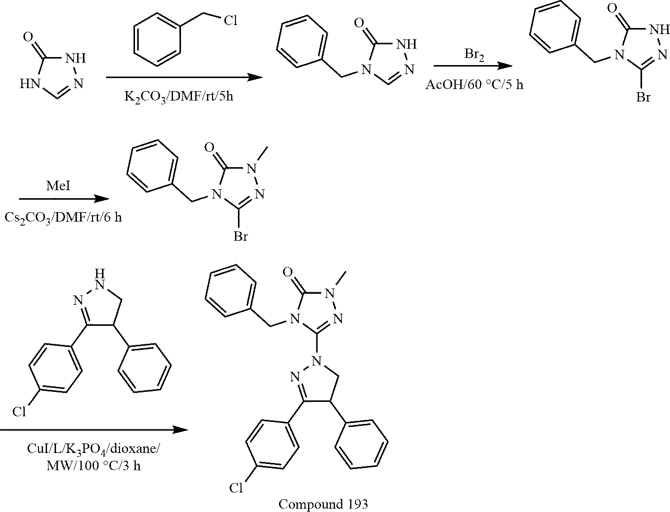

### 4-Benzyl-2H-1,2,4-Triazol-3-One

To a stirred mixture of 2,4-dihydro-1,2,4-triazol-3-one (1.00 g, 11.756 mmol, 1.00 equiv.) and K₂CO₃ (2.44 g, 17.633 mmol, 1.50 equiv.) in DMF (10 mL) was added benzyl chloride (0.74 g, 5.878 mmol, 0.50 equiv.) in portions at room temperature under N₂ atmosphere. The mixture was stirred for 4 h at room temperature. The reaction was quenched with water (100 mL) at room temperature. The resulting mixture was extracted with EA (3 × 100 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B - 20% B gradient in 0 min; 20% B -80% B gradient in 25 min; 80% B - 95% B gradient in 5 min; Detector: 220 nm. The fractions containing the desired product were collected at 42% B) to afford 4-benzyl-2H-1,2,4-triazol-3-one (1.2 g, 58.27%) as an off-white solid.

### 4-Benzyl-5-Bromo-2H-1,2,4-Triazol-3-One

To a stirred solution of 4-benzyl-2H-1,2,4-triazol-3-one (1.20 g, 6.850 mmol, 1.00 equiv.) in AcOH (15 mL) was added Br₂ (1.00 mL) dropwise at room temperature under N₂ atmosphere. The mixture was stirred for 4 h at 60° C. The mixture was allowed to cool down to room temperature. The mixture was basified to pH = 9 with Na₂CO₃ aqueous solution. Desired product could be detected by LCMS. The resulting mixture was extracted with EA (3 × 200 mL). The combined organic layers were washed with water (1 × 100 mL), dried over anhydrous Na₂SO₄. After filtration, the filtrate was concentrated under reduced pressure. The residue was purified by reverse phase flash with the following conditions (Column: Spherical C₁₈, 20 - 40 um, 330 g; Mobile Phase A: Water (plus 5 mM FA); Mobile Phase B: ACN; Flow rate: 70 mL/min; Gradient: 0% - 5% B, 8 min, 5% B - 60% B gradient in 25 min; 60% B - 95% B gradient in 5 min; 95% B - 95% B gradient in 5 min; Detector: 220 nm. The fractions containing the desired product were collected at 35% B) to afford 4-benzyl-5-bromo-2H-1,2,4-triazol-3-one (1.12 g, 64.35%) as a white solid.

### 4-Benzyl-5-Bromo-2-Methyl-1,2,4-Triazol-3-One

To a stirred mixture of 4-benzyl-5-bromo-2H-1,2,4-triazol-3-one (500.00 mg, 1.968 mmol, 1.00 equiv.) and C_{S2}CO₃ (770.00 mg, 2.363 mmol, 1.20 equiv.) in DMF (10.00 mL) was added MeI (420.00 mg, 2.959 mmol, 1.50 equiv.) at room temperature under N2 atmosphere. The mixture was stirred for 5 h at room temperature. Desired product could be detected by LCMS. The mixture was purified by reverse phase flash with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B -25% B gradient in 0 min; 25% B - 80% B gradient in 25 min; 80% B - 95% B gradient in 5 min; Detector: 220 nm. The fractions containing the desired product were collected at 55% B) to afford 4-benzyl-5-bromo-2-methyl-1,2,4-triazol-3-one (370 mg, 70.13%) as a white solid.

### 4-Benzyl-5-[3-(4-Chlorophenyl)-4-Phenyl-4,5-Dihydropyrazol-1-yl]-2-Methyl-1,2,4-Triazol-3-One

A mixture of N¹,N²-dimethylcyclohexane-1,2-diamine (31.83 mg, 0.224 mmol, 0.40 equiv.), 3-(4-chlorophenyl)-4-phenyl-4,5-dihydro-1H-pyrazole (215.45 mg, 0.839 mmol, 1.50 equiv.), 4-benzyl-5-bromo-2-methyl-1,2,4-triazol-3-one (150.00 mg, 0.559 mmol, 1.00 equiv.), K₃PO₄ (356.27 mg, 1.678 mmol, 3.00 equiv.), and CuI (21.31 mg, 0.112 mmol, 0.20 equiv.) in dioxane (10 mL) was irradiated with microwave radiation for 2 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 0 min, 5% B - 15% B gradient in 0 min; 15% B -85% B gradient in 35 min; 85% B - 95% B gradient in 10 min; Detector: 220 nm. The fractions containing the desired product were collected at 75% B) to afford 4-benzyl-5-[3-(4-chlorophenyl)-4-phenyl-4,5-dihydropyrazol-1-yl]-2-methyl-1,2,4-triazol-3-one (35 mg, 14.09%) as light yellow solid.

### Example 29. Preparation of Compound 202

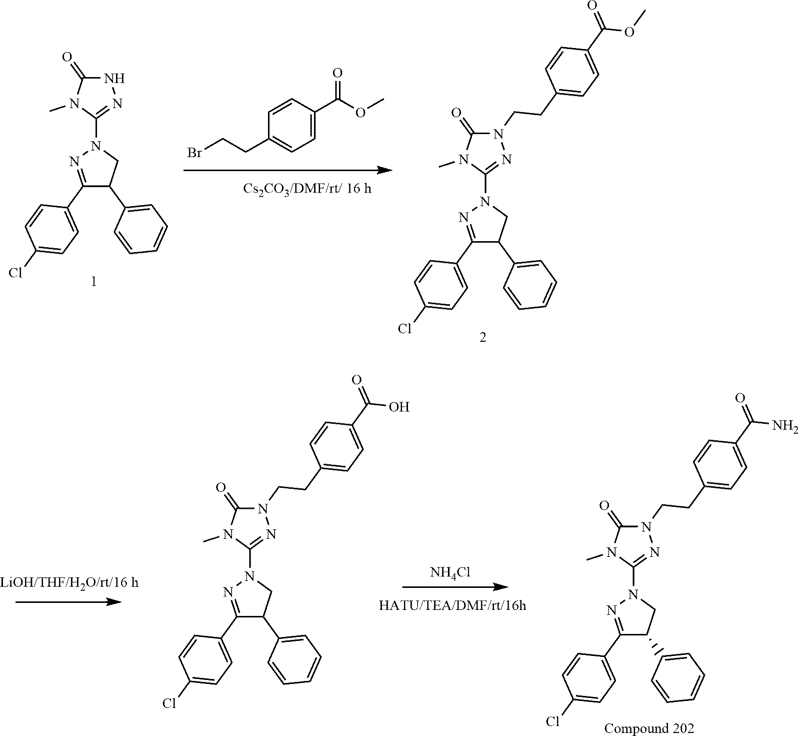

### Methyl 4-(2-[3-[3-(4-Chlorophenyl)-4-Phenyl-4,5-Dihydropyrazol-1-yl]-4-Methyl-5-Oxo-1,2,4-Triazol-1-yl]Ethyl)Benzoate

To a stirred solution of methyl 4-(2-bromoethyl)benzoate (691.04 mg, 2.843 mmol, 4.00 equiv.) and 5-[3-(4-chlorophenyl)-4-phenylpyrazol-1-yl]-4-methyl-2H-1,2,4-triazol-3-one (250.00 mg, 0.711 mmol, 1.00 equiv.) in DMF (4.00 mL, 51.687 mmol, 72.73 equiv.) was added C_{S2}CO₃ (463.09 mg, 1.421 mmol, 2.00 equiv.) at room temperature. The solution was stirred at rt for 4 h. The residue was purified by reverse phase flash chromatography with the following conditions: Column: Spherical C₁₈, 20 - 40 um, 330 g; Mobile Phase A: Water (plus 10 mM NH₄HCO₃); Mobile Phase B: ACN; Flow rate: 80 mL/min; Gradient: 5% - 5% B, 5 min, 75% B - 95% B gradient in 20 min; Detector: 254 nm. The fractions containing the desired product were collected at 90% B and concentrated under reduced pressure to afford methyl 4-(2-[3-[3-(4-chlorophenyl)-4-phenyl-4,5-dihydropyrazol-1-yl]-4-methyl-5-oxo-1,2,4-triazol-1-yl]ethyl)benzoate (200 mg, 54.54%) as yellow oil.

### 4-[3-[3-(4-Chlorophenyl)-4-Phenyl-4,5-Dihydropyrazol-1-yl]-4-Methyl-5-Oxo-1,2,4-Triazol-1-yl]Ethyl)Benzoic Acid

To a stirred solution of methyl 4-(2-[3-[3-(4-chlorophenyl)-4-phenyl-4,5-dihydropyrazol-1-yl]-4-methyl-5-oxo-1,2,4-triazol-1-yl]ethyl)benzoate (230.00 mg, 0.446 mmol, 1.00 equiv.) in THF (4.00 mL, 49.372 mmol, 110.76 equiv.) and H₂O (4.00 mL, 222.033 mmol, 498.13 equiv.) was added LiOH (106.74 mg, 4.457 mmol, 10.00 equiv.) at room temperature. The solution was stirred at rt for 16 h. The mixture was concentrated under reduced pressure. The residue was purified by reverse phase flash chromatography with the following conditions: Column: Spherical C₁₈, 20 - 40 um, 120 g; Mobile Phase A: Water (plus 10 mM NH₄HCO₃); Mobile Phase B: ACN; Flow rate: 60 mL/min; Gradient: 5% - 5% B, 5 min, 45% B - 65% B gradient in 20 min; Detector: 254 nm. The fractions containing the desired product were collected at 58% B and concentrated under reduced pressure to afford 4-(2-[3-[3-(4-chlorophenyl)-4-phenyl-4,5-dihydropyrazol-1-yl]-4-methyl-5-oxo-1,2,4-triazol-1-yl]ethyl)benzoic acid (170 mg, 75.98%) as yellow oil.

### 4-[3-[(4S)-3-(4-Chlorophenyl)-4-Phenyl-4,5-Dihydropyrazol-1-yl]-4-Methyl-5-Oxo-1,2,4-Triazol-1-yl]Ethyl)Benzamide

To a stirred solution of 4-(2-[3-[3-(4-chlorophenyl)-4-phenyl-4,5-dihydropyrazol-1-yl]-4-methyl-5-oxo-1,2,4-triazol-1-yl]ethyl)benzoic acid (90.00 mg, 0.179 mmol, 1.00 equiv.) in DMA (5.00 mL, 53.776 mmol, 299.93 equiv.) was added HATU (102.26 mg, 0.269 mmol, 1.50 equiv.) at room temperature. The solution was stirred at rt for 0.5 h. To the mixture was added NH₄C1 (47.95 mg, 0.896 mmol, 5.00 equiv.) and TEA (54.43 mg, 0.538 mmol, 3.00 equiv.) at rt. The mixture was stirred at rt for 4 h. The residue was purified by reverse phase flash chromatography with the following conditions: Column: Spherical C₁₈, 20 - 40 um, 120 g; Mobile Phase A: Water (plus 10 mM NH₄HCO₃); Mobile Phase B: ACN; Flow rate: 60 mL/min; Gradient: 5% - 5% B, 5 min, 45% B - 75% B gradient in 20 min; Detector: 254 nm. The fractions containing the desired product were collected at 62% B and concentrated under reduced pressure to afford 4-(2-[3-[3-(4-chlorophenyl)-4-phenyl-4,5-dihydropyrazol-1-yl]-4-methyl-5-oxo-1,2,4-triazol-1-yl]ethyl)benzamide (50 mg, 55.66%) as yellow solid. The mixture product (50 mg) was purified by PREP CHIRAL HPLC with the following conditions (Column: Chiralpak IC, 2 ^{∗}25cm, 5 um; Mobile Phase A: Hex, Mobile Phase B: EtOH; Flow rate: 18 mL/min; Gradient: 40% B to 40% B in 23 min; 220/254 nm; RT1:17.993; RT2:20.62; Injection Volumn:0.8 mL; Number Of Runs: 10;) to afford 4-(2-[3-[(4S)-3-(4-chlorophenyl)-4-phenyl-4,5-dihydropyrazol-1-yl]-4-methyl-5-oxo-1,2,4-triazol-1-yl]ethyl)benzamide (19 mg) as an off-white solid. Compounds 195, 200, 210, and 212 were prepared by methods as described for Compound 202 using the appropriate reagents.

### Example 30. Preparation of Compounds 216 and 218

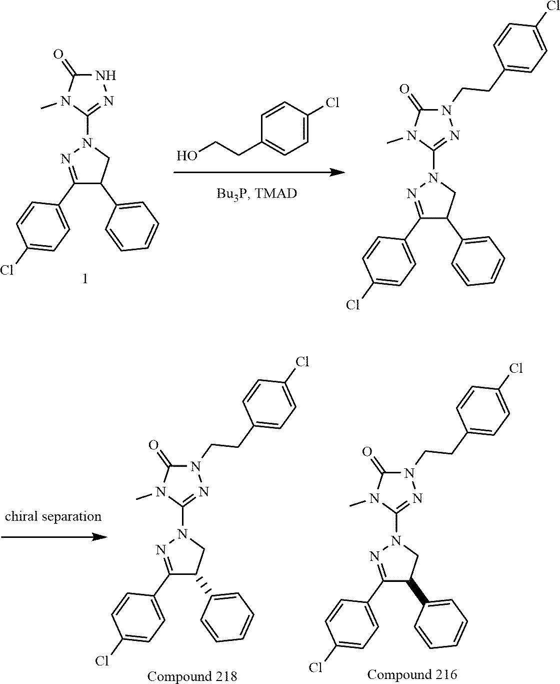

To a stirred solution of 5-[3-(4-chlorophenyl)-4-phenyl-4,5-dihydropyrazol-1-yl]-4-methyl-2H-1,2,4-triazol-3-one (150.00 mg, 0.424 mmol, 1.00 equiv.) and 4-(2-hydroxyethyl)-chlorobenzol (79.67 mg, 0.509 mmol, 1.20 equiv.) in DMF (4.00 mL, 51.687 mmol, 121.92 equiv.) was added Bu₃P (256.92 mg, 1.272 mmol, 3.00 equiv.) at rt. To the solution was added TMAD (219.00 mg, 1.272 mmol, 3.00 equiv.) at 0° C. under nitrogen atmosphere. The solution was stirred at rt for 4 h. The residue was purified by reverse phase flash chromatography with the following conditions: Column: Spherical C₁₈, 20 - 40 um, 330 g; Mobile Phase A: Water (plus 10 mM NH4HCO3); Mobile Phase B: ACN; Flow rate: 80 mL/min; Gradient: 5% - 5% B, 5 min, 75% B - 95% B gradient in 20 min; Detector: 254 nm. The fractions containing the desired product were collected at 80 % B and concentrated under reduced pressure to afford 5-[3-(4-chlorophenyl)-4-phenyl-4,5-dihydropyrazol-1-yl]-2-[2-(4-chlorophenyl)ethyl]-4-methyl-1,2,4-triazol-3-one (140 mg, 67.06%) as an off-white solid. The mixture product (130 mg) was purified by PREP CHIRAL HPLC with the following conditions (Column: CHIRALPAK IG, 20 ^{∗}250mm, 5 um; Mobile Phase A:, Mobile Phase B:; Flow rate: 18 mL/min; Gradient:% B; 220/254 nm; RT1:10.663; RT2:14.359; Injection Volume:0.8 mL; Number Of Runs:7) to afford 5-[(4S)-3-(4-chlorophenyl)-4-phenyl-4,5-dihydropyrazol-1-yl]-2-[2-(4-chlorophenyl)ethyl]-4-methyl-1,2,4-triazol-3-one (52.5 mg) as an off-white solid and afford 5-[(4R)-3-(4-chlorophenyl)-4-phenyl-4,5-dihydropyrazol-1-yl]-2-[2-(4-chlorophenyl)ethyl]-4-methyl-1,2,4-triazol-3-one (66.6 mg) as an off-white solid.

### Example 31. Preparation of Compounds 214, 220, 221, and 223

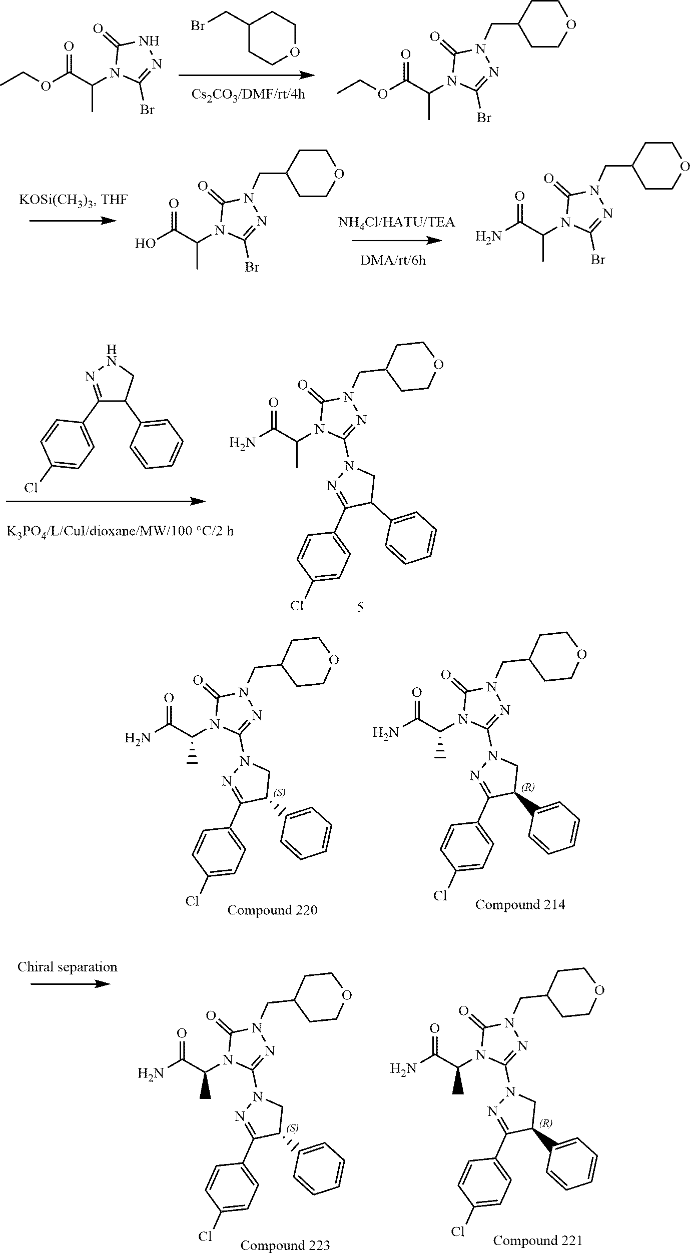

### Ethyl 2-[3-Bromo-1-(Oxan-4-Ylmethyl)-5-Oxo-1,2,4-Triazol-4-yl]Propanoate

A mixture of ethyl 2-(3-bromo-5-oxo-1H-1,2,4-triazol-4-yl)propanoate (1.00 g, 3.787 mmol, 1.00 equiv.), 4-(bromomethyl)oxane (813.70 mg, 4.544 mmol, 1.20 equiv.) and C_{S2}CO₃ (1.85 g, 5.678 mmol, 1.50 equiv.) in DMF (12 mL) was stirred for 4 h at room temperature under nitrogen atmosphere. Desired product could be detected by LCMS. The mixture was neutralized to pH 7 with AcOH. The resulting mixture was extracted with EtOAc (3 × 250 mL), dried over anhydrous Na₂SO₄. After filtration, the filtrate was concentrated under reduced pressure. The residue was purified by reverse phase flash chromatography with the following conditions: Column: Spherical C₁₈, 20-35 um, 330; Mobile Phase A: water (plus 1.7 mM FA); Mobile Phase B: ACN; Flow rate: 90 mL/min; Gradient: 0%-0% B, 8 min, 25%-50% B gradient in 25 min; 98%-98% B, 8 min, Detector: 220 nm. The fractions containing the desired product were collected at 44% B and concentrated under reduced pressure to afford ethyl 2-[3-bromo-1-(oxan-4-ylmethyl)-5-oxo-1,2,4-triazol-4-yl]propanoate (1.18 g, 86.03%) as a yellow oil.

### 2-Bromo-1-(Oxan-4-Ylmethyl)-5-Oxo-1,2,4-Triazol-4-yl]Propanoic Acid

A solution of ethyl 2-[3-bromo-1-(oxan-4-ylmethyl)-5-oxo-1,2,4-triazol-4-yl]propanoate (688.00 mg, 1.899 mmol, 1.00 equiv.) and trimethyl(potassiooxy)silane (243.70 mg, 1.900 mmol, 1.00 equiv.) in THF (12.00 mL) was stirred for 2 h at room temperature under nitrogen atmosphere. Desired product could be detected by LCMS. The solution was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 13%-40% B gradient in 25 min; 98%-98% B, 8 min, Detector: 220 nm. The fractions containing the desired product were collected at 28% B and concentrated under reduced pressure to afford 2-[3-bromo-1-(oxan-4-ylmethyl)-5-oxo-1,2,4-triazol-4-yl]propanoic acid (465 mg, 73.26%) as a white solid. The resulting product was used in the next step directly.

2-bromo-5-oxo-1-((tetrahydro-2H-pyran-4-yl)methyl)-1,5-dihydro-4H-1,2,4-triazol-4-yl)propanamide A solution of 2-[3-bromo-1-(oxan-4-ylmethyl)-5-oxo-1,2,4-triazol-4-yl]propanoic acid (465.00 mg, 1.392 mmol, 1.00 equiv.) and HATU (793.70 mg, 2.087 mmol, 1.50 equiv.) in DMA (10.00 mL) was stirred for 25 min at room temperature under nitrogen atmosphere. Then the solution was added with NH₄Cl (372.20 mg, 6.958 mmol, 5.00 equiv.) and TEA (422.40 mg, 4.174 mmol, 3.00 equiv.) and stirred for 6 h at room temperature under nitrogen atmosphere. Desired product could be detected by LCMS. The solution was purified by reverse phase flash chromatography with the following conditions: Column: Spherical C₁₈, 20-40 um, 120; Mobile Phase A: water (plus 1.7 mM FA); Mobile Phase B: ACN; Flow rate: 70 mL/min; Gradient: 0%-0% B, 8 min, 12%-40% B gradient in 20 min; 98%-98% B, 8 min, Detector: 220 nm. The fractions containing the desired product were collected at 28% B and concentrated under reduced pressure to afford 2-(3-bromo-5-oxo-1-((tetrahydro-2H-pyran-4-yl)methyl)-1,5-dihydro-4H-1,2,4-triazol-4-yl)propanamide (350 mg, 98.91%) as a yellow oil. H-NMR analysis indicated the yellow oil was the desired product.

### (2S)[3-[(4S)-3-(4-Chlorophenyl)-4-Phenyl-4,5-Dihydropyrazol-1-yl]-1-(Oxan-4-Ylmethyl)-5-Oxo-1,2,4-Triazol-4-yl]Propanamide, and (2S)[3-[(4R)-3-(4-Chlorophenyl)-4-Phenyl-4,5-Dihydropyrazol-1-yl]-1-(Oxan-4-Ylmethyl)-5-Oxo-1,2,4-Triazol-4-yl]Propanamide, (2R)[3-[(4R)-3-(4-Chlorophenyl)-4-Phenyl-4,5-Dihydropyrazol-1-yl]-1-(Oxan-4-Ylmethyl)-5-Oxo-1,2,4-triaZol-4-yl]Propanamide and (2R)-2-[3-[(4S)-3-(4-Chlorophenyl)-4-Phenyl-4,5-Dihydropyrazol-1-yl]-1-(Oxan-4-Ylmethyl)-5-Oxo-1,2,4-Triazol-4-yl] propaNamide

Into a 25 mL sealed tube were added 2-[3-bromo-1-(oxan-4-ylmethyl)-5-oxo-1,2,4-triazol-4-yl]propanamide (350.00 mg, 1.050 mmol, 1.00 equiv.), dioxane (10 mL), CuI (40.03 mg, 0.210 mmol, 0.20 equiv.), K₃PO₄ (669.00 mg, 3.152 mmol, 3.00 equiv.), 3-(4-chlorophenyl)-4-phenyl-4,5-dihydro-1H-pyrazole (404.80 mg, 1.577 mmol, 1.50 equiv.) and N¹,N²-dimethylcyclohexane-1,2-diamine (59.80 mg, 0.420 mmol, 0.40 equiv.) at room temperature under nitrogen atmosphere. The resulting mixture was then heated for 3 h at 100° C. under nitrogen atmosphere. The mixture was allowed to cool down to room temperature. Desired product could be detected by LCMS. The mixture was added with 4 g silica gel and concentrated under reduced pressure. The residue was purified by silica gel column chromatography, eluted with PE/EtOAc (1:1) then CH₂Cl₂/MeOH (10:1) to afford 2-[3-[3-(4-chlorophenyl)-4-phenyl-4,5-dihydropyrazol-1-yl]-1-(oxan-4-ylmethyl)-5-oxo-1,2,4-triazol-4-yl]propanamide (250 mg). The residue was purified by reverse phase flash chromatography with the following conditions: Column: Spherical C18, 20-40 um, 120; Mobile Phase A: water (plus 1.7 mM FA); Mobile Phase B: ACN; Flow rate: 70 mL/min; Gradient: 0%-0% B, 8 min, 28%-53% B gradient in 22 min; 98%-98% B, 8 min, Detector: 220 nm. The fractions containing the desired product were collected at 46% B and concentrated under reduced pressure to afford 2-[3-[3-(4-chlorophenyl)-4-phenyl-4,5-dihydropyrazol-1-yl]-1-(oxan-4-ylmethyl)-5-oxo-1,2,4-triazol-4-yl]propanamide (65 mg, 12.16%) as an off-white solid.

The above mixture was purified by Prep-Chiral-HPLC with the following conditions (Column: CHIRALPAK IE, 2 ^{∗}25 cm,5 um; Mobile Phase A: Hex(0.2%IPA), Mobile Phase B: EtOH; Flow rate:20 mL/min; Gradient: 35% B to 35% B in 26 min; Detector: 220/254 nm; RT1: 16.471 min; RT2: 18.046 min; RT3: 20.454 min; RT4: 24.118 min; Injection Volume:0.3 mL; Number Of Runs: 16) to afford (2R)-2-[3-[(4R)-3-(4-chlorophenyl)-4-phenyl-4,5-dihydropyrazol-1-y1]-1-(oxan-4-ylmethyl)-5-oxo-1,2,4-triazol-4-yl]propanamide (7.8 mg, 11.36%) as an off-white solid, (2S)-2-[3-[(4S)-3-(4-chlorophenyl)-4-phenyl-4,5-dihydropyrazol-1-yl]-1-(oxan-4-ylmethyl)-5-oxo-1,2,4-triazol-4-yl]propanamide (12.4 mg, 17.90%) as an off-white solid, (2R)-2-[3-[(4S)-3-(4-chlorophenyl)-4-phenyl-4,5-dihydropyrazol-1-yl]-1-(oxan-4-ylmethyl)-5-oxo-1,2,4-triazol-4-yl]propanamide (10.4 mg, 14.88%) as an off-white solid and (2S)-2-[3-[(4R)-3-(4-chlorophenyl)-4-phenyl-4,5-dihydropyrazol-1-yl]-1-(oxan-4-ylmethyl)-5-oxo-1,2,4-triazol-4-yl]propanamide (13.5 mg, 18.98%) as an off-white solid.

### Example 32. Preparation of Compounds 222 and 224

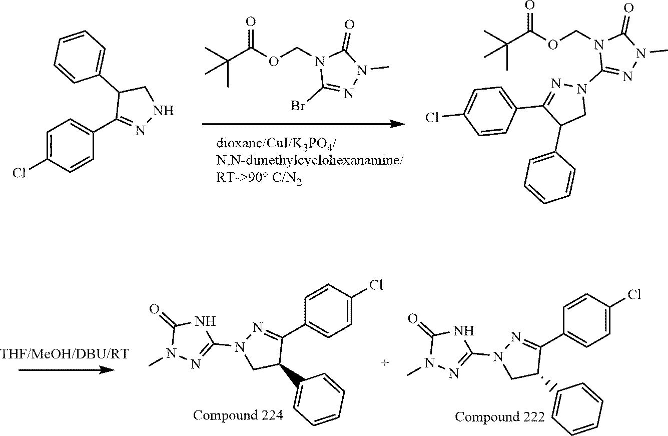

### (4-Chlorophenyl)-4-Phenyl-4,5-Dihydropyrazol-1-yl]-1-Methyl-5-Oxo-1,2,4-Triazol-4-yl]Methyl 2,2-Dimethylpropanoate

To a stirred mixture of (3-bromo-1-methyl-5-oxo-1,2,4-triazol-4-yl)methyl 2,2-dimethylpropanoate (150.00 mg, 0.513 mmol, 1.00 equiv.) and 3-(4-chlorophenyl)-4-phenyl-4,5-dihydro-1H-pyrazole (131.82 mg, 0.513 mmol, 1.00 equiv.) in 1,4-dioxane (10.00 mL) were added CuI (19.56 mg, 0.103 mmol, 0.2 equiv.), K₃PO₄ (326.97 mg, 1.540 mmol, 3.00 equiv.) and N,N-dimethylcyclohexanamine (26.13 mg, 0.205 mmol, 0.4 equiv.) at room temperature under nitrogen atmosphere. The resulting mixture was stirred for 2 h at 90° C. under nitrogen atmosphere. The reaction was monitored by LCMS. The mixture was allowed to cool down to room temperature. The resulting mixture was concentrated under reduced pressure. The residue was purified by Prep-TLC (PE/EtOAc 1:1) to afford [3-[3-(4-chlorophenyl)-4-phenyl-4,5-dihydropyrazol-1-yl]-1-methyl-5-oxo-1,2,4-triazol-4-yl]methyl 2,2-dimethylpropanoate (110 mg, 45.78%) as a brown solid.

### 5-[(4S)(4-Chlorophenyl)-4-Phenyl-4,5-Dihydropyrazol-1-yl]-2-Methyl-4H-1,2,4-TriazolOne and 5-[(4R)(4-Chlorophenyl)-4-Phenyl-4,5-Dihydropyrazol-1-yl]-2-Methyl-4H-1,2,4-TriazolOne

To a stirred mixture of [3-[3-(4-chlorophenyl)-4-phenyl-4,5-dihydropyrazol-1-yl]-1-methyl-5-oxo-1,2,4-triazol-4-yl]methyl 2,2-dimethylpropanoate (110.00 mg, 0.235 mmol, 1.00 equiv.) in THF (1.00 mL) and MeOH (2.00 mL) was added DBU (71.57 mg, 0.470 mmol, 2.00 equiv.) at room temperature. The resulting mixture was stirred for 2 h at room temperature. The reaction was monitored by LCMS. The resulting mixture was concentrated under reduced pressure. The resulting mixture was extracted with EtOAc (2 × 5 mL). The combined organic layers were washed with brine (2 × 10 mL), dried over anhydrous Na₂SO₄. After filtration, the filtrate was concentrated under reduced pressure. The residue was purified by Prep-TLC (PE/EtOAc 1:1) to afford 5-[(4S)-3-(4-chlorophenyl)-4-phenyl-4,5-dihydropyrazol-1-yl]-2-methyl-4H-1,2,4-triazol-3-one (5.5 mg) as a light yellow solid and 5-[(4R)-3-(4-chlorophenyl)-4-phenyl-4,5-dihydropyrazol-1-yl]-2-methyl-4H-1,2,4-triazol-3-one (5.8 mg) as a light yellow solid.

### Example 33. Preparation of Compounds 215 and 217

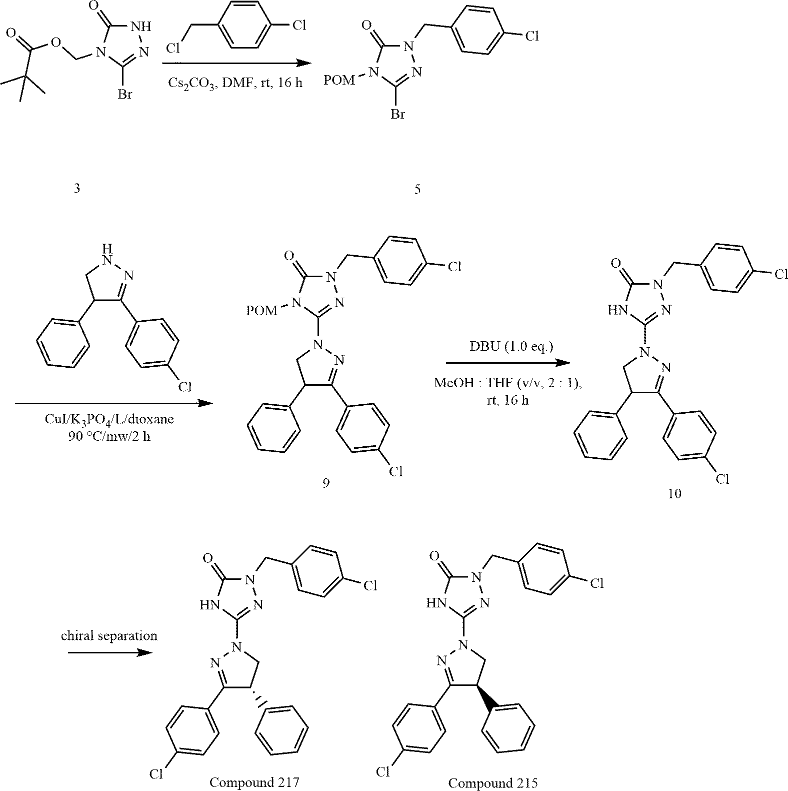

Compounds 217 and 215 were prepared in a similar manner to Compounds 222 and 224 in the previous example, with the modifications indicated in the above scheme.

### Example 34. Preparation of Compounds 115 and 116

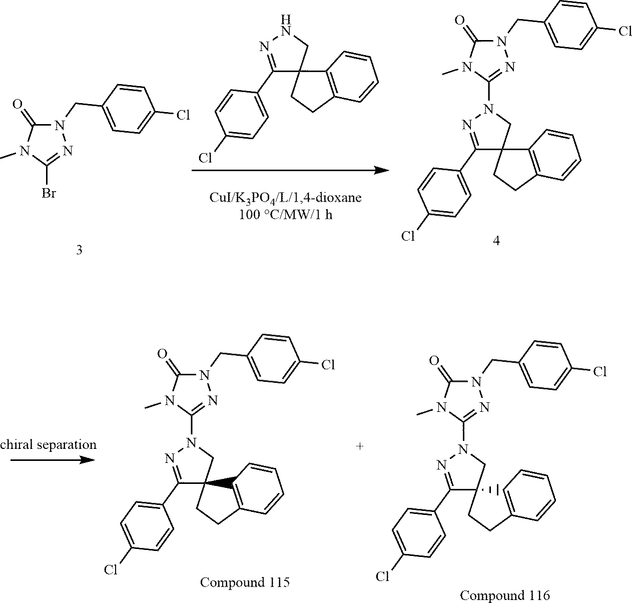

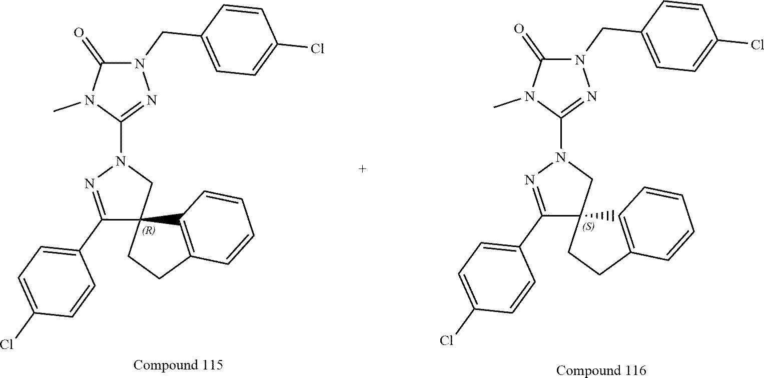

A mixture of 3-(4-chlorophenyl)-1,2,3,5-tetrahydrospiro[indene-1,4-pyrazole] (238.32 mg, 0.843 mmol, 1.5 equiv.), 3-bromo-1-[(4-chlorophenyl)methyl]-4-methyl-4,5-dihydro-1H-1,2,4-triazol-5-one (170 mg, 0.562 mmol, 1 equiv.), N¹,N²-dimethylcyclohexane-1,2-diamine (31.97 mg, 0.225 mmol, 0.4 equiv.), K₃PO₄ (357.80 mg, 1.686 mmol, 3.00 equiv.) and CuI (21.40 mg, 0.112 mmol, 0.20 equiv.) in dioxane (10 mL) was irradiated with microwave radiation for 1.5 h at 100° C. under N₂ atmosphere. The mixture was allowed to cool down to room temperature. Desired product could be detected by LCMS. The reaction was quenched by the addition of water (50 mL) at room temperature. The resulting mixture was extracted with EA (3 × 200 mL). The combined organic layers were washed with water (1 × 100 mL), dried over anhydrous Na₂SO₄. After filtration, the filtrate was concentrated under reduced pressure. The crude product was purified by Prep-HPLC with the following conditions (Column: Spherical C₁₈, 20 - 40 um, 120 g; Mobile Phase A: Water (plus 5 mM FA); Mobile Phase B: ACN; Flow rate: 70 mL/min; Gradient: 0% - 5% B, 8 min, 5% B -35% B gradient in 0 min;35% B - 90% B gradient in 40 min; 90% B - 95% B gradient in 5 min; Detector: 220 nm. The fractions containing the desired product were collected at 82% B) to afford (137 mg) as a white solid. The solid was separated by CHIRAL-HPLC with the following conditions (Column: CHIRALPAK IG, 20 ^{∗}250mm, 5 um; Mobile Phase A: Hex., Mobile Phase B: IPA; Flow rate: 20 mL/min; Gradient: 30% B to 30% B in 14 min; 220/254 nm; RT1:8.096 min; RT2:11.662 min) to afford 3-[(1R)-3-(4-chlorophenyl)-1,2,3,5-tetrahydrospiro[indene-1,4-pyrazol]-1-y1]-1-[(4-chlorophenyl)methyl]-4-methyl-4,5-dihydro-1H-1,2,4-triazol-5-one (29.7 mg, 10.48%) as a white solid and 3-[(1S)-3-(4-chlorophenyl)-1,2,3,5-tetrahydrospiro[indene-1,4-pyrazol]-1-yl]-1-[(4-chlorophenyl)methyl]-4-methyl-4,5-dihydro-1H-1,2,4-triazol-5-one (29.9 mg, 10.55%) as a white solid.

### Example 35. Preparation of Compounds 203 and 209

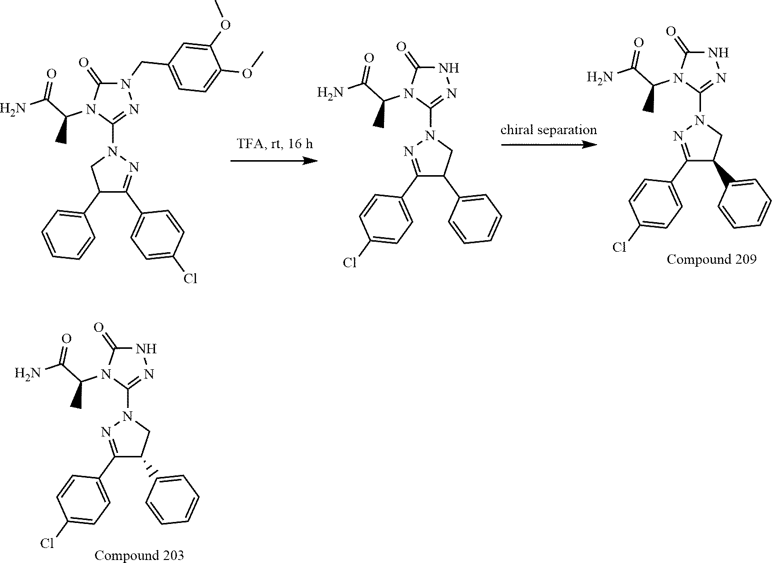

A solution of (2S)-2-[3-[3-(4-chlorophenyl)-4-phenyl-4,5-dihydropyrazol-1-yl]-1-[(3,4-dimethoxyphenyl)methyl]-5-oxo-1,2,4-triazol-4-yl]propanamide (130.00 mg, 0.232 mmol, 1.00 equiv.) in TFA (20.00 mL) was reflux for 30 h at 85° C. under N₂ atmosphere. The mixture was allowed to cool down to room temperature. Desired product could be detected by LCMS. The resulting mixture was concentrated under reduced pressure. The crude product was purified by Prep-HPLC to afford (2S)-2-[3-(4-chlorophenyl)-4-phenyl-4,5-dihydropyrazol-1-yl]-5-oxo-1H-1,2,4-triazol-4-yl]propanamide (60 mg) as a white solid. The solid was separated by CHIRAL-HPLC to afford as a white solid (2S)-2-[3-[(4S)-3-(4-chlorophenyl)-4-phenyl-4,5-dihydropyrazol-1-yl]-5-oxo-1H-1,2,4-triazol-4-yl]propanamide (22 mg, 23.11%) and (2S)-2-[3-[(4R)-3-(4-chlorophenyl)-4-phenyl-4,5-dihydropyrazol-1-yl]-5-oxo-1H-1,2,4-triazol-4-yl]propanamide (22 mg, 23.11%) as a white solid. Compounds 199 and 206 were prepared by similar methods described above for Compounds 203 and 209 using the appropriate reagent.

### Example 36. Preparation of Compounds 208 and 211

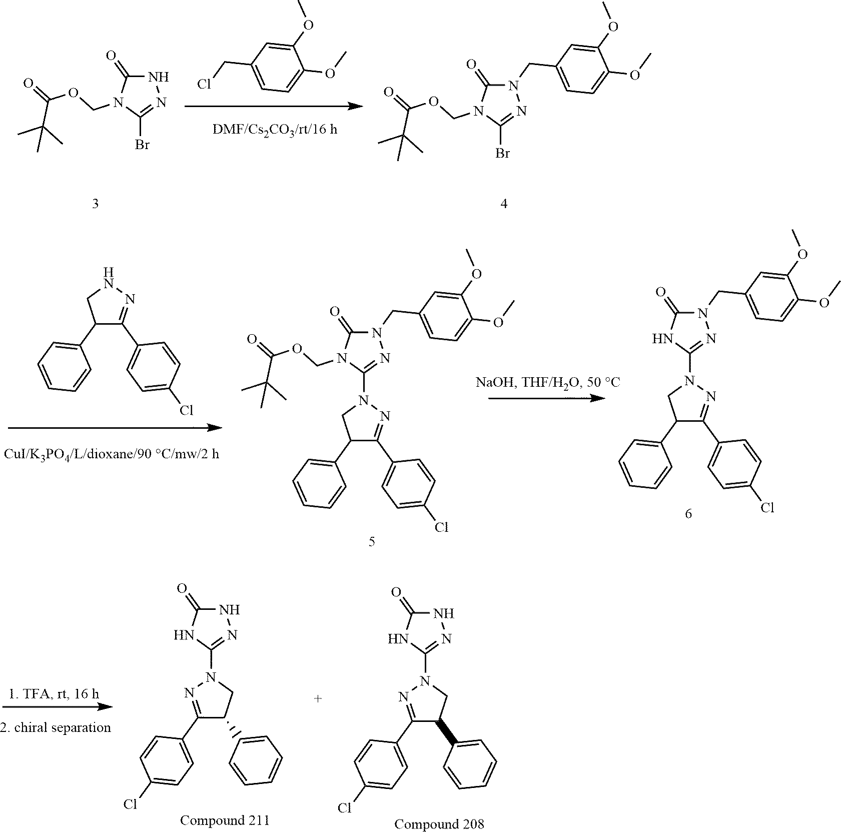

### Bromo-1-[(3,4-Dimethoxyphenyl)Methyl]-5-Oxo-1,2,4-Triazol-4-yl]Methyl 2,2-Dimethylpropanoate

To a stirred mixture of (3-bromo-5-oxo-1H-1,2,4-triazol-4-yl)methyl 2,2-dimethylpropanoate (2.20 g, 7.911 mmol, 1.00 equiv.) and 4-(chloromethyl)-1,2-dimethoxybenzene (2.95 g, 15.806 mmol, 2.00 equiv.) in DMF (20.00 mL) was added C_{S2}CO₃ (7.73 g, 23.725 mmol, 3.00 equiv.) at room temperature. The resulting mixture was stirred for 16 h at room temperature. The reaction was monitored by LCMS. The resulting mixture was extracted with EtOAc (3 × 300 mL). The combined organic layers were washed with brine (3 × 50 mL), dried over anhydrous Na₂SO₄. After filtration, the filtrate was concentrated under reduced pressure. The residue was purified by silica gel column chromatography, eluted with PE/EtOAc (20:1 to 1:1) to afford [3-bromo-1-[(3,4-dimethoxyphenyl)methyl]-5-oxo-1,2,4-triazol-4-yl]methyl 2,2-dimethylpropanoate (2 g, 59.03%) as a brown solid.

### (4-Chlorophenyl)-4-Phenyl-4,5-Dihydropyrazol-1-yl]-1-[(3,4-Dimethoxyphenyl)Methyl]-5-Oxo-1,2,4-Triazol-4-yl]Methyl 2,2-Dimethylpropanoate

To a stirred mixture of [3-bromo-1-[(3,4-dimethoxyphenyl)methyl]-5-oxo-1,2,4-triazol-4-yl]methyl 2,2-dimethylpropanoate (2.00 g, 4.670 mmol, 1.00 equiv.) and 3-(4-chlorophenyl)-4-phenyl-4,5-dihydro-1H-pyrazole (1.20 g, 4.670 mmol, 1.0 equiv.) in 1,4-dioxane (10.00 mL) were added CuI (0.18 g, 0.945 mmol, 0.20 equiv.), N,N-dimethylcyclohexanamine (0.24 g, 1.868 mmol, 0.4 equiv.) and K3PO4 (2.97 g, 14.009 mmol, 3.0 equiv.) at room temperature under nitrogen atmosphere. The resulting mixture was stirred for 2 h at 90° C. under nitrogen atmosphere. The reaction was monitored by LCMS. The mixture was allowed to cool down to room temperature. The resulting mixture was concentrated under reduced pressure. The residue was purified by silica gel column chromatography, eluted with PE/EtOAc (50:1 to 1:1) to afford [3-[3-(4-chlorophenyl)-4-phenyl-4,5-dihydropyrazol-1-yl]-1-[(3,4-dimethoxyphenyl)methyl]-5-oxo-1,2,4-triazol-4-yl]methyl 2,2-dimethylpropanoate (1 g, 35.45%) as a light blue solid.

### 5-(4-Chlorophenyl)-4-Phenyl-4,5-Dihydropyrazol-1-yl]-2-[(3,4-Dimethoxyphenyl)Methyl]-4H-1,2,4-Triazol-3-One

To a stirred mixture of [3-[3-(4-chlorophenyl)-4-phenyl-4,5-dihydropyrazol-1-yl]-1-[(3,4-dimethoxyphenyl)methyl]-5-oxo-1,2,4-triazol-4-yl]methyl 2,2-dimethylpropanoate (1.10 g, 1.821 mmol, 1.00 equiv.) and DBU (0.55 g, 3.642 mmol, 2.0 equiv.) in THF (5.00 mL) was added MeOH (10.00 mL) at room temperature. The resulting mixture was stirred for 16 h at room temperature. The reaction was monitored by TLC. The resulting mixture was concentrated under reduced pressure. The residue was purified by silica gel column chromatography, eluted with PE/EtOAc (10:1 to 1:1) to afford 5-[3-(4-chlorophenyl)-4-phenyl-4,5-dihydropyrazol-1-yl]-2-[(3,4-dimethoxyphenyl)methyl]-4H-1,2,4-triazol-3-one (450 mg, 50.44%) as a brown solid.

### 5-[(4R)(4-Chlorophenyl)-4-Phenyl-4,5-Dihydropyrazol-1-yl]-2,4-Dihydro-1,2,4-TriazolOne and 5-[(4S)(4-Chlorophenyl)-4-Phenyl-4,5-Dihydropyrazol-1-yl]-2,4-Dihydro-1,2,4-TriazolOne

A solution of 5-[3-(4-chlorophenyl)-4-phenyl-4,5-dihydropyrazol-1-yl]-2-[(3,4-dimethoxyphenyl)methyl]-4H-1,2,4-triazol-3-one (200.00 mg) in TFA (20.00 mL) was stirred for 16 h at 80° C. under nitrogen atmosphere. The resulting mixture was concentrated under reduced pressure. The resulting mixture was diluted with EtOAc (20 mL). The reaction was washed with sat. NaHCO₃ (aq.) at room temperature. The organic phase was concentrated under vacuum. The crude product was purified by Chiral-HPLC with the following conditions (Column: CHIRALPAK IG, 2 ^{∗}25cm,5 um; Mobile Phase A:Hex, Mobile Phase B:EtOH; Flow rate:20 mL/min; Gradient:50 B to 50 B in 18 min; 220/254 nm; RT1:7.989; RT2:15.898; Injection Volumn: 1.66 mL; Number Of Runs:5) to afford 5-[(4R)-3-(4-chlorophenyl)-4-phenyl-4,5-dihydropyrazol-1-yl]-2,4-dihydro-1,2,4-triazol-3-one (18.9 mg) as an off-white solid and 5-[(4S)-3-(4-chlorophenyl)-4-phenyl-4,5-dihydropyrazol-1-yl]-2,4-dihydro-1,2,4-triazol-3-one (19.8 mg) as an off-white solid.

The following compounds were prepared using the methods described above:

| # | Structure | MW | NMR |
|---|---|---|---|
| 100 | 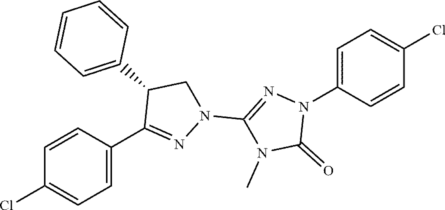 [SMILES: Cn1c(N2C[C@@H](c3ccccc3)C(c3ccc(Cl)cc3)=N2)nn(-c2ccc(Cl)cc2)c1=O] | 464.4 | ¹H NMR (400 MHz, DMSO-d6) chemical shifts 7.95 -7.88 (m, 2H), 7.74 -7.68 (m, 2H), 7.53 -7.42 (m, 4H), 7.35 (d, J = 4.3 Hz, 4H), 7.27 (q, J = 4.3 Hz, 1H), 5.08 (dd, J = 11.0, 4.4 Hz, 1H), 4.43 (t, J = 10.8 Hz, 1H), 3.88 (dd, J = 10.5, 4.4 Hz, 1H), 3.56 (s, 3H). |
| 101 | 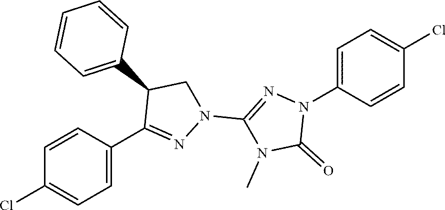 [SMILES: Cn1c(N2C[C@H](c3ccccc3)C(c3ccc(Cl)cc3)=N2)nn(-c2ccc(Cl)cc2)c1=O] | 464.4 | ¹H NMR (400 MHz, DMSO-d6) chemical shifts 7.95 -7.86 (m, 2H), 7.71 (d, J = 8.4 Hz, 2H), 7.47 (dd, J = 20.4, 8.5 Hz, 4H), 7.35 (d, J = 4.4 Hz, 4H), 7.31 ^{··}C 7.22 (m, 1H), 5.08 (dd, J = 11.0, 4.4 Hz, 1H), 4.43 (t, J = 10.8 Hz, 1H), 3.88 (dd, J = 10.5, 4.4 Hz, 1H), 3.56 (s, 3H). |
| 102 | 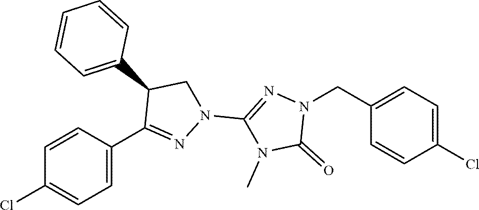 [SMILES: Cn1c(N2C[C@H](c3ccccc3)C(c3ccc(Cl)cc3)=N2)nn(Cc2ccc(Cl)cc2)c1=O] | 478.4 | ¹H NMR (400 MHz, DMSO-d6) chemical shifts 7.71 -7.64 (m, 2H), 7.46 -7.35 (m, 4H), 7.29 (q, J = 8.2, 7.1 Hz, 7H), 4.98 (dd, J = 10.9, 4.0 Hz, 1H), 4.85 (d, J = 3.7 Hz, 2H), 4.24 (t, J = 10.7 Hz, 1H), 3.69 (dd, J = 10.4, 4.0 Hz, 1H), 3.47 (s, 3H). [a]25D = +278. |
| 103 | 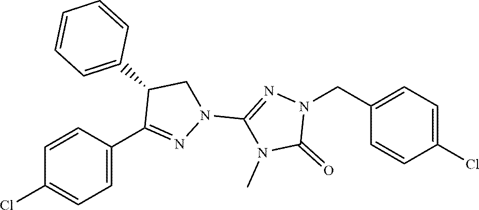 [SMILES: Cn1c(N2C[C@@H](c3ccccc3)C(c3ccc(Cl)cc3)=N2)nn(Cc2ccc(Cl)cc2)c1=O] | 478.4 | ¹H NMR (400 MHz, DMSO-d6) chemical shifts 7.71 - 7.63 (m, 2H), 7.47 -7.35 (m, 4H), 7.36 -7.20 (m, 7H), 4.98 (dd, J = 11.0, 4.0 Hz, 1H), 4.85 (d, J = 3.7 Hz, 2H), 4.24 (t, J= 10.7 Hz, 1H), 3.69 (dd, J = 10.5, 4.0 Hz, 1H), 3.47 (s, 3H). [a]25D = -139. |
| 104 | 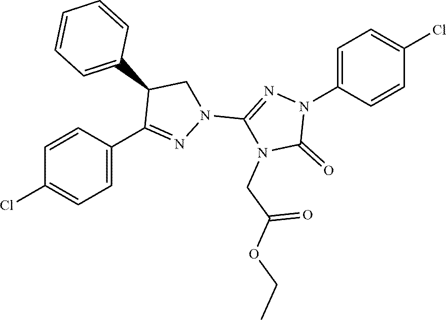 [SMILES: CCOC(=O)Cn1c(N2C[C@H](c3ccccc3)C(c3ccc(Cl)cc3)=N2)nn(-c2ccc(Cl)cc2)c1=O] | 536.4 | ¹H NMR (400 MHz, DMSO-d6) chemical shifts 7.94 - 7.88 (m, 2H), 7.66 - 7.59 (m, 2H), 7.56 -7.44 (m, 4H), 7.37 - 7.23 (m, 5H), 5.09 (dd, J = 11.1, 4.1 Hz, 1H), 4.96 - 4.79 (m, 2H), 4.43 (t, J = 10.9 Hz, 1H), 4.28 - 4.12 (m, 2H), 3.91 (dd, J= 10.6, 4.1 Hz, 1H), 1.21 (t, J = 7.1 Hz, 3H). |
| 105 | 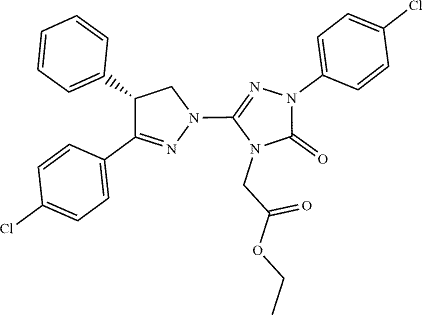 [SMILES: CCOC(=O)Cn1c(N2C[C@@H](c3ccccc3)C(c3ccc(Cl)cc3)=N2)nn(-c2ccc(Cl)cc2)c1=O] | 536.4 | ¹H NMR (400 MHz, DMSO-d6) chemical shifts 7.96 - 7.88 (m, 2H), 7.67 - 7.60 (m, 2H), 7.56 -7.44 (m, 4H), 7.38 - 7.23 (m, 5H), 5.09 (dd, J = 11.1, 4.1 Hz, 1H), 4.95 - 4.79 (m, 2H), 4.43 (t, J = 10.9 Hz, 1H), 4.27 - 4.14 (m, 2H), 3.91 (dd, J= 10.6, 4.1 Hz, 1H), 1.21 (t, J = 7.1 Hz, 3H). |
| 106 | 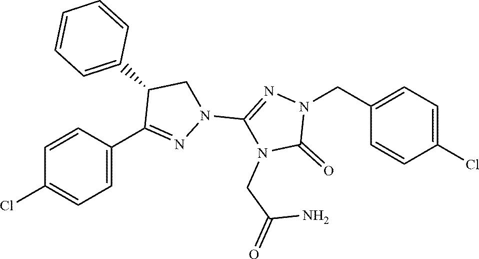 [SMILES: NC(=O)Cn1c(N2C[C@@H](c3ccccc3)C(c3ccc(Cl)cc3)=N2)nn(Cc2ccc(Cl)cc2)c1=O] | 521.4 | ¹H NMR (400 MHz, DMSO-d6) chemical shifts 7.75 - 7.63 (m, 3H), 7.43 - 7.35 (m, 4H), 7.34 -7.20 (m, 8H), 4.96 (dd, J = 11.0, 4.0 Hz, 1H), 4.86 (d, J = 2.7 Hz, 2H), 4.56 (s, 2H), 4.23 (t, J = 10.9 Hz, 1H), 3.69 (dd, J = 10.5, 4.1 Hz, 1H). |
| 107 | 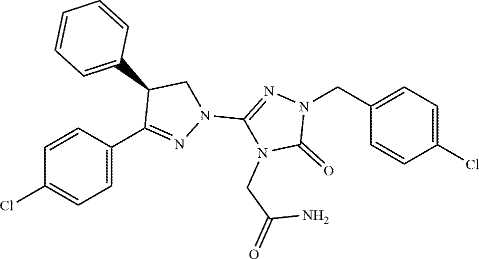 [SMILES: NC(=O)Cn1c(N2C[C@H](c3ccccc3)C(c3ccc(Cl)cc3)=N2)nn(Cc2ccc(Cl)cc2)c1=O] | 521.4 | ¹H NMR (400 MHz, DMSO-d6) chemical shifts 7.74 - 7.65 (m, 3H), 7.44 - 7.36 (m, 4H), 7.33 -7.22 (m, 8H), 4.96 (dd, J = 11.1, 4.0 Hz, 1H), 4.86 (d, J = 2.8 Hz, 2H), 4.56 (s, 2H), 4.23 (t, J = 10.8 Hz, 1H), 3.69 (dd, J = 10.6, 4.0 Hz, 1H). |
| 108 | 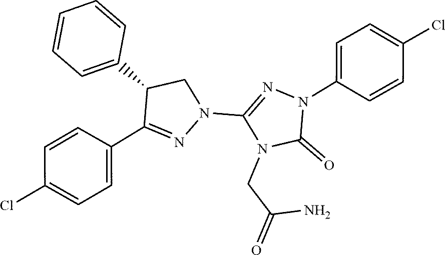 [SMILES: NC(=O)Cn1c(N2C[C@@H](c3ccccc3)C(c3ccc(Cl)cc3)=N2)nn(-c2ccc(Cl)cc2)c1=O] | 507.4 | ¹H NMR (400 MHz, DMSO-d6) chemical shifts 7.96 - 7.89 (m, 2H), 7.81 (s, 1H), 7.75 - 7.68 (m, 2H), 7.55 - 7.48 (m, 2H), 7.44 -7.21 (m, 8H), 5.06 (dd, J = 11.1, 4.2 Hz, 1H), 4.64 (s, 2H), 4.41 (t, J = 10.9 Hz, 1H), 3.88 (dd, J = 10.6, 4.2 Hz, 1H). |
| 109 | 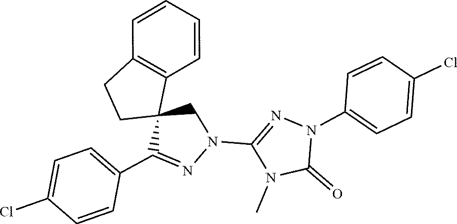 [SMILES: Cn1c(N2C[C@]3(CCc4ccccc43)C(c3ccc(Cl)cc3)=N2)nn(-c2ccc(Cl)cc2)c1=O] | 490.4 | ¹H NMR (400 MHz, DMSO-d6) chemical shifts 7.93 - 7.88 (m, 2H), 7.53 - 7.47 (m, 2H), 7.39 (s, 5H), 7.37 - 7.31 (m, 1H), 7.23 (dd, J = 6.4, 1.4 Hz, 2H), 4.33 (d, J = 10.4 Hz, 1H), 3.88 (d, J = 10.3 Hz, 1H), 3.54 (s, 3H), 3.29 - 3.17 (m, 1H), 3.09 (dt, J = 16.4, 6.6 Hz, 1H), 2.49 - 2.43 (m, 2H). |
| 110 | 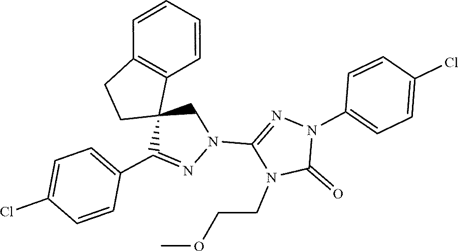 [SMILES: COCCn1c(N2C[C@]3(CCc4ccccc43)C(c3ccc(Cl)cc3)=N2)nn(-c2ccc(Cl)cc2)c1=O] | 534.4 | ¹H NMR (400 MHz, DMSO-d6) chemical shifts 7.93 - 7.88 (m, 2H), 7.52 - 7.48 (m, 2H), 7.43 -7.38 (m, 3H), 7.37 - 7.31 (m, 3H), 7.27 - 7.20 (m, 2H), 4.34 (d, J = 10.3 Hz, 1H), 4.30 - 4.18 (m, 2H), 3.89 (d, J = 10.3 Hz, 1H), 3.74 (t, J = 5.9 Hz, 2H), 3.31 (s, 3H), 3.26 -3.18 (m, 1H), 3.13 - 3.04 (m, 1H), 2.49 - 2.43 (m, 2H). |
| 111 | 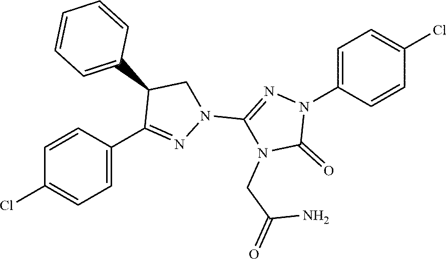 [SMILES: NC(=O)Cn1c(N2C[C@H](c3ccccc3)C(c3ccc(Cl)cc3)=N2)nn(-c2ccc(Cl)cc2)c1=O] | 507.4 | ¹H NMR (400 MHz, DMSO-d6) chemical shifts 7.95 - 7.89 (m, 2H), 7.81 (s, 1H), 7.74 - 7.69 (m, 2H), 7.54 - 7.48 (m, 2H), 7.44 -7.23 (m, 8H), 5.06 (dd, J = 11.1, 4.2 Hz, 1H), 4.64 (s, 2H), 4.41 (t, J = 10.9 Hz, 1H), 3.88 (dd, J = 10.7, 4.2 Hz, 1H). |
| 112 | 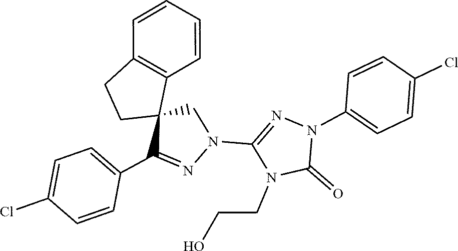 [SMILES: O=c1n(-c2ccc(Cl)cc2)nc(N2C[C@@]3(CCc4ccccc43)C(c3ccc(Cl)cc3)=N2)n1CCO] | 520.4 | ¹H NMR (400 MHz, DMSO-d6) chemical shifts 7.93 - 7.89 (m, 2H), 7.52 - 7.47 (m, 2H), 7.43 -7.31 (m, 6H), 7.26 - 7.19 (m, 2H), 5.00 (t, J = 5.7 Hz, 1H), 4.32 (d, J = 10.3 Hz, 1H), 4.19 - 4.09 (m, 2H), 3.89 (d, J= 10.3 Hz, 1H), 3.78 (q, J = 6.0 Hz, 2H), 3.26 -3.18 (m, 1H), 3.14 - 3.05 (m, 1H), 2.45 (d, J = 8.2 Hz, 2H). |
| 113 | 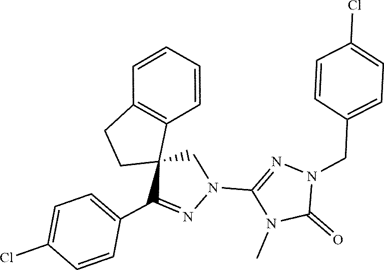 [SMILES: Cn1c(N2C[C@@]3(CCc4ccccc43)C(c3ccc(Cl)cc3)=N2)nn(Cc2ccc(Cl)cc2)c1=O] | 504.4 | ¹H NMR (400 MHz, DMSO-d6) chemical shifts 7.45 - 7.27 (m, 10H), 7.24 - 7.15 (m, 2H), 4.84 (d, J = 1.7 Hz, 2H), 4.14 (d, J = 10.2 Hz, 1H), 3.68 (d, J = 10.2 Hz, 1H), 3.46 (s, 3H), 3.17 (dt, J = 16.8, 8.6 Hz, 1H), 3.05 (dt, J = 16.3, 6.4 Hz, 1H), 2.40 (t, J = 8.4 Hz, 2H). |
| 114 | 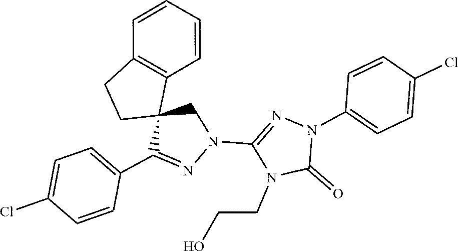 [SMILES: O=c1n(-c2ccc(Cl)cc2)nc(N2C[C@]3(CCc4ccccc43)C(c3ccc(Cl)cc3)=N2)n1CCO] | 520.4 | ¹H NMR (400 MHz, DMSO-d6) chemical shifts 7.94 - 7.88 (m, 2H), 7.53 - 7.48 (m, 2H), 7.44 -7.31 (m, 6H), 7.26 - 7.19 (m, 2H), 5.00 (t, J = 5.8 Hz, 1H), 4.33 (d, J = 10.3 Hz, 1H), 4.18 - 4.09 (m, 2H), 3.89 (d, J= 10.3 Hz, 1H), 3.78 (q, J = 6.0 Hz, 2H), 3.26 -3.18 (m, 1H), 3.13 - 3.06 (m, 1H), 2.45 (d, J = 8.3 Hz, 2H). |
| 115 | 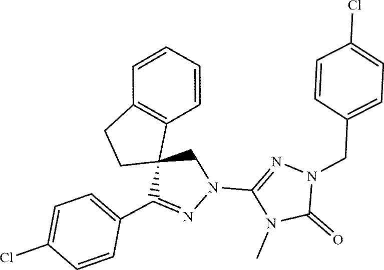 [SMILES: Cn1c(N2C[C@]3(CCc4ccccc43)C(c3ccc(Cl)cc3)=N2)nn(Cc2ccc(Cl)cc2)c1=O] | 504.4 | ¹H NMR (400 MHz, DMSO-d6) chemical shifts 7.43 - 7.25 (m, 10H), 7.24 - 7.15 (m, 2H), 4.84 (d, J = 1.8 Hz, 2H), 4.14 (d, J = 10.3 Hz, 1H), 3.68 (d, J = 10.2 Hz, 1H), 3.46 (s, 3H), 3.22 - 3.13 (m, 1H), 3.08 - 3.01 (m, 1H), 2.40 (t, J = 8.4 Hz, 2H). |
| 116 | 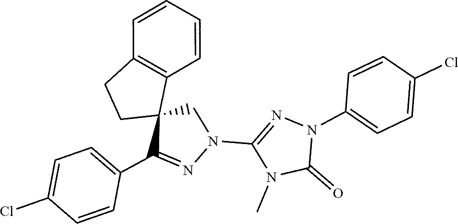 [SMILES: Cn1c(N2C[C@@]3(CCc4ccccc43)C(c3ccc(Cl)cc3)=N2)nn(-c2ccc(Cl)cc2)c1=O] | 490.4 | ¹H NMR (400 MHz, DMSO-d6) chemical shifts 7.96 - 7.86 (m, 2H), 7.53 - 7.47 (m, 2H), 7.38 (s, 5H), 7.33 (ddd, J = 7.6, 6.4, 2.0 Hz, 1H), 7.28 - 7.19 (m, 2H), 4.33 (d, J = 10.4 Hz, 1H), 3.88 (d, J = 10.3 Hz, 1H), 3.54 (s, 3H), 3.22 (dt, J = 16.9, 8.7 Hz, 1H), 3.09 (dt, J = 16.3, 6.6 Hz, 1H), 2.46 (dd, J = 8.5, 5.1 Hz, 2H). |
| 117 | 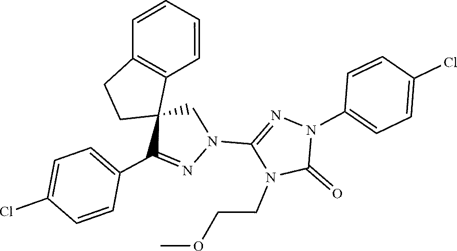 [SMILES: COCCn1c(N2C[C@@]3(CCc4ccccc43)C(c3ccc(Cl)cc3)=N2)nn(-c2ccc(Cl)cc2)c1=O] | 534.4 | ¹H NMR (400 MHz, DMSO-d6) chemical shifts 7.94 - 7.87 (m, 2H), 7.53 - 7.46 (m, 2H), 7.44 -7.37 (m, 3H), 7.39 - 7.29 (m, 3H), 7.28 -7.18 (m, 2H), 4.34 (d,J= 10.4 Hz, 1H), 4.32 - 4.17 (m, 2H), 3.89 (d, J = 10.3 Hz, 1H), 3.74 (t, J = 5.9 Hz, 2H), 3.31 (s, 3H), 3.23 (m, 1H), 3.15 - 3.03 (m, 1H), 2.46 (t, J = 7.5 Hz, 2H). |
| 118 | 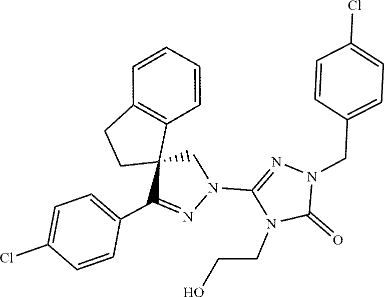 [SMILES: O=c1n(Cc2ccc(Cl)cc2)nc(N2C[C@@]3(CCc4ccccc43)C(c3ccc(Cl)cc3)=N2)n1CCO] | 534.4 | ¹H NMR (400 MHz, DMSO-d6) chemical shifts 7.47 - 7.26 (m, 10H), 7.24 -7.10 (m, 2H), 4.90 (d, J = 50.6 Hz, 3H), 4.26 - 3.98 (m, 3H), 3.84 - 3.61 (m, 3H), 3.26 -2.98 (m, 2H), 2.40 (t, J = 7.0 Hz, 2H). |
| 119 | 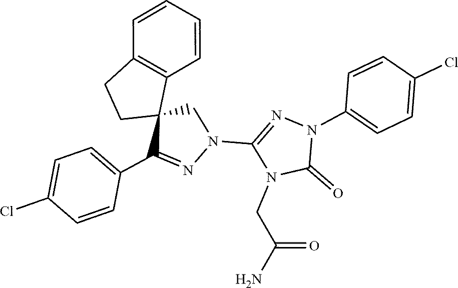 [SMILES: NC(=O)Cn1c(N2C[C@@]3(CCc4ccccc43)C(c3ccc(Cl)cc3)=N2)nn(-c2ccc(Cl)cc2)c1=O] | 533.4 | ¹H NMR (400 MHz, DMSO-d6) chemical shifts 7.95 - 7.88 (m, 2H), 7.76 (s, 1H), 7.51 (d, J = 8.9 Hz, 2H), 7.43 - 7.30 (m, 7H), 7.23 (t, J = 7.4 Hz, 1H), 7.14 (d, J = 7.6 Hz, 1H), 4.68 - 4.54 (m, 2H), 4.36 (d, J = 10.5 Hz, 1H), 3.87 (d, J = 10.5 Hz, 1H), 3.29 - 3.17 (m, 1H), 3.15 - 3.05 (m, 1H), 2.44 (d, J = 9.3 Hz, 2H). |
| 120 | 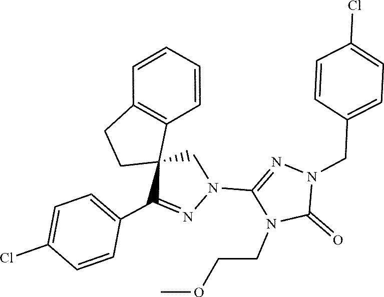 [SMILES: COCCn1c(N2C[C@@]3(CCc4ccccc43)C(c3ccc(Cl)cc3)=N2)nn(Cc2ccc(Cl)cc2)c1=O] | 548.5 | ¹H NMR (400 MHz, Methanol-d4) chemical shifts 7.63 - 6.99 (m, 12H), 4.95 (s, 2H), 4.30 (tq, J = 14.0, 7.0, 5.8 Hz, 2H), 4.12 (d, J = 10.2 Hz, 1H), 3.89 - 3.75 (m, 3H), 3.40 (s, 3H), 3.27 - 3.05 (m, 2H), 2.45 - 2.36 (m, 2H). |
| 121 | 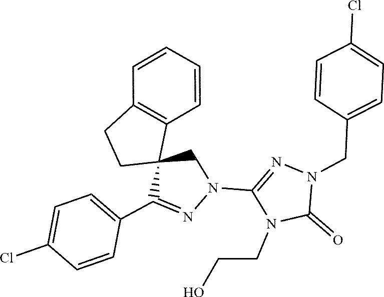 [SMILES: O=c1n(Cc2ccc(Cl)cc2)nc(N2C[C@]3(CCc4ccccc43)C(c3ccc(Cl)cc3)=N2)n1CCO] | 534.4 | ¹H NMR (400 MHz, DMSO-d6) chemical shifts 7.44 - 7.26 (m, 10H), 7.23 - 7.13 (m, 2H), 4.96 (t, J = 5.7 Hz, 1H), 4.84 (s, 2H), 4.16 - 3.96 (m, 3H), 3.77 - 3.65 (m, 3H), 3.17 (dt, J = 17.0, 8.7 Hz, 1H), 3.10 - 3.00 (m, 1H), 2.40 (t, J = 7.4 Hz, 2H). |
| 122 | 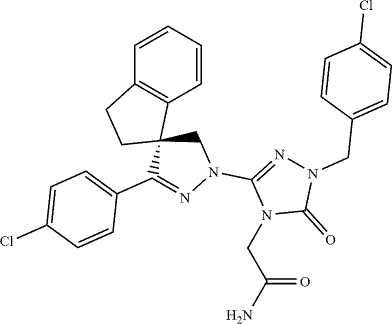 [SMILES: NC(=O)Cn1c(N2C[C@]3(CCc4ccccc43)C(c3ccc(Cl)cc3)=N2)nn(Cc2ccc(Cl)cc2)c1=O] | 547.4 | ¹H NMR (400 MHz, DMSO-d6) chemical shifts 7.67 (s, 1H), 7.43 -7.35 (m, 3H), 7.35 - 7.26 (m, 8H), 7.19 (t, J = 7.3 Hz, 1H), 7.09 (d, J = 7.6 Hz, 1H), 4.85 (d, J = 4.0 Hz, 2H), 4.52 (d, J = 6.7 Hz, 2H), 4.15 (d, J = 10.5 Hz, 1H), 3.67 (d, J = 10.4 Hz, 1H), 3.21 - 3.13 (m, 1H), 3.08 - 3.01 (m, 1H), 2.43 - 2.33 (m, 2H). |
| 123 | 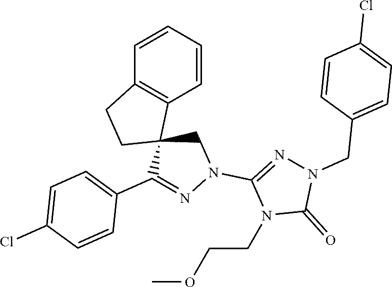 [SMILES: COCCn1c(N2C[C@]3(CCc4ccccc43)C(c3ccc(Cl)cc3)=N2)nn(Cc2ccc(Cl)cc2)c1=O] | 548.5 | ¹H NMR (400 MHz, DMSO-d6) chemical shifts 7.43 - 7.24 (m, 10H), 7.24 - 7.12 (m, 2H), 4.84 (d, J = 2.0 Hz, 2H), 4.24 - 4.06 (m, 3H), 3.7 - 3.65 (m, 3H), 3.29 (s, 3H), 3.22 - 3.00 (m, 2H), 2.45 -2.36 (m, 2H). |
| 124 | 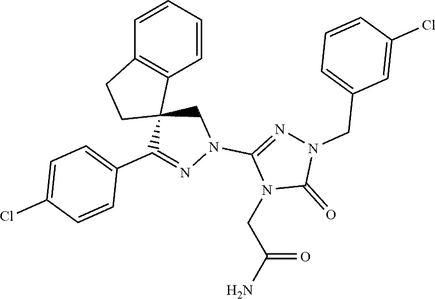 [SMILES: NC(=O)Cn1c(N2C[C@]3(CCc4ccccc43)C(c3ccc(Cl)cc3)=N2)nn(Cc2cccc(Cl)c2)c1=O] | 533.4 | ¹H NMR (400 MHz, DMSO-d6) chemical shifts 7.95 - 7.89 (m, 2H), 7.76 (s, 1H), 7.54 - 7.48 (m, 2H), 7.43 - 7.30 (m, 7H), 7.22 (t, J = 7.5 Hz, 1H), 7.14 (d, J = 7.6 Hz, 1H), 4.67 - 4.55 (m, 2H), 4.35 (d, J = 10.5 Hz, 1H), 3.87 (d, J= 10.5 Hz, 1H), 3.29 - 3.18 (m, 1H), 3.17 - 3.04 (m, 1H), 2.44 (q, J = 7.2, 5.6 Hz, 2H). |
| 125 | 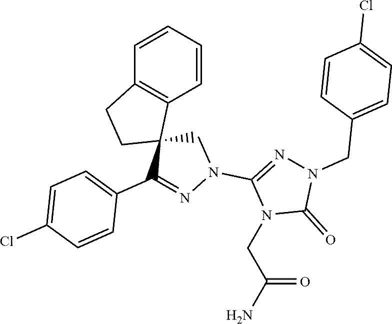 [SMILES: NC(=O)Cn1c(N2C[C@@]3(CCc4ccccc43)C(c3ccc(Cl)cc3)=N2)nn(Cc2ccc(Cl)cc2)c1=O] | 547.4 | ¹H NMR (400 MHz, DMSO-d6) chemical shifts 7.67 (s, 1H), 7.43 -7.36 (m, 3H), 7.35 - 7.27 (m, 8H), 7.19 (t, J = 7.4 Hz, 1H), 7.09 (d, J = 7.6 Hz, 1H), 4.85 (d, J = 4.0 Hz, 2H), 4.52 (d, J = 6.7 Hz, 2H), 4.15 (d, J = 10.4 Hz, 1H), 3.67 (d, J = 10.3 Hz, 1H), 3.23 - 3.13 (m, 1H), 3.09 - 3.01 (m, 1H), 2.45 - 2.35 (m, 2H). |
| 126 | 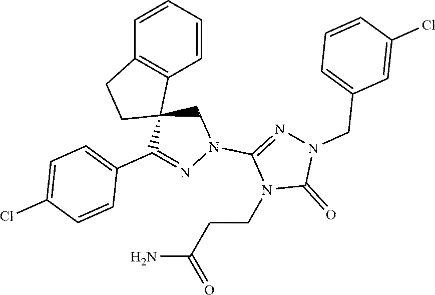 [SMILES: NC(=O)CCn1c(N2C[C@]3(CCc4ccccc43)C(c3ccc(Cl)cc3)=N2)nn(Cc2cccc(Cl)c2)c1=O] | 547.4 | ¹H NMR (400 MHz, DMSO-d6) chemical shifts 7.93 - 7.88 (m, 2H), 7.53 - 7.47 (m, 3H), 7.43 -7.31 (m, 6H), 7.24 (dd, J = 4.0, 1.7 Hz, 2H), 6.97 (s, 1H), 4.33 (d, J = 10.4 Hz, 1H), 4.22 (dd, J = 9.4, 6.2 Hz, 2H), 3.88 (d, J = 10.4 Hz, 1H), 3.28 - 3.17 (m, 1H), 3.13 - 3.03 (m, 1H), 2.71 - 2.64 (m, 2H), 2.48 -2.40 (m, 2H). |
| 127 | 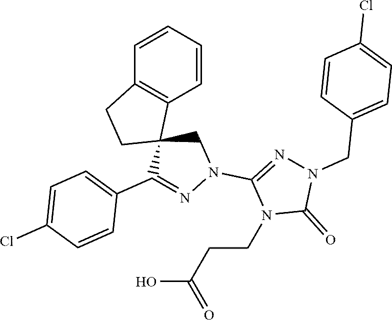 [SMILES: O=C(O)CCn1c(N2C[C@]3(CCc4ccccc43)C(c3ccc(Cl)cc3)=N2)nn(Cc2ccc(Cl)cc2)c1=O] | 562.5 | ¹H NMR (400 MHz, DMSO-d6) chemical shifts 12.53 (s, 1H), 7.55 - 7.27 (m, 10H), 7.20 (q, J = 8.0 Hz, 2H), 4.97 - 4.77 (m, 2H), 4.28 - 4.10 (m, 3H), 3.69 (d, J= 10.2 Hz, 1H), 3.17 (dt, J= 16.8, 8.7 Hz, 1H), 3.05 (dt, J = 16.2, 6.2 Hz, 1H), 2.87 - 2.73 (m, 2H), 2.41 (t, J = 7.5 Hz, 2H). |
| 128 | 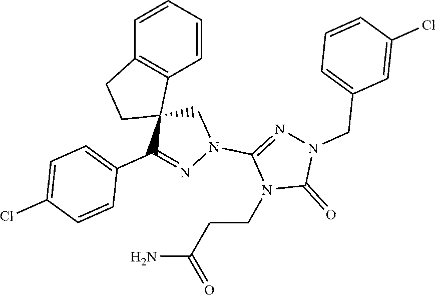 [SMILES: NC(=O)CCn1c(N2C[C@@]3(CCc4ccccc43)C(c3ccc(Cl)cc3)=N2)nn(Cc2cccc(Cl)c2)c1=O] | 547.4 | ¹H NMR (400 MHz, DMSO-d6) chemical shifts 7.93 - 7.88 (m, 2H), 7.53 - 7.47 (m, 3H), 7.43 -7.31 (m, 6H), 7.24 (dd, J = 4.0, 1.7 Hz, 2H), 6.97 (s, 1H), 4.33 (d, J = 10.4 Hz, 1H), 4.22 (dd, J = 9.4, 6.2 Hz, 2H), 3.88 (d, J = 10.4 Hz, 1H), 3.28 - 3.17 (m, 1H), 3.13 - 3.03 (m, 1H), 2.71 - 2.64 (m, 2H), 2.48 -2.40 (m, 2H). |
| 129 | 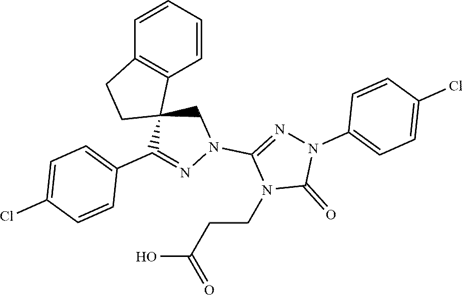 [SMILES: O=C(O)CCn1c(N2C[C@]3(CCc4ccccc43)C(c3ccc(Cl)cc3)=N2)nn(-c2ccc(Cl)cc2)c1=O] | 548.4 | ¹H NMR (400 MHz, DMSO-d6) chemical shifts 12.57 (s, 1H), 7.93 - 7.87 (m, 2H), 7.52 - 7.47 (m, 2H), 7.43 - 7.30 (m, 6H), 7.23 (q, J = 4.6, 3.5 Hz, 2H), 4.33 (d, J = 10.4 Hz, 1H), 4.25 (t, J = 7.6 Hz, 2H), 3.89 (d, J = 10.3 Hz, 1H), 3.26 - 3.17 (m, 1H), 3.09 (dt, J = 16.4, 6.1 Hz, 1H), 2.82 (td, J = 7.3, 2.8 Hz, 2H), 2.48 - 2.43 (m, 2H). |
| 130 | 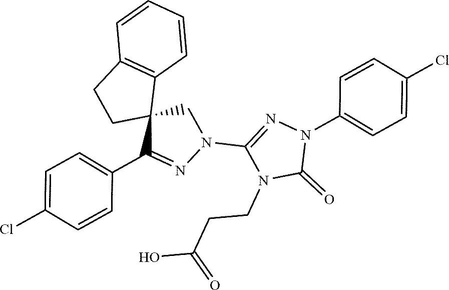 [SMILES: O=C(O)CCn1c(N2C[C@@]3(CCc4ccccc43)C(c3ccc(Cl)cc3)=N2)nn(-c2ccc(Cl)cc2)c1=O] | 548.4 | ¹H NMR (400 MHz, Methanol-d4) chemical shifts 7.93 (d, J = 8.8 Hz, 2H), 7.44 - 7.33 (m, 6H), 7.30 -7.22 (m, 4H), 4.40 (t, J = 7.7 Hz, 2H), 4.28 (dd, J= 10.5, 1.4 Hz, 1H), 4.00 (d, J = 10.4 Hz, 1H), 3.28 - 3.19 (m, 1H), 3.19 - 3.10 (m, 1H), 2.95 (dd, J = 9.0, 6.5 Hz, 2H), 2.65 - 2.46 (m, 2H). |
| 131 | 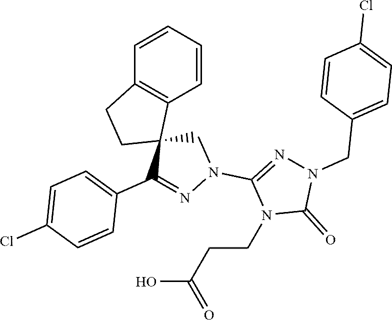 [SMILES: O=C(O)CCn1c(N2C[C@@]3(CCc4ccccc43)C(c3ccc(Cl)cc3)=N2)nn(Cc2ccc(Cl)cc2)c1=O] | 562.5 | ¹H NMR (400 MHz, DMSO-d6) chemical shifts 12.51 (s, 1H), 7.42 -7.27 (m, 10H), 7.23 -7.16 (m, 2H), 4.83 (d, J = 2.8 Hz, 2H), 4.21 - 4.10 (m, 3H), 3.69 (d, J= 10.3 Hz, 1H), 3.17 (dt, J= 16.9, 8.6 Hz, 1H), 3.04 (dt, J = 16.4, 6.1 Hz, 1H), 2.83 - 2.72 (m, 2H), 2.41 (dd, J = 8.6, 6.2 Hz, 2H). |
| 132 | 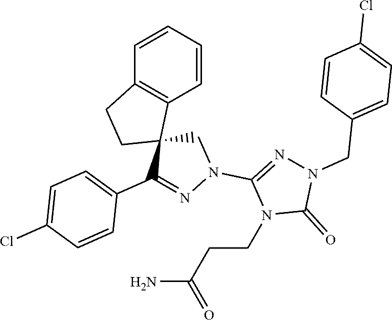 [SMILES: NC(=O)CCn1c(N2C[C@@]3(CCc4ccccc43)C(c3ccc(Cl)cc3)=N2)nn(Cc2ccc(Cl)cc2)c1=O] | 561.5 | ¹H NMR (400 MHz, DMSO-d6) chemical shifts 7.48 (s, 1H), 7.42 -7.25 (m, 10H), 7.23 - 7.14 (m, 2H), 6.96 (s, 1H), 4.83 (d, J = 2.2 Hz, 2H), 4.18 - 4.06 (m, 3H), 3.68 (d, J = 10.2 Hz, 1H), 3.23 - 3.00 (m, 2H), 2.67 - 2.58 (m, 2H), 2.44 -2.35 (m, 2H). |
| 133 | 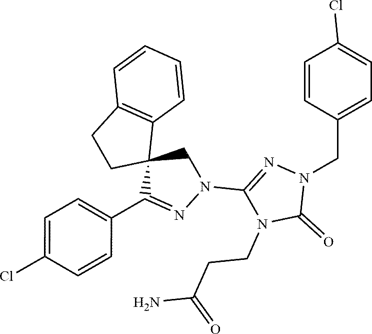 [SMILES: NC(=O)CCn1c(N2C[C@]3(CCc4ccccc43)C(c3ccc(Cl)cc3)=N2)nn(Cc2ccc(Cl)cc2)c1=O] | 561.5 | ¹H NMR (400 MHz, DMSO-d6) chemical shifts 7.48 (s, 1H), 7.41 -7.27 (m, 10H), 7.23 - 7.16 (m, 2H), 6.96 (s, 1H), 4.83 (d, J = 2.3 Hz, 2H), 4.17 - 4.08 (m, 3H), 3.68 (d, J = 10.2 Hz, 1H), 3.23 - 3.00 (m, 2H), 2.66 - 2.59 (m, 2H), 2.44 -2.37 (m, 2H). |
| 134 | 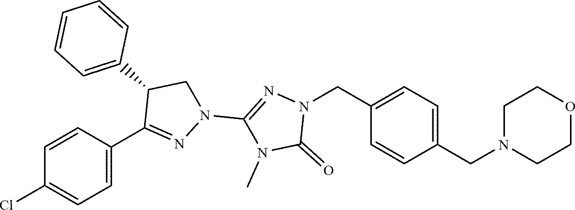 [SMILES: Cn1c(N2C[C@@H](c3ccccc3)C(c3ccc(Cl)cc3)=N2)nn(Cc2ccc(CN3CCOCC3)cc2)c1=O] | 543.1 | ¹H NMR (400 MHz, Methanol-d4) chemical shifts 7.66 (dd, J = 8.8, 2.7 Hz, 2H), 7.36 -C 7.23 (m, 11H), 4.92 (d, J = 2.3 Hz, 2H), 4.87 (s, 1H), 4.31 (td, J= 10.5, 3.7 Hz, 1H), 3.78 (dd, J = 10.2, 4.2 Hz, 1H), 3.67 (t, J = 4.5 Hz, 4H), 3.61 (d, J = 2.2 Hz, 3H), 3.52 (s, 2H), 2.46 (d, J = 6.0 Hz, 4H). |
| 135 | 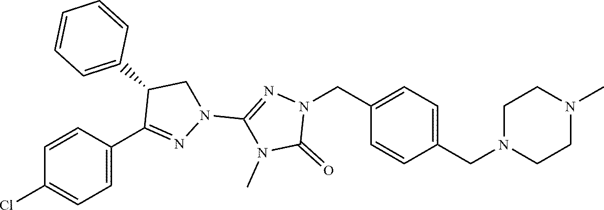 [SMILES: CN1CCN(Cc2ccc(Cn3nc(N4C[C@@H](c5ccccc5)C(c5ccc(Cl)cc5)=N4)n(C)c3=O)cc2)CC1] | 556.1 | ¹H NMR (400 MHz, DMSO-d6) chemical shifts 7.70 - 7.65 (m, 2H), 7.45 - 7.40 (m, 2H), 7.31 (d, J = 5.9 Hz, 4H), 7.22 (q, J = 8.0 Hz, 5H), 4.98 (dd, J= 10.9, 4.1 Hz, 1H), 4.88 - 4.77 (m, 2H), 4.25 (t, J = 10.7 Hz, 1H), 3.70 (dd, J = 10.4, 4.1 Hz, 1H), 3.48 (s, 3H), 3.40 (s, 2H), 2.31 (s, 8H), 2.14 (s, 3H). |
| 136 | 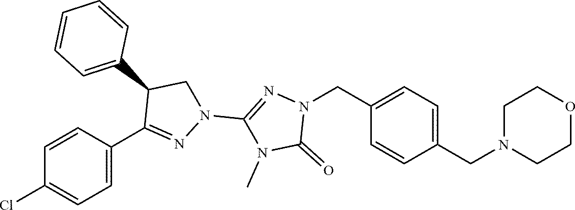 [SMILES: Cn1c(N2C[C@H](c3ccccc3)C(c3ccc(Cl)cc3)=N2)nn(Cc2ccc(CN3CCOCC3)cc2)c1=O] | 543.1 | ¹H NMR (400 MHz, Methanol-d4) chemical shifts 7.69 - 7.62 (m, 2H), 7.36 - 7.24 (m, 11H), 4.91 (t, J = 2.0 Hz, 2H), 4.87 (s, 1H), 4.35 - 4.26 (m, 1H), 3.78 (dd, J = 10.3, 5.1 Hz, 1H), 3.67 (q, J = 4.2 Hz, 4H), 3.62 - 3.57 (m, 3H), 3.52 (d, J = 3.1 Hz, 2H), 2.45 (s, 4H). |
| 137 | 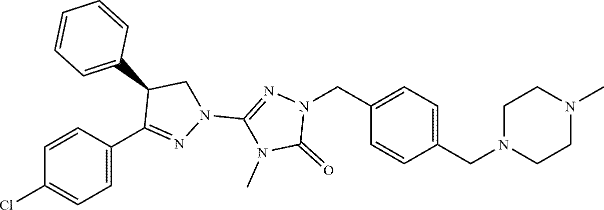 [SMILES: CN1CCN(Cc2ccc(Cn3nc(N4C[C@H](c5ccccc5)C(c5ccc(Cl)cc5)=N4)n(C)c3=O)cc2)CC1] | 556.1 | ¹H NMR (400 MHz, DMSO-d6) chemical shifts 7.71 - 7.65 (m, 2H), 7.45 - 7.40 (m, 2H), 7.36 -7.28 (m, 4H), 7.22 (q, J = 8.0 Hz, 5H), 4.99 (dd, J= 10.9, 4.1 Hz, 1H), 4.88 - 4.77 (m, 2H), 4.25 (t, J = 10.7 Hz, 1H), 3.70 (dd, J = 10.4, 4.1 Hz, 1H), 3.48 (s, 3H), 3.40 (s, 2H), 2.33 (s, 8H), 2.14 (s, 3H). |
| 138 | 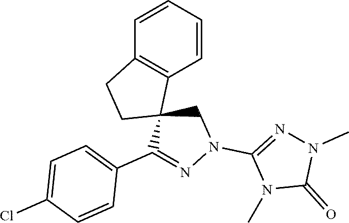 [SMILES: Cn1nc(N2C[C@]3(CCc4ccccc43)C(c3ccc(Cl)cc3)=N2)n(C)c1=O] | 393.9 | ¹H NMR (400 MHz, DMSO-d6) chemical shifts 7.42 - 7.29 (m, 6H), 7.26 - 7.16 (m, 2H), 4.17 (d, J = 10.3 Hz, 1H), 3.74 (d, J= 10.2 Hz, 1H), 3.42 (s, 3H), 3.27 (s, 3H), 3.20 (dt, J = 16.9, 8.8 Hz, 1H), 3.07 (ddd, J = 16.4, 7.8, 4.5 Hz, 1H), 2.47 - 2.35 (m, 2H). |
| 139 | 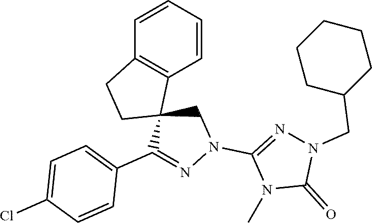 [SMILES: Cn1c(N2C[C@]3(CCc4ccccc43)C(c3ccc(Cl)cc3)=N2)nn(CC2CCCCC2)c1=O] | 476 | ¹H NMR (400 MHz, Methanol-d4) chemical shifts 7.43 - 7.32 (m, 4H), 7.28 -7.18 (m, 4H), 4.13 (d, J = 10.2 Hz, 1H), 3.86 (d, J= 10.2 Hz, 1H), 3.57 (d, J = 7.6 Hz, 5H), 3.28 - 3.08 (m, 2H), 2.60 - 2.43 (m, 2H), 1.89 - 1.64 (m, 6H), 1.24 (q, J = 12.3, 11.3 Hz, 3H), 1.02 (q, J = 13.1, 12.6 Hz, 2H). |
| 140 | 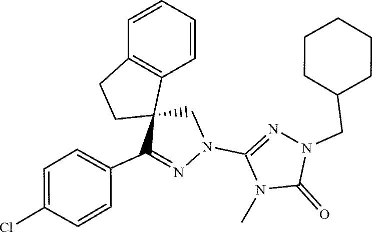 [SMILES: Cn1c(N2C[C@@]3(CCc4ccccc43)C(c3ccc(Cl)cc3)=N2)nn(CC2CCCCC2)c1=O] | 476 | ¹H NMR (400 MHz, Methanol-d4) chemical shifts 7.41 - 7.31 (m, 4H), 7.27 - 7.18 (m, 4H), 4.13 (dd, J = 10.2, 5.0 Hz, 1H), 3.86 (dd, J = 10.3, 3.7 Hz, 1H), 3.59 - 3.53 (m, 5H), 3.27 - 3.06 (m, 2H), 2.61 -2.42 (m, 2H), 1.70 (t, J = 15.5 Hz, 6H), 1.24 (s, 3H), 1.01 (q, J = 12.1 Hz, 2H). |
| 141 | 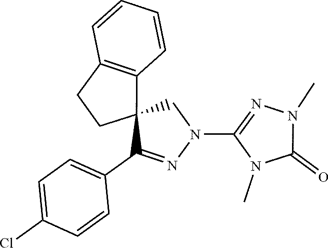 [SMILES: Cn1nc(N2C[C@@]3(CCc4ccccc43)C(c3ccc(Cl)cc3)=N2)n(C)c1=O] | 393.9 | ¹H NMR (400 MHz, DMSO-d6) chemical shifts 7.45 - 7.30 (m, 6H), 7.29 - 7.15 (m, 2H), 4.18 (d, J = 10.3 Hz, 1H), 3.74 (d, J= 10.2 Hz, 1H), 3.42 (s, 3H), 3.24 (s, 3H), 3.19 (q, J = 8.2 Hz, 1H), 3.07 (d, J = 17.4 Hz, 1H), 2.44 (d, J = 8.4 Hz, 2H) |
| 142 | 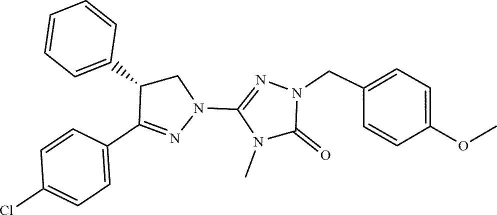 [SMILES: COc1ccc(Cn2nc(N3C[C@@H](c4ccccc4)C(c4ccc(Cl)cc4)=N3)n(C)c2=O)cc1] | 474 | ¹H NMR (400 MHz, DMSO-d6) chemical shifts 7.67 (d, J = 8.4 Hz, 2H), 7.45 - 7.39 (m, 2H), 7.35 -7.27 (m, 4H), 7.26 - 7.17 (m, 3H), 6.88 (d, J = 8.5 Hz, 2H), 4.98 (dd, J = 10.9, 4.0 Hz, 1H), 4.83 - 4.71 (m, 2H), 4.29 - 4.19 (m, 1H), 3.74 - 3.65 (m, 4H), 3.46 (s, 3H) |
| 143 | 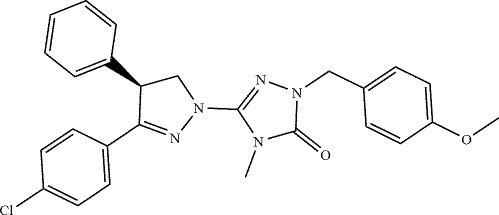 [SMILES: COc1ccc(Cn2nc(N3C[C@H](c4ccccc4)C(c4ccc(Cl)cc4)=N3)n(C)c2=O)cc1] | 474 | ¹H NMR (400 MHz, DMSO-d6) chemical shifts 7.71 - 7.62 (m, 2H), 7.45 - 7.38 (m, 2H), 7.35 -7.28 (m, 4H), 7.26 - 7.18 (m, 3H), 6.91 - 6.84 (m, 2H), 4.98 (dd, J = 10.9, 4.0 Hz, 1H), 4.82 - 4.71 (m, 2H), 4.24 (t, J = 10.7 Hz, 1H), 3.73 - 3.66 (m, 4H), 3.46 (s, 3H) |
| 144 | 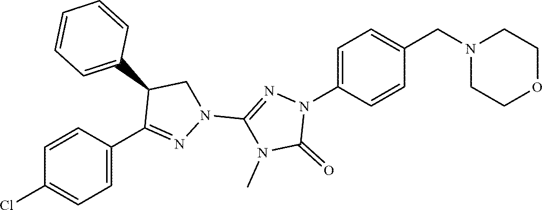 [SMILES: Cn1c(N2C[C@H](c3ccccc3)C(c3ccc(Cl)cc3)=N2)nn(-c2ccc(CN3CCOCC3)cc2)c1=O] | 529 | ¹H NMR (400 MHz, DMSO-d6) chemical shifts 7.82 (d, J = 8.4 Hz, 2H), 7.71 (d, J = 8.5 Hz, 2H), 7.47-7.42 (m, 2H), 7.35 (d, J = 4.3 Hz, 6H), 7.27 (q, J = 4.3 Hz, 1H), 5.08 (dd, J = 11.1, 4.4 Hz, 1H), 4.42 (t, J = 10.7 Hz, 1H), 3.87 (dd, J = 10.5, 4.4 Hz, 1H), 3.58 - 3.56 (m, 7H), 3.44 (s, 2H), 2.34 (s, 4H) |
| 145 | 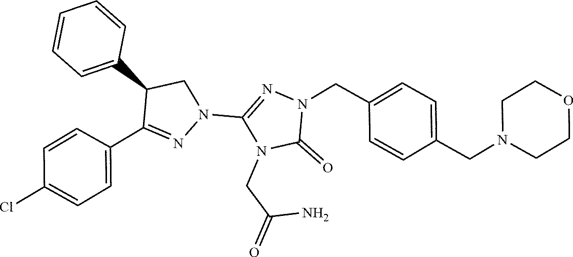 [SMILES: NC(=O)Cn1c(N2C[C@H](c3ccccc3)C(c3ccc(Cl)cc3)=N2)nn(Cc2ccc(CN3CCOCC3)cc2)c1=O] | 586.1 | ¹H NMR (400 MHz, DMSO-d6) chemical shifts 7.77 - 7.64 (m, 3H), 7.39 (d, J = 8.5 Hz, 2H), 7.34 - 7.19 (m, 10H), 4.95 (dd, J = 11.1, 4.0 Hz, 1H), 4.83 (d, J = 3.4 Hz, 2H), 4.56 (s, 2H), 4.23 (t, J = 10.8 Hz, 1H), 3.69 (dd, J = 10.5, 4.1 Hz, 1H), 3.55 (t, J = 4.5 Hz, 4H), 3.42 (s, 2H), 2.32 (t, J = 4.6 Hz, 4H) |
| 146 | 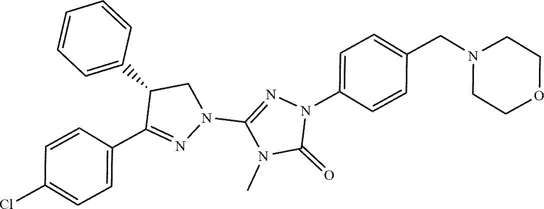 [SMILES: Cn1c(N2C[C@@H](c3ccccc3)C(c3ccc(Cl)cc3)=N2)nn(-c2ccc(CN3CCOCC3)cc2)c1=O] | 529 | ¹H NMR (400 MHz, DMSO-d6) chemical shifts 7.82 (d, J = 8.4 Hz, 2H), 7.74 - 7.68 (m, 2H), 7.47 -7.42 (m, 2H), 7.38 - 7.31 (m, 6H), 7.26 (p, J = 4.3 Hz, 1H), 5.08 (dd, J = 11.0, 4.4 Hz, 1H), 4.42 (t, J = 10.7 Hz, 1H), 3.87 (dd, J = 10.4, 4.3 Hz, 1H), 3.57 (d, J = 4.2 Hz, 7H), 3.44 (s, 2H), 2.35 (d, J = 5.9 Hz, 4H). |
| 147 | 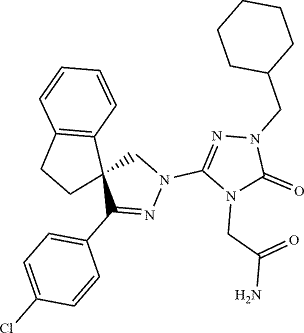 [SMILES: NC(=O)Cn1c(N2C[C@@]3(CCc4ccccc43)C(c3ccc(Cl)cc3)=N2)nn(CC2CCCCC2)c1=O] | 519 | ¹H NMR (400 MHz, DMSO-d6) chemical shifts 7.64 (s, 1H), 7.39 (d, J = 7.5 Hz, 1H), 7.34 (s, 5H), 7.26 (s, 1H), 7.21 (t, J = 7.4 Hz, 1H), 7.11 (d, J = 7.5 Hz, 1H), 4.54 -4.41 (m, 2H), 4.19 (d, J = 10.4 Hz, 1H), 3.70 (d, J = 10.3 Hz, 1H), 3.46 (d, J = 6.9 Hz, 2H), 3.26 -3.15 (m, 1H), 3.10 - 3.00 (m, 1H), 2.46 - 2.32 (m, 2H), 1.72 - 1.55 (m, 6H), 1.15 (d, J = 8.6 Hz, 3H), 1.01 - 0.83 (m, 2H). |
| 149 | 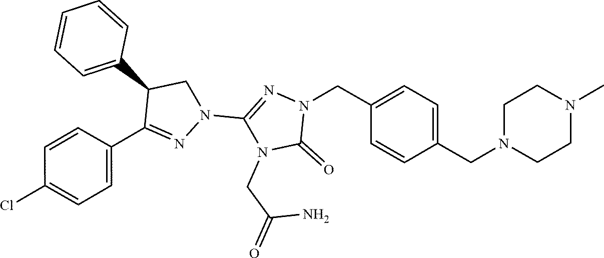 [SMILES: CN1CCN(Cc2ccc(Cn3nc(N4C[C@H](c5ccccc5)C(c5ccc(Cl)cc5)=N4)n(CC(N)=O)c3=O)cc2)CC1] | 599.1 | ¹H NMR (400 MHz, DMSO-d6) chemical shifts 7.72 (s, 1H), 7.70 -7.66 (m, 2H), 7.42 - 7.36 (m, 2H), 7.33 - 7.29 (m, 3H), 7.27- 7.21 (m, 7H), 4.97 - 4.79 (m, 3H), 4.56 (s, 2H), 4.23 (t, J = 10.8 Hz, 1H), 3.69 (dd, J = 10.5, 4.0 Hz, 1H), 3.42 (s, 2H), 2.42-2.33 (m, 8H), 2.20 (s, 3H) |
| 150 | 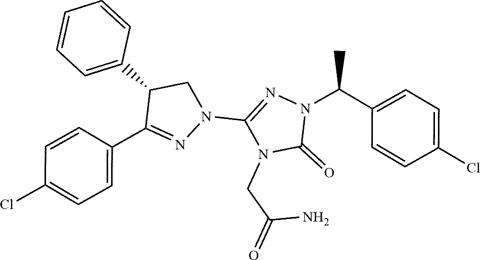 [SMILES: C[C@@H](c1ccc(Cl)cc1)n1nc(N2C[C@@H](c3ccccc3)C(c3ccc(Cl)cc3)=N2)n(CC(N)=O)c1=O] | 535.4 | ¹H NMR (400 MHz, DMSO-d6) chemical shifts 7.75 - 7.60 (m, 3H), 7.39 (d, J = 10.3 Hz, 6H), 7.34 - 7.21 (m, 6H), 5.37 (q, J = 7.0 Hz, 1H), 4.97 (dd, J = 11.0, 4.0 Hz, 1H), 4.52 (s, 2H), 4.29 (t, J = 10.8 Hz, 1H), 3.71 (dd, J = 10.5, 4.0 Hz, 1H), 1.63 (d, J = 7.1 Hz, 3H). |
| 152 | 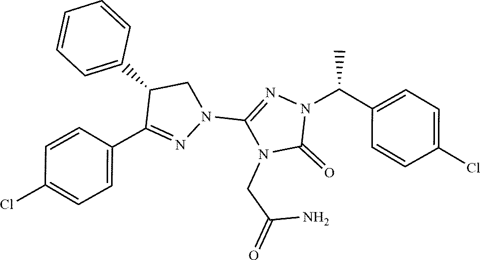 [SMILES: C[C@H](c1ccc(Cl)cc1)n1nc(N2C[C@@H](c3ccccc3)C(c3ccc(Cl)cc3)=N2)n(CC(N)=O)c1=O] | 535.4 | ¹H NMR (400 MHz, DMSO-d6) chemical shifts 7.73 - 7.61 (m, 3H), 7.39 (d, J = 6.0 Hz, 6H), 7.35 - 7.22 (m, 6H), 5.38 (q, J = 7.0 Hz, 1H), 4.96 (dd, J = 11.0, 4.1 Hz, 1H), 4.54 (d, J = 2.9 Hz, 2H), 4.27 (t, J = 10.8 Hz, 1H), 3.73 (dd, J = 10.5, 4.1 Hz, 1H), 1.62 (d, J = 7.1 Hz, 3H). |
| 153 | 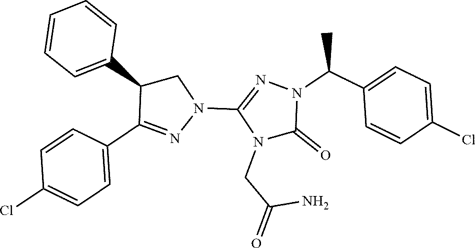 [SMILES: C[C@@H](c1ccc(Cl)cc1)n1nc(N2C[C@H](c3ccccc3)C(c3ccc(Cl)cc3)=N2)n(CC(N)=O)c1=O] | 535.4 | ¹H NMR (400 MHz, DMSO-d6) chemical shifts 7.68 (dd, J = 9.0, 2.4 Hz, 3H), 7.39 (d, J = 5.6 Hz, 6H), 7.34 - 7.20 (m, 6H), 5.38 (q, J = 7.0 Hz, 1H), 4.96 (dd, J = 11.0, 4.1 Hz, 1H), 4.54 (d, J = 2.8 Hz, 2H), 4.27 (t, J = 10.8 Hz, 1H), 3.73 (dd, J = 10.6, 4.1 Hz, 1H), 1.62 (d, J = 7.0 Hz, 3H). |
| 154 | 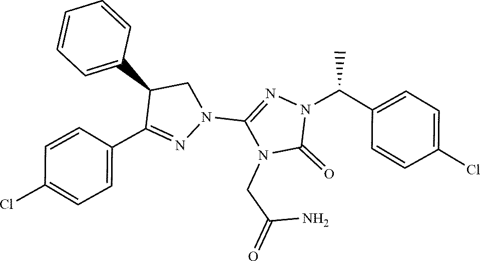 [SMILES: C[C@H](c1ccc(Cl)cc1)n1nc(N2C[C@H](c3ccccc3)C(c3ccc(Cl)cc3)=N2)n(CC(N)=O)c1=O] | 535.4 | ¹H NMR (400 MHz, DMSO-d6) chemical shifts 7.75 - 7.61 (m, 3H), 7.39 (d, J = 10.3 Hz, 6H), 7.35 - 7.20 (m, 6H), 5.37 (q, J = 7.0 Hz, 1H), 4.97 (dd, J = 11.0, 4.0 Hz, 1H), 4.52 (s, 2H), 4.29 (t, J = 10.8 Hz, 1H), 3.71 (dd, J = 10.5, 4.0 Hz, 1H), 1.63 (d, J = 7.1 Hz, 3H). |
| 158 | 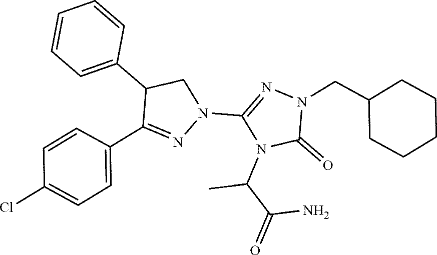 [SMILES: CC(C(N)=O)n1c(N2CC(c3ccccc3)C(c3ccc(Cl)cc3)=N2)nn(CC2CCCCC2)c1=O] | 507 | ¹H NMR (400 MHz, DMSO-d6) chemical shifts 7.71 - 7.64 (m, 2H), 7.51 - 7.11 (m, 9H), 5.01 -4.93 (m, 2H), 4.25 (dt, J = 15.4, 10.7 Hz, 1H), 3.70 (d, J = 10.5 Hz, 1H), 3.45 (d, J = 6.8 Hz, 2H), 1.77 - 1.52 (m, 9H), 1.15 (d, J = 8.6 Hz, 3H), 0.94 (d, J = 11.9 Hz, 2H). |
| 159 | 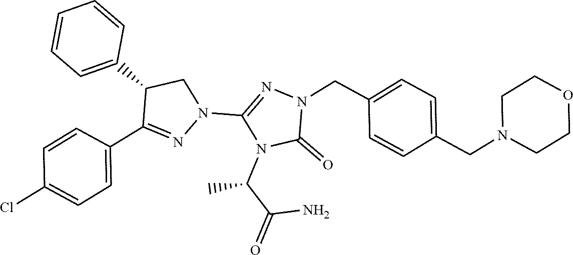 [SMILES: C[C@@H](C(N)=O)n1c(N2C[C@@H](c3ccccc3)C(c3ccc(Cl)cc3)=N2)nn(Cc2ccc(CN3CCOCC3)cc2)c1=O] | 600.1 | ¹H NMR (400 MHz, DMSO-d6) chemical shifts 7.71 - 7.65 (m, 2H), 7.46 (s, 1H), 7.43 - 7.38 (m, 2H), 7.35 - 7.21 (m, 10H), 5.02 (q, J = 7.2 Hz, 1H), 4.96 (dd, J = 10.8, 3.4 Hz, 1H), 4.82 (s, 2H), 4.21 (t, J = 10.7 Hz, 1H), 3.67 (dd, J = 10.5, 3.4 Hz, 1H), 3.55 (t, J = 4.6 Hz, 4H), 3.42 (s, 2H), 2.33 (d, J = 4.5 Hz, 4H), 1.72 (d, J = 7.2 Hz, 3H). |
| 160 | 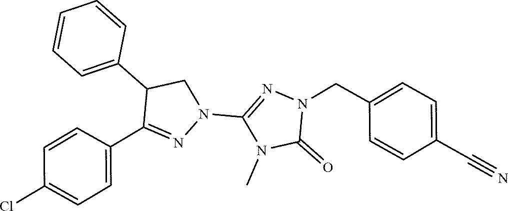 [SMILES: Cn1c(N2CC(c3ccccc3)C(c3ccc(Cl)cc3)=N2)nn(Cc2ccc(C#N)cc2)c1=O] | 469 | ¹H NMR (400 MHz, DMSO-d6) chemical shifts 7.84 - 7.78 (m, 2H), 7.72 - 7.66 (m, 2H), 7.49 -7.40 (m, 4H), 7.36 - 7.21 (m, 5H), 5.03 - 4.91 (m, 3H), 4.25 (t, J = 10.6 Hz, 1H), 3.71 (dd, J = 10.4, 4.1 Hz, 1H), 3.50 (s, 3H). |
| 161 | 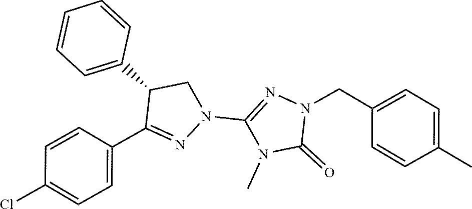 [SMILES: Cc1ccc(Cn2nc(N3C[C@@H](c4ccccc4)C(c4ccc(Cl)cc4)=N3)n(C)c2=O)cc1] | 458 | ¹H NMR (400 MHz, DMSO-d6) chemical shifts 7.76 - 7.64 (m, 2H), 7.47 - 7.40 (m, 2H), 7.37 -7.27 (m, 4H), 7.27 - 7.22 (m, 1H), 7.18 - 7.10 (m, 4H), 4.98 (dd, J = 10.9, 4.1 Hz, 1H), 4.86 - 4.73 (m, 2H), 4.29 - 4.20 (m, 1H), 3.69 (dd, J = 10.4, 4.2 Hz, 1H), 3.47 (s, 3H), 2.26 (s, 3H). |
| 163 | 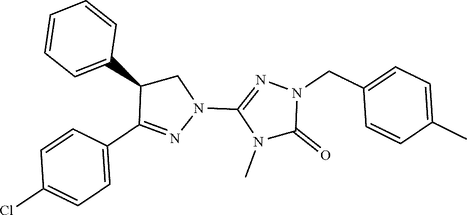 [SMILES: Cc1ccc(Cn2nc(N3C[C@H](c4ccccc4)C(c4ccc(Cl)cc4)=N3)n(C)c2=O)cc1] | 458 | ¹H NMR (400 MHz, Methanol-d4) chemical shifts 7.70 - 7.64 (m, 2H), 7.43 (d, J = 8.5 Hz, 2H), 7.31 (d, J = 6.4 Hz, 4H), 7.25 (td, J = 5.9, 2.4 Hz, 1H), 7.14 (d, J = 1.8 Hz, 4H), 4.98 (dd, J = 10.9, 4.1 Hz, 1H), 4.85 - 4.74 (m, 2H), 4.24 (t, J = 10.6 Hz, 1H), 3.69 (dd, J = 10.4, 4.2 Hz, 1H), 3.47 (s, 3H), 2.27 (s, 3H). |
| 164 | 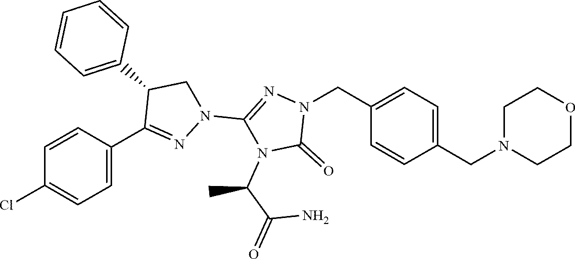 [SMILES: C[C@H](C(N)=O)n1c(N2C[C@@H](c3ccccc3)C(c3ccc(Cl)cc3)=N2)nn(Cc2ccc(CN3CCOCC3)cc2)c1=O] | 600.1 | ¹H NMR (400 MHz, DMSO-d6) chemical shifts 7.70 - 7.62 (m, 2H), 7.54 (s, 1H), 7.39 (d, J = 8.3 Hz, 2H), 7.35 - 7.20 (m, 10H), 5.03 (q, J = 7.1 Hz, 1H), 4.96 (dd, J = 11.0, 4.3 Hz, 1H), 4.88 - 4.73 (m, 2H), 4.24 (t, J = 10.8 Hz, 1H), 3.68 (dd, J = 10.5, 4.3 Hz, 1H), 3.55 (t, J = 4.6 Hz, 4H), 3.42 (s, 2H), 2.32 (t, J = 4.6 Hz, 4H), 1.71 (d, J = 7.2 Hz, 3H). |
| 166 | 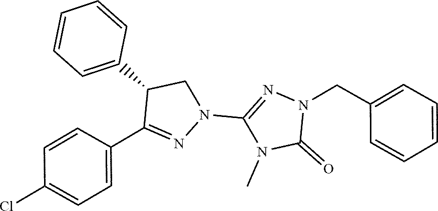 [SMILES: Cn1c(N2C[C@@H](c3ccccc3)C(c3ccc(Cl)cc3)=N2)nn(Cc2ccccc2)c1=O] | 443.9 | ¹H NMR (400 MHz, DMSO-d6) chemical shifts 7.71 - 7.64 (m, 2H), 7.45 - 7.40 (m, 2H), 7.36 -7.22 (m, 10H), 4.99 (dd, J = 10.9, 4.1 Hz, 1H), 4.91 - 4.79 (m, 2H), 4.26 (t, J = 10.7 Hz, 1H), 3.70 (dd, J = 10.4, 4.1 Hz, 1H), 3.48 (s, 3H). |
| 167 | 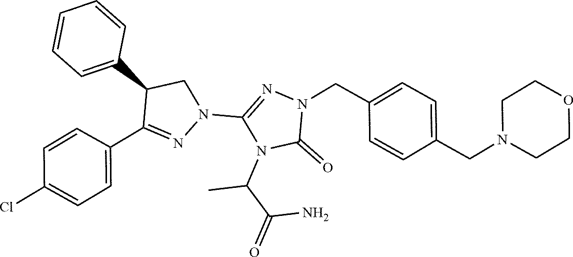 [SMILES: CC(C(N)=O)n1c(N2C[C@H](c3ccccc3)C(c3ccc(Cl)cc3)=N2)nn(Cc2ccc(CN3CCOCC3)cc2)c1=O] | 600.1 | ¹H NMR (400 MHz, DMSO-d6) chemical shifts 7.71 - 7.65 (m, 2H), 7.57 - 7.37 (m, 3H), 7.35 -7.20 (m, 10H), 5.07 - 4.93 (m, 2H), 4.89 - 4.74 (m, 2H), 4.22 (dt, J = 14.5, 10.7 Hz, 1H), 3.67 (dt, J = 10.4, 3.6 Hz, 1H), 3.55 (t, J = 4.6 Hz, 4H), 3.42 (s, 2H), 2.32 (t, J = 4.7 Hz, 4H), 1.72 (dd, J = 7.3, 5.4 Hz, 3H). |
| 168 | 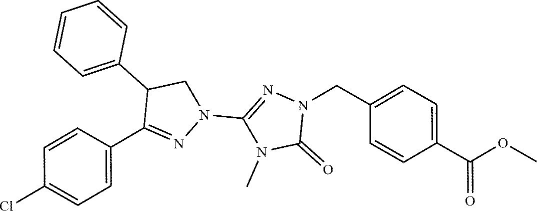 [SMILES: COC(=O)c1ccc(Cn2nc(N3CC(c4ccccc4)C(c4ccc(Cl)cc4)=N3)n(C)c2=O)cc1] | 502 | ¹H NMR (400 MHz, DMSO-d6) chemical shifts 7.96 - 7.90 (m, 2H), 7.71 - 7.64 (m, 2H), 7.46 -7.37 (m, 4H), 7.32 (d, J = 6.4 Hz, 4H), 7.32 - 7.21 (m, 1H), 5.03 -4.95 (m, 1H), 4.95 (d, J = 3.6 Hz, 2H), 4.25 (t, J = 10.7 Hz, 1H), 3.84 (s, 3H), 3.70 (dd, J = 10.5, 4.1 Hz, 1H), 3.50 (s, 3H). |
| 169 | 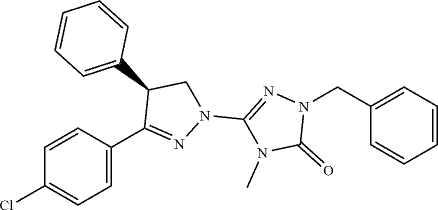 [SMILES: Cn1c(N2C[C@H](c3ccccc3)C(c3ccc(Cl)cc3)=N2)nn(Cc2ccccc2)c1=O] | 443.9 | ¹H NMR (400 MHz, DMSO-d6) chemical shifts 7.71 - 7.65 (m, 2H), 7.47 - 7.40 (m, 2H), 7.37 -7.21 (m, 10H), 4.99 (dd, J = 10.9, 4.1 Hz, 1H), 4.91 - 4.80 (m, 2H), 4.26 (t, J = 10.7 Hz, 1H), 3.70 (dd, J = 10.4, 4.1 Hz, 1H), 3.48 (s, 3H). |
| 170 | 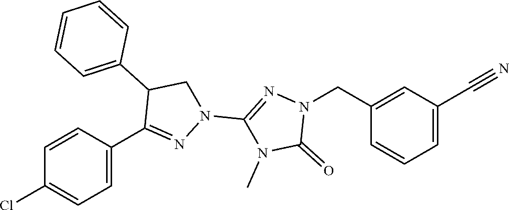 [SMILES: Cn1c(N2CC(c3ccccc3)C(c3ccc(Cl)cc3)=N2)nn(Cc2cccc(C#N)c2)c1=O] | 469 | ¹H NMR (400 MHz, DMSO-d6) chemical shifts 7.78 - 7.72 (m, 2H), 7.70 - 7.65 (m, 2H), 7.63 -7.53 (m, 2H), 7.46 - 7.40 (m, 2H), 7.36 - 7.28 (m, 4H), 7.24 (ddd, J = 8.4, 5.5, 2.3 Hz, 1H), 5.03 - 4.87 (m, 3H), 4.26 (t, J = 10.7 Hz, 1H), 3.71 (dd, J = 10.4, 4.1 Hz, 1H), 3.50 (s, 3H). |
| 171 | 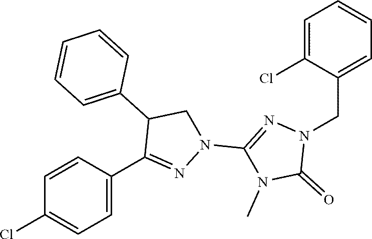 [SMILES: Cn1c(N2CC(c3ccccc3)C(c3ccc(Cl)cc3)=N2)nn(Cc2ccccc2Cl)c1=O] | 478.4 | ¹H NMR (400 MHz, DMSO-d6) chemical shifts 7.72 - 7.65 (m, 2H), 7.50 - 7.40 (m, 3H), 7.37 -7.28 (m, 6H), 7.28 - 7.18 (m, 2H), 5.03 - 4.88 (m, 3H), 4.25 (t, J = 10.7 Hz, 1H), 3.70 (dd, J = 10.4, 4.1 Hz, 1H), 3.50 (s, 3H). |
| 172 | 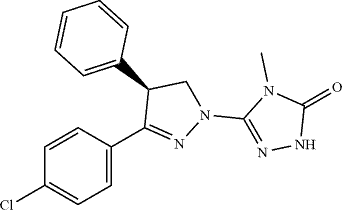 [SMILES: Cn1c(N2C[C@H](c3ccccc3)C(c3ccc(Cl)cc3)=N2)n[nH]c1=O] | 353.8 | ¹H NMR (400 MHz, DMSO-d6) chemical shifts 11.22 (s, 1H), 7.74 - 7.62 (m, 2H), 7.43 (d, J = 8.5 Hz, 2H), 7.33 (d, J = 5.5 Hz, 4H), 7.28 - 7.19 (m, 1H), 4.99 (dd, J = 10.9, 4.2 Hz, 1H), 4.27 (t, J = 10.7 Hz, 1H), 3.72 (dd, J = 10.4,4.2 Hz, 1H), 3.41 (s, 3H). |
| 173 | 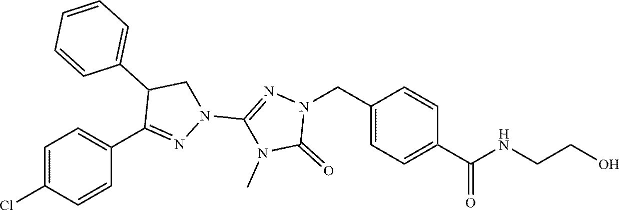 [SMILES: Cn1c(N2CC(c3ccccc3)C(c3ccc(Cl)cc3)=N2)nn(Cc2ccc(C(=O)NCCO)cc2)c1=O] | 531 | ¹H NMR (400 MHz, DMSO-d6) chemical shifts 8.38 (t, J = 5.6 Hz, 1H), 7.84 - 7.77 (m, 2H), 7.72 -7.64 (m, 2H), 7.47 - 7.39 (m, 2H), 7.37 - 7.28 (m, 6H), 7.24 (ddd, J = 8.5, 5.5, 2.3 Hz, 1H), 4.99 (dd, J = 10.9, 4.1 Hz, 1H), 4.97 - 4.84 (m, 2H), 4.70 (t, J = 5.6 Hz, 1H), 4.25 (t, J = 10.7 Hz, 1H), 3.70 (dd, J = 10.4, 4.1 Hz, 1H), 3.50 (d, J = 4.9 Hz, 5H), 3.32 (s, 1H), 3.30 (d, J = 5.9 Hz, 1H). |
| 174 | 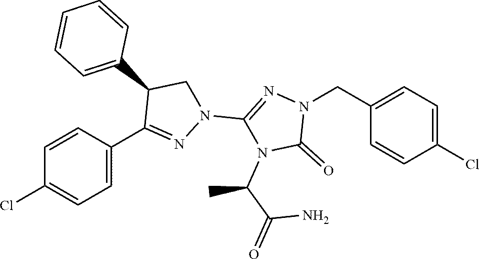 [SMILES: C[C@H](C(N)=O)n1c(N2C[C@H](c3ccccc3)C(c3ccc(Cl)cc3)=N2)nn(Cc2ccc(Cl)cc2)c1=O] | 535.4 | ¹H NMR (400 MHz, DMSO-d6) chemical shifts 7.72 - 7.65 (m, 2H), 7.48 - 7.36 (m, 5H), 7.36 -7.21 (m, 8H), 5.06 - 4.93 (m, 2H), 4.84 (d, J = 1.9 Hz, 2H), 4.20 (t, J = 10.7 Hz, 1H), 3.67 (dd, J = 10.5, 3.4 Hz, 1H), 1.72 (d, J = 7.2 Hz, 3H). |
| 175 | 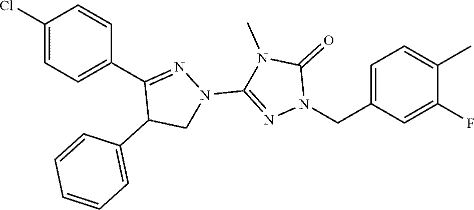 [SMILES: Cc1ccc(Cn2nc(N3CC(c4ccccc4)C(c4ccc(Cl)cc4)=N3)n(C)c2=O)cc1F] | 476 | ¹H NMR (400 MHz, DMSO-d6) chemical shifts 6.85 (d, J = 8.4 Hz, 2H), 6.56 - 6.43 (m, 7H), 6.39 (t, J = 7.8 Hz, 1H), 6.19 (t, J = 10.4 Hz, 2H), 4.09 (dd, J = 13.9, 6.7 Hz, 3H), 3.51 (t, J = 10.7 Hz, 1H), 2.99 (dd, J = 10.4, 4.6 Hz, 1H), 2.80 (d, J = 1.4 Hz, 3H), 1.43 (s, 3H). |
| 176 | 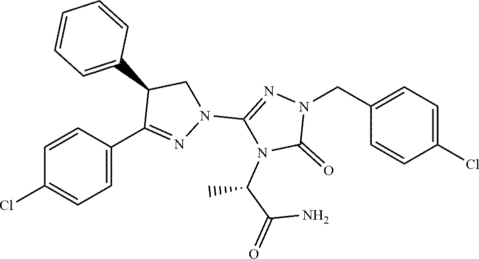 [SMILES: C[C@@H](C(N)=O)n1c(N2C[C@H](c3ccccc3)C(c3ccc(Cl)cc3)=N2)nn(Cc2ccc(Cl)cc2)c1=O] | 535.4 | ¹H NMR (400 MHz, DMSO-d6) chemical shifts 7.70 - 7.64 (m, 2H), 7.55 (s, 1H), 7.43 - 7.37 (m, 4H), 7.36 - 7.20 (m, 8H), 5.07 -4.93 (m, 2H), 4.90 - 4.77 (m, 2H), 4.24 (t, J = 10.8 Hz, 1H), 3.68 (dd, J = 10.5, 4.3 Hz, 1H), 1.71 (d, J = 7.2 Hz, 3H). |
| 177 | 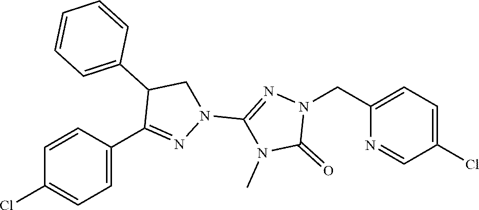 [SMILES: Cn1c(N2CC(c3ccccc3)C(c3ccc(Cl)cc3)=N2)nn(Cc2ccc(Cl)cn2)c1=O] | 479.4 | ¹H NMR (400 MHz, DMSO-d6) chemical shifts 8.57 (d, J = 2.6 Hz, 1H), 7.90 (dd, J = 8.4, 2.6 Hz, 1H), 7.72 - 7.66 (m, 2H), 7.43 (d, J = 8.6 Hz, 2H), 7.36 - 7.28 (m, 5H), 7.25 (dd, J = 8.1, 5.3 Hz, 1H), 5.02 -4.90 (m, 3H), 4.24 (t, J = 10.7 Hz, 1H), 3.69 (dd, J = 10.5, 4.0 Hz, 1H), 3.50 (s, 3H). |
| 178 | 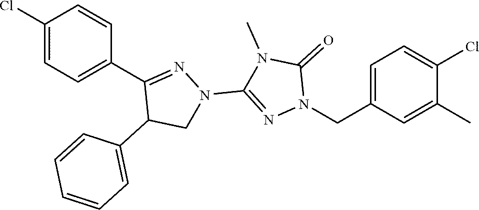 [SMILES: Cc1cc(Cn2nc(N3CC(c4ccccc4)C(c4ccc(Cl)cc4)=N3)n(C)c2=O)ccc1Cl] | 492.4 | ¹H NMR (400 MHz, Methanol-d4) chemical shifts 7.70 - 7.62 (m, 2H), 7.38 - 7.22 (m, 9H), 7.16 -7.09 (m, 1H), 4.93 - 4.87 (m, 2H), 4.83 (s, 1H), 4.32 (t, J = 10.7 Hz, 1H), 3.79 (dd, J = 10.3, 4.6 Hz, 1H), 3.61 (s, 3H), 2.35 (s, 3H). |
| 179 | 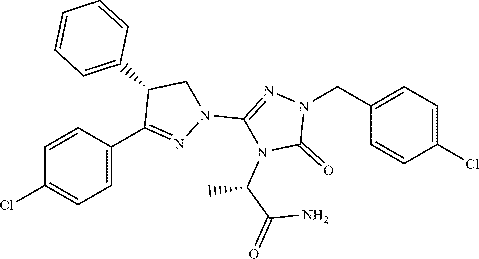 [SMILES: C[C@@H](C(N)=O)n1c(N2C[C@@H](c3ccccc3)C(c3ccc(Cl)cc3)=N2)nn(Cc2ccc(Cl)cc2)c1=O] | 535.4 | ¹H NMR (400 MHz, DMSO-d6) chemical shifts 7.72 - 7.65 (m, 2H), 7.46 (s, 1H), 7.43 - 7.37 (m, 4H), 7.35 - 7.29 (m, 4H), 7.28 -7.21 (m, 4H), 5.07 - 4.92 (m, 2H), 4.84 (d, J = 1.9 Hz, 2H), 4.20 (t, J = 10.7 Hz, 1H), 3.67 (dd, J = 10.5, 3.4 Hz, 1H), 1.72 (d, J = 7.3 Hz, 3H). |
| 180 | 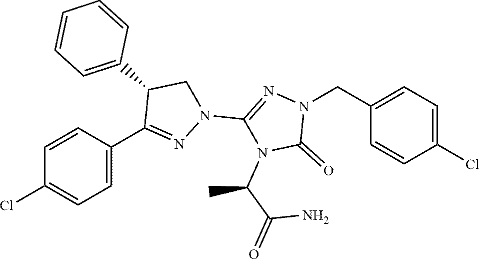 [SMILES: C[C@H](C(N)=O)n1c(N2C[C@@H](c3ccccc3)C(c3ccc(Cl)cc3)=N2)nn(Cc2ccc(Cl)cc2)c1=O] | 535.4 | ¹H NMR (400 MHz, DMSO-d6) chemical shifts 7.70 - 7.64 (m, 2H), 7.55 (s, 1H), 7.43 - 7.37 (m, 4H), 7.35 - 7.21 (m, 8H), 5.07 -4.93 (m, 2H), 4.91 - 4.77 (m, 2H), 4.24 (t, J = 10.7 Hz, 1H), 3.68 (dd, J = 10.5, 4.3 Hz, 1H), 1.71 (d, J = 7.2 Hz, 3H). |
| 181 | 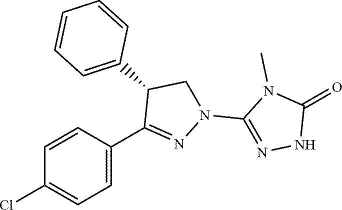 [SMILES: Cn1c(N2C[C@@H](c3ccccc3)C(c3ccc(Cl)cc3)=N2)n[nH]c1=O] | 353.8 | ¹H NMR (400 MHz, DMSO-d6) chemical shifts 11.22 (s, 1H), 7.72 - 7.61 (m, 2H), 7.47 - 7.39 (m, 2H), 7.33 (d, J = 5.6 Hz, 4H), 7.25 (ddd, J = 8.5, 6.2, 2.9 Hz, 1H), 4.99 (dd, J = 10.9, 4.2 Hz, 1H), 4.27 (t, J = 10.7 Hz, 1H), 3.72 (dd, J = 10.4, 4.2 Hz, 1H), 3.40 (s, 3H). |
| 182 | 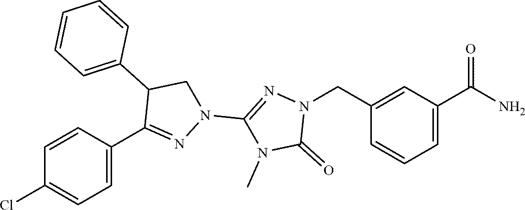 [SMILES: Cn1c(N2CC(c3ccccc3)C(c3ccc(Cl)cc3)=N2)nn(Cc2cccc(C(N)=O)c2)c1=O] | 487 | ¹H NMR (400 MHz, DMSO-d6) chemical shifts 7.97 (s, 1H), 7.78 (d, J = 3.6 Hz, 2H), 7.71 - 7.64 (m, 2H), 7.46 - 7.38 (m, 4H), 7.38 -7.28 (m, 5H), 7.28 - 7.20 (m, 1H), 4.99 (dd, J = 10.9, 4.0 Hz, 1H), 4.95 - 4.83 (m, 2H), 4.26 (t, J = 10.7 Hz, 1H), 3.70 (dd, J = 10.4, 4.1 Hz, 1H), 3.49 (s, 3H). |
| 183 | 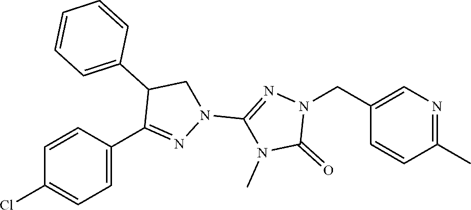 [SMILES: Cc1ccc(Cn2nc(N3CC(c4ccccc4)C(c4ccc(Cl)cc4)=N3)n(C)c2=O)cn1] | 459 | ¹H NMR (400 MHz, Methanol-d4) chemical shifts 8.40 (s, 1H), 7.71 (dd, J = 8.0, 2.3 Hz, 1H), 7.66 (d, J = 8.7 Hz, 2H), 7.37 - 7.22 (m, 8H), 4.94 (s, 2H), 4.91 - 4.87 (m, 1H), 4.32 (t, J = 10.7 Hz, 1H), 3.79 (dd, J = 10.3, 4.7 Hz, 1H), 3.61 (s, 3H), 2.52 (s, 3H). |
| 184 | 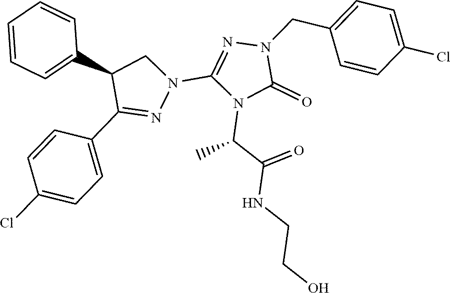 [SMILES: C[C@@H](C(=O)NCCO)n1c(N2C[C@H](c3ccccc3)C(c3ccc(Cl)cc3)=N2)nn(Cc2ccc(Cl)cc2)c1=O] | 579.5 | ¹H NMR (400 MHz, DMSO-d6) chemical shifts 8.01 (t, J = 5.6 Hz, 1H), 7.70 - 7.61 (m, 2H), 7.44 -7.38 (m, 4H), 7.35 - 7.20 (m, 7H), 5.08 (q, J = 7.1 Hz, 1H), 4.99 -4.77 (m, 3H), 4.65 (t, J = 5.5 Hz, 1H), 4.24 (t, J = 10.8 Hz, 1H), 3.69 (dd, J = 10.5, 4.0 Hz, 1H), 3.42 (q, J = 5.8 Hz, 2H), 3.26 - 3.11 (m, 2H), 1.71 (d, J = 7.2 Hz, 3H). |
| 186 | 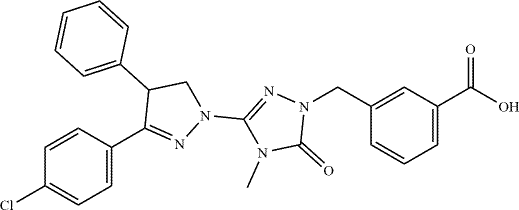 [SMILES: Cn1c(N2CC(c3ccccc3)C(c3ccc(Cl)cc3)=N2)nn(Cc2cccc(C(=O)O)c2)c1=O] | 487.9 | ¹H NMR (400 MHz, DMSO-d6) chemical shifts 13.00 (s, 1H), 7.85 (dd, J = 4.8, 2.6 Hz, 2H), 7.72 -7.64 (m, 2H), 7.54 - 7.39 (m, 4H), 7.31 (d, J = 6.0 Hz, 4H), 7.24 (ddd, J = 8.4, 5.5, 2.4 Hz, 1H), 4.99 (dd, J = 11.0, 4.2 Hz, 1H), 4.92 (d, J = 5.1 Hz, 2H), 4.26 (t, J = 10.7 Hz, 1H), 3.70 (dd, J = 10.4, 4.1 Hz, 1H), 3.49 (s, 3H). |
| 187 | 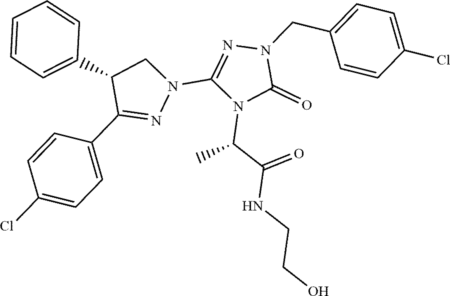 [SMILES: C[C@@H](C(=O)NCCO)n1c(N2C[C@@H](c3ccccc3)C(c3ccc(Cl)cc3)=N2)nn(Cc2ccc(Cl)cc2)c1=O] | 579.5 | ¹H NMR (400 MHz, DMSO-d6) chemical shifts 8.01 (t, J = 5.6 Hz, 1H), 7.69 - 7.62 (m, 2H), 7.44 -7.38 (m, 4H), 7.35 - 7.20 (m, 7H), 5.08 (q, J = 7.1 Hz, 1H), 4.96 (dd, J = 11.1, 4.0 Hz, 1H), 4.85 (q, J = 16.0 Hz, 2H), 4.65 (t, J = 5.5 Hz, 1H), 4.24 (t, J = 10.8 Hz, 1H), 3.69 (dd, J = 10.5, 4.0 Hz, 1H), 3.42 (q, J = 6.1 Hz, 2H), 3.20 (ddq, J = 19.0, 13.1, 6.1 Hz, 2H), 1.71 (d, J = 7.2 Hz, 3H). |
| 189 | 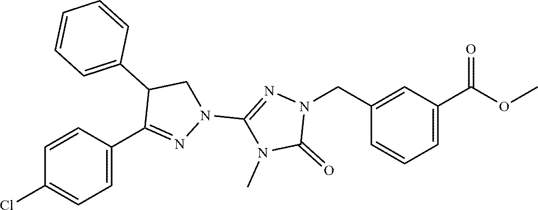 [SMILES: COC(=O)c1cccc(Cn2nc(N3CC(c4ccccc4)C(c4ccc(Cl)cc4)=N3)n(C)c2=O)c1] | 502 | ¹H NMR (400 MHz, DMSO-d6) chemical shifts 7.91 - 7.84 (m, 2H), 7.72 - 7.64 (m, 2H), 7.59 -7.52 (m, 1H), 7.51 (q, J = 8.0, 7.5 Hz, 1H), 7.46 - 7.39 (m, 2H), 7.31 (d, J = 6.2 Hz, 4H), 7.25 (td, J = 5.8, 2.5 Hz, 1H), 4.99 (dd, J = 11.0, 4.1 Hz, 1H), 4.93 (d, J = 4.4 Hz, 2H), 4.25 (t, J = 10.7 Hz, 1H), 3.84 (s, 3H), 3.70 (dd, J = 10.4, 4.2 Hz, 1H), 3.32 (s, 3H). |
| 190 | 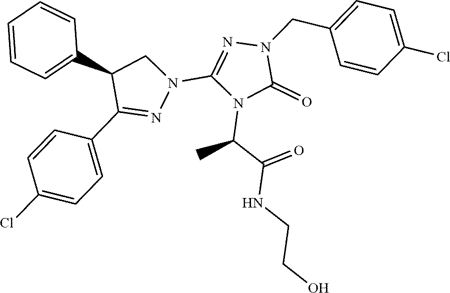 [SMILES: C[C@H](C(=O)NCCO)n1c(N2C[C@H](c3ccccc3)C(c3ccc(Cl)cc3)=N2)nn(Cc2ccc(Cl)cc2)c1=O] | 579.5 | ¹H NMR (400 MHz, DMSO-d6) chemical shifts 7.89 (t, J = 5.7 Hz, 1H), 7.69 - 7.63 (m, 2H), 7.45 -7.38 (m, 4H), 7.35 - 7.28 (m, 4H), 7.24 (d, J = 6.8 Hz, 3H), 5.06 (q, J = 7.1 Hz, 1H), 4.98 (dd, J = 10.9, 3.4 Hz, 1H), 4.91 - 4.79 (m, 2H), 4.61 (t, J = 5.5 Hz, 1H), 4.21 (t, J = 10.7 Hz, 1H), 3.68 (dd, J = 10.5, 3.5 Hz, 1H), 3.39 (d, J = 6.1 Hz, 2H), 3.16 (q, J = 6.1 Hz, 2H), 1.73 (d, J = 7.2 Hz, 3H). |
| 191 | 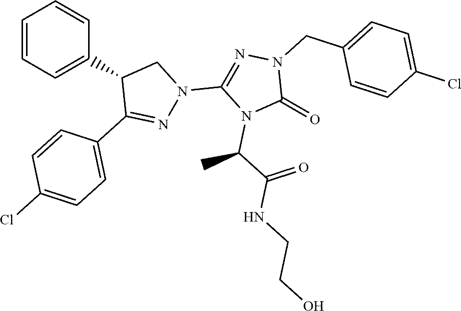 [SMILES: C[C@H](C(=O)NCCO)n1c(N2C[C@@H](c3ccccc3)C(c3ccc(Cl)cc3)=N2)nn(Cc2ccc(Cl)cc2)c1=O] | 579.5 | ¹H NMR (400 MHz, DMSO-d6) chemical shifts 7.88 (t, J = 5.5 Hz, 1H), 7.70 - 7.63 (m, 2H), 7.47 -7.37 (m, 4H), 7.35 - 7.28 (m, 4H), 7.24 (d, J = 7.1 Hz, 3H), 5.06 (q, J = 7.2 Hz, 1H), 4.98 (dd, J = 11.0, 3.4 Hz, 1H), 4.91 - 4.79 (m, 2H), 4.61 (t, J = 5.6 Hz, 1H), 4.21 (t, J = 10.7 Hz, 1H), 3.68 (dd, J = 10.4, 3.5 Hz, 1H), 3.39 (d, J = 6.0 Hz, 2H), 3.16 (q, J = 6.2 Hz, 2H), 1.73 (d, J = 7.2 Hz, 3H). |
| 192 | 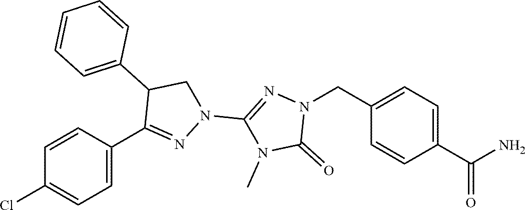 [SMILES: Cn1c(N2CC(c3ccccc3)C(c3ccc(Cl)cc3)=N2)nn(Cc2ccc(C(N)=O)cc2)c1=O] | 487 | ¹H NMR (400 MHz, DMSO-d6) chemical shifts 7.92 (s, 1H), 7.86 -7.79 (m, 2H), 7.72 - 7.64 (m, 2H), 7.47 - 7.39 (m, 2H), 7.37 - 7.29 (m,7H), 7.32 - 7.20 (m, 1H), 4.99 (dd, J = 10.9, 4.1 Hz, 1H), 4.97 -4.84 (m, 2H), 4.25 (t, J = 10.7 Hz, 1H), 3.71 (dd, J = 10.4, 4.1 Hz, 1H), 3.32 (s, 3H). |
| 193 | 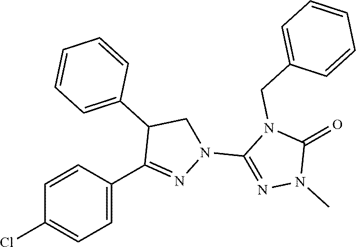 [SMILES: Cn1nc(N2CC(c3ccccc3)C(c3ccc(Cl)cc3)=N2)n(Cc2ccccc2)c1=O] | 443.9 | ¹H NMR (400 MHz, DMSO-d6) chemical shifts 7.57 - 7.51 (m, 2H), 7.42 - 7.31 (m, 5H), 7.31 -7.26 (m, 2H), 7.21 (dd, J = 5.2, 1.9 Hz, 3H), 7.05 - 6.98 (m, 2H), 5.20 (s, 2H), 4.89 (dd, J = 10.7, 3.4 Hz, 1H), 4.21 (t, J = 10.6 Hz, 1H), 3.62 (dd, J = 10.4, 3.4 Hz, 1H), 3.33 (s, 3H). |
| 194 | 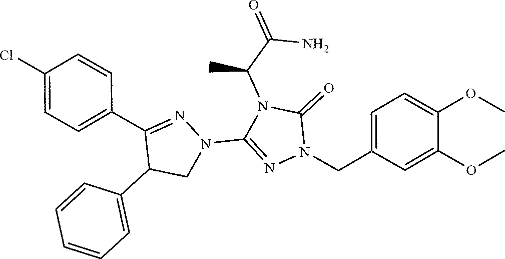 [SMILES: COc1ccc(Cn2nc(N3CC(c4ccccc4)C(c4ccc(Cl)cc4)=N3)n([C@@H](C)C(N)=O)c2=O)cc1OC] | 561 | ¹H NMR (400 MHz, DMSO-d6) chemical shifts 7.67 (dd, J = 8.5, 5.0 Hz, 2H), 7.56 - 7.37 (m, 3H), 7.35 - 7.20 (m, 6H), 6.93 - 6.79 (m, 3H), 5.08 - 4.93 (m, 2H), 4.84 -4.67 (m, 2H), 4.23 (dt, J = 14.2, 10.6 Hz, 1H), 3.72 (s, 7H), 1.72 (dd, J = 7.2, 4.8 Hz, 3H). |
| 195 | 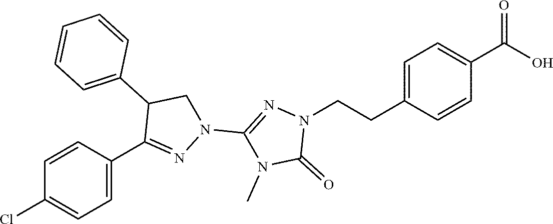 [SMILES: Cn1c(N2CC(c3ccccc3)C(c3ccc(Cl)cc3)=N2)nn(CCc2ccc(C(=O)O)cc2)c1=O] | 502 | ¹H NMR (400 MHz, DMSO-d6) chemical shifts 12.83 (s, 1H), 7.84 (d, J = 7.9 Hz, 2H), 7.68 (d, J = 8.3 Hz, 2H), 7.43 (d, J = 8.3 Hz, 2H), 7.33 (q, J = 4.1, 3.1 Hz, 6H), 7.26 (t, J = 5.9 Hz, 1H), 5.02 (dd, J = 10.9, 4.2 Hz, 1H), 4.30 (t, J = 10.7 Hz, 1H), 3.90 (t, J = 7.1 Hz, 2H), 3.75 (dd, J = 10.4, 4.3 Hz, 1H), 3.40 (s, 3H), 3.01 (t, J = 7.1 Hz, 2H). |
| 196 | 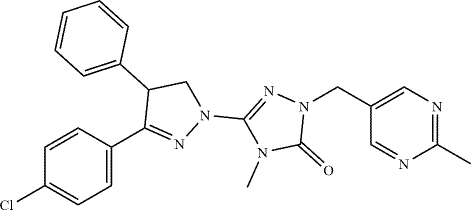 [SMILES: Cc1ncc(Cn2nc(N3CC(c4ccccc4)C(c4ccc(Cl)cc4)=N3)n(C)c2=O)cn1] | 459.9 | ¹H NMR (400 MHz, DMSO-d6) chemical shifts 8.62 (s, 2H), 7.67 (d, J = 8.6 Hz, 2H), 7.43 (d, J = 8.6 Hz, 2H), 7.35 - 7.24 (m, 5H), 4.99 -4.89 (m, 3H), 4.26 (t, J = 10.8 Hz, 1H), 3.71 (dd, J = 10.2, 4.3 Hz, 1H), 3.47 (s, 3H), 2.59 (s, 3H). |
| 197 | 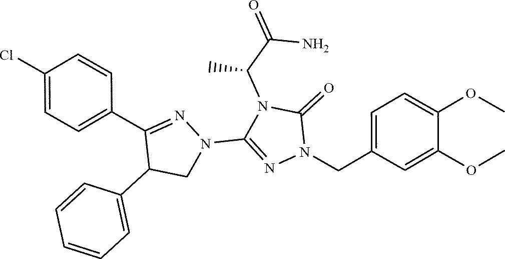 [SMILES: COc1ccc(Cn2nc(N3CC(c4ccccc4)C(c4ccc(Cl)cc4)=N3)n([C@H](C)C(N)=O)c2=O)cc1OC] | 561 | ¹H NMR (400 MHz, DMSO-d6) chemical shifts 7.67 (dd, J = 8.5, 4.9 Hz, 2H), 7.54 - 7.37 (m, 3H), 7.35 - 7.20 (m, 6H), 6.94 - 6.78 (m, 3H), 5.06 - 4.94 (m, 2H), 4.83 -4.69 (m, 2H), 4.28 - 4.18 (m, 1H), 3.72 (s, 7H), 1.72 (dd, J = 7.3, 4.8 Hz, 3H). |
| 199 | 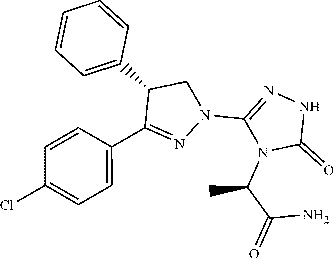 [SMILES: C[C@H](C(N)=O)n1c(N2C[C@@H](c3ccccc3)C(c3ccc(Cl)cc3)=N2)n[nH]c1=O] | 410.9 | ¹H NMR (400 MHz, DMSO-d6) chemical shifts 11.21 (s, 1H), 7.73 - 7.59 (m, 2H), 7.43 - 7.30 (m, 5H), 7.29 - 7.22 (m, 3H), 7.18 (s, 1H), 5.00 - 4.88 (m, 2H), 4.23 (t, J = 10.7 Hz, 1H), 3.70 (dd, J = 10.5, 3.5 Hz, 1H), 1.67 (d, J = 7.3 Hz, 3H). |
| 200 | 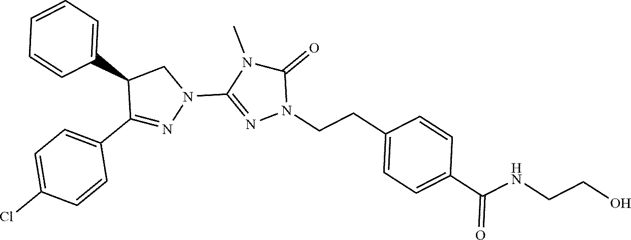 [SMILES: Cn1c(N2C[C@H](c3ccccc3)C(c3ccc(Cl)cc3)=N2)nn(CCc2ccc(C(=O)NCCO)cc2)c1=O] | 545 | ¹H NMR (400 MHz, DMSO-d6) chemical shifts 8.34 (t, J = 5.6 Hz, 1H), 7.79 - 7.73 (m, 2H), 7.72 -7.64 (m, 2H), 7.46 - 7.40 (m, 2H), 7.39 - 7.29 (m, 4H), 7.27 (dd, J = 7.3, 5.8 Hz, 3H), 5.02 (dd, J = 10.9, 4.2 Hz, 1H), 4.69 (t, J = 5.6 Hz, 1H), 4.31 (t, J = 10.7 Hz, 1H), 3.94 - 3.85 (m, 2H), 3.75 (dd, J = 10.4, 4.3 Hz, 1H), 3.50 (q, J = 6.1 Hz, 2H), 3.41 (s, 3H), 3.32 (s,1H), 3.30 (s, 1H), 2.99 (t, J = 7.1 Hz, 2H). |
| 201 | 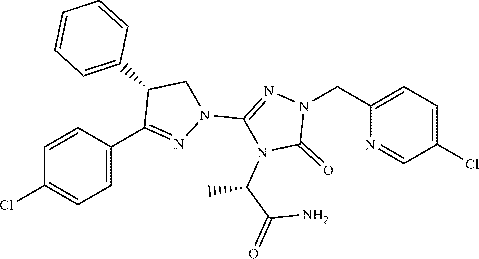 [SMILES: C[C@@H](C(N)=O)n1c(N2C[C@@H](c3ccccc3)C(c3ccc(Cl)cc3)=N2)nn(Cc2ccc(Cl)cn2)c1=O] | 536.4 | ¹H NMR (400 MHz, DMSO-d6) chemical shifts 8.57 (d, J = 2.4 Hz, 1H), 7.91 (d, J = 6.2 Hz, 1H), 7.69 (d, J = 8.4 Hz, 2H), 7.43 (t, J = 9.7 Hz, 3H), 7.33 (dd, J = 13.9, 7.6 Hz, 3H), 7.25 (d, J = 7.7 Hz, 4H), 5.03 (d, J = 7.6 Hz, 1H), 4.94 (s, 2H), 4.20 (t, J = 10.6 Hz, 1H), 3.67 (d, J = 13.8 Hz, 1H), 3.30 (s, 1H), 1.73 (d, J = 7.3 Hz, 3H). |
| 202 | 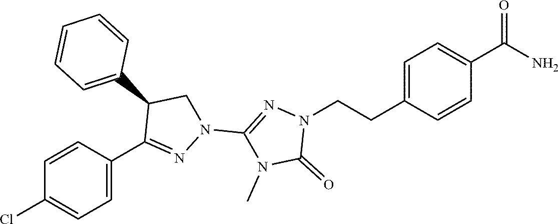 [SMILES: Cn1c(N2C[C@H](c3ccccc3)C(c3ccc(Cl)cc3)=N2)nn(CCc2ccc(C(N)=O)cc2)c1=O] | 501 | ¹H NMR (400 MHz, DMSO-d6) chemical shifts 7.89 (s, 1H), 7.81 -7.75 (m, 2H), 7.72 - 7.64 (m, 2H), 7.47 - 7.39 (m, 2H), 7.39 - 7.21 (m, 8H), 5.02 (dd, J = 10.9,4.2 Hz, 1H), 4.31 (t, J = 10.7 Hz, 1H), 3.94 - 3.84 (m, 2H), 3.75 (dd, J = 10.4, 4.2 Hz, 1H), 3.41 (s, 3H), 2.99 (t, J = 7.2 Hz, 2H). |
| 203 | 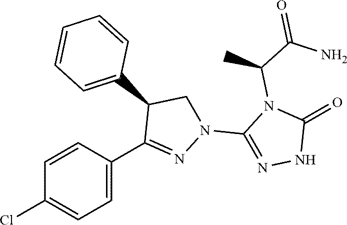 [SMILES: C[C@@H](C(N)=O)n1c(N2C[C@H](c3ccccc3)C(c3ccc(Cl)cc3)=N2)n[nH]c1=O] | 410.9 | ¹H NMR (400 MHz, DMSO-d6) chemical shifts 11.21 (s, 1H), 7.67 (d, J = 8.3 Hz, 2H), 7.38 (dd, J = 17.1, 7.4 Hz, 3H), 7.32 (d, J = 7.3 Hz, 2H), 7.30 - 7.23 (m, 3H), 7.18 (s, 1H), 5.00 - 4.88 (m, 2H), 4.23 (t, J = 10.7 Hz, 1H), 3.70 (dd, J = 10.5, 3.4 Hz, 1H), 1.68 (d, J = 7.3 Hz, 3H). |
| 205 | 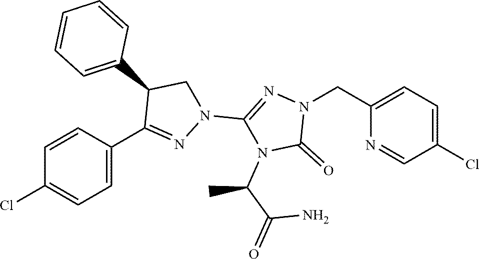 [SMILES: C[C@H](C(N)=O)n1c(N2C[C@H](c3ccccc3)C(c3ccc(Cl)cc3)=N2)nn(Cc2ccc(Cl)cn2)c1=O] | 536.4 | ¹H NMR (400 MHz, DMSO-d6) chemical shifts 8.57 (d, J = 2.5 Hz, 1H), 7.91 (dd, J = 8.4, 2.6 Hz, 1H), 7.69 (d, J = 8.5 Hz, 2H), 7.47 -7.37 (m, 3H), 7.33 (dd, J = 13.9, 7.7 Hz, 3H), 7.25 (d, J = 7.7 Hz, 4H), 5.03 (d, J = 7.2 Hz, 1H), 4.95 (d, J = 6.4 Hz, 2H), 4.20 (t, J = 10.7 Hz, 1H), 3.67 (dd, J = 10.5, 3.3 Hz, 1H), 3.30 (s, 1H), 1.73 (d, J = 7.2 Hz, 3H). |
| 206 | 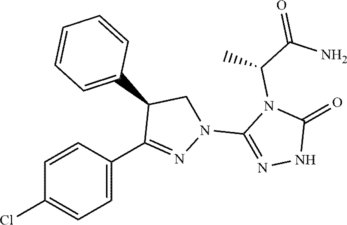 [SMILES: C[C@H](C(N)=O)n1c(N2C[C@H](c3ccccc3)C(c3ccc(Cl)cc3)=N2)n[nH]c1=O] | 410.9 | ¹H NMR (400 MHz, DMSO-d6) chemical shifts 11.21 (s, 1H), 7.69 - 7.64 (m, 2H), 7.47 (s, 1H), 7.42 -7.37 (m, 2H), 7.35 - 7.21 (m, 6H), 5.01 - 4.90 (m, 2H), 4.27 (t, J = 10.8 Hz, 1H), 3.71 (dd, J = 10.5, 4.5 Hz, 1H), 1.66 (d, J = 7.2 Hz, 3H), |
| 207 | 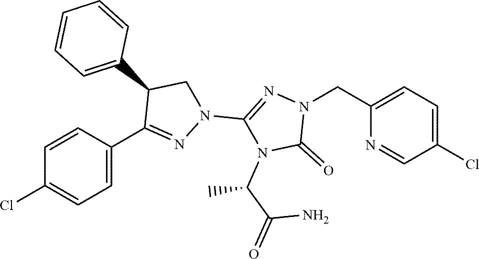 [SMILES: C[C@@H](C(N)=O)n1c(N2C[C@H](c3ccccc3)C(c3ccc(Cl)cc3)=N2)nn(Cc2ccc(Cl)cn2)c1=O] | 536.4 | ¹H NMR (400 MHz, DMSO-d6) chemical shifts 8.58 (d, J = 2.5 Hz, 1H), 7.91 (dd, J = 8.4, 2.6 Hz, 1H), 7.68 (d, J = 8.6 Hz, 2H), 7.54 (s, 1H), 7.40 (d, J = 8.5 Hz, 2H), 7.37 - 7.31 (m, 3H), 7.30 - 7.28 (m, 2H), 7.25 (dd, J = 13.6,6.2 Hz, 2H), 5.04 (q, J = 7.2 Hz, 1H), 4.94 (d, J = 6.3 Hz, 2H), 4.24 (t, J = 10.7 Hz, 1H), 3.68 (dd, J = 10.5, 4.2 Hz, 1H), 3.30 (s, 1H), 1.72 (d, J = 7.3 Hz, 3H). |
| 208 | 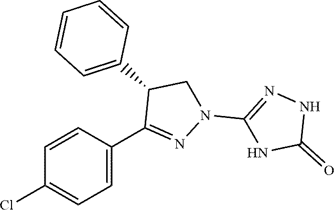 [SMILES: O=c1[nH]nc(N2C[C@@H](c3ccccc3)C(c3ccc(Cl)cc3)=N2)[nH]1] | 339.8 | ¹H NMR (400 MHz, DMSO-d6) chemical shifts 11.60 (s, 1H), 10.80 (s, 1H), 7.68 (d, J = 8.2 Hz, 2H), 7.42 (d, J = 8.2 Hz, 2H), 7.29 (dd, J = 24.4, 7.4 Hz, 5H), 5.00 (dd, J = 11.2, 4.5 Hz, 1H), 4.19 (t, J = 10.8 Hz, 1H), 3.71 (dd, J = 10.3, 4.5 Hz, 1H). |
| 209 | 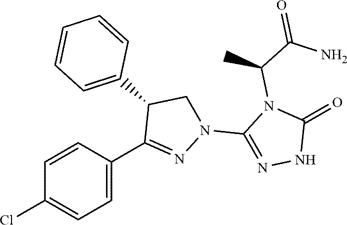 [SMILES: C[C@@H](C(N)=O)n1c(N2C[C@@H](c3ccccc3)C(c3ccc(Cl)cc3)=N2)n[nH]c1=O] | 410.9 | ¹H NMR (400 MHz, DMSO-d6) chemical shifts 11.21 (s, 1H), 7.69 - 7.63 (m, 2H), 7.47 (s, 1H), 7.43 -7.37 (m, 2H), 7.36 - 7.28 (m, 4H), 7.25 (d, J = 5.7 Hz, 2H), 5.01 -4.90 (m, 2H), 4.27 (t, J = 10.7 Hz, 1H), 3.71 (dd, J = 10.5, 4.5 Hz, 1H), 1.66 (d, J = 7.2 Hz, 3H). |
| 210 | 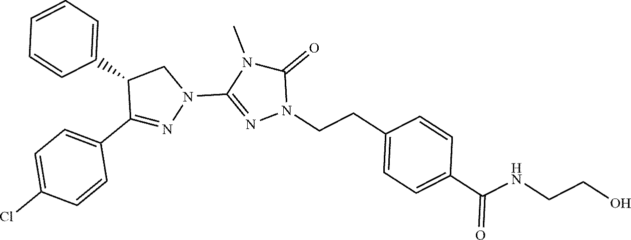 [SMILES: Cn1c(N2C[C@@H](c3ccccc3)C(c3ccc(Cl)cc3)=N2)nn(CCc2ccc(C(=O)NCCO)cc2)c1=O] | 545 | ¹H NMR (400 MHz, DMSO-d6) chemical shifts 8.34 (t, J = 5.6 Hz, 1H), 7.76 (d, J = 8.2 Hz, 2H), 7.72 - 7.65 (m, 2H), 7.47 - 7.40 (m, 2H), 7.34 (d, J = 6.5 Hz, 4H), 7.33 - 7.22 (m, 3H), 5.02 (dd, J = 11.0, 4.2 Hz, 1H), 4.69 (t, J = 5.6 Hz, 1H), 4.31 (t, J = 10.7 Hz, 1H), 3.89 (t, J = 7.1 Hz, 2H), 3.75 (dd, J = 10.4, 4.3 Hz, 1H), 3.50 (q, J = 6.1 Hz, 2H), 3.41 (s, 3H), 3.31 (s,1H), 3.30 (s,1H), 2.99 (t, J = 7.1 Hz, 2H). |
| 211 | 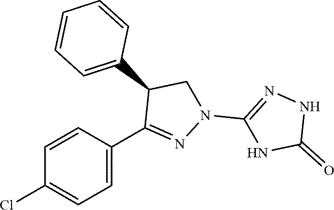 [SMILES: O=c1[nH]nc(N2C[C@H](c3ccccc3)C(c3ccc(Cl)cc3)=N2)[nH]1] | 339.8 | ¹H NMR (400 MHz, DMSO-d6) chemical shifts 11.60 (s, 1H), 10.80 (s, 1H), 7.68 (d, J = 8.5 Hz, 2H), 7.42 (d, J = 8.5 Hz, 2H), 7.29 (dd, J = 24.5, 7.4 Hz, 5H), 5.00 (dd, J = 11.2, 4.5 Hz, 1H), 4.19 (t, J = 10.8 Hz, 1H), 3.71 (dd, J = 10.4, 4.5 Hz, 1H). |
| 212 | 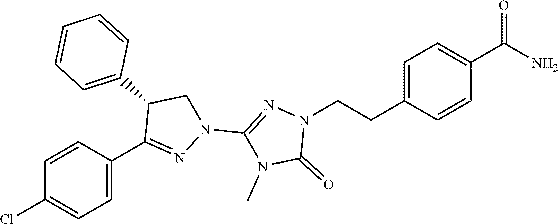 [SMILES: Cn1c(N2C[C@@H](c3ccccc3)C(c3ccc(Cl)cc3)=N2)nn(CCc2ccc(C(N)=O)cc2)c1=O] | 501 | ¹H NMR (400 MHz, DMSO-d6) chemical shifts 7.89 (s, 1H), 7.81 -7.75 (m, 2H), 7.72 - 7.64 (m, 2H), 7.47 - 7.39 (m, 2H), 7.39 - 7.22 (m, 8H), 5.02 (dd, J = 10.8, 4.2 Hz, 1H), 4.31 (t, J = 10.7 Hz, 1H), 3.90 (t, J = 7.1 Hz, 2H), 3.75 (dd, J = 10.4, 4.3 Hz, 1H), 3.41 (s, 3H), 2.99 (t, J = 7.2 Hz, 2H). |
| 213 | 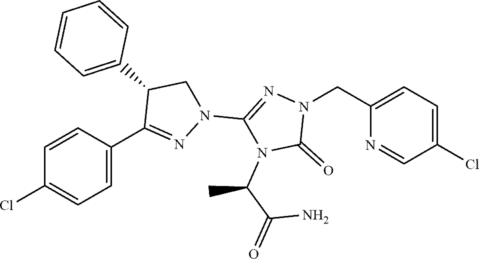 [SMILES: C[C@H](C(N)=O)n1c(N2C[C@@H](c3ccccc3)C(c3ccc(Cl)cc3)=N2)nn(Cc2ccc(Cl)cn2)c1=O] | 536.4 | ¹H NMR (400 MHz, DMSO-d6) chemical shifts 8.58 (d, J = 2.5 Hz, 1H), 7.91 (dd, J = 8.5, 2.5 Hz, 1H), 7.68 (d, J = 8.5 Hz, 2H), 7.54 (s, 1H), 7.43 -7.38 (m, 2H), 7.37 -7.31 (m, 3H), 7.31 - 7.28 (m, 2H), 7.28 - 7.20 (m, 2H), 5.04 (q, J = 7.0 Hz, 1H), 4.95 (t, J = 5.9 Hz, 2H), 4.24 (t, J = 10.7 Hz, 1H), 3.68 (dd, J = 10.6, 4.2 Hz, 1H), 3.30 (s, 1H), 1.72 (d, J = 7.2 Hz, 3H). |
| 214 | 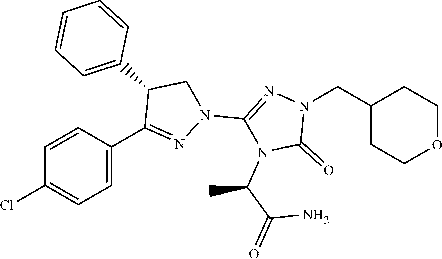 [SMILES: C[C@H](C(N)=O)n1c(N2C[C@@H](c3ccccc3)C(c3ccc(Cl)cc3)=N2)nn(CC2CCOCC2)c1=O] | 509 | ¹H NMR (400 MHz, DMSO-d6) chemical shifts 7.71 - 7.64 (m, 2H), 7.49 (s, 1H), 7.42 - 7.37 (m, 2H), 7.35 - 7.23 (m, 6H), 5.04 -4.92 (m, 2H), 4.28 (t, J = 10.8 Hz, 1H), 3.83 (d, J = 11.3 Hz, 2H), 3.71 (dd, J = 10.4, 4.3 Hz, 1H), 3.58 - 3.45 (m, 2H), 3.25 (td, J = 9.2, 4.6 Hz, 2H), 1.94 (dd, J = 7.8, 4.2 Hz, 1H), 1.67 (d, J = 7.2 Hz, 3H), 1.54 (s, 2H), 1.28 - 1.18 (m, 2H). |
| 215 | 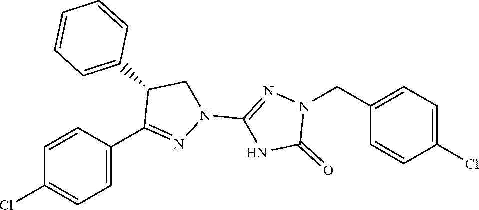 [SMILES: O=c1[nH]c(N2C[C@@H](c3ccccc3)C(c3ccc(Cl)cc3)=N2)nn1Cc1ccc(Cl)cc1] | 464.4 | ¹H NMR (400 MHz, DMSO-d6) chemical shifts 11.71 (s, 1H), 7.68 (d, J = 8.3 Hz, 2H), 7.46 - 7.35 (m, 4H), 7.29 (dq, J = 15.7, 7.5, 7.1 Hz, 7H), 5.01 (dd, J = 11.2, 4.5 Hz, 1H), 4.78 (s, 2H), 4.19 (t, J = 10.8 Hz, 1H), 3.70 (dd, J = 10.4, 4.5 Hz, 1H). |
| 216 | 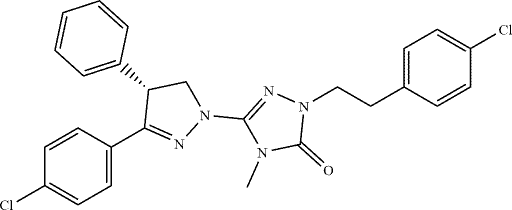 [SMILES: Cn1c(N2C[C@@H](c3ccccc3)C(c3ccc(Cl)cc3)=N2)nn(CCc2ccc(Cl)cc2)c1=O] | 492.4 | ¹H NMR (400 MHz, DMSO-d6) chemical shifts 7.71 - 7.64 (m, 2H), 7.46 - 7.40 (m, 2H), 7.39 -7.28 (m, 6H), 7.26 (td, J = 6.3, 2.6 Hz, 1H), 7.26 - 7.19 (m, 2H), 5.02 (dd, J = 11.0, 4.2 Hz, 1H), 4.29 (t, J = 10.6 Hz, 1H), 3.86 (t, J = 7.1 Hz, 2H), 3.74 (dd, J = 10.4, 4.2 Hz, 1H), 3.41 (s, 3H), 2.94 (t, J = 7.1 Hz, 2H). |
| 217 | 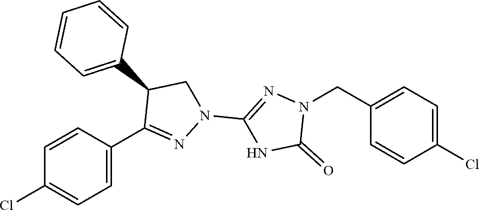 [SMILES: O=c1[nH]c(N2C[C@H](c3ccccc3)C(c3ccc(Cl)cc3)=N2)nn1Cc1ccc(Cl)cc1] | 464.4 | ¹H NMR (400 MHz, DMSO-d6) chemical shifts 11.90 (s, 1H), 7.68 (d, J = 8.3 Hz, 2H), 7.50 - 7.37 (m, 4H), 7.29 (dq, J = 15.8, 7.5 Hz, 7H), 5.01 (dd, J = 11.1, 4.5 Hz, 1H), 4.78 (s, 2H), 4.19 (t, J = 10.7 Hz, 1H), 3.70 (dd, J = 10.4, 4.5 Hz, 1H). |
| 218 | 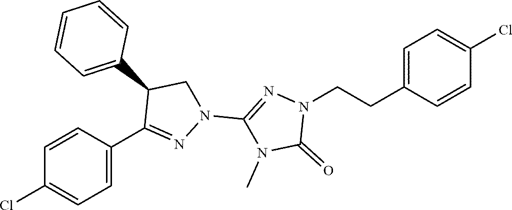 [SMILES: Cn1c(N2C[C@H](c3ccccc3)C(c3ccc(Cl)cc3)=N2)nn(CCc2ccc(Cl)cc2)c1=O] | 492.4 | ¹H NMR (400 MHz, DMSO-d6) chemical shifts 7.71 - 7.63 (m, 2H), 7.46 - 7.35 (m, 2H), 7.39 -7.29 (m, 6H), 7.26 (s, 1H), 7.22 (d, J = 8.4 Hz, 2H), 5.02 (dd, J = 10.9, 4.2 Hz, 1H), 4.29 (t, J = 10.7 Hz, 1H), 3.86 (t, J = 7.1 Hz, 2H), 3.74 (dd, J = 10.3, 4.2 Hz, 1H), 3.41 (s, 3H), 2.94 (t, J = 7.1 Hz, 2H). |
| 219 | 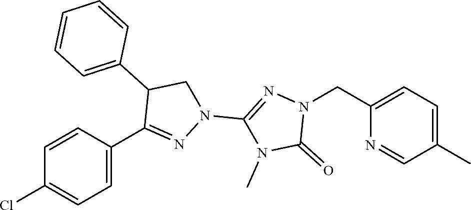 [SMILES: Cc1ccc(Cn2nc(N3CC(c4ccccc4)C(c4ccc(Cl)cc4)=N3)n(C)c2=O)nc1] | 459 | ¹H NMR (400 MHz, DMSO-d6) chemical shifts 8.37 - 8.29 (m, 1H), 7.74 - 7.64 (m, 2H), 7.57 (dd, J = 7.9, 2.3 Hz, 1H), 7.46 - 7.40 (m, 2H), 7.37 - 7.22 (m, 5H), 7.12 (d, J = 8.0 Hz, 1H), 5.05 - 4.94 (m, 1H), 4.90 (d, J = 3.9 Hz, 2H), 4.24 (t, J = 10.7 Hz, 1H), 3.69 (dd, J = 10.5, 4.1 Hz, 1H), 3.50 (s, 3H), 2.27 (s, 3H). |
| 220 | 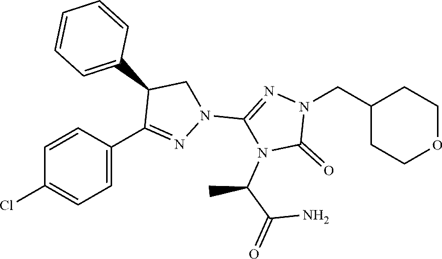 [SMILES: C[C@H](C(N)=O)n1c(N2C[C@H](c3ccccc3)C(c3ccc(Cl)cc3)=N2)nn(CC2CCOCC2)c1=O] | 509 | ¹H NMR (400 MHz, DMSO-d6) chemical shifts 7.71 - 7.62 (m, 2H), 7.49 (s, 1H), 7.42 - 7.36 (m, 2H), 7.35 - 7.21 (m, 6H), 5.03 -4.90 (m, 2H), 4.28 (t, J = 10.8 Hz, 1H), 3.87 - 3.77 (m, 2H), 3.71 (dd, J = 10.5, 4.3 Hz, 1H), 3.57 - 3.44 (m, 2H), 3.29 - 3.20 (m, 2H), 1.94 (t, J = 3.8 Hz, 1H), 1.67 (d, J = 7.2 Hz, 3H), 1.54 (s, 2H), 1.22 (qd, J = 14.6, 13.6, 5.4 Hz, 2H). |
| 221 | 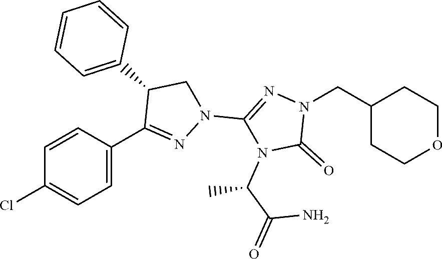 [SMILES: C[C@@H](C(N)=O)n1c(N2C[C@@H](c3ccccc3)C(c3ccc(Cl)cc3)=N2)nn(CC2CCOCC2)c1=O] | 509 | ¹H NMR (400 MHz, DMSO-d6) chemical shifts 7.73 - 7.65 (m, 2H), 7.45 - 7.38 (m, 3H), 7.33 (dd, J = 8.1, 6.8 Hz, 2H), 7.29 - 7.17 (m, 4H), 5.02 - 4.91 (m, 2H), 4.24 (t, J = 10.7 Hz, 1H), 3.82 (d, J = 11.3 Hz, 2H), 3.70 (dd, J = 10.5, 3.4 Hz, 1H), 3.51 (d, J = 7.0 Hz, 2H), 3.25 (t, J = 11.5 Hz, 2H), 2.01 - 1.86 (m, 1H), 1.69 (d, J = 7.2 Hz, 3H), 1.54 (d, J = 13.0 Hz, 2H), 1.27 - 1.15 (m, 2H). |
| 222 | 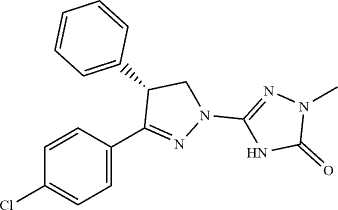 [SMILES: Cn1nc(N2C[C@@H](c3ccccc3)C(c3ccc(Cl)cc3)=N2)[nH]c1=O] | 353.8 | ¹H NMR (400 MHz, DMSO-d6) chemical shifts 11.74 (s, 1H), 7.68 (d, J = 8.5 Hz, 2H), 7.43 (d, J = 8.5 Hz, 2H), 7.34 - 7.17 (m, 5H), 5.02 (dd, J = 11.2, 4.5 Hz, 1H), 4.21 (t, J = 10.8 Hz, 1H), 3.73 (dd, J = 10.4, 4.5 Hz, 1H), 3.20 (s, 3H). |
| 223 | 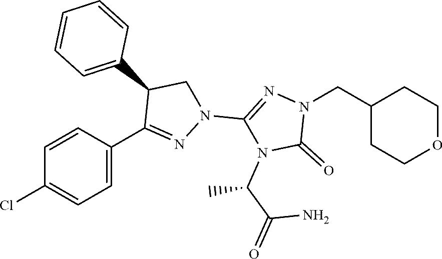 [SMILES: C[C@@H](C(N)=O)n1c(N2C[C@H](c3ccccc3)C(c3ccc(Cl)cc3)=N2)nn(CC2CCOCC2)c1=O] | 509 | ¹H NMR (400 MHz, DMSO-d6) chemical shifts 7.72 - 7.65 (m, 2H), 7.40 (dq, J = 8.6, 2.5 Hz, 3H), 7.36 - 7.29 (m, 2H), 7.26 (dt, J = 8.0, 1.9 Hz, 3H), 7.22 (d, J = 7.5 Hz, 1H), 5.03 - 4.91 (m, 2H), 4.24 (t, J = 10.7 Hz, 1H), 3.82 (d, J = 10.7 Hz, 2H), 3.70 (dd, J = 10.5, 3.3 Hz, 1H), 3.51 (d, J = 6.9 Hz, 2H), 3.25 (s, 2H), 1.94 (dt, J = 10.2, 5.9 Hz, 1H), 1.69 (d, J = 7.2 Hz, 3H), 1.54 (d, J = 12.6 Hz, 2H), 1.22 (q, J = 11.8 Hz, 2H). |
| 224 | 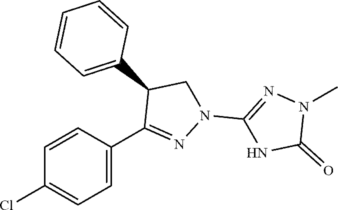 [SMILES: Cn1nc(N2C[C@H](c3ccccc3)C(c3ccc(Cl)cc3)=N2)[nH]c1=O] | 353.8 | ¹H NMR (400 MHz, DMSO-d6) chemical shifts 11.68 (s, 1H), 7.68 (d, J = 8.4 Hz, 2H), 7.43 (d, J = 8.4 Hz, 2H), 7.39 - 7.15 (m, 5H), 5.02 (dd, J = 11.2, 4.5 Hz, 1H), 4.21 (t, J = 10.8 Hz, 1H), 3.73 (dd, J = 10.3, 4.5 Hz, 1H), 3.32 (s, 2H), 3.20 (s, 3H). |

### Example 37. CB1 cAMP Antagonist Assay

LANCE Ultra cAMP kit (Perkin Elmer) was used to quantitate the amount of 3′,5′-cyclic adenosine monophosphate (cAMP) produced in Flp-In CHO (Invitrogen) cells stably expressing the CB1 receptor.

Forskolin was initially titrated to determine the response of the cells. The EC₉₀ of forskolin was used for compound testing. CP55940 was titrated and used with EC₉₀ of forskolin to determine the level of agonist stimulation. EC₉₀ of the agonist was used for subsequent compound testing.

Forskolin (Sigma), CP55940 (Cayman Chemicals), and AM251 (MCE) were diluted in 100% DMSO, starting at 100 mM, 1 mM, and 1 mM respectively, in 3-fold serial dilutions. Test compounds were diluted in 100% DMSO starting from 10 mM, 3-fold dilutions. The cAMP assay buffer contains 1x Hank’s Buffered Saline Solution with Ca²⁺ and Mg²⁺ (Invitrogen), 5.3 mM HEPES (Invitrogen), 0.05% BSA, 0.5 mM IBMX (Sigma).

For all assays, cells were harvested, counted, and diluted in cAMP assay buffer to 1× 10⁵ cells/mL. Only cells with viability >85% were used for the assay. Cells were seeded at 1000 cells/well in 384-well plates and 10 nL/well AM251 or test compound was added and incubated at 37° C. for 10 min. Then forskolin and agonist were added to reach their EC₉₀ and incubated at 25° C. for 30 min. To detect the amount of cAMP produced, 5 µL of a 100x diluted stock of Eu-cAMP tracer and 5 µL of 200x diluted stock of Ulight-anti-cAMP were added to each well, and the plate incubated at 25° C. for 15 min. The FRET signal was read using an EnVision microplate reader (λₑₓ=320 nm, λₑₘ=615 nm and 665 nm).

The results are expressed as % Inhibition, where % inhibition = 100 - 100 × (U -C2)/(C1 - C2), where U is the FRET ratio (λₑₘ (665 nm)/λₑₘ (615 nm)) of sample, C1 is the average of the high controls (signal with no antagonist added), and C2 is the average of low controls (signal with the highest concentration of AM251 antagonist). The IC₅₀ is determined by fitting the percentage of inhibition as a function of compound concentrations with the Hill equation, using a 4-parameter fit in either XLfit or GraphPad Prism.

The following IC₅₀ data were obtained for the compounds using the assay described above (A < 100 nM; 100 nM ≤ B < 1 µM; 1 µM ≤ C < 5 µM; D ≥ 5 µM; NT - not tested):

| Compound | CB1 IC₅₀ |
|---|---|
| 100 | D |
| 101 | A |
| 102 | B |
| 103 | A |
| 104 | A |
| 105 | A |
| 106 | C |
| 107 | A |
| 108 | A |
| 109 | D |
| 110 | D |
| 111 | A |
| 112 | D |
| 113 | C |
| 114 | B |
| 115 | C |
| 116 | D |
| 117 | D |
| 118 | B |
| 119 | D |
| 120 | C |
| 121 | B |
| 122 | B |
| 123 | B |
| 124 | B |
| 125 | C |
| 126 | B |
| 127 | D |
| 128 | D |
| 129 | D |
| 130 | D |
| 131 | D |
| 132 | C |
| 133 | C |
| 134 | C |
| 135 | D |
| 136 | B |
| 137 | C |
| 138 | D |
| 139 | B |
| 140 | C |
| 141 | C |
| 142 | C |
| 143 | A |
| 144 | B |
| 145 | B |
| 146 | D |
| 147 | C |
| 149 | D |
| 150 | B |
| 152 | C |
| 153 | A |
| 154 | A |
| 158 | A |
| 159 | C |
| 160 | A |
| 161 | C |
| 163 | A |
| 164 | D |
| 166 | B |
| 167 | B |
| 168 | A |
| 169 | A |
| 170 | A |
| 171 | A |
| 172 | C |
| 173 | C |
| 174 | A |
| 175 | A |
| 176 | A |
| 177 | A |
| 178 | A |
| 179 | D |
| 180 | D |
| 181 | D |
| 182 | B |
| 183 | B |
| 184 | A |
| 186 | D |
| 187 | D |
| 189 | B |
| 190 | A |
| 191 | B |
| 192 | B |
| 193 | B |
| 194 | B |
| 195 | D |
| 196 | B |
| 197 | B |
| 199 | D |
| 200 | B |
| 201 | B |
| 202 | B |
| 203 | D |
| 205 | A |
| 206 | D |
| 207 | A |
| 208 | D |
| 209 | D |
| 210 | D |
| 211 | D |
| 212 | D |
| 213 | D |
| 214 | NT |
| 215 | NT |
| 216 | NT |
| 217 | NT |
| 218 | NT |
| 219 | NT |
| 220 | NT |
| 221 | NT |
| 222 | NT |
| 223 | NT |
| 224 | NT |

### By Reference

All of the U.S. patents and U.S. and PCT published patent applications cited herein are hereby incorporated by reference.

### Equivalents

The foregoing written specification is sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A compound of structural formula I: 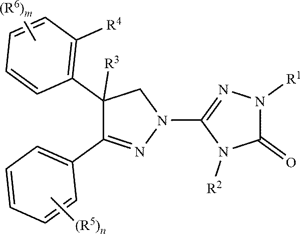
, or a pharmaceutically acceptable salt thereof, wherein:
R¹ is hydrogen, -C₁-C₄ alkyl, -(C₀-C₂ alkylene)-aryl, -(C₀-C₂ alkylene)-heteroaryl, -(C₀-C₂ alkylene)-heterocyclyl and -(C₀-C₂ alkylene)-carbocyclyl, wherein any alkyl, alkylene, aryl, heteroaryl, heterocyclyl, or carbocyclyl portion of R¹ is optionally substituted with 1 to 3 independently selected substituents;
R² is benzyl, -C₁-C₄ alkyl, -(C₁-C₄ alkylene)-C(O)-NH₂, -(C₁-C₄ alkylene)-C(O)-NH-(C₁-C₄ alkyl), -(C₁-C₄ alkylene)-S(O)₂-NH₂, -(C₁-C₄ alkylene)-S(O)₂-NH-(C₁-C₄ alkyl), -(C₁-C₄ alkylene)-O-(C₁-C₄ alkyl), or -(C₁-C₄ alkylene)-C(O)-O-(C₁-C₄ alkyl), wherein any alkyl portion of R² is optionally substituted with halo, —CN, —OH, or NO₂.
R³ is hydrogen; and
R⁴ is hydrogen, halo, or —CN; or
R³ and R⁴ are taken together with the carbon atoms to which they are bound and intervening atoms to form a 3-7 membered cycloalkyl moiety that is fused to the phenyl moiety bearing R⁴ and spirofused to the 4,5-dihydropyrazol-1,3,4-triyl moiety bearing R³;
each R⁵, if present, is independently halo, —CN, —OH, —NH₂, -NH(C₁-C₃ unsubstituted alkyl), -N(C₁-C₃ unsubstituted alkyl)₂, —NO₂, C₁-C₄ alkyl, C₂-C₄ alkenyl, C₂-C₄ alkynyl, or -O-C₁-C₄ alkyl, wherein the alkyl, alkenyl or alkynyl of R⁵ is optionally substituted with one or more substituents independently selected from halo, —CN, —OH, —O—(C₁-C₃ unsubstituted alkyl), —NH₂, -NH(C₁-C₃ unsubstituted alkyl), -N(C₁-C₃ unsubstituted alkyl)₂, and —NO₂;
each R⁶, if present is independently halo, —CN, —OH, —NH₂, -NH(C₁-C₃ unsubstituted alkyl), -N(C₁-C₃ unsubstituted alkyl)₂, —NO₂, C₁-C₄ alkyl, C₂-C₄ alkenyl, C₂-C₄ alkynyl, or - O-C₁-C₄ alkyl, wherein the alkyl, alkenyl or alkynyl of R⁶ is optionally substituted with one or more substituents independently selected from halo, —CN, —OH, -O-(C₁-C₃ unsubstituted alkyl), —NH₂, -NH(C₁-C₃ unsubstituted alkyl), -N(C₁-C₃ unsubstituted alkyl)₂, and -NO₂;
n is 0, 1, 2, 3, or 4;
m is 0, 1, 2, 3, or 4; and
m+n is greater than 0.

2. The compound of claim 1, wherein R¹ is hydrogen, -CH₃, phenyl, -CH₂-phenyl, -(CH₂)2-phenyl, -CH(CH₃)-phenyl, -CH₂-pyridinyl, -CH₂-pyrimidinyl, or -CH₂-cyclohexyl; and wherein the phenyl, pyridinyl, pyrimidinyl, or cyclohexyl portion of R¹ is optionally substituted with up to 3 substituents independently selected from halo, —CN, C₁-C₄ alkyl, -O-(C₁-C₄ alkyl),
—C(O)OH, -C(O)O-(C₁-C₄ alkyl), —C(O)NH₂, -C(O)NH-(C₁-C₄ alkyl) -(C₀-C₁ alkylene)- heterocyclyl, and -(C₀-C₁ alkylene)-O-heterocyclyl, wherein any alkyl, alkylene, or heterocyclyl portion of the R¹ substituent is further substituted with up to 3 substituents independently selected from halo, —OH, C₁-C₄ alkyl, C₁-C₄ hydroxyalkyl, C₁-C₄ haloalkyl, and -S(C₁-C₄ alkyl).

3. The compound of claim 2, wherein R¹ is hydrogen, -CH₃, benzyl, 2-(4-(2-hydroxyethan-1-ylcarbamoyl)phenyl)ethan-1-yl, 2-(4-carbamoylphenyl)ethan-1-yl, 2-(4-carboxyphenyl)ethan-1-yl, 2-(4-chlorophenyl)ethan-1-yl, 2-(4-chlorophenyl)ethan-2-yl, 2-chlorobenzyl, 2-methylpyrimidin-5-ylmethyl, 3,4-dimethoxybenzyl, 3-carbamoylbenzyl, 3-carboxybenzyl, 3-cyanobenzyl, 3-fluoro-4-methylbenzyl, 3-methoxycarbonylbenzyl, 3-methyl-4-chlorobenzyl, 4-(2-hydroxyethancarbamoyl)benzyl, 4-(4-methylpiperazin-1-ylmethyl)benzyl, 4-(morpholin-4-ylmethyl)benzyl, 4-(morpholin-4-ylmethyl)phenyl, 4-carbamoylbenzyl, 4-chlorobenzyl, 4-chlorophenyl, 4-cyanobenzyl, 4-methoxybenzyl, 4-methoxycarbonylbenzyl, 4-methylbenzyl, 5-chloropyridin-2-ylmethyl, 5-methylpyridin-2-yl, 6-methylpyridin-3-ylmethyl, cyclohexylmethyl, or tetrahydropyran-4-ylmethyl.

4. The compound of any one of claims 1-3, wherein R² is hydrogen, C₁-C₄ alkyl, -(Ci-C₄ alkylene)—C(O)—NH₂, -(C₁-C₄ alkylene)-C(O)-NH-(C₁-C₄ alkyl), -(C₁-C₄ alkylene)-C(O)-OH, -(C₁-C₄ alkylene)-C(O)-O-(C₁-C₄ alkyl), -(C₁-C₄ alkylene)-C(O)-(C₁-C₄ alkyl), or (C₀-C₁ alkylene)-aryl, wherein any alkylene, alkyl, or aryl portion of R² is optionally substituted with up to 3 substituents independently selected from halo, -OH, or —CN.

5. The compound of claim 4, wherein R² is hydrogen, —CH₃, —CH(CH₃)—C(O)—NH₂, —CH(CH₃)—C(O)—NH—(CH₂)₂—OH, —(CH₂)₂—C(O)OH, —(CH₂)₂C(O)—NH₂, —CH₂—C(O)—NH₂, —(CH₂)₂—O—CH₃, —(CH₂)₂—OH, —CH₂—C(O)—O—CH₂CH₃, or benzyl.

6. The compound of any one of claims 1-5, wherein R³ and R⁴ are hydrogen.

7. The compound of any one of claims 1-5, wherein R³ and R⁴ are taken together with the carbon atoms to which they are bound and intervening atoms to form a cyclopentyl moiety that is fused to the phenyl moiety bearing R⁴ and spirofused to the 4,5-dihydropyrazol-1,3,4-triyl moiety bearing R³.

8. The compound of any one of claims 1-7, wherein one R⁵ or one R⁶ is chloro.

9. The compound of claim 8, wherein m is 0, n is 1, and R⁵ is chloro.

10. The compound of claim 8 or 9, wherein the chloro is in the para position.

11. The compound of claim 1, wherein the compound is any one of the compounds set forth in the following table:

| # | Structure |
|---|---|
| 100 | 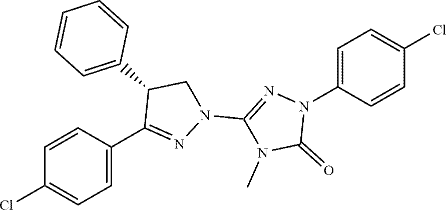 [SMILES: Cn1c(N2C[C@@H](c3ccccc3)C(c3ccc(Cl)cc3)=N2)nn(-c2ccc(Cl)cc2)c1=O] |
| 101 | 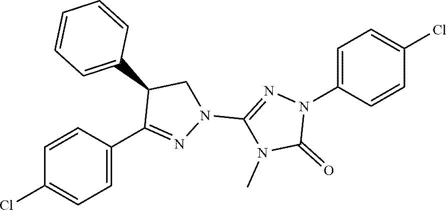 [SMILES: Cn1c(N2C[C@H](c3ccccc3)C(c3ccc(Cl)cc3)=N2)nn(-c2ccc(Cl)cc2)c1=O] |
| 102 | 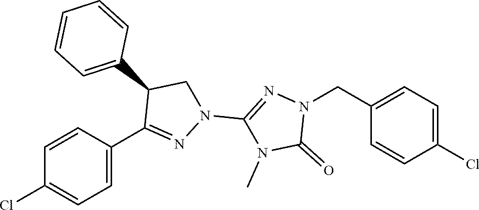 [SMILES: Cn1c(N2C[C@H](c3ccccc3)C(c3ccc(Cl)cc3)=N2)nn(Cc2ccc(Cl)cc2)c1=O] |
| 103 | 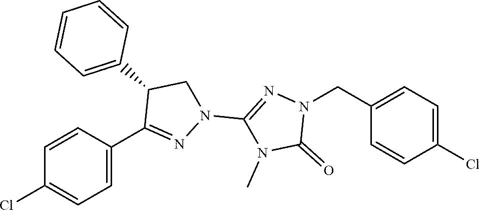 [SMILES: Cn1c(N2C[C@@H](c3ccccc3)C(c3ccc(Cl)cc3)=N2)nn(Cc2ccc(Cl)cc2)c1=O] |
| 104 | 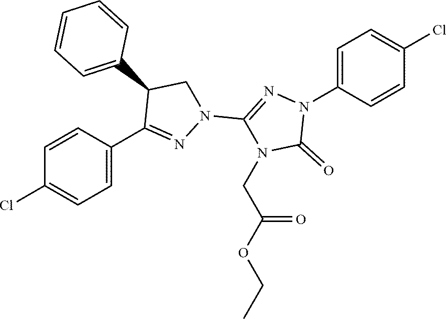 [SMILES: CCOC(=O)Cn1c(N2C[C@H](c3ccccc3)C(c3ccc(Cl)cc3)=N2)nn(-c2ccc(Cl)cc2)c1=O] |
| 105 | 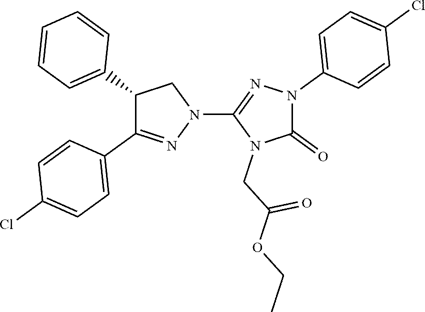 [SMILES: CCOC(=O)Cn1c(N2C[C@@H](c3ccccc3)C(c3ccc(Cl)cc3)=N2)nn(-c2ccc(Cl)cc2)c1=O] |
| 106 | 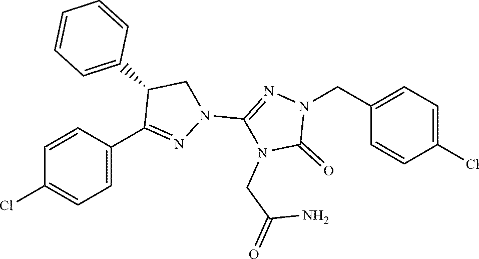 [SMILES: NC(=O)Cn1c(N2C[C@@H](c3ccccc3)C(c3ccc(Cl)cc3)=N2)nn(Cc2ccc(Cl)cc2)c1=O] |
| 107 | 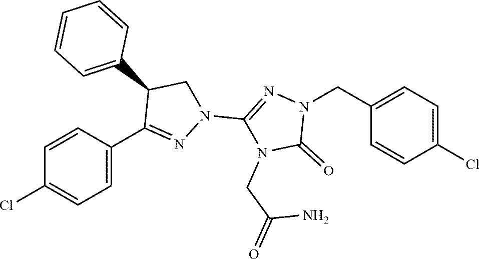 [SMILES: NC(=O)Cn1c(N2C[C@H](c3ccccc3)C(c3ccc(Cl)cc3)=N2)nn(Cc2ccc(Cl)cc2)c1=O] |
| 108 | 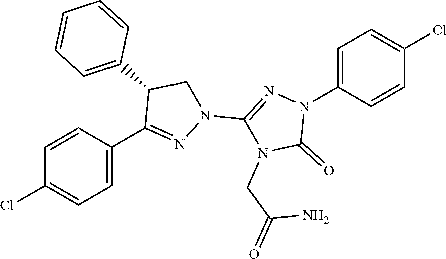 [SMILES: NC(=O)Cn1c(N2C[C@@H](c3ccccc3)C(c3ccc(Cl)cc3)=N2)nn(-c2ccc(Cl)cc2)c1=O] |
| 109 | 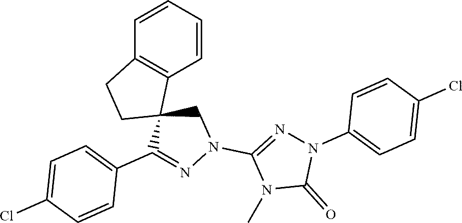 [SMILES: Cn1c(N2C[C@]3(CCc4ccccc43)C(c3ccc(Cl)cc3)=N2)nn(-c2ccc(Cl)cc2)c1=O] |
| 110 | 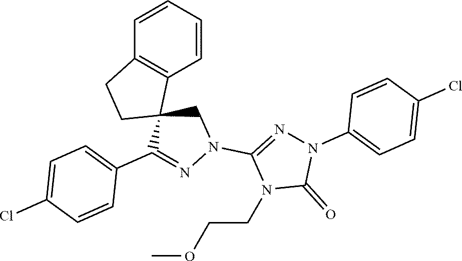 [SMILES: COCCn1c(N2C[C@]3(CCc4ccccc43)C(c3ccc(Cl)cc3)=N2)nn(-c2ccc(Cl)cc2)c1=O] |
| 111 | 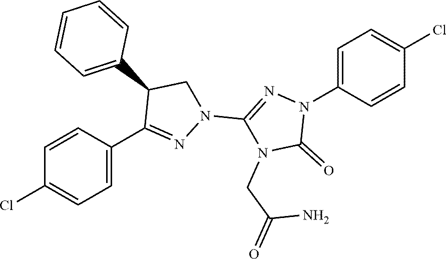 [SMILES: NC(=O)Cn1c(N2C[C@H](c3ccccc3)C(c3ccc(Cl)cc3)=N2)nn(-c2ccc(Cl)cc2)c1=O] |
| 112 | 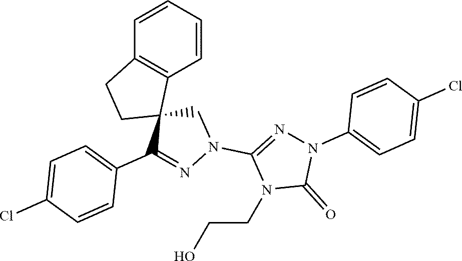 [SMILES: O=c1n(-c2ccc(Cl)cc2)nc(N2C[C@@]3(CCc4ccccc43)C(c3ccc(Cl)cc3)=N2)n1CCO] |
| 113 | 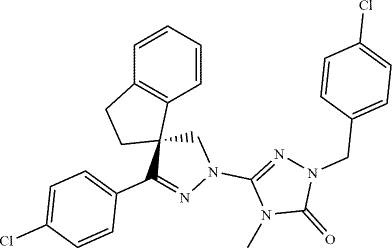 [SMILES: Cn1c(N2C[C@@]3(CCc4ccccc43)C(c3ccc(Cl)cc3)=N2)nn(Cc2ccc(Cl)cc2)c1=O] |
| 114 | 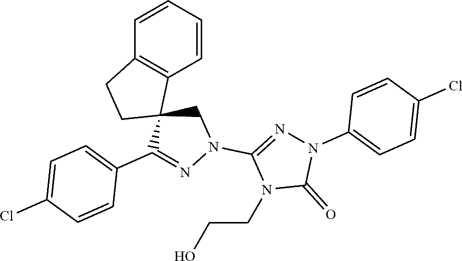 [SMILES: O=c1n(-c2ccc(Cl)cc2)nc(N2C[C@]3(CCc4ccccc43)C(c3ccc(Cl)cc3)=N2)n1CCO] |
| 115 | 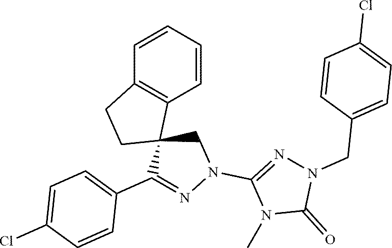 [SMILES: Cn1c(N2C[C@]3(CCc4ccccc43)C(c3ccc(Cl)cc3)=N2)nn(Cc2ccc(Cl)cc2)c1=O] |
| 116 | 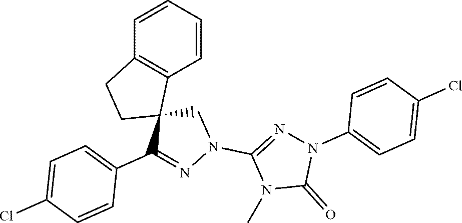 [SMILES: Cn1c(N2C[C@@]3(CCc4ccccc43)C(c3ccc(Cl)cc3)=N2)nn(-c2ccc(Cl)cc2)c1=O] |
| 117 | 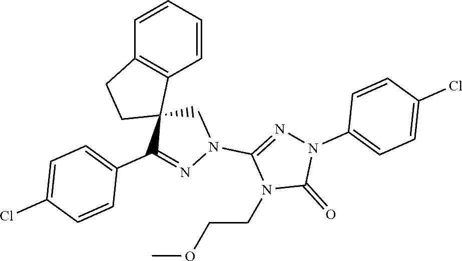 [SMILES: COCCn1c(N2C[C@@]3(CCc4ccccc43)C(c3ccc(Cl)cc3)=N2)nn(-c2ccc(Cl)cc2)c1=O] |
| 118 | 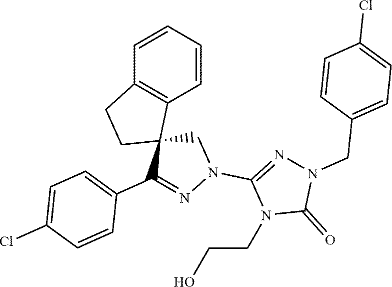 [SMILES: O=c1n(Cc2ccc(Cl)cc2)nc(N2C[C@@]3(CCc4ccccc43)C(c3ccc(Cl)cc3)=N2)n1CCO] |
| 119 | 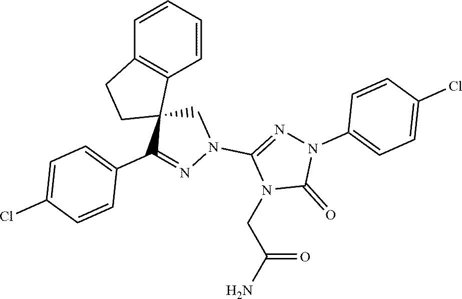 [SMILES: NC(=O)Cn1c(N2C[C@@]3(CCc4ccccc43)C(c3ccc(Cl)cc3)=N2)nn(-c2ccc(Cl)cc2)c1=O] |
| 120 | 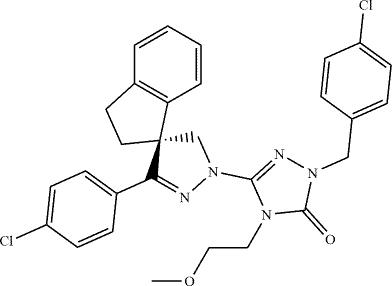 [SMILES: COCCn1c(N2C[C@@]3(CCc4ccccc43)C(c3ccc(Cl)cc3)=N2)nn(Cc2ccc(Cl)cc2)c1=O] |
| 121 | 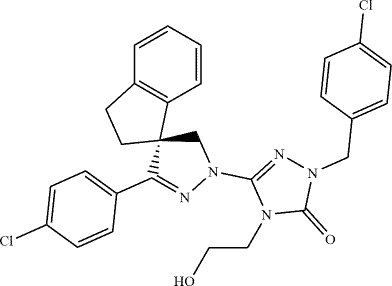 [SMILES: O=c1n(Cc2ccc(Cl)cc2)nc(N2C[C@]3(CCc4ccccc43)C(c3ccc(Cl)cc3)=N2)n1CCO] |
| 122 | 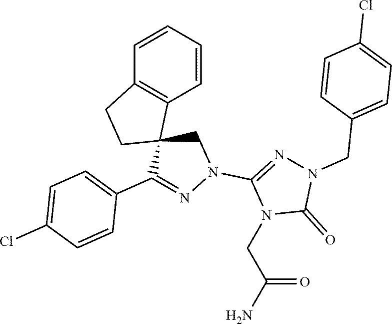 [SMILES: NC(=O)Cn1c(N2C[C@]3(CCc4ccccc43)C(c3ccc(Cl)cc3)=N2)nn(Cc2ccc(Cl)cc2)c1=O] |
| 123 | 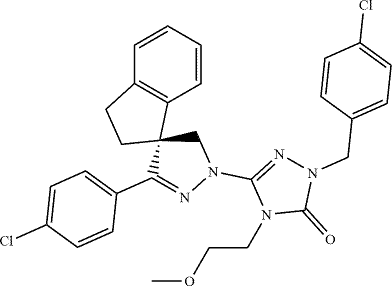 [SMILES: COCCn1c(N2C[C@]3(CCc4ccccc43)C(c3ccc(Cl)cc3)=N2)nn(Cc2ccc(Cl)cc2)c1=O] |
| 124 | 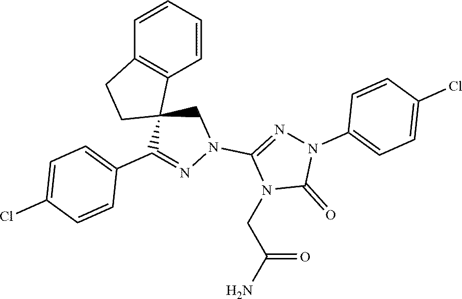 [SMILES: NC(=O)Cn1c(N2C[C@]3(CCc4ccccc43)C(c3ccc(Cl)cc3)=N2)nn(-c2ccc(Cl)cc2)c1=O] |
| 125 | 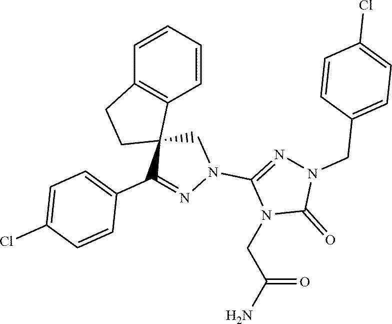 [SMILES: NC(=O)Cn1c(N2C[C@@]3(CCc4ccccc43)C(c3ccc(Cl)cc3)=N2)nn(Cc2ccc(Cl)cc2)c1=O] |
| 126 | 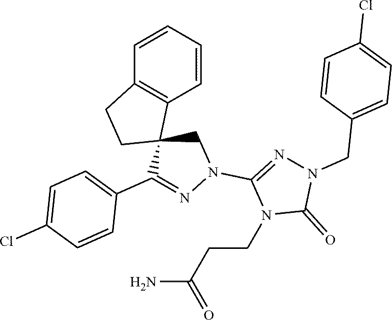 [SMILES: NC(=O)CCn1c(N2C[C@]3(CCc4ccccc43)C(c3ccc(Cl)cc3)=N2)nn(Cc2ccc(Cl)cc2)c1=O] |
| 127 | 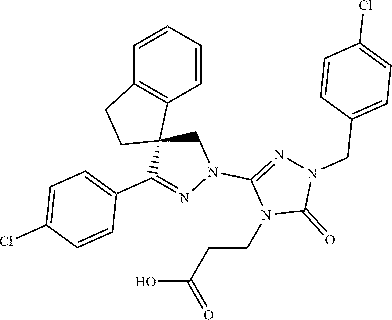 [SMILES: O=C(O)CCn1c(N2C[C@]3(CCc4ccccc43)C(c3ccc(Cl)cc3)=N2)nn(Cc2ccc(Cl)cc2)c1=O] |
| 128 | 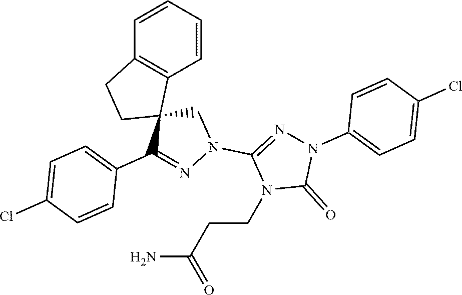 [SMILES: NC(=O)CCn1c(N2C[C@@]3(CCc4ccccc43)C(c3ccc(Cl)cc3)=N2)nn(-c2ccc(Cl)cc2)c1=O] |
| 129 | 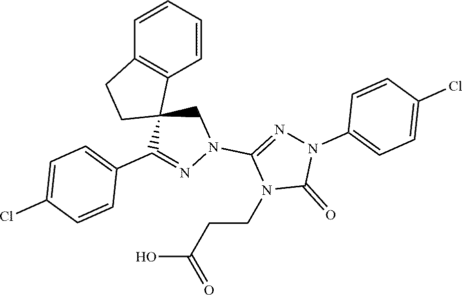 [SMILES: O=C(O)CCn1c(N2C[C@]3(CCc4ccccc43)C(c3ccc(Cl)cc3)=N2)nn(-c2ccc(Cl)cc2)c1=O] |
| 130 | 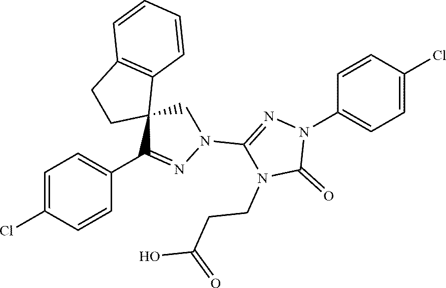 [SMILES: O=C(O)CCn1c(N2C[C@@]3(CCc4ccccc43)C(c3ccc(Cl)cc3)=N2)nn(-c2ccc(Cl)cc2)c1=O] |
| 131 | 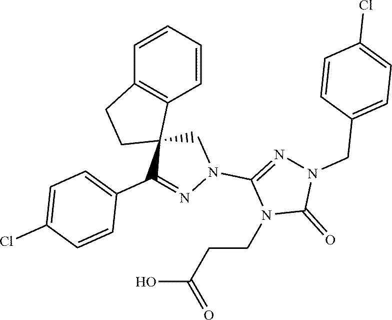 [SMILES: O=C(O)CCn1c(N2C[C@@]3(CCc4ccccc43)C(c3ccc(Cl)cc3)=N2)nn(Cc2ccc(Cl)cc2)c1=O] |
| 132 | 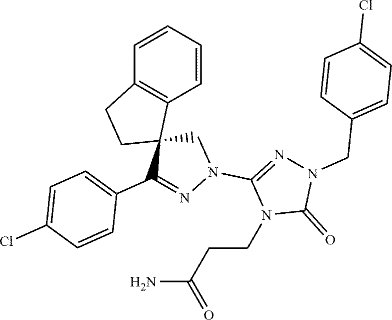 [SMILES: NC(=O)CCn1c(N2C[C@@]3(CCc4ccccc43)C(c3ccc(Cl)cc3)=N2)nn(Cc2ccc(Cl)cc2)c1=O] |
| 133 | 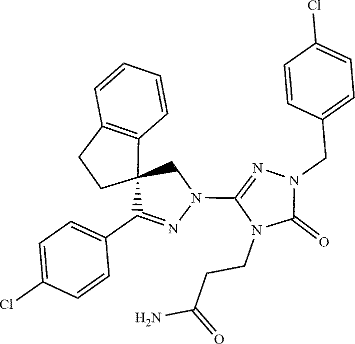 [SMILES: NC(=O)CCn1c(N2C[C@]3(CCc4ccccc43)C(c3ccc(Cl)cc3)=N2)nn(Cc2ccc(Cl)cc2)c1=O] |
| 134 | 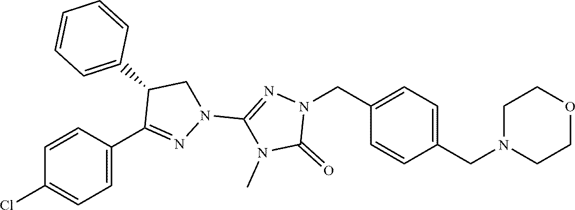 [SMILES: Cn1c(N2C[C@@H](c3ccccc3)C(c3ccc(Cl)cc3)=N2)nn(Cc2ccc(CN3CCOCC3)cc2)c1=O] |
| 135 | 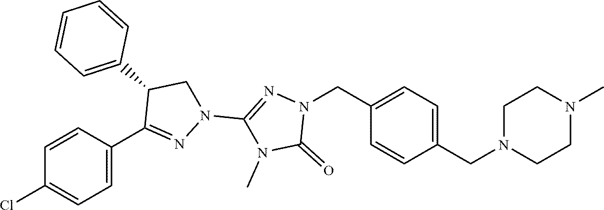 [SMILES: CN1CCN(Cc2ccc(Cn3nc(N4C[C@@H](c5ccccc5)C(c5ccc(Cl)cc5)=N4)n(C)c3=O)cc2)CC1] |
| 136 | 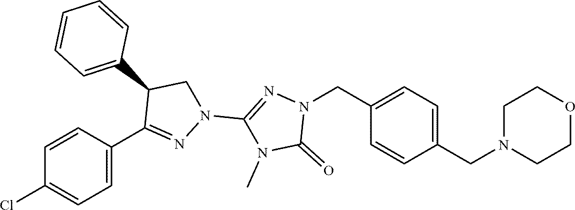 [SMILES: Cn1c(N2C[C@H](c3ccccc3)C(c3ccc(Cl)cc3)=N2)nn(Cc2ccc(CN3CCOCC3)cc2)c1=O] |
| 137 | 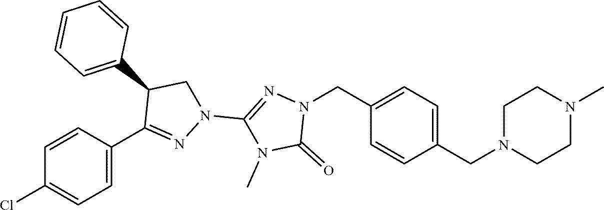 [SMILES: CN1CCN(Cc2ccc(Cn3nc(N4C[C@H](c5ccccc5)C(c5ccc(Cl)cc5)=N4)n(C)c3=O)cc2)CC1] |
| 138 | 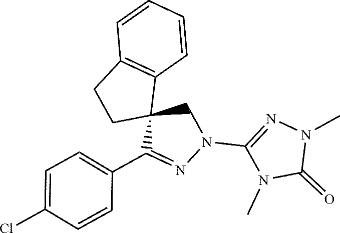 [SMILES: Cn1nc(N2C[C@]3(CCc4ccccc43)C(c3ccc(Cl)cc3)=N2)n(C)c1=O] |
| 139 | 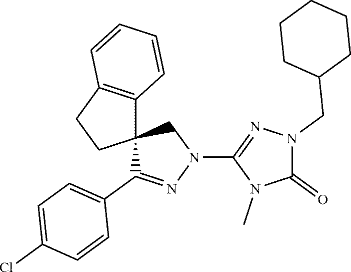 [SMILES: Cn1c(N2C[C@]3(CCc4ccccc43)C(c3ccc(Cl)cc3)=N2)nn(CC2CCCCC2)c1=O] |
| 140 | 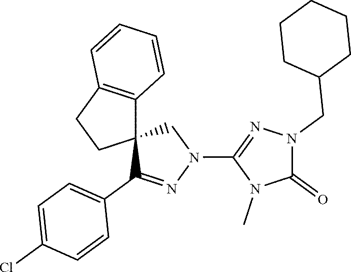 [SMILES: Cn1c(N2C[C@@]3(CCc4ccccc43)C(c3ccc(Cl)cc3)=N2)nn(CC2CCCCC2)c1=O] |
| 141 | 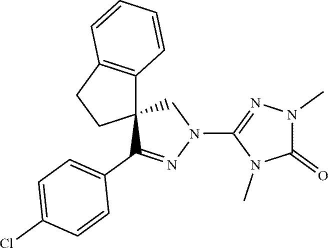 [SMILES: Cn1nc(N2C[C@@]3(CCc4ccccc43)C(c3ccc(Cl)cc3)=N2)n(C)c1=O] |
| 142 | 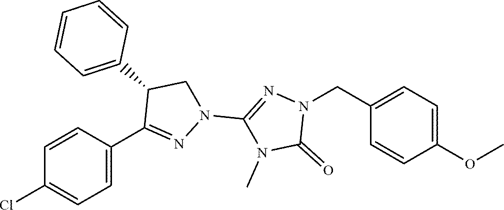 [SMILES: COc1ccc(Cn2nc(N3C[C@@H](c4ccccc4)C(c4ccc(Cl)cc4)=N3)n(C)c2=O)cc1] |
| 143 | 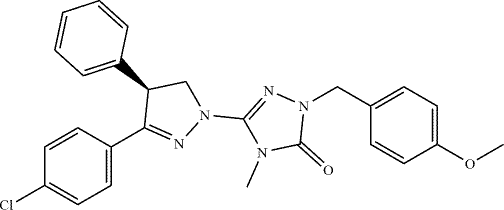 [SMILES: COc1ccc(Cn2nc(N3C[C@H](c4ccccc4)C(c4ccc(Cl)cc4)=N3)n(C)c2=O)cc1] |
| 144 | 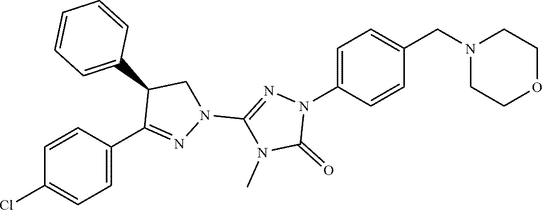 [SMILES: Cn1c(N2C[C@H](c3ccccc3)C(c3ccc(Cl)cc3)=N2)nn(-c2ccc(CN3CCOCC3)cc2)c1=O] |
| 145 | 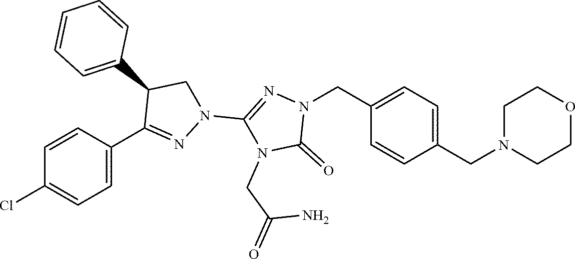 [SMILES: NC(=O)Cn1c(N2C[C@H](c3ccccc3)C(c3ccc(Cl)cc3)=N2)nn(Cc2ccc(CN3CCOCC3)cc2)c1=O] |
| 146 | 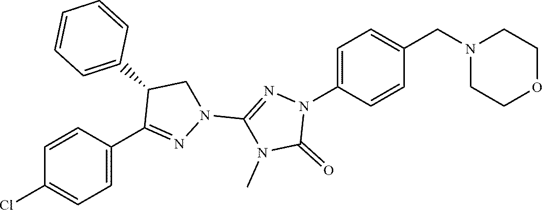 [SMILES: Cn1c(N2C[C@@H](c3ccccc3)C(c3ccc(Cl)cc3)=N2)nn(-c2ccc(CN3CCOCC3)cc2)c1=O] |
| 147 | 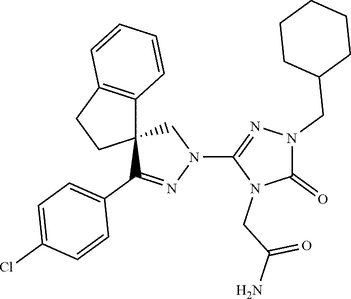 [SMILES: NC(=O)Cn1c(N2C[C@@]3(CCc4ccccc43)C(c3ccc(Cl)cc3)=N2)nn(CC2CCCCC2)c1=O] |
| 149 | 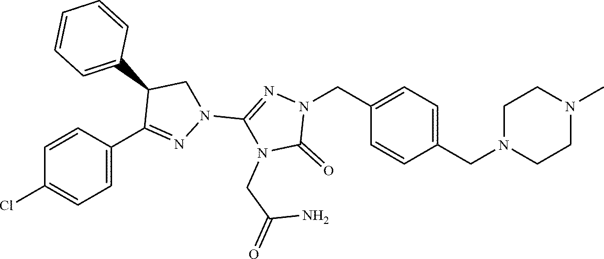 [SMILES: CN1CCN(Cc2ccc(Cn3nc(N4C[C@H](c5ccccc5)C(c5ccc(Cl)cc5)=N4)n(CC(N)=O)c3=O)cc2)CC1] |
| 150 | 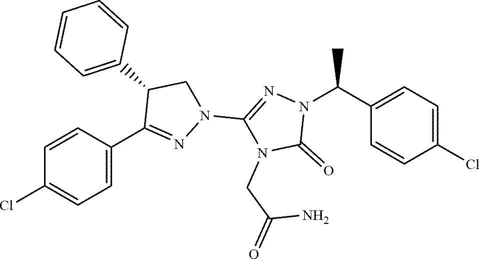 [SMILES: C[C@@H](c1ccc(Cl)cc1)n1nc(N2C[C@@H](c3ccccc3)C(c3ccc(Cl)cc3)=N2)n(CC(N)=O)c1=O] |
| 152 | 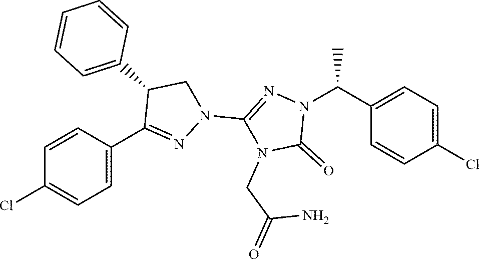 [SMILES: C[C@H](c1ccc(Cl)cc1)n1nc(N2C[C@@H](c3ccccc3)C(c3ccc(Cl)cc3)=N2)n(CC(N)=O)c1=O] |
| 153 | 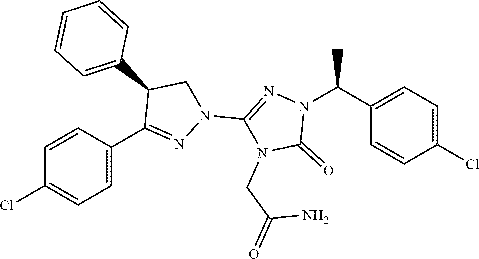 [SMILES: C[C@@H](c1ccc(Cl)cc1)n1nc(N2C[C@H](c3ccccc3)C(c3ccc(Cl)cc3)=N2)n(CC(N)=O)c1=O] |
| 154 | 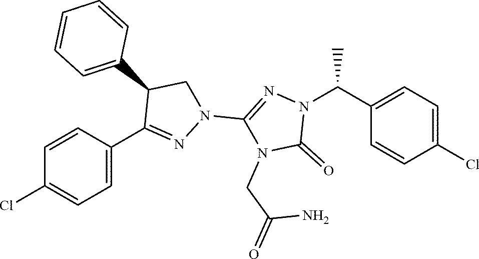 [SMILES: C[C@H](c1ccc(Cl)cc1)n1nc(N2C[C@H](c3ccccc3)C(c3ccc(Cl)cc3)=N2)n(CC(N)=O)c1=O] |
| 158 | 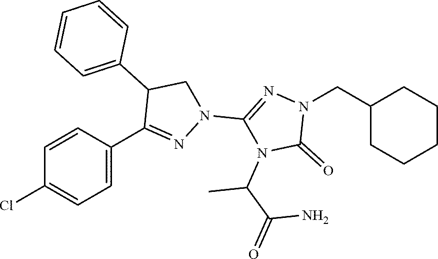 [SMILES: CC(C(N)=O)n1c(N2CC(c3ccccc3)C(c3ccc(Cl)cc3)=N2)nn(CC2CCCCC2)c1=O] |
| 159 | 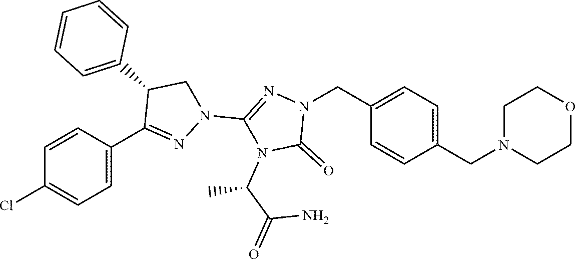 [SMILES: C[C@@H](C(N)=O)n1c(N2C[C@@H](c3ccccc3)C(c3ccc(Cl)cc3)=N2)nn(Cc2ccc(CN3CCOCC3)cc2)c1=O] |
| 160 | 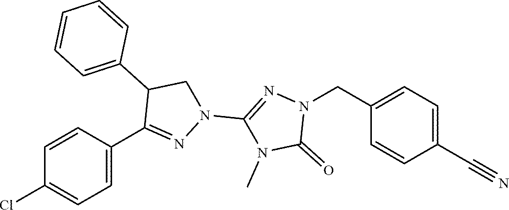 [SMILES: Cn1c(N2CC(c3ccccc3)C(c3ccc(Cl)cc3)=N2)nn(Cc2ccc(C#N)cc2)c1=O] |
| 161 | 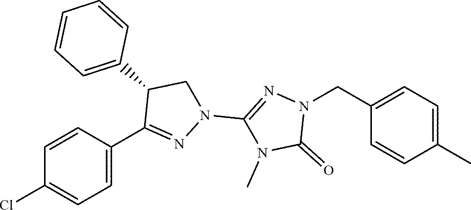 [SMILES: Cc1ccc(Cn2nc(N3C[C@@H](c4ccccc4)C(c4ccc(Cl)cc4)=N3)n(C)c2=O)cc1] |
| 163 | 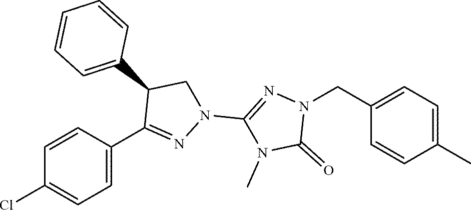 [SMILES: Cc1ccc(Cn2nc(N3C[C@H](c4ccccc4)C(c4ccc(Cl)cc4)=N3)n(C)c2=O)cc1] |
| 164 | 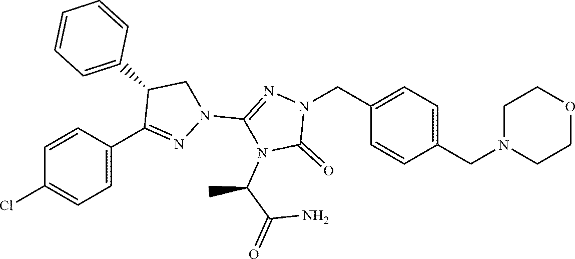 [SMILES: C[C@H](C(N)=O)n1c(N2C[C@@H](c3ccccc3)C(c3ccc(Cl)cc3)=N2)nn(Cc2ccc(CN3CCOCC3)cc2)c1=O] |
| 166 | 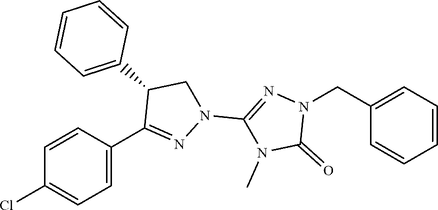 [SMILES: Cn1c(N2C[C@@H](c3ccccc3)C(c3ccc(Cl)cc3)=N2)nn(Cc2ccccc2)c1=O] |
| 167 | 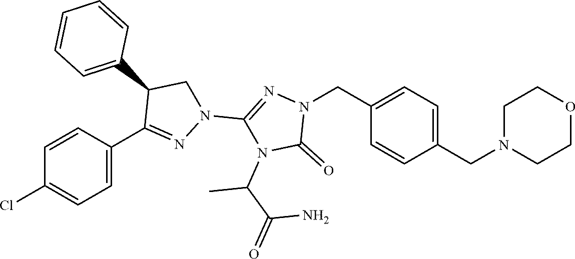 [SMILES: CC(C(N)=O)n1c(N2C[C@H](c3ccccc3)C(c3ccc(Cl)cc3)=N2)nn(Cc2ccc(CN3CCOCC3)cc2)c1=O] |
| 168 | 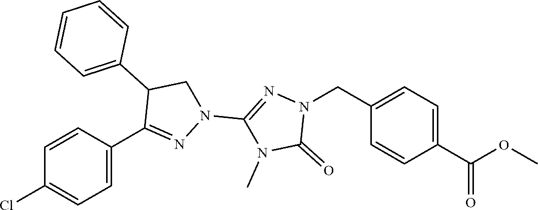 [SMILES: COC(=O)c1ccc(Cn2nc(N3CC(c4ccccc4)C(c4ccc(Cl)cc4)=N3)n(C)c2=O)cc1] |
| 169 | 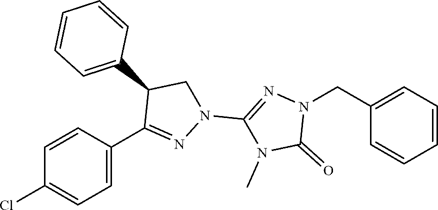 [SMILES: Cn1c(N2C[C@H](c3ccccc3)C(c3ccc(Cl)cc3)=N2)nn(Cc2ccccc2)c1=O] |
| 170 | 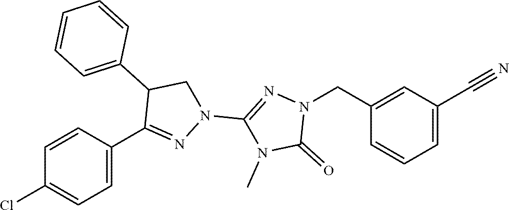 [SMILES: Cn1c(N2CC(c3ccccc3)C(c3ccc(Cl)cc3)=N2)nn(Cc2cccc(C#N)c2)c1=O] |
| 171 | 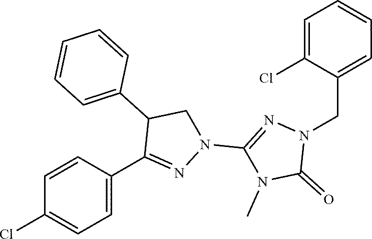 [SMILES: Cn1c(N2CC(c3ccccc3)C(c3ccc(Cl)cc3)=N2)nn(Cc2ccccc2Cl)c1=O] |
| 172 | 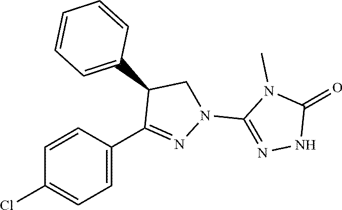 [SMILES: Cn1c(N2C[C@H](c3ccccc3)C(c3ccc(Cl)cc3)=N2)n[nH]c1=O] |
| 173 | 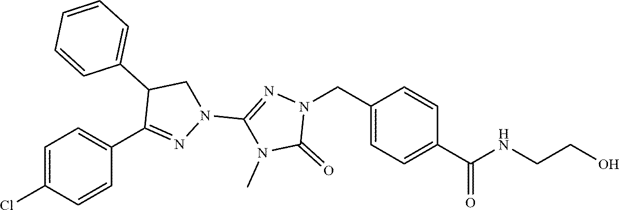 [SMILES: Cn1c(N2CC(c3ccccc3)C(c3ccc(Cl)cc3)=N2)nn(Cc2ccc(C(=O)NCCO)cc2)c1=O] |
| 174 | 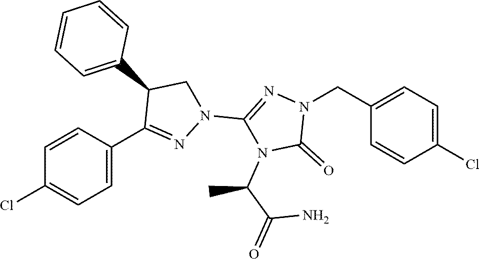 [SMILES: C[C@H](C(N)=O)n1c(N2C[C@H](c3ccccc3)C(c3ccc(Cl)cc3)=N2)nn(Cc2ccc(Cl)cc2)c1=O] |
| 175 | 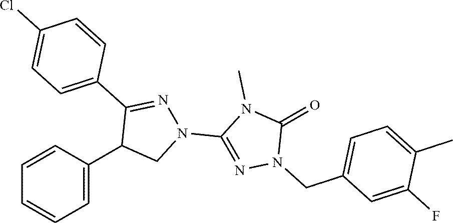 [SMILES: Cc1ccc(Cn2nc(N3CC(c4ccccc4)C(c4ccc(Cl)cc4)=N3)n(C)c2=O)cc1F] |
| 176 | 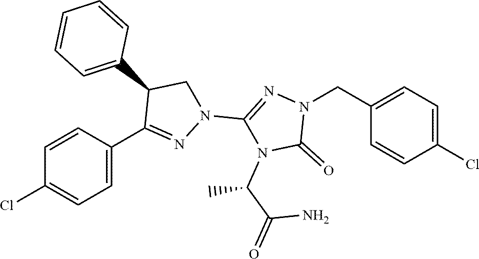 [SMILES: C[C@@H](C(N)=O)n1c(N2C[C@H](c3ccccc3)C(c3ccc(Cl)cc3)=N2)nn(Cc2ccc(Cl)cc2)c1=O] |
| 177 | 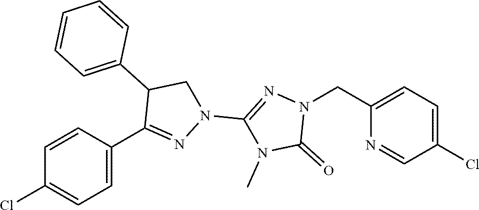 [SMILES: Cn1c(N2CC(c3ccccc3)C(c3ccc(Cl)cc3)=N2)nn(Cc2ccc(Cl)cn2)c1=O] |
| 178 | 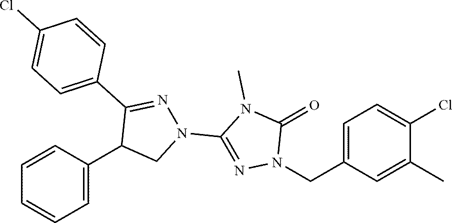 [SMILES: Cc1cc(Cn2nc(N3CC(c4ccccc4)C(c4ccc(Cl)cc4)=N3)n(C)c2=O)ccc1Cl] |
| 179 | 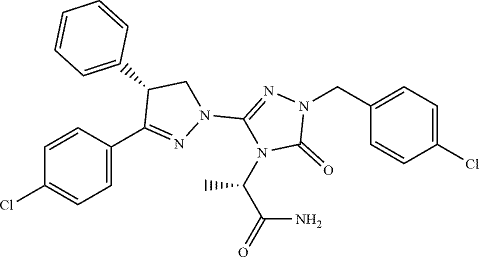 [SMILES: C[C@@H](C(N)=O)n1c(N2C[C@@H](c3ccccc3)C(c3ccc(Cl)cc3)=N2)nn(Cc2ccc(Cl)cc2)c1=O] |
| 180 | 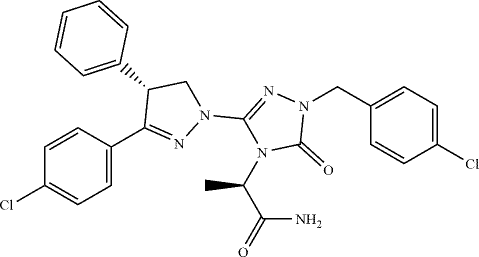 [SMILES: C[C@H](C(N)=O)n1c(N2C[C@@H](c3ccccc3)C(c3ccc(Cl)cc3)=N2)nn(Cc2ccc(Cl)cc2)c1=O] |
| 181 | 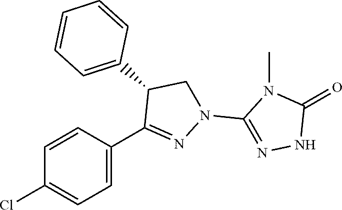 [SMILES: Cn1c(N2C[C@@H](c3ccccc3)C(c3ccc(Cl)cc3)=N2)n[nH]c1=O] |
| 182 | 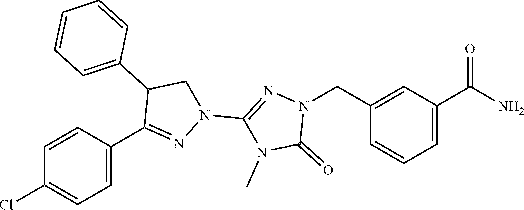 [SMILES: Cn1c(N2CC(c3ccccc3)C(c3ccc(Cl)cc3)=N2)nn(Cc2cccc(C(N)=O)c2)c1=O] |
| 183 | 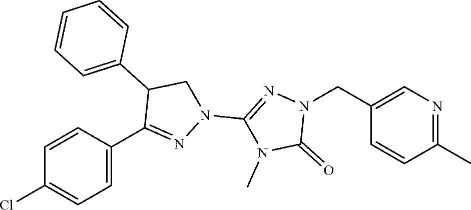 [SMILES: Cc1ccc(Cn2nc(N3CC(c4ccccc4)C(c4ccc(Cl)cc4)=N3)n(C)c2=O)cn1] |
| 184 | 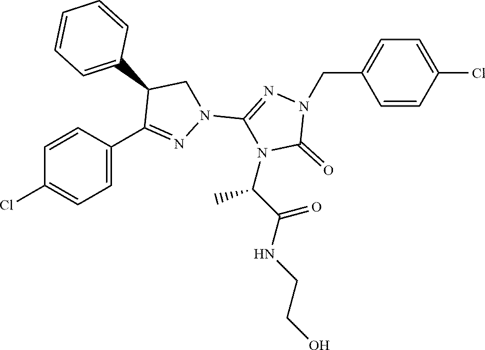 [SMILES: C[C@@H](C(=O)NCCO)n1c(N2C[C@H](c3ccccc3)C(c3ccc(Cl)cc3)=N2)nn(Cc2ccc(Cl)cc2)c1=O] |
| 186 | 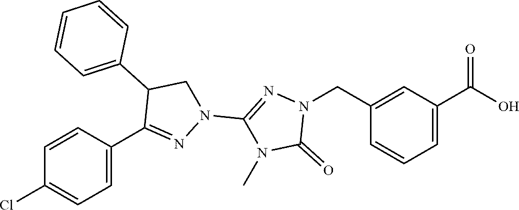 [SMILES: Cn1c(N2CC(c3ccccc3)C(c3ccc(Cl)cc3)=N2)nn(Cc2cccc(C(=O)O)c2)c1=O] |
| 187 | 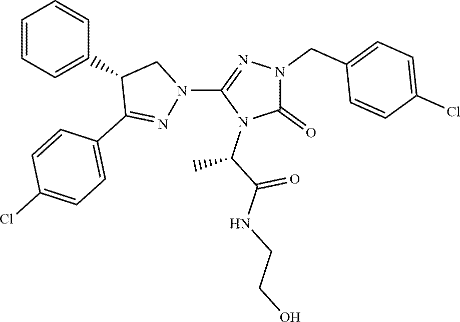 [SMILES: C[C@@H](C(=O)NCCO)n1c(N2C[C@@H](c3ccccc3)C(c3ccc(Cl)cc3)=N2)nn(Cc2ccc(Cl)cc2)c1=O] |
| 189 | 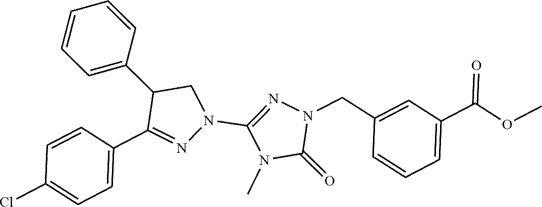 [SMILES: COC(=O)c1cccc(Cn2nc(N3CC(c4ccccc4)C(c4ccc(Cl)cc4)=N3)n(C)c2=O)c1] |
| 190 | 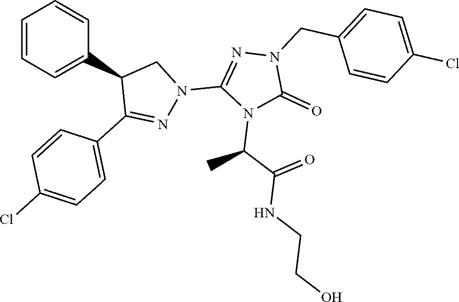 [SMILES: C[C@H](C(=O)NCCO)n1c(N2C[C@H](c3ccccc3)C(c3ccc(Cl)cc3)=N2)nn(Cc2ccc(Cl)cc2)c1=O] |
| 191 | 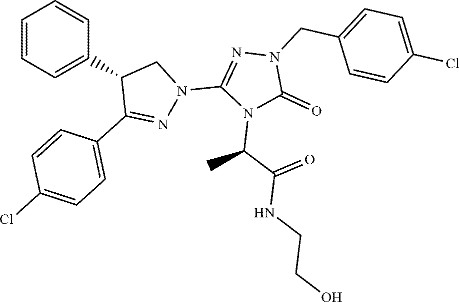 [SMILES: C[C@H](C(=O)NCCO)n1c(N2C[C@@H](c3ccccc3)C(c3ccc(Cl)cc3)=N2)nn(Cc2ccc(Cl)cc2)c1=O] |
| 192 | 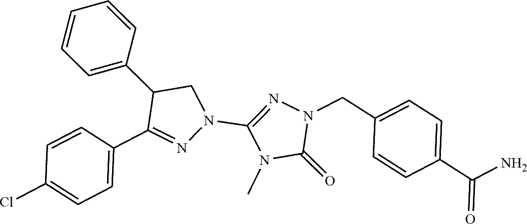 [SMILES: Cn1c(N2CC(c3ccccc3)C(c3ccc(Cl)cc3)=N2)nn(Cc2ccc(C(N)=O)cc2)c1=O] |
| 193 | 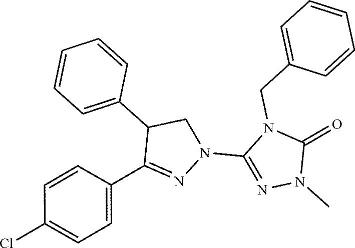 [SMILES: Cn1nc(N2CC(c3ccccc3)C(c3ccc(Cl)cc3)=N2)n(Cc2ccccc2)c1=O] |
| 194 | 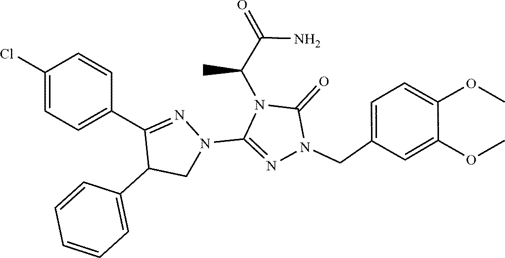 [SMILES: COc1ccc(Cn2nc(N3CC(c4ccccc4)C(c4ccc(Cl)cc4)=N3)n([C@@H](C)C(N)=O)c2=O)cc1OC] |
| 195 | 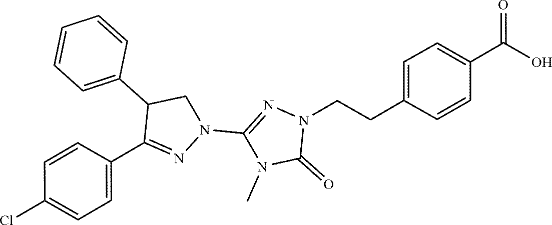 [SMILES: Cn1c(N2CC(c3ccccc3)C(c3ccc(Cl)cc3)=N2)nn(CCc2ccc(C(=O)O)cc2)c1=O] |
| 196 | 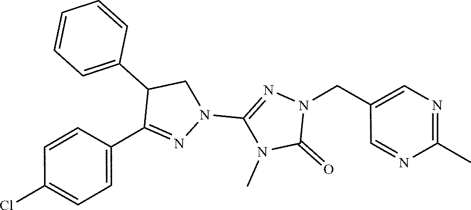 [SMILES: Cc1ncc(Cn2nc(N3CC(c4ccccc4)C(c4ccc(Cl)cc4)=N3)n(C)c2=O)cn1] |
| 197 | 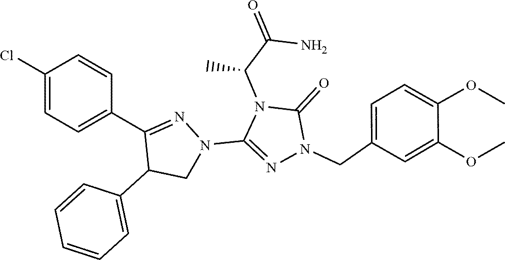 [SMILES: COc1ccc(Cn2nc(N3CC(c4ccccc4)C(c4ccc(Cl)cc4)=N3)n([C@H](C)C(N)=O)c2=O)cc1OC] |
| 199 | 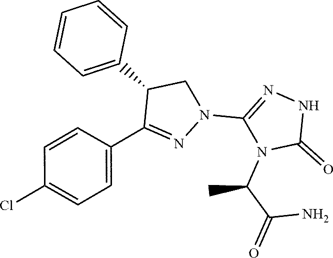 [SMILES: C[C@H](C(N)=O)n1c(N2C[C@@H](c3ccccc3)C(c3ccc(Cl)cc3)=N2)n[nH]c1=O] |
| 200 | 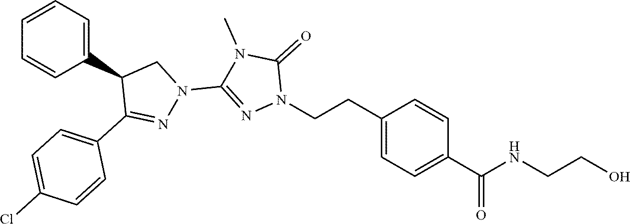 [SMILES: Cn1c(N2C[C@H](c3ccccc3)C(c3ccc(Cl)cc3)=N2)nn(CCc2ccc(C(=O)NCCO)cc2)c1=O] |
| 201 | 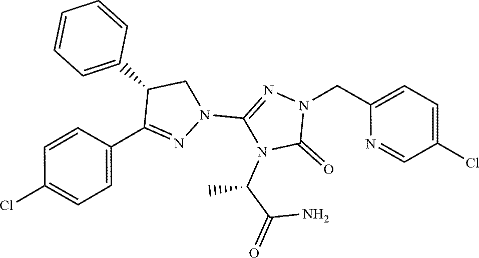 [SMILES: C[C@@H](C(N)=O)n1c(N2C[C@@H](c3ccccc3)C(c3ccc(Cl)cc3)=N2)nn(Cc2ccc(Cl)cn2)c1=O] |
| 202 | 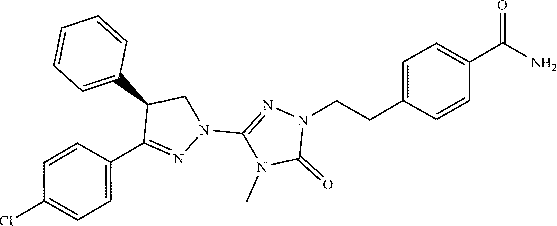 [SMILES: Cn1c(N2C[C@H](c3ccccc3)C(c3ccc(Cl)cc3)=N2)nn(CCc2ccc(C(N)=O)cc2)c1=O] |
| 203 | 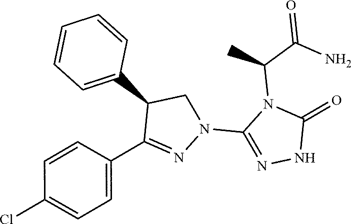 [SMILES: C[C@@H](C(N)=O)n1c(N2C[C@H](c3ccccc3)C(c3ccc(Cl)cc3)=N2)n[nH]c1=O] |
| 205 | 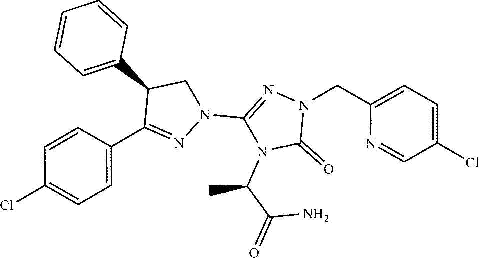 [SMILES: C[C@H](C(N)=O)n1c(N2C[C@H](c3ccccc3)C(c3ccc(Cl)cc3)=N2)nn(Cc2ccc(Cl)cn2)c1=O] |
| 206 | 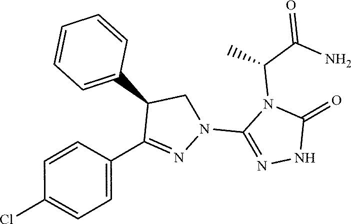 [SMILES: C[C@H](C(N)=O)n1c(N2C[C@H](c3ccccc3)C(c3ccc(Cl)cc3)=N2)n[nH]c1=O] |
| 207 | 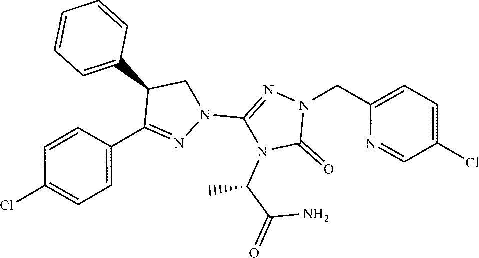 [SMILES: C[C@@H](C(N)=O)n1c(N2C[C@H](c3ccccc3)C(c3ccc(Cl)cc3)=N2)nn(Cc2ccc(Cl)cn2)c1=O] |
| 208 | 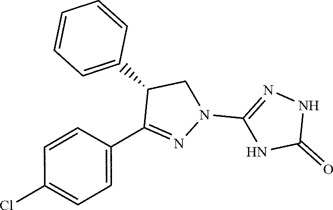 [SMILES: O=c1[nH]nc(N2C[C@@H](c3ccccc3)C(c3ccc(Cl)cc3)=N2)[nH]1] |
| 209 | 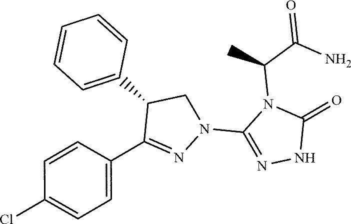 [SMILES: C[C@@H](C(N)=O)n1c(N2C[C@@H](c3ccccc3)C(c3ccc(Cl)cc3)=N2)n[nH]c1=O] |
| 210 | 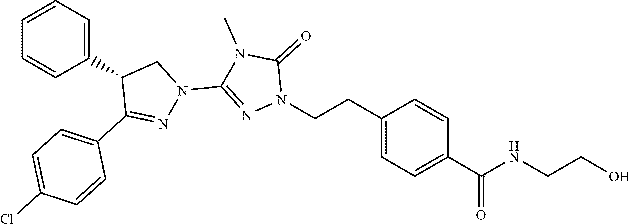 [SMILES: Cn1c(N2C[C@@H](c3ccccc3)C(c3ccc(Cl)cc3)=N2)nn(CCc2ccc(C(=O)NCCO)cc2)c1=O] |
| 211 | 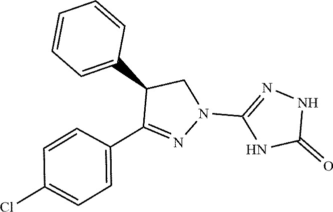 [SMILES: O=c1[nH]nc(N2C[C@H](c3ccccc3)C(c3ccc(Cl)cc3)=N2)[nH]1] |
| 212 | 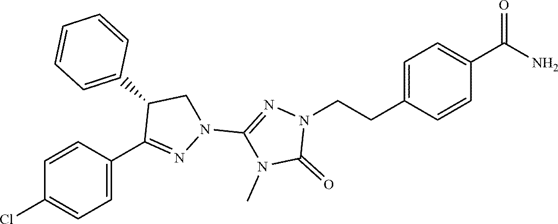 [SMILES: Cn1c(N2C[C@@H](c3ccccc3)C(c3ccc(Cl)cc3)=N2)nn(CCc2ccc(C(N)=O)cc2)c1=O] |
| 213 | 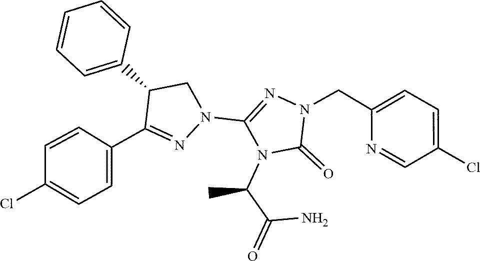 [SMILES: C[C@H](C(N)=O)n1c(N2C[C@@H](c3ccccc3)C(c3ccc(Cl)cc3)=N2)nn(Cc2ccc(Cl)cn2)c1=O] |
| 214 | 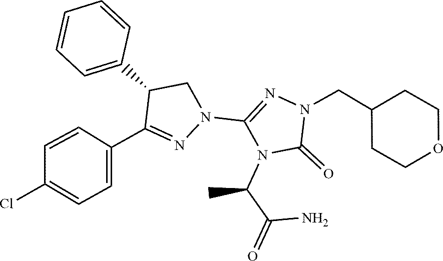 [SMILES: C[C@H](C(N)=O)n1c(N2C[C@@H](c3ccccc3)C(c3ccc(Cl)cc3)=N2)nn(CC2CCOCC2)c1=O] |
| 215 | 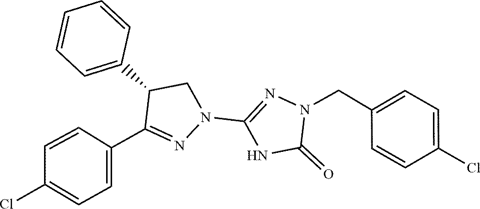 [SMILES: O=c1[nH]c(N2C[C@@H](c3ccccc3)C(c3ccc(Cl)cc3)=N2)nn1Cc1ccc(Cl)cc1] |
| 216 | 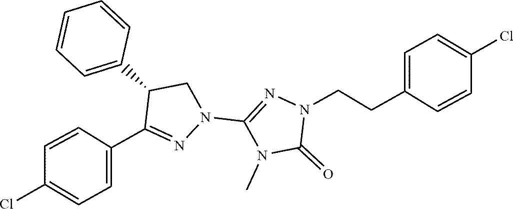 [SMILES: Cn1c(N2C[C@@H](c3ccccc3)C(c3ccc(Cl)cc3)=N2)nn(CCc2ccc(Cl)cc2)c1=O] |
| 217 | 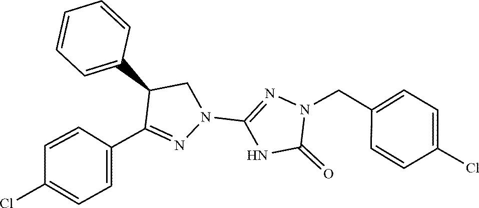 [SMILES: O=c1[nH]c(N2C[C@H](c3ccccc3)C(c3ccc(Cl)cc3)=N2)nn1Cc1ccc(Cl)cc1] |
| 218 | 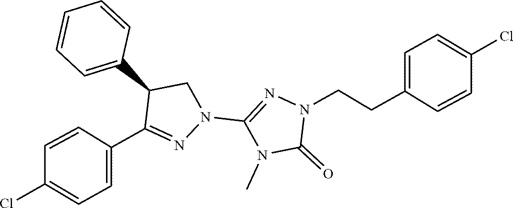 [SMILES: Cn1c(N2C[C@H](c3ccccc3)C(c3ccc(Cl)cc3)=N2)nn(CCc2ccc(Cl)cc2)c1=O] |
| 219 | 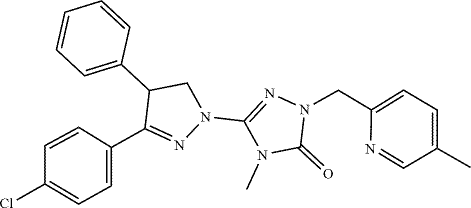 [SMILES: Cc1ccc(Cn2nc(N3CC(c4ccccc4)C(c4ccc(Cl)cc4)=N3)n(C)c2=O)nc1] |
| 220 | 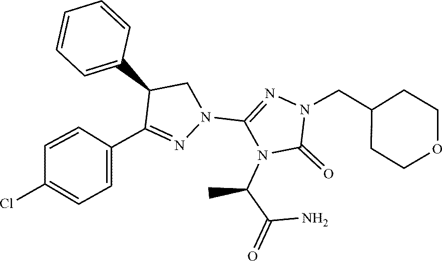 [SMILES: C[C@H](C(N)=O)n1c(N2C[C@H](c3ccccc3)C(c3ccc(Cl)cc3)=N2)nn(CC2CCOCC2)c1=O] |
| 221 | 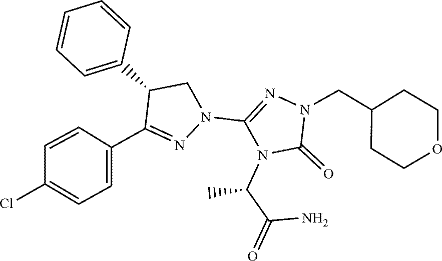 [SMILES: C[C@@H](C(N)=O)n1c(N2C[C@@H](c3ccccc3)C(c3ccc(Cl)cc3)=N2)nn(CC2CCOCC2)c1=O] |
| 222 | 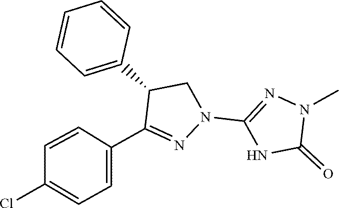 [SMILES: Cn1nc(N2C[C@@H](c3ccccc3)C(c3ccc(Cl)cc3)=N2)[nH]c1=O] |
| 223 | 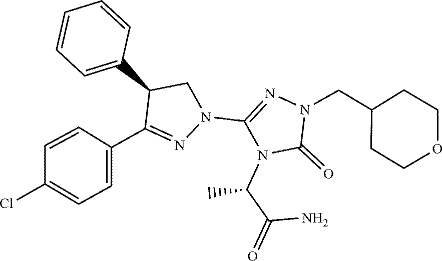 [SMILES: C[C@@H](C(N)=O)n1c(N2C[C@H](c3ccccc3)C(c3ccc(Cl)cc3)=N2)nn(CC2CCOCC2)c1=O] |
| 224 | 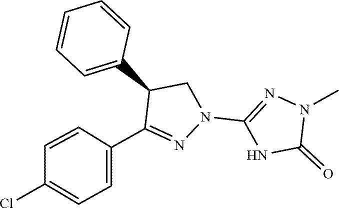 [SMILES: Cn1nc(N2C[C@H](c3ccccc3)C(c3ccc(Cl)cc3)=N2)[nH]c1=O] |

.

12. A composition, comprising a compound of any one of claims 1-11; and a pharmaceutically acceptable carrier.

13. A method of treating a disease or condition characterized by aberrant CB1 activity comprising the step of administering to a subject in need thereof a compound of any one of claims 1-11, or a composition of claim 12.

14. The method of claim 13, wherein the disease or condition is diabetic kidney disease, diabetic nephropathy, obesity-related kidney disease, focal segmental glomerular sclerosis, IgA nephropathy, nephrotic syndrome, kidney fibrosis, Prader Willi syndrome, metabolic syndrome, gastrointestinal diseases, non-alcoholic liver disease, alcoholic liver disease, or non-alcoholic fatty liver disease.

15. The method of claim 14, wherein the disease or condition is diabetic nephropathy.

16. The method of claim 14, wherein the disease or condition is focal segmental glomerular sclerosis.

17. The method of claim 14, wherein the disease or conditions is nonalcoholic steatohepatitis.
